(19) United States
(12) Patent Application Publication
(10) Pub. No.: US 2026/0327592 A1
(43) Pub. Date: Oct. 22, 2026

(54) ARCHAEAL PEPTIDE RECOMBINASE – A NOVEL PEPTIDE LIGATING ENZYME

(71) Applicant: MAX-PLANCK-GESELLSCHAFT ZUR FÖRDERUNG DER WISSENSCHAFTEN E.V., München (DE)

(72) Inventors: Adrian FUCHS, Tübingen (DE); Moritz AMMELBURG, Münsing (DE); Marcus D. HARTMANN, Tübingen (DE)

(73) Assignee: MAX-PLANCK-GESELLSCHAFT ZUR FÖRDERUNG DER WISSENSCHAFTEN E.V., München (DE)

(21) Appl. No.: 19/438,554

(22) Filed: Dec. 31, 2025

Related U.S. Application Data
(62) Division of application No. 17/778,328, filed on Nov. 19, 2020.

(30) Foreign Application Priority Data
Nov. 20, 2019 (EP) 19210430.5
Jul. 7, 2020 (EP) 20184421.4

(51) Int. Cl. C12N 9/48; C07K 17/00

(52) U.S. Cl. CPC C12N 9/485; C07K 17/00

(57) ABSTRACT

The present invention relates to the provision of new means and methods for enzymatic peptide-peptide ligation. In particular, the present invention provides a novel family of transpeptidase enzymes, herein subsequently also referred to as Adriase (Archaeal Peptide Recombinase), transpeptidase or polypeptide recombinase. The members of the Adriase family, which are characterized by an N-terminal DUF2121 domain with an N-terminal serine or threonine residue were surprisingly found to recombine and ligate substrate peptides in a sequence specific manner via a short DUF2121 recognition motif. This way, compounds like proteins, synthetic compounds and/or whole cells may be linked specifically as long as they contain the motif or the parts thereof recognized by an Adriase enzyme. The ligation reaction described herein can be used to engineer novel molecules in a modular way, with broad applications in both research and pharmacology.

This application is a divisional of U.S. patent application Ser. No. 17/778,328, filed on May 19, 2022, which is a National Stage Entry of International Application No. PCT/EP2020/082721, filed on Nov. 19, 2020, which claims priority to European Patent Application No. 2018442.4, filed Jul. 7, 2020, and European Patent Application No. 19210430.5, filed Nov. 20, 2019.

### SEQUENCE LISTING

The instant application contains a Sequence Listing which has been submitted via Patent Center and is hereby incorporated by reference in its entirety. Said .xml copy, created on Jun. 10, 2026, is named 028622-0352, and is 890,648 bytes in size.

The present invention relates to the provision of new means and methods for enzymatic peptide-peptide ligation. In particular, the present invention provides a novel family of transpeptidase enzymes, herein subsequently also referred to as Adriase (Archaeal Peptide Recombinase), Jugase, Conectase, Connectase, transpeptidase or polypeptide recombinase. The members of the Adriase family, which are characterized by an N-terminal DUF2121 domain with an N-terminal serine or threonine residue were surprisingly found to recombine and ligate substrate peptides in a sequence specific manner via a DUF2121 recognition motif. This way, compounds like proteins, synthetic compounds and/or whole cells may be linked specifically as long as they contain the motif, or the parts thereof recognized by an Adriase enzyme. The ligation reaction described herein can be used to engineer novel molecules in a modular way, with broad applications in both research and pharmacology.

DNA modifying enzymes allow, in principle, for the construction of any gene and hence for the production of any protein of interest. Yet, this indirect approach is limited to the production of linear amino acid sequences and produces the full-length construct in one step, i.e. does not allow for a post-translational assembly of new fusion proteins. However, many experiments require proteins that are modified upon demand and/or include non-proteinaceous components. Unfortunately, compared to the possibilities for DNA editing, the molecular toolbox for protein modifications is rather limited.

Only a small set of protein ligation/modification methods have been developed so far. These can be divided into chemical, split intein, split domain and enzymatic protein ligations. However, all of these technologies have caveats and disadvantages.

For example, chemical methods are frequently used for synthetic or small and pure peptides. However, these methods typically require the introduction of non-proteinaceous chemical groups and often do not provide a pronounced chemoselectivity. Thus, they are not suited for reactions within complex solutions (Chen (2015) Amino Acids 47:1283-99; Schmidt (2017) Curr Opin Chem Biol 38:1-7).

Another approach relates to the use of split inteins, which are a subset of inteins that are expressed in two separate halves and catalyze splicing of the associated protein domains in trans upon association of the two split-intein halves. Split inteins can be fused genetically to the nucleotide sequence encoding the proteins to be fused. They can, however, only catalyze terminal ligations (between N- and C-termini), are not always efficient, require the maintenance of reducing conditions throughout their production and their considerable size can cause solubility issues (Li (2015) Biotechnol Lett 37:2121-37).

Further approaches relate to the use of Split domains such as the SpyTag-SpyCatcher system. This technology is based on a modified bacterial domain (SpyCatcher), which recognizes a cognate 13-amino-acid peptide (SpyTag). Upon recognition, the two form a covalent isopeptide bond between the side chains of a lysine in SpyCatcher and an aspartate in SpyTag (Sutherland (2019) Chembiochem 20:319-28). However, these bulky bacterial domain pairs (>100 aa) can be immunogenic and induce steric hindrances in the ligation products.

Another approach for linking proteins involves ligase enzymes, such as Butelase, Trypsiligase or Subtiligase. These enzymes recognize and fuse proteins via short recognition motifs. Yet, these enzymes have low substrate specificity (Schmidt (2017) loc. cit.). Enzymatic protein ligations are typically reversible, which typically limits the maximum ligation yield. Moreover, these enzymes bind their substrate via hydroxy- or thioesters that are prone to hydrolysis. This irreversible side reaction further decreases ligation yields and necessitates timely removal of the ligation product from the equilibrium.

The most prominent protein ligase enzyme known in the art is Sortase A (Antos (2016) Curr Opin Struct Biol 38:111-8). Originally isolated from Staphylococcus aureus, where it anchors surface proteins to the cell wall, Sortase is nowadays the most commonly used protein ligase as indicated by hundreds of publications listed in PubMed. Though many homologs of Sortase A derived from different organisms have been studied, the representative from S. aureus remains the most active. In presence of Ca²⁺, Sortase A binds substrates with a so-called LPXTG (SEQ ID NO: 680)-motif via thioester formation and cleaves off the terminal glycine. This process is reversible and therefore any compound featuring an N-terminal glycine can be ligated to the LPXTG (SEQ ID NO: 680) substrate.

Compared to the other above discussed protein ligase enzymes, Sortase A provides a higher specificity however, at the cost of decreased catalytic efficiency (Schmidt (2017) loc. cit.). Despite improvements of Sortase through directed evolution approaches, substrate KM values remain in the millimolar range, far off the micromolar concentrations typically used for in vitro protein assays. This results in poor ligation rates and necessitates the use of high Sortase A concentrations and long incubation times (Theile (2013) Nat Protoc 8:1800-7; Fottner (2019) Nat Chem Biol 15:276-84). Furthermore, since Sortase A binds substrates via thioester the Sortase A-substrate intermediate is prone to hydrolysis. The irreversible hydrolysis side reaction further decreases ligation yields and necessitates timely removal of the ligation product from the equilibrium.

The prior art enzymes employed in peptide ligation were investigated and/or developed either with respect to substrate specificity or catalytic activity. Thus, there is a particular need to provide new enzyme systems for peptide/protein ligation that offer advantageous specificity in combination with a high reaction rate. Moreover, there is a need to minimize undesired side reactions. In particular, there is a need to avoid an irreversible hydrolysis of reaction intermediates such as observed for the currently known protein ligation enzymes.

Thus, the technical problem underlying the present invention is the provision of means and methods that allow the enzymatic fusion of polypeptides and/or peptide-containing compounds, preferably in an easy, specific and/or efficient manner, even more preferably with a minimum of unwanted side reactions, such as irreversible hydrolysis of reaction intermediates.

The technical problem is solved and the above mentioned needs are addressed by the provision of the embodiments characterized in the claims and as provided herein below.

In a first aspect the invention provides for a polypeptide comprising an N-terminal DUF2121 domain having an N-terminal serine or threonine residue. The DUF2121 domain is annotated in the Pfam database under PF09894 and described as a conserved domain of unknown function. In context of the present invention it has been surprisingly found that the DUF2121 domain has transpeptidase activity when the annotated N-terminal methionine residue is removed thereby exposing a serine or threonine residue N-terminally. In context of the invention “N-terminal DUF2121 domain” means that the amino acid sequence of the DUF2121 domain forms the N-terminus of the polypeptide and is further defined herein below. “N-terminal serine or threonine residue” means that the first amino acid of the polypeptide is a serine or threonine residue. In other words the starting amino acid of the polypeptide is a serine or threonine residue.

A DUF2121 domain is further described herein below and preferably comprises or consists of an amino acid sequence having transpeptidase activity selected from the group consisting of
(i) SEQ ID NOs: 2 and 4 to 143;
(ii) an amino acid sequence having at least 60% sequence identity to the amino acid sequences of (i); and
(iii) an amino acid sequence as defined in (i) or (ii) wherein one to 10 amino acid residues are deleted, inserted or added;

As demonstrated by the appended Figures and Examples the N-terminal serine or threonine residue of the DUF2121 domain is crucial for catalytic activity and is, thus, herein also referred to as catalytic serine or threonine residue.

It has been surprisingly found that the polypeptide of the invention has transpeptidase activity, more specifically sequence-specific transpeptidase activity. Due to its function the polypeptide of the invention can also be referred to as transpeptidase, preferably a sequence-specific transpeptidase.

Thus, the present invention relates to a (sequence-specific) transpeptidase comprising an N-terminal DUF2121 domain, wherein the amino acid sequence of the transpeptidase has an N-terminal serine or threonine residue. “N-terminal DUF2121 domain” means that the amino acid sequence of the DUF2121 domain forms the N-terminus of the polypeptide and is further defined herein below. “N-terminal serine or threonine residue” means that the first amino acid of the transpeptidase is a serine or threonine residue.

The polypeptide of the invention is particularly useful in methods for producing fusion proteins. In particular, the transpeptidase activity allows its use in post-translational protein engineering by protein-protein ligation. Substrate recognition is achieved by an amino acid sequence motif, herein referred to as “DUF2121 recognition motif” and defined further below. Within this motif, a sequence of at least 10, of at least 11, of at least 12, of at least 13, of at least 14 or of at least 15 amino acids may be required to achieve at least 10% of the maximum velocity of the transpeptidase reaction (FIG. 9C). Thus, the polypeptide of the invention has the advantage of being specific regarding substrate recognition. The polypeptide of the invention has the further advantage that it has a high reaction rate; i.e. a high number of ligations per time (k_{cat}). In one specific experiment of the appended examples the polypeptide of the invention shows a k_{cat} of around 1.4 s⁻¹ (Example 7, FIG. 9B). In context of the present invention a high number of ligations per time and, thus, a high reaction rate may be a k_{cat} of at least 0.4 s⁻¹, of at least 0.5 s⁻¹, of at least 0.6 s⁻¹, of at least 0.7 s⁻¹, of at least 0.8 s⁻¹, of at least 0.9 s⁻¹, of at least 1 s⁻¹, of at least 1.1 s⁻¹, preferably of at least 1.2 s⁻¹, more preferably of at least 1.3 s⁻¹, and most preferably of at least 1.4 s⁻¹. It is clear to the skilled person that there may be a need to determine the optimal reaction conditions for a certain transpeptidase of the present invention in order to observe high reaction rates. Furthermore, the polypeptide of the invention minimizes irreversible side reactions that hamper reaction efficiency, in particular hydrolysis, that are frequently observed for other protein ligases (e.g. Sortase A).

Accordingly, the present invention further provides a method for producing a fusion protein using the polypeptide with transpeptidase activity provided herein. The present invention also provides for uses of the polypeptide of the invention in protein engineering. Also provided is the use of the polypeptide of the invention in protein ligation or protein recombination. Thus, the present application provides for a new and advantageous transpeptidase system. As mentioned above, the system is characterized by the combination of a high substrate specificity in combination with a high reaction rate, especially also in vitro. A schematic overview of potential applications is provided in FIG. 16.

As illustrated herein it has been surprisingly found that the DUF2121 domain requires to be positioned N-terminally and requires an N-terminal serine or threonine residue to have transpeptidase activity. Accordingly, provided is a novel transpeptidase also called polypeptide recombinase, Jugase, Conectase or Adriase. Also provided is a method for recombinantly producing the polypeptide of the invention with the N-terminal DUF2121 domain and with the N-terminal serine and threonine residue.

Also preparations with N-terminal modifications may be used as long the catalytic serine or threonine residue gets exposed by enzymatic or autocatalytic removal of the residues N-terminally of the catalytic serine or threonine residue.

Accordingly, the invention further relates to a polypeptide having transpeptidase activity and comprising an DUF2121 domain having an N-terminal serine or threonine residue, and wherein said polypeptide has
(i) an amino acid sequence as depicted in SEQ ID NO: 2 or an amino acid sequence having at least 20% sequence identity thereto; and/or
(ii) an amino acid sequence selected from the group consisting of SEQ ID NOs: 4 to 225 or an amino acid sequence having at least 60% sequence identity thereto; and
wherein said polypeptide having transpeptidase activity further comprises at least one additional amino acid residue N-terminally of the sequences as defined in (i) or (ii); and
wherein the residue(s) N-terminally of the sequences as defined in (i) or (ii) is/are removed to obtain transpeptidase activity.

Furthermore, the invention relates to a transpeptidase comprising or consisting of an DUF2121 domain having an N-terminal serine or threonine residue,
(i) wherein said DUF2121 domain has an amino acid sequence as depicted in SEQ ID NO: 2 or an amino acid sequence having at least 20% sequence identity thereto; and/or
(ii) wherein said transpeptidase has an amino acid sequence selected from the group consisting of SEQ ID NOs: 4 to 225 or an amino acid sequence having at least 60% sequence identity thereto; and
said transpeptidase further comprises at least one additional amino acid residue N-terminally of the sequences as defined in (i) or (ii).

As shown in the appended examples herein provided are also transpeptidases which comprise indeed additional amino acids N-terminally of the herein recited catalytic serine or threonine residue. It is documented that such transpeptidase preparations are considerably less active in their enzymatic activity. Accordingly, the herein described transpeptidases with a N-terminal serine or threonine residue as a first amino acid of the polypeptide are the more preferred embodiments.

Also covered herein are variants of the polypeptide of the present invention in which the catalytic serine or threonine is exchanged by another amino acid as long as the polypeptide has transpeptidase activity. The catalytic residue may be exchanged to cysteine or an unnatural amino acid containing a hydroxyl group.

The proteasome is a large multi-subunit barrel-shaped complex that plays an important role in eukaryotic cells as main protease in the targeted protein degradation pathway. Prokaryotes encode for several uncharacterized proteasome homologs, many of which are not yet recognized due to their considerable sequence diversity. Such distant relationships are usually only detectable by combining information from sequence profiles with structure comparisons. In a prime example, a very distant proteasome homolog was identified denoted as domain of unknown function 2121 (PFAM (v 32.0) family DUF2121 (PF09894); InterPro v76.0 entry IPR016754) in public databases and as Adriase (Archaeal Peptide Recombinase) in the following. In a structure-based sequence alignment (FIG. 1) Adriase and the proteasome β subunit from Methanocaldococcus jannaschii share only 10.7% sequence identity (not counting the gaps)—a value that would actually be expected for an alignment of two random, unrelated sequences (Weidmann (2019) bioRxiv 706119). Nevertheless, both proteins assume a similar fold, with two notable differences: Proteasome R subunits are typically encoded with a propeptide that is cleaved off autocatalytically upon complex assembly. Furthermore, Adriase sequences lack a helical section found in proteasome subunits, but encode for an insertion of two helices at a different position.

Adriase is found in most archaea capable of producing methane from carbon dioxide and molecular hydrogen (hydrogenotrophic methanogenesis; (Costa (2014) Curr Opin Biotechnol 29:70-5). Amongst those, two Adriase variants exist: While being composed of just the DUF2121 domain in class II methanogens (Bapteste (2005) Archaea 1:353-63), such as Methanosarcina mazei, Adriase from class I methanogens, such as Methanocaldococcus jannaschii, features an extra C-terminal OB-like domain (oligosaccharide binding; FIG. 1).

So far to the best of our knowledge only a single PhD thesis has studied this domain and identified a certain structural homology between the DUF2121 domain and the NTN-domain (N-terminal nucleophile domain) of the proteolytic proteasome β-subunits (Moritz Ammelburg, “AAA Proteins and the Origins of Proteasomal Protein Degradation”, doctoral thesis, Eberhard Karls University Tubingen, 2011; https://publikationen.uni-tuebingen.de/xmlui/bitstream/handle/10900/49675/pdf/Dissertation_Ammelburg.pdf). The author of this PhD thesis suggested that the DUF2121 containing proteins have caseinolytic activity comparable to the proteasome; i.e. may have a proteolytic activity with a broad substrate spectrum. Yet, this study did not provide any insight in the precise mechanism of action of the family of DUF2121 domain containing proteins and the proposed activity relied only on a single in vitro protease assay conducted with a DUF2121 containing protein from M. jannaschii referred to as MjMPM (GI: 15668728, locus tag MJ_0548) expressed with an N-terminal His-tag that was cleaved of by thrombin cleavage, yet with leaving three amino acids N-terminally fused to the MjMPM sequence.

The previously proposed caseinolytic activity of DUF2121 domain could not be confirmed and instead it was convincingly demonstrated in the appended Examples and Figures that the DUF2121 domain surprisingly has transpeptidase activity suitable for ligating protein fragments in a sequence specific manner and with a high reaction rate. The appended Examples show that transpeptidase activity requires positioning the DUF2121 domain N-terminally within the polypeptide of the invention and having a threonine or serine residue positioned N-terminally in the DUF2121 containing transpeptidase polypeptide. A mutant variant of DUF2121 in which the N-terminal serine/threonine residue is replaced by alanine showed no transpeptidase activity, as demonstrated in the appended Examples and Figures. Thus, the N-terminal threonine and serine residue of the DUF2121 is part of the active site of the transpeptidase and is, thus, herein also referred to as catalytic serine or threonine residue. This is in line with this residue being highly conserved as threonine or serine throughout the currently predicted DUF2121 domain containing proteins.

In contrast to the findings of the present invention that the transpeptidase activity requires an N-terminal serine or threonine residue, DUF2121 domain containing proteins were previously annotated to start with a methionine and having the serine or threonine residue found to be conserved at position 2 of the amino acid sequence. The present invention demonstrates that the post-translational removal of the N-terminal methionine is required for DUF2121 activity. The above-mentioned PhD thesis merely speculated that the N-terminal methionine may be cleaved off in analogy to the proteasomal NTN domain, yet the PhD thesis failed to provide any experimental evidence for this hypothesis and failed to provide a teaching how such cleavage can be practically achieved. In fact, the experimental data of this PhD thesis even suggested to the contrary that the serine or threonine residue does not necessarily have to be N-terminal for the alleged caseinolytic activity. The M. jannaschii DUF2121 containing protein used in the experimental analysis, referred to as MjMPM in this PhD thesis, was produced such that an N-terminal Gly-Ser-His stretch from a thrombin cleavage site remained before the methionine residue of MjMPM when the N-terminal His-tag linker was removed by thrombin cleavage. This MjMPM protein was found to have caseinolytic activity in an in vitro assay performed in the PhD thesis, thus, indicating that the alleged caseinolytic activity does not require removal of the start methionine.

As demonstrated by appended Example 8 an N-terminally modified M. jannaschii Adriase shows no transpeptidase activity under the standard transpeptidase assay conditions. Also appended Example 13 reveals that the MjMPM protein construct employed in the above-mentioned PhD thesis is catalytically inactive under the standard transpeptidase assay conditions. Only when impracticable high enzyme concentrations are used the MjMPM protein construct shows transpeptidase activity. Example 13 illustrates that the MjMPM protein construct has a 200-fold reduced transpeptidase activity compared to a M. jannaschii Adriase variant harboring an N-terminal serine/threonine residue. A mass spectrometrical analysis revealed that the MjMPM protein construct preparation contains a small amount of N-terminal truncated Adriase protein in which the catalytical serine residue is exposed (Example 13, FIG. 15). Said truncated fraction is responsible for the slight catalytic activity of the protein preparation used in Example 13. This illustrates that the present invention reveals the catalytic activity and the catalytic active amino acid sequence of DUF2121 for the first time. The new sequence-specific transpeptidase of the invention is useful in numerous applications that involve post-translational protein engineering by generating new peptide bonds.

By identifying methyltransferase A (MtrA), which is part of a membrane-bound MtrA-MtrH complex as a novel endogenous interactor of the DUF2121 protein of Methanosarcina mazei and studying the mechanism of this interaction, the present invention reveales that active DUF2121 domains recognize substrate proteins comprising a DUF2121 recognition motif comprising X₁DPX₂A sequence motif (with X₁ being selected from K and R and X₂ being selected from G and A; see SEQ ID NOs: 308 to 311), preferably a X₁DPGA sequence motif (with X₁ being selected from K and R; see SEQ ID NOs: 310 and 311) and most preferably a KDPGA sequence motif (see SEQ ID NO: 311). This motif is highly conserved in MtrA proteins of DUF2121 comprising archaea. The terms “DUF2121 recognition motif” and “DUF2121 recognition sequence” are used interchangeably herein. Further it has been surprisingly found that catalytically active DUF2121 with an N-terminal serine or threonine residue cuts the substrate protein MtrA between the aspartate (D) and proline (P) residues in positions 2 and 3 of SEQ ID NOs: 308-311 of the DUF2121 recognition motif and that DUF2121 forms a covalent conjugate with the N-terminal portion of the substrate protein (ending with the amino acids as defined by positions 1 and 2 of any one of SEQ ID NOs: 308, 309, 310 and 311). Dimethyl-labeling mass spectrometry experiments suggest that the covalent conjugate between DUF2121 and the substrate protein surprisingly appears to involve amino group of the N-terminal serine/threonine, with strong evidence for the formation of a peptide bond between the N-terminal DUF2121 serine/threonine residue and the aspartate residue in position 2 of SEQ ID NO: 308, 309, 310 and 311 as comprised in the DUF2121 recognition motif, respectively. It has been surprisingly found that the formation of the covalent conjugate formed between the aspartate residue of the N-terminal MtrA portion and the DUF2121 N-terminus is reversible and that the reverse reaction occurs at a significant and at a robustly detectable rate. This is unexpected since such reversible reaction restoring the previously cut substrate protein has not been previously observed for the proteasome or proteasome homologues at considerable rates. Instead, proteasomal activity involves an irreversible hydrolysis reaction releasing the substrate attached protein fragment irreversibly. In the reverse reaction catalyzed by the DUF2121 transpeptidase, a new peptide bond is formed between the aspartate residue which was previously covalently attached to the DUF2121 serine/threonine residue and the proline residue corresponding to position 3 of SEQ ID NO: 308, 309, 310 and 311, respectively and defining the N-terminal residue of the C-terminal portion of the DUF2121 recognition motif. It has been surprisingly found that when the reaction is performed in presence of two different substrates comprising a DUF2121 recognition motif or one substrate having the full recognition motif and a second substrate mimicking the C-terminal cut product by bearing the C-terminal portion of the recognition motif at its N-terminus (starting with PGA), chimerical protein fusions are formed, i.e. DUF2121 acts as transpeptidase and/or recombinase forming new fusion proteins comprising the N-terminal portion of the first substrate and the C-terminal portion of the second substrate and/or vice versa. This demonstrates that DUF2121 can act as transpeptidase and/or peptide recombinase. Transpeptidase activity is a very rare, yet commercially very attractive enzymatic activity with numerous uses such as in protein engineering (e.g. the production of multivalent antibodies), site-specific or segmental protein labeling, protein localization studies and immunotherapeutic applications (e.g. the production of virus particles fused to a variety of antigens).

It is noted that the terms “amino acid” and “amino acid residue” are used interchangeably herein.

The polypeptide of the present invention and the use thereof in protein-protein ligation are linked to a number of advantages vis-à-vis the prior art enzymatic peptide ligation systems, in particular also vis-à-vis the most frequently employed sortase A peptide ligation system. These advantages make the polypeptide of the invention particularly suitable for the use in the above-mentioned applications.

A first advantage of the new transpeptidase system of the present invention is that the transpeptidase specifically recognizes substrate proteins via a short recognition motif or the C-terminal portion thereof (PGA . . . ). Such short recognition motifs allow for engineering substrate proteins by adding only a minimum of additional amino acids and such minimizes the risk of interference with proper folding and activity of substrate proteins vis-à-vis other protein ligation systems as discussed above. The flexibility of using the transpeptidases provided herein is further facilitated by the fact that the DUF2121 recognition motif can be placed N-terminally, C-terminally or internally. This is different from other peptide ligation systems such as the split intein system which are limited to N-terminal and C-terminal fusions of the intein sequences.

A further advantage of the transpeptidase (system) provided herein is that the transpeptidase of the present invention catalyzes peptide ligation with a surprisingly higher specificity compared to other peptide ligases of the prior art, like Sortase A, which uses shorter peptide sequences as recognition sequence. This higher substrate specificity of the transpeptidase is particularly advantageous since it allows the reaction to occur also in presence of other proteins (i.e. in complex mixtures or in vivo). The half-maximum reaction rate of the transpeptidase is observed at substrate concentrations as low as about 2.2 μM when the ligation is performed with equimolar concentrations of the substrates, the first substrate comprising a DUF2121 recognition motif and the second substrate having the C-terminal portion of the DUF2121 recognition motif starting with PGA at its N-terminus (see appended Example 7). This value is lower than previously reported K_{M} values for Sortase A and an evolved tetramutant thereof. Sortase A shows a K_{M} value of 7333 μM for the primary substrate when the secondary substrate is used in excess and a K_{M} value of 196 μM for the secondary substrate when the primary substrate is used in excess (Frankel (2005) Biochemistry 44:11188-200). An evolved tetramutant of sortase shows a K_{M} value of 170 μM for the primary substrate when the secondary substrate is used in excess and a K_{M} value of 4800 μM for the secondary substrate when the primary substrate is used in excess (Chen (2011) Proc Natl Acad Sci USA 108:11399-404). Thus, the transpeptidase of the invention combines sequence specificity and high reaction rates. As described herein above a further advantage of the inventive polypeptide is that the half maximum velocity of the transpeptidase reaction is reached already at low substrate concentrations. In context of the present invention low substrate concentration may relate to substrate concentrations below 20 μM, below 30 μM, below 40 μM, below 50 μM, below 60 μM, below 70 μM, below 80, below 90 μM, below 100 μM, below 110 μM or below 120 μM. It is evident to the skilled person that there may be the need to determine the optimal reaction conditions for a certain transpeptidase of the present invention in order to observe the half maximum velocity of the transpeptidase reaction already at low substrate concentrations. The reaction paramters, which may be adjusted to determine optimal reaction conditions are described herein below. The appended examples demonstrate how the half maximum velocity of the transpeptidase reaction may be determined (Example 7, FIG. 9B).

Another particular advantage of the transpeptidases of the invention is that the DUF2121 catalyzed reaction involves a highly hydrolysis resistant reaction intermediate (i.e. a peptide bond, see FIG. 8) rather than more labile thioesters that are prone to hydrolysis. Hydrolysis is an irreversible side reaction decreasing the production rate of the desired ligation products observed for prior art transpeptidases such as sortase A (Frankel (2005) loc. cit.; Heck (2014) Bioconjug Chem 25:1492-500). As demonstrated by appended Example 11 no products arising from undesired hydrolysis side reactions could be detected in context of the present invention.

A comparison of Adriase with Sortase (SrtA) and an evolved Sortase A pentamutant (SrtA5*) shown in appended example 16 demonstrates that Adriase is particularly advantageous at low (3 μM) substrate concentrations. However, also at high substrate concentrations (100 μM), Adriase ligates the used substrates at >4000× higher rates compared to SrtA and >40× compared to SrtA5*, even when used at 32× lower substrate concentrations, and produces substantially (~1.7×) higher yields without detectable side reactions.

A further advantage of the transpeptidases provided herein is that these proteins are thermostable which is favorable for protein storage and stability. It has also been shown in the present invention that the polypeptides of the invention can be efficiently recombinantly expressed in E. coli and purified at high yields in soluble form. In an experiment depicted in the appended examples the polypeptide of the invention was purified with a yield of at least 5 mg soluble protein per liter of culture. Accordingly, high yield in context of the present invention may be at least 1 mg soluble protein per liter of culture, at least 2 mg soluble protein per liter of culture, at least 3 mg soluble protein per liter of culture, at least 4 mg soluble protein per liter of culture or at least 5 mg soluble protein per liter of culture. Importantly, it has been found that the polypeptides can be expressed, for example, in E. coli in an active form, because the N-terminal methionine encoded by the start codon is removed in this expression system so as to produce the polypeptide with transpeptidase activity as provided herein.

Accordingly, the transpeptidases of the invention have the advantage of being specific for a recognition sequence motif and having a high reaction rate. This allows specific peptide and protein ligations at high reaction rates also in presence of low substrate peptide/protein levels in vitro and/or in vivo.

As mentioned above, according to a first aspect, the present invention relates to a polypeptide comprising an N-terminal DUF2121 domain having an N-terminal serine or threonine residue.

The polypeptide of the invention has transpeptidase activity, preferably sequence-specific transpeptidase activity.

As used herein “N-terminal DUF2121 domain” means that the amino acid sequence of the DUF2121 domain forms the N-terminus of the polypeptide. In other words the first amino acid of the DUF2121 domain, which in context of the invention is a threonine or a serine residue, forms the N-terminus with a free amino-group (N-terminus of the polypeptide).

As used herein “N-terminal serine or threonine residue” means that a serine or threonine residue forms the N-terminus of a DUF2121 domain. In other words the first amino acid of DUF2121 domain is a serine or threonine residue. Preferably, said serine or threonine residue also forms the N-terminus of the polypeptide comprising the DUF2121 domain with a free amino group.

Note that also preparations of polypeptides of the present invention having additional amino acid residues N-terminally of the catalytic serine or threonine residue of the DUF2121 domain can have transpeptidase activity. As shown in appended Example 13 said preparations may contain a fraction of truncated polypeptides with N-terminal catalytic serine/threonine residue.

A “transpeptidase”, as used herein, is an enzyme or a catalytic domain of an enzyme or a polypeptide that is able to catalyze the breakage of one or more peptide bonds and subsequently the formation of one or more novel peptide bonds. By this activity novel peptide bonds can be formed between two originally not connected polypeptides or fragments thereof, i.e. two polypeptides or fragments thereof can be “ligated” in a posttranslational manner. Due to the formation of a new peptide bond by the transpeptidase, the polypeptides of the invention may also be referred to as “protein ligases” or “peptide ligases”.

As used herein, the term “sequence-specific transpeptidase” defines a transpeptidase which requires the substrate peptides or proteins to comprise a recognition sequence to act on the substrates as transpeptidase. The DUF2121 domain-containing transpeptidase of the invention recognizes its substrates via an amino acid sequence motif referred to as “DUF2121 recognition motif” or “DUF2121 recognition sequence” herein. As demonstrated in the appended examples and as described in detail below one of two substrate polypeptides may only comprise the C-terminal portion of the DUF2121 recognition motif. What is understood under C-terminal portion is specified herein below. The DUF2121 recognition sequence may be positioned N-terminally, internally or C-terminally in a substrate protein. In substrate proteins comprising only the C-terminal portion of the DUF2121 recognition sequence, the C-terminal portion of the DUF2121 recognition motif must be positioned at the N-terminus. In principle, it is also possible that a substrate protein comprises two or more DUF2121 recognition motifs. In this event multiple transpeptidase reactions linking different parts of polypeptides are generated.

If the polypeptide of the invention acts on two substrate proteins comprising the DUF2121 recognition motif internally, the transpeptidase activity leads to an exchange of protein portions between the two substrate proteins. Specifically, the N-terminal portion of the first substrate protein and the C-terminal portion of the second substrate protein are ligated. In the same reaction also the N-terminal portion of the second substrate protein may be ligated with the C-terminal portion of the first substrate protein. Due to this capability to replace portions of a substrate protein, the polypeptide of the invention may also be referred to as “peptide recombinase”. The term “peptide recombinase”, as used herein, means that a fragment of a first substrate polypeptide is replaced by a portion of a second substrate polypeptide. A recombination reaction furthermore encompasses the capability to replace a portion of a first substrate polypeptide with the entire sequence of a second substrate polypeptide.

The polypeptide of the invention provided herein has a DUF2121 domain at its N-terminus with an N-terminal serine or threonine. DUF2121 domains are known in the art and annotated in databases (see Pfam: PF09894; InterPro: IPR016754). Thus, amino acid sequences of annotated DUF2121 domains are readily derivable from these databases. Moreover, DUF2121 sequences are enclosed herein. Based on sequence alignments of these amino acid sequences also the conserved catalytic threonine or serine residue now found herein to form the N-terminal amino acid in the active form of DUF2121 can be identified with routine measures, i.e. amino acid sequence alignments. The threonine and serine residue which corresponds to the amino acid in position 1 of SEQ ID NOs: 4 to 143, forms the N-terminal amino acid residue of the polypeptide of the invention. In the annotated DUF2121 sequences, which typically also comprise the N-terminal methionine encoded by the ATG start codon, this serine/threonine residue is in most of the annotated sequences (more than 50%) found in position 2 of the annotated sequences. Only in some of the annotated sequences the serine and threonine residue is not found in position 2 but further downstream behind another methionine residue (as it becomes apparent from an alignment with all annotated DUF2121 domains). In these sequences the start codon is most likely misannotated in the database and the actual amino acid sequence starts with the methionine before the conserved threonine and serine residue. However, a skilled person can identify using routine sequence alignment method, e.g. as described herein below, to identify the serine or threonine residue corresponding to position two of the majority of the annotated DUF2121 sequences and to the active site. Based on the already annotated DUF2121 domains a skilled person can also identify DUF2121 domains in not yet annotated sequences with routine methods, such as sequence alignments and BLAST analysis, preferably as mentioned herein below. To identify potential DUF2121 domains the skilled person can run a protein BLAST search against the non-redundant protein sequence database, using default parameters and the DUF2121 consensus (SEQ ID NO: 2) as query sequence. When used in context of the present invention the default parameters were: Max target sequences: 500/Expect threshold: 10/Word size: 6/Max matches in a query range: 0/Scoring Matrix: BLOSUM62/Gap Costs: Existence: 11 Extension: 1/Conditional compositional score matrix adjustment/No filters or masking. The skilled artisan may adopt these parameters for his/her purposes. But standards, values, parameters provided herein were established using these parameters and may be considered as reference.

An e-value of the Blast alignment of 1×10⁻¹⁰ or less indicates that the sequence of interest is with high likelihood a DUF2121 domain. Exemplary and preferred DUF2121 domains are disclosed herein below.

In order to determine whether a nucleotide residue/position or an amino acid residue/position in a given nucleotide sequence or amino acid sequence, respectively, corresponds to a certain position compared to another nucleotide sequence or amino acid sequence, respectively, the skilled person can use means and methods well known in the art, e.g., alignments, either manually or by using computer programs such as those mentioned herein. For example, BLAST 2.0 can be used to search for local sequence alignments. BLAST or BLAST 2.0, as discussed above, produces alignments of nucleotide or protein sequences to determine sequence similarity. Because of the local nature of the alignments, BLAST or BLAST 2.0 is especially useful in determining exact matches or in identifying similar or identical sequences. Similarly, alignments may also be based on the CLUSTALW computer program (Thompson (1994) Nucl. Acids Res. 2:4673-4680) or CLUSTAL Omega (Sievers (2014) Curr. Protoc. Bioinformatics 48:3.13.1-3.13.16).

Using these methods a skilled person is readily in the position to identify the serine or threonine residue corresponding to the serine or threonine residue forming the N-terminal amino acid in position 1 of any one of the DUF2121 sequences depicted in SEQ ID NOs: 4 to 143.

As mentioned above, the DUF2121 domain as comprised in the polypeptide of the invention is characterized in that it has sequence-specific transpeptidase activity. The sequence specific transpeptidase activity of a DUF2121 domain or DUF2121 domain containing protein provided herein can be assessed with routine assays as defined herein and used in the appended Examples. Such transpeptidase assays may involve the provision of two substrate proteins comprising a DUF2121 recognition motif and bringing the same into contact with the polypeptide to be tested for transpeptidase activity. The DUF2121 recognition motif may be the same or different in the two substrates. Preferably, the same DUF2121 recognition motif is employed in both substrates. The DUF2121 recognition motif may be positioned anywhere in the two substrate proteins (e.g. internally, N-terminal or C-terminal). In an embodiment the assay for testing transpeptidase activity may be performed in (several) parallel reactions, each of the reactions using a different pair of substrates, wherein the two substrates of a pair comprise the same DUF2121 recognition sequences, and wherein the substrate pairs of the different reactions have different DUF2121 recognition motifs. The number of different DUF2121 recognition sequences and substrate pairs employed in these testings/assays for transpeptidase activity may be varied. A DUF2121 domain is found to have transpeptidase activity in the event that transpeptidase activity is measured with the read out used for at least one of the tested substrate pairs. In an illustrative assay at least 5 different substrate pairs may be tested. It is also envisaged that, at least 10, at least 15, at least 20, at least 25, at least 30, at least 35, at least, 40, at least 45, at least 50, at least 55, at least 60, at least 65, at least 70, at least 75, at least 80, at least 85, at least 90, at least 95 or at least 100 substrate pairs may be tested. Herein provided and exemplified are 214 DUF2121 recognition motifs. Said recognition motifs are depicted in SEQ ID NOs: 315-366, 460-510 and 551-661. Accordingly, said at least 100 substrate pairs, for example the 103 or 214 substrate pairs comprising the DUF2121 recognition motifs as provided in context of the invention and its priorities may be analyzed. Said substrate pairs may comprise one of the DUF2121 recognition motifs depicted in SEQ ID NOs: 315-366, 460-510 and 551-661, wherein every substrate pair comprises a different DUF2121 recognition motif and wherein within a substrate pair the same DUF2121 recognition motif is used. In other words, a test for sequence-specific transpeptidase activity according to the invention may involve the assessment whether a polypeptide acts as a transpeptidase on any one of the DUF2121 recognition motifs depicted in SEQ ID NOs: 315-366, 460-510 and 551-661. A tested polypeptide is considered as a sequence-specific transpeptidase according to the invention, if at least for one of the tested substrate pairs/DUF2121 recognition motifs transpeptidase activity can be measured. The measurement of the transpeptidase activity can be directly or indirectly. “Direct” measurement means that the newly generated fusion protein resulting from the transpeptidase reaction is detected (e.g. by SDS-PAGE and/or size exclusion chromatography and/or mass spectrometry). “Indirect” measurement means that a side product, e.g. an amino acid fragment released by the transpeptidase reaction (e.g., a labeled amino acid fragment released by the transpeptidase reaction) is detected. In other words, a tested polypeptide acts as a sequence-specific transpeptidase according to the invention if the polypeptide shows at least transpeptidase activity according to the read-out of the detection method used for at least one of the DUF2121 recognition motifs as depicted in SEQ ID NOs: 315-366, 460-510 and 551-661. The DUF2121 recognition motifs are sequences derived from MtrA protein sequences of DUF2121 domain expressing organisms. Table 1 shows the origin of the DUF2121 recognition motifs and the growth conditions for the corresponding organisms. Suitable detection methods are described herein below and the appended Examples.

The substrate protein pairs used in the assay can in principle be any proteins as long as the selection of proteins allows for a read out of the transpeptidase reaction.

One read out to measure transpeptidase activity of a polypeptide when brought into contact with a substrate polypeptide pair is SDS-PAGE. When using this read out, the substrate protein molecular weights and the position of the DUF2121 recognition motif therein (which determines the weight of the N-terminal and C-terminal portion) need to be selected such that at least one of the chimeric substrate proteins resulting from DUF2121 transpeptidase activity (i.e. fusion of N-terminal portion of first substrate protein and C-terminal portion of second substrate protein and vice versa) can be distinguished in its SDS-PAGE migration behavior from the two substrate proteins. This difference in migration behavior allows detecting the production of a chimeric substrate protein by sequence-specific transpeptidase reaction by detecting a band in the SDS-PAGE corresponding to the migration behavior of the formed chimeric substrate protein. SDS-PAGE analysis is a routine method known in the art. A skilled person can define the SDS gel to be used and the buffers to be used depending on the molecular weight of the protein fragments to be analyzed. Instead of SDS PAGE also LCMS (Liquid Chromatography-Mass Specotroscopy) may be used as read out, e.g., as described in the following and the appended Examples.

Accordingly, to test for the sequence-specific transpeptidase activity of a DUF2121 domain one may incubate the polypeptide to be tested for enzymatic activity (e.g. 0.5 g/l) with a first substrate protein comprising a DUF2121 recognition and a second substrate protein (e.g. 0.5 g/l) comprising a DUF2121 recognition motif, e.g. the same as the first substrate protein). The second substrate protein is preferably different from the first substrate protein. For instance, the first substrate protein may be based on a MtrA fragment comprising the DUF2121 recognition sequence (e.g. SEQ ID NO.420) and the second substrate protein is based on an artificial ubiquitin with a DUF2121 recognition motif C-terminally fused thereto (e.g. SEQ ID NO:392). The mixture may be incubated over night (e.g. at room temperature) in 20 mM HEPES-NaOH pH 7.5, 100 mM NaCl, 50 mM KCl, 0.5 mM TCEP. Subsequently, samples can then be analyzed by SDS-PAGE. Alternatively, or additionally samples may be desalted and subjected to a Phenomenex Aeris Widepore 3.6 μm C4 200 Å (100×2.1 mm) column, eluted with a 30-80% H2O/acetonitrile gradient over 15 min in the presence of 0.05% trifluoroacetic acid and analyzed with a Bruker Daltonik microTOF. Data processing may be performed with Bruker Compass DataAnalysis 4.2 and the m/z deconvoluted with the MaxEnt module to obtain the protein mass. The relevant read out in both read out methods is whether the chimeric polypeptide expected as product of the transpeptidase activity is formed.

Very similar the skilled person is also able to identify additional DUF2121 recognition sequences. Based on the already identified DUF2121 recognition motifs a skilled person can also identify DUF2121 recognition sequences in not yet annotated sequences with routine methods, such as sequence alignments and protein BLAST analysis, preferably as mentioned herein. The protein BLAST search may be performed using default parameters and the consensus DUF2121 recognition motif (SEQ ID NO: 366) as a query sequence. When used in context of the present invention the default parameters were: Max target sequences: 500/Expect threshold: 100/Word size: 2/Max matches in a query range: 0/Scoring Matrix: PAM30/Gap Costs: Existence: 9 Extension: 1/No compositional score matrix adjustment/No filters or masking, see in this context also the comments herein above. An e-value of 1 or less indicates that the sequence of interest is with high likelihood a DUF2121 recognition motif.

To assess whether a given sequence is a DUF2121 recognition motif, routine assays as defined herein and used in the appended Examples may be performed. These assays may involve the provision of two substrate polypeptides comprising the sequence to be tested, i.e. the potential DUF2121 recognition motif and bringing the same into contact with a DUF2121 domain containing polypeptide having transpeptidase activity as described herein. The potential DUF2121 recognition motif may be positioned at any sterically accessible position within the two substrate proteins (e.g. internally, N-terminal or C-terminal). The skilled person is able to identify sterically accessible positions in a substrate through structure prediction tools, such as HHPred. In an embodiment the assay for identifying a DUF2121 recognition motif is performed in (several) parallel reactions, each of the reactions using the two substrate polypeptides comprising the sequence to be tested and each of the reaction using different polypeptides having transpeptidase activity as described herein. The number of different DUF2121 domain containing polypeptides having transpeptidase activity employed may be varied. A certain sequence is found to be (or determined as) a DUF2121 recognition sequence in the event that transpeptidase activity is measured with the read out used for at least one of the DUF2121 containing polypeptides.

In an illustrative assay for identifying a DUF2121 recognition motif at least 5 different reactions may be tested. It is also envisaged that at least 10, at least 15, at least 20, at least 25, at least 30, at least 35, at least, 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190 or at least 200 reactions may bet tested. Herein provided and exemplified are 222 DUF2121 domain containing polypeptides. Said DUF2121 domain containing polypeptides are depicted in SEQ ID NOs: 4-225.

Accordingly, said at least 200 reaction, for example 222 reactions comprising the DUF2121 domain containing polypeptides as provided in context of the invention may be analyzed. The reactions may comprise one of the DUF2121 domain containing polypeptides depicted in SEQ ID NOs: 4-225, wherein every reaction comprises a different DUF2121 domain containing polypeptide and the two substrate polypeptides comprising the sequence to be tested. In other words, a test whether a sequence is a DUF2121 recognition motif according to the invention may involve the assessment whether a DUF2121 domain containing polypeptide depicted in SEQ ID NOs: 4-225 acts as a transpeptidase on the two substrate polypeptides containing the sequence to be tested. A tested sequence is considered to be a DUF2121 recognition motif according to the invention, if at least one of the DUF2121 domain containing polypeptides acts as a transpeptidase on the two substrate polypeptides comprising the sequence to be tested. The DUF2121 domain containing polypeptides that might be used for said test reactions were identified in different microorganisms. Table 1 depicts the corresponding microorganisms and the corresponding growth conditions. The skilled person is well aware that the required reaction conditions for the DUF2121 containing polypeptide to show transpeptidase activity may be not identical for different DUF2121 containing polypeptides. Accordingly, the skilled person knows how to adjust reaction parameters such as temperature, salt concentration, pH etc. to test for maximal transpeptidase activity of the DUF2121 containing polypeptide. The skilled person is also aware that the reaction condition required by the DUF2121 containing polypeptide may resemble the growth condition of the microorganism where said DUF2121 containing polypeptide is derived from. However, it is evident for the skilled person that a transpeptidase of the present invention may also work well at conditions different from the growth conditions of the corresponding organism. It is evident for the skilled person that a transpeptidase of the present invention may work well at temperatures lower (or higher) than the optimal growth temperature of the organism said transpeptidase is derived from. Accordingly, a transpeptidase of the present invention may be isolated from a hyperthermophilic organism but may work well at ambient temperatures or physiologic temperatures of mesophilic organisms (e.g. 25 °C or 37 °C). Also, a transpeptidase of the present invention may be isolated from a thermophilic organism but may work well at ambient temperatures or physiologic temperatures of mesophilic organisms (e.g. 25 °C or 37 °C). Accordingly, said transpeptidases of the present invention may be used at 10 °C to 40 °C and all digits inbetween, such as 15 °C, 20 °C, 25 °C, 30 °C, 35 °C or 37 °C. Accordingly, in the appended Examples it is shown that Adriase of M. mazei works well at about 37 °C.

Although evident for the skilled person it is pointed out that not for all DUF2121 recognition motifs depicted in SEQ ID NOs: 315-366, 460-510 and 551-661 transpeptidase activity may be measured when contacted with any polypeptide of the invention. Transpeptidase activity may only be measured when certain DUF2121 recognition motifs are contacted with certain polypeptides of the invention. Transpeptidase activity may be measured when a DUF2121 recognition motifs of a certain species will be contacted with the DUF2121 domain of the same species or a polypeptide comprising the DUF2121 domain of the same species. It is also evident for the skilled artisan that the length of a DUF2121 recognition motif may be optimized. The skilled artisan may apply the assays described herein to identify a certain combination of (a) DUF2121 domain(s) and (a) DUF2121 recognition motif(s) as, inter alia, depicted in SEQ ID NOs: 315-366, 460-510 and 551-661, wherein said combination exhibits transpeptidase activity. To optimize the DUF2121 recognition motif the skilled person may subject several variants of the DUF2121 recognition motif to the transpeptidase assay, wherein the variants may be characterized that one or more amino acid residues starting from the N-terminus of the motif and/or starting from the C-terminus of the motif are removed. The DUF2121 recognition motif variant that leads to the highest transpeptidase activity in a corresponding assay may be used for subsequent applications. It is also possible to optimize the DUF2121 recognition motifs by substituting one or more amino acids of the motif by other amino acids. The amino acid substitution may be conservative or non-conservative. “Conservative amino acid substitution” as used herein means that the amino acid is substituted by an amino acid of similar chemical properties. “Non-conservative amino acid substitution” as used herein means that the amino acid is substituted by an amino acid of different chemical properties. Preferably, the amino acid residues in the X₁DPX₂A sequence motif as described above in the DUF2121 recognition motif is not substituted.

Without being bound by theory it is envisaged that the “amino acid environment” of the DUF2121 recognition motif may have influence on the effectivity of the DUF2121 recognition motif Pronounced and/or transpeptidase activity may be observed when a certain DUF2121 recognition motif embedded in a certain polypeptide or used isolated is contacted with a certain DUF2121 domain. However, no transpeptidase activity or a reduced transpeptidase activity may be observed when the same DUF2121 recognition motif is contacted with the same DUF2121 domain but wherein the DUF2121 recognition motif is embedded in a different polypeptide. In other words, the amino acid residues N-terminally and/or C-terminally of the DUF2121 recognition motif may have influence on the transpeptidase activity observed when said DUF2121 recognition motif is contacted with a DUF2121 domain. Without being bound by theory it is also envisaged that sterically more demanding substrates for the transpeptidase reaction require elongated DUF2121 recognition motifs. For the DUF2121 recognition motif from M. mazei for example it is demonstrated in the appended examples that (5)KDPGA (SEQ ID NO: 311) (10) (the number in brackets denote the number of amino acids N-terminally and C-terminally of the KDPGA (SEQ ID NO: 311) motif) may be a useful DUF2121 recognition motif for sterically accessible substrates, such as peptides, and that sterically more demanding protein-protein ligations are catalyzed most efficiently via the (5)KDPGA (SEQ ID NO: 311) (15) motif. However, it is pointed out that this observation may not be true for DUF2121 recognition motifs of other organisms.

The measurement of the transpeptidase activity can be directly or indirectly. “Direct” measurement means that the newly generated fusion protein resulting from the transpeptidase reaction is detected (e.g. by SDS PAGE and/or size exclusion chromatography and/or mass spectrometry). “Indirect” measurement means that a side product, e.g. an amino acid fragment released by the transpeptidase reaction (e.g., a labeled amino acid fragment released by the transpeptidase reaction) is detected. In other words, a tested sequence is a DUF2121 recognition motif according to the invention if at least one DUF2121 containing polypeptide depicted in SEQ ID NOs: 4-225 acts as a transpeptidase on the two substrate polypeptides comprising the sequence to be tested according to the read-out of the detection method used. Suitable detection methods are described herein below and the appended Examples.

The substrate polypeptides comprising the sequence to be tested used in the assay described above can in principle be any polypeptides as long as the selection of proteins allows for a read out of the transpeptidase reaction.

One read out to measure transpeptidase activity of a polypeptide when brought into contact with a substrate polypeptide pair is SDS-PAGE. When using this read out, the substrate polypeptide molecular weights and the position of the sequence to be tested, i.e. the potential DUF2121 recognition motif therein (which determines the weight of the N-terminal and C-terminal portion) need to be selected such that at least one of the chimeric substrate proteins resulting from DUF2121 transpeptidase activity (i.e. fusion of N-terminal portion of first substrate protein and C-terminal portion of second substrate protein and vice versa) can be distinguished in its migration behavior from the two substrate proteins. This difference in migration behavior allows detecting the production of a chimeric substrate protein by sequence-specific transpeptidase reaction by detecting a band in the SDS PAGE corresponding to the migration behavior of the formed chimeric substrate protein. SDS PAGE analysis is a routine method known in the art. A skilled person can define the SDS gel to be used and the buffers to be used depending on the molecular weight of the protein fragments to be analyzed. Instead of SDS PAGE also LCMS may be used as read out, e.g., as described in the appended Examples.

Accordingly, to test for whether a given sequence is a DUF2121 recognition motif one may incubate the first substrate polypeptide comprising the sequence to be tested (e.g. 0.5 g/l) and a second substrate polypeptide comprising the sequence to be tested (e.g. 0.5 g/l) with a DUF2121 domain containing polypeptide (e.g. 0.5 g/l), preferable a polypeptide as depicted in SEQ ID NOs: 4-225. The mixture may be incubated over night (e.g. at room temperature) in 20 mM HEPES-NaOH pH 7.5, 100 mM NaCl, 50 mM KCl, 0.5 mM TCEP. Subsequently, samples may be desalted. These samples can then be analyzed by SDS-PAGE. Alternatively, or additionally desalted samples may be subjected to a Phenomenex Aeris Widepore 3.6 μm C4 200 Å (100×2.1 mm) column, eluted with a 30-80% H2O/acetonitrile gradient over 15 min in the presence of 0.05% trifluoroacetic acid and analyzed with a Bruker Daltonik microTOF. Data processing may be performed with Bruker Compass DataAnalysis 4.2 and the m/z deconvoluted with the MaxEnt module to obtain the protein mass. The relevant read out in both read out methods is whether the chimeric polypeptide expected as product of the transpeptidase activity is formed. Although obvious for the skilled person and mentioned herein above it is again pointed out that the substrate polypeptides have to be chosen that a read out of transpeptidase reaction is possible, e.g. that the molecular weight of the fusion polypeptide is different from the molecular weight of the substrate polypeptides.

In principle, in the polypeptide of the invention any DUF2121 domain can be employed as long as it has the conserved serine or threonine residue (corresponding to position 2 of the correctly annotated DUF2121 sequence) at its N-terminus and has transpeptidase activity. In a preferred embodiment the polypeptide of the invention comprises a DUF2121 domain that has the amino acid sequence as depicted in SEQ ID NO: 2 or an amino acid sequence having at least 20%, preferably at least 25%, even more preferably at least 30%, even more preferably at least 30%, even more preferably at least 40%, even more preferably at least 50%, even more preferably at least 60%, even more preferably at least 70%, even more preferably at least 80%, even more preferably at least 90%, even more preferably at least 95%, even more preferably at least 98% and most preferably at least 99% sequence identity thereto and having sequence-specific transpeptidase activity according to the invention. The amino acid sequence depicted in SEQ ID NO: 2 is a consensus sequence prepared based on SEQ IDs NO: 4-143. These sequences were aligned with MUSCLE (https://toolkit.tuebingen.mpg.de/tools/muscle; 1 iteration) and filtered for a maximum sequence identity of 60% using Hhfilter (https://toolkit.tuebingen.mpg.de/tools/hhfilter). The resulting alignment of the remaining sequences (SEQ IDs NO: 4-7, 10, 14, 19, 22, 30, 31, 33, 39, 43, 53, 61, 69, 72, 73, 78, 86, 87, 92, 93, 96-99, 115, 126, 135, 140, 141 and 143) was then used to create said consensus sequence with the advanced consensus maker tool (https://www.hiv.lanl.gov/content/sequence/CONSENSUS/AdvCon.html; consensus is always the most common letter setting). The consensus sequence preferably shares a sequence identity of at least 25%, preferably at least 30% and most preferably at least 35% identity with DUF2121 domain sequences. The appended Examples demonstrate that the DUF2121 domains of Methanosarcina mazei (SEQ ID NO: 106) and Methanocaldococcus jannaschii (SEQ ID NO: 17) have sequence specific transpeptidase activity according to the invention if expressed so as to have an N-terminal threonine or serine residue.

Again it has to be pointed out that also preparations of the polypeptide of the present invention with amino acid residues N-terminally of the catalytic serine or threonine residue can exhibit transpeptidase activity. As demonstrated in the appended Examples such preparations may contain truncated variants of the polypeptide exposing the catalytic serine or threonine at the N-terminus leading to transpeptidase activity. Accordingly, the invention also relates to a polypeptide having transpeptidase activity as described herein above wherein said polypeptide may further comprise at least one additional amino acid residue N-terminally of the catalytical serine or threonine residue.

Furthermore, the N-terminal serine/threonine residue corresponding to position 1 of SEQ ID NO: 2 has been identified as crucial for the transpeptidase activity and defines the catalytically active form of a DUF2121 domain. Further, it has been found that deletion of the positions of the DUF2121 corresponding to positions 28 to 57 of the DUF2121 consensus sequence of SEQ ID NO: 2 in DUF2121 domains interferes with transpeptidase activity. Without being bound by theory, these residues that are present in the DUF2121 domain comprised in the polypeptide of the invention may also be involved in maintaining DUF2121 sequence-specific transpeptidase activity. Accordingly, the polypeptide of the invention may comprise an N-terminal DUF2121 domain that has an N-terminal serine or threonine residue and an amino acid sequence as defined by positions 28 to 57 of SEQ ID NO: 2, the amino acid sequence fragment of any one of SEQ ID NOs: 4 to 143 corresponding in an alignment to positions 28 to 57 of SEQ ID NO: 2, or a sequence having at least 30%, preferably at least 60% and most preferably at least 90% sequence identity to positions 28 to 57 of SEQ ID NO: 2 or the corresponding fragments of any one of SEQ ID NOs: 4 to 143.

In a preferred embodiment, the polypeptide of the invention comprises or consists of a DUF2121 domain having an amino acid sequence selected from the group consisting of SEQ ID NOs: 4 to 143 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having transpeptidase activity according to the invention. In a particularly preferred embodiment the polypeptide of the invention may comprise or consist of a DUF2121 domain having an amino acid sequence selected from the group consisting of SEQ ID NOs: 4 to 143 and having sequence-specific transpeptidase activity according to the invention. In one embodiment, the polypeptide of the invention may comprise or consist of a DUF2121 domain having an amino acid sequence selected from the group consisting of SEQ ID NOs: 4 to 85 or an amino acid sequence having at least 20%, preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having sequence specific transpeptidase activity according to the invention. The DUF2121 sequences depicted in SEQ ID NOs: 4 to 85 correspond to DUF2121 domains annotated in the PFAM database, yet differ from the database entries in that they lack the N-terminal methionine removal of which is required for transpeptidase activity. The DUF2121 domains of SEQ ID NOs: 4 to 85 form part of DUF2121 domain-containing proteins comprising additional protein sequences and domains. As demonstrated throughout the appended examples also shortened version of said DUF2121 domain-containing proteins show transpeptidase activity as long as they comprise the N-terminal DUF2121 domain (see e.g. Example 8). The corresponding full length proteins are depicted in SEQ ID NOs: 144 to 225. The protein sequences comprised in the DUF2121 domain containing proteins of SEQ ID Nos: 144 to 225 include an additional GB-domain like fold as identified from the structure of a DUF2121 domain-containing protein solved in the appended examples (see FIG. 6). The OB-like domains in these DUF2121 domain-containing proteins are not mandatory for DUF2121 transpeptidase activity as demonstrated in the appended examples. However, their presence may facilitate substrate binding and thus the transpeptidase reaction. In another aspect, the polypeptide of the invention comprises or consists of a DUF2121 domain having an amino acid sequence selected from the group consisting of SEQ ID NOs: 86 to 143 or an amino acid sequence having at least 20%, even more preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having sequence-specific transpeptidase activity according to the invention. The DUF2121 domains annotated in SEQ ID NOs: 86 to 143 are annotated in the PFAM database, yet with an additional N-terminal methionine. The DUF2121 domains of SEQ ID NOs: 86 to 143 represent the entire amino acid sequences of the annotated proteins, i.e. these proteins lack further protein domains.

In a preferred embodiment of the invention, the DUF2121 domain of the polypeptide of the invention may consist of an amino acid sequence selected from SEQ ID NOs: 17 and 106 or an amino acid sequence having at least 20%, even more preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having sequence-specific transpeptidase activity according to the invention. In one embodiment the DUF2121 domain of the polypeptide of the invention may consist of an amino acid sequence selected from SEQ ID NOs: 17 and 106.

Preferably, the polypeptide of the invention may comprise an amino acid sequence selected from the group consisting of SEQ ID NOs: 86 to 225 or an amino acid sequence having at least 20%, preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and has a sequence specific transpeptidase activity according to the invention. More preferably, the polypeptide of the invention may consist of an amino acid sequence selected from the group consisting of SEQ ID NOs: 86 to 225 or an amino acid sequence having at least 20%, preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and has a sequence specific transpeptidase activity according to the invention. Additionally, the polypeptide of the invention may comprise or consist of an amino acid sequence selected from the group consisting of SEQ ID NOs: 86 to 143 or an amino acid sequence having at least 20%, preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and has transpeptidase activity according to the invention. The polypeptide of the invention may also comprise or consist of an amino acid sequence selected from the group consisting of SEQ ID NOs: 144 to 225 or an amino acid sequence having at least 20%, preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and has a sequence specific transpeptidase activity according to the invention.

In a preferred embodiment of the invention, the polypeptide of the invention may comprise an amino acid sequence selected from the group consisting of an amino acid sequence selected from SEQ ID NOs: 106 and 159 or an amino acid sequence having at least 20%, even more preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having sequence-specific transpeptidase activity according to the invention. In one embodiment the DUF2121 domain of the polypeptide of the invention may comprise the amino acid sequence selected from SEQ ID NOs: 17 and 106. In a particularly preferred embodiment of the invention, the polypeptide of the invention may consist of an amino acid sequence selected from the group consisting of an amino acid sequence selected from SEQ ID NOs: 106 and 159 or an amino acid sequence having at least 20%, even more preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having sequence-specific transpeptidase activity according to the invention. The DUF2121 domain of the polypeptide of the invention may consist of the amino acid sequence selected from SEQ ID NOs: 17 and 106.

The polypeptide of the invention may optionally comprise an OB-like domain, preferably C-terminally of the DUF2121 domain. An “OB-like domain” in the context of the invention relates to an amino acid sequence having a fold similar to the GB-fold. Preferably, an OB-like domain in the context of the invention has an amino acid sequence selected from the group consisting of SEQ ID NOs: 1 and 226 to 307 or an amino acid sequence having at least 60% sequence identity, preferably at least 70%, even more preferably at least 80%, even more preferably at least 90%, even more preferably at least 95% and most preferably at least 99% sequence identity to said amino acid sequence. SEQ ID NO: 1 corresponds to an OB-like consensus sequence based on SEQ IDs NO: 226-307. These sequences were aligned with MUSCLE (https://toolkit.tuebingen.mpg.de/tools/muscle; 3 iterations) and filtered for a maximum sequence identity of 60% using Hhfilter (https://toolkit.tuebingen.mpg.de/tools/hhfilter). The resulting alignment of the remaining sequences was then used to create said consensus sequence with the advanced consensus maker tool (https://www.hiv.lanl.gov/content/sequence/CONSENSUS/AdvCon.html; consensus is always the most common letter setting). The SEQ ID NOs: 226 to 307 represent the OB-like domains of the DUF2121 domain containing proteins annotated in the PFAM database. As shown in the appended Examples an OB-like domain is not required for the DUF2121 sequence specific transpeptidase activity. However, the presence of the OB-like domain may facilitate catalytic activity as transpeptidase. Without being bound by theory, the structural data presented in the appended Examples suggests that the OB-like domain promotes substrate binding. In a preferred embodiment the polypeptide of the invention may comprise an N-terminal DUF2121 domain consisting of an amino acid sequence selected from the group consisting of SEQ ID NOs: 4 to 85 and further comprises an OB-like domain, preferably an GB-like domain consisting of an amino acid sequence selected from the group consisting of SEQ ID NOs 226 to 307 or an amino acid sequence having at least 60% sequence identity, preferably at least 70%, even more preferably at least 80%, even more preferably at least 90%, even more preferably at least 95% and most preferably at least 99% sequence identity to said amino acid sequence. The OB-like domain is positioned more C-terminally in the polypeptide of the invention, preferably directly C-terminally of the DUF2121 domain. Particularly preferred is a polypeptide of the invention consisting of a DUF2121 domain and a OB-like domain C-terminally, preferably directly C-terminal thereof, wherein the OB-like domain consists of an amino acid sequence selected from the group consisting of SEQ ID NOs: 1 and 226 to 307 or an amino acid sequence having at least 60% sequence identity, preferably at least 70%, even more preferably at least 80%, even more preferably at least 90%, even more preferably at least 95% and most preferably at least 99% sequence identity to said amino acid sequence. Even more preferably the OB-like domain consists of an amino acid sequence selected from the group consisting of SEQ ID NOs: 1 and 226 to 307.

SEQ ID NO: 3 represents an artificial polypeptide, wherein the consensus sequence of an OB-like domain is C-terminally fused to the consensus sequence of the DUF2121 domain. Accordingly, the polypeptide of the invention may comprise an amino acid sequence as depicted in SEQ ID NO: 3 or an amino acid sequence having at least 20%, even more preferably at least 30%, preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said amino acid sequence and having sequence-specific transpeptidase activity according to the invention.

The polypeptide of the invention, the DUF2121 domain comprised therein or other domains or amino acid sequence stretches comprised therein are defined by sequence identity to a certain amino acid sequence in some embodiments. Those having skill in the art will know how to determine percent identity between/among sequences using, for example, algorithms such as those based on CLUSTALW computer program (Thompson (1994) Nucl. Acids Res. 2:4673-4680), CLUSTAL Omega (Sievers (2014) Curr. Protoc. Bioinformatics 48:3.13.1-3.13.16) or FASTDB (Brutlag (1990) Comp App Biosci 6:237-245). Also available to those having skill in this art are the BLAST, which stands for Basic Local Alignment Search Tool, and BLAST 2.0 algorithms (Altschul, (1997) Nucl. Acids Res. 25:3389-3402; Altschul (1990) J. Mol. Biol. 215:403-410) and related tools. The BLASTN program for nucleic acid sequences uses as defaults a word length (W) of 11, an expectation I of 10, M=5, N=4, and a comparison of both strands. The BLOSUM62 scoring matrix (Henikoff (1992) Proc. Natl. Acad. Sci. U.S.A. 89:10915-10919) uses alignments (B) of 50, expectatiI(E) of 10, M=5, N=4, and a comparison of both strands.

The polypeptide of the invention may be provided as isolated or purified protein. Isolated or purified in the context of the invention means that the polypeptide is substantially free from other proteins or contaminants. This may be achieved by the purification methods as described herein in the appended examples for exemplary transpeptidases of the invention.

It is clear for the skilled person that the polypeptide of the invention may comprise an affinity tag. The affinity tag is positioned C-terminally or internally (i.e. not N-terminal).

As evident from the description herein, the polypeptide of the invention may be non-natural and may be recombinantly expressed and generated by genetic engineering. In particular, the polypeptide of the invention may also be a non-naturally occurring fusion protein.

The polypeptide of the invention may be attached to a solid carrier. Said attachment is made in a manner that preserves the sequence specific transpeptidase activity according to the invention. Methods to test the transpeptidase activity of a polypeptide attached to a solid carrier are similar to the assays for testing transpeptidase activity of polypeptides as described herein elsewhere and disclosed in the appended Examples. The only difference is that instead of the polypeptide of the invention a solid carrier with a protein of the invention attached thereto (or multiple copies thereof) is contacted with the substrate proteins. To maintain catalytic activity of the polypeptide of the invention, the attachment of the polypeptide to the solid carrier is preferably mediated by a residue different from the N-terminal serine or threonine residue. For instance, the attachment to the solid carrier may be mediated via an internal residue (not the N-terminal and C-terminal residue) or via the C-terminus. Methods for attaching the polypeptide of the invention to a solid carrier are known in the art. In a preferred embodiment multiple copies of the polypeptide of the invention are attached to a solid carrier. In this context the different polypeptides attached to the solid carrier may be identical or different. In a preferred embodiment the polypeptides are identical. Non-limiting examples for a solid carrier according to the present invention are a polymer, a hydrogel, a microparticle, a nanoparticle, a sphere (e.g. a nano- or microsphere), beads (e.g. microbeads), quantum dots, prosthetics and a solid surface. In a preferred embodiment the carrier is a bead (e.g. a microbead), such as an agarose bead. Accordingly, the invention relates to beads (e.g. microbeads) having the polypeptide of the present invention attached thereto. Such beads with the polypeptide of the invention may represent a ready-to-use reagent for producing a fusion polypeptide

Accordingly, the invention also relates to kits comprising the polypeptide of the invention having transpeptidase activity. Said kit may comprise the polypeptide of the invention in a ready to use reaction mixture for producing a fusion polypeptide.

As mentioned herein above, the polypeptide of the invention has sequence specific transpeptidase activity. In the context of the DUF2121-containing polypeptide of the invention the sequence specificity is conferred by the recognition of a DUF2121 recognition motif or the C-terminal portion thereof in a substrate protein by the DUF2121 domain of the polypeptide of the invention. Thus, the sequence-specific transpeptidase activity according to the invention may comprise the capability of catalyzing the formation of a peptide bond between the most C-terminally positioned residue of an N-terminal portion of a first substrate polypeptide and the most N-terminally positioned residue of a C-terminal portion of a second substrate polypeptide so as to form a fusion polypeptide comprising the N-terminal portion of the first substrate polypeptide and the C-terminal portion of the second substrate polypeptide C-terminally fused thereto. The first and the second substrate polypeptide in this context each comprise a DUF2121 recognition motif comprising a sequence selected from the group consisting of SEQ ID NOs: 308, 309, 310 and 311, preferably SEQ ID NOs: 310 and 311, and most preferably SEQ ID NO: 311. The N-terminal portion of the first substrate peptide is preferably defined from the N-terminus of the first substrate peptide to the aspartate residue in position 2 of SEq ID NOs: 308, 309, 310 and 311, respectively. The C-terminal portion of the first substrate polypeptide is preferably defined from the proline residue in position 3 of SEQ ID NOs: 308, 309, 310 and 311, respectively, to the C-terminus of the sequence of the first substrate polypeptide. The N-terminal portion of the second substrate peptide is preferably defined from the N-terminus of the second substrate peptide to the aspartate residue in position 2 of SEQ ID NOs: 308, 309, 310 and 311 comprised therein, respectively. The C-terminal portion of the second substrate polypeptide is preferably defined from the proline residue in position 3 of SEQ ID NOs: 308, 309, 310 and 311, respectively, to the C-terminus of the sequence of the second substrate polypeptide.

The expression “most N-terminally positioned” as used herein means that an amino acid forms the first amino acid counted from the N-terminus defining a certain amino acid domain, fragment or portion. This first amino acid defining the start of the domain, fragment or portion does not form an N-terminus with a free amino group if the defined domain, fragment or portion is positioned internally in a protein.

The expression “most C-terminally positioned” as used herein means that an amino acid forms the last amino acid counted from the N-terminus defining a certain amino acid domain, fragment or portion. This last amino acid defining the end of the domain, fragment or portion does not form a C-terminus with a free carboxyl group if the defined domain, fragment or portion is positioned internally in a protein.

The appended Examples demonstrate that the polypeptide of the invention can also catalyze the formation of a peptide bond between the most C-terminally positioned residue of an N-terminal portion of a first substrate polypeptide comprising a DUF2121 recognition motif and the N-terminal amino acid of a second substrate polypeptide having at its N-terminus the C-terminal portion of a DUF2121 recognition motif (starting with the proline in position 3 of SEQ ID NOs: 308, 309, 310 and 311, respectively). Thus, the sequence-specific transpeptidase activity according to the invention may comprise the capability of catalyzing the formation of a peptide bond between the most C-terminally positioned residue of an N-terminal portion of a first substrate polypeptide and the N-terminal residue of a second substrate polypeptide so as to form a fusion polypeptide comprising the N-terminal portion of the first substrate polypeptide and the second substrate polypeptide C-terminally fused thereto. The first substrate polypeptide in this context preferably comprises a DUF2121 recognition motif, said DUF2121 recognition motif comprising an amino acid sequence selected from the group consisting of SEQ ID NOs: 308, 309, 310 and 311, preferably SEQ ID NOs: 310 and 311, most preferably SEQ ID NO: 311. The N-terminal portion of the first substrate polypeptide is preferably defined from the N-terminus thereof to the aspartate residue in position 2 of SEQ ID NOs: 308, 309, 310 and 311, respectively. The second substrate polypeptide preferably has at its N-terminus the C-terminal portion of a DUF2121 recognition motif. The C-terminal portion of a DUF2121 recognition motif starts with the amino acids defined in positions 3 to 5 of any one of SEQ ID NOs: 308 to 311.

It has been surprisingly found that the polypeptides of the invention have a sequence-specific transpeptidase activity; i.e. can be used for post-translational protein ligations.

Thus, in one aspect, the present invention relates to the use of the polypeptide of the invention as a sequence specific transpeptidase. The use as transpeptidase may specifically comprise catalyzing the post-translational ligation of two peptide or protein portions. Preferred sequence specific transpeptidase reactions that can be catalyzed by the polypeptide of the invention are disclosed herein in the context of the methods described. The disclosures in context of the methods described herein are disclosed as corresponding use mutatis mutandis.

The invention further relates to a nucleic acid encoding the polypeptide as described herein above. The terms “nucleic acid”, “polynucleotide”, “nucleic acid sequence”, “nucleic acid molecule” or “nucleotide sequence” are used interchangeably herein and refer to DNA, such as cDNA or genomic DNA, and RNA (e.g. messenger RNA). The polynucleotides used in accordance with the present invention may be of natural as well as of (semi) synthetic origin. The nucleic acids of the invention can e.g. be synthesized by standard chemical synthesis methods and/or recombinant methods, or produced semi-synthetically, e.g. by combining chemical synthesis and recombinant methods. Ligation of the coding sequences to transcriptional regulatory elements and/or to other amino acid encoding sequences can be carried out using established methods, such as restriction digests, ligations and molecular cloning.

The person skilled in the art is familiar with the preparation and the use of polynucleotides (see, e.g., Sambrook and Russel “Molecular Cloning, A Laboratory Manual” (2001), Cold Spring Harbor Laboratory, N.Y.).

The terms “encode” or “encoding” are used interchangeably with the terms “encode for” or “encoding for”, respectively. These terms mean that according nucleic acid sequence may serve as template for production of the “encoded amino acid sequence” according to the known rules of the genetic code. If organisms with a modified genetic code are used, the “encoding” nucleic acids may also include sequences adapted to such modifications in the genetic code.

The nucleic acid provided herein may be an open reading frame; i.e. a continuous stretch of codons capable of being translated to an amino acid sequence that starts with a translation start codon (including alternative start codons known in the art) and ends with a translation stop codon. The term “open reading frame” is interchangeably used with “coding sequence” herein.

Accordingly, the nucleic acid of the invention may comprise further features required to express the nucleic acid sequence encoding the polypeptide of the invention in a host cell. For instance, the nucleic acid sequence may be operably linked to a promoter sequence. The nucleic acid molecule of the invention may further comprise regulatory sequences. Regulatory sequences are well known to those skilled in the art and include, without being limiting, regulatory sequences ensuring the initiation of transcription, internal ribosomal entry sites (IRES) (Owens (2001) Proc. Natl. Acad. Sci. U.S.A. 98:1471-1476) and optionally regulatory elements ensuring termination of transcription and stabilization of the transcript. Non-limiting examples for such regulatory elements ensuring the initiation of transcription comprise promoters, a translation initiation codon, enhancers, insulators and/or regulatory elements ensuring transcription termination, which are to be included downstream of the nucleic acid molecules of the invention. Further examples include Kozak sequences and intervening sequences flanked by donor and acceptor sites for RNA splicing, nucleotide sequences encoding secretion signals or, depending on the expression system used, signal sequences capable of directing the expressed protein to a cellular compartment or to the culture medium.

The present invention further relates to a vector comprising a nucleic acid of the invention; i.e. encoding a transpeptidase polypeptide as provided herein. Many suitable vectors are known to those skilled in molecular biology, the choice of which depends on the desired function. Non-limiting examples of vectors include plasmids, cosmids, viruses, bacteriophages and other vectors used conventionally in e.g. genetic engineering. Methods which are well known to those skilled in the art can be used to construct various plasmids and vectors (see for example Sambrook and Russel (2001) loc cit.; Ausubel (1989) Current Protocols in Molecular Biology, Green Publishing Associates and Wiley Interscience, N.Y.).

The vector preferably comprises a promoter being operably linked to the nucleic acid. “Operably linked” means that the promoter is positioned so that it drives the expression of the nucleic acid. Preferably, the vector of the invention is an expression vector. An expression vector according to this invention is capable of directing the replication and the expression of the nucleic acid molecule of the invention in a host or host cell and, accordingly, provides for the expression of the polypeptide of the present invention encoded thereby in the selected host or host cell. Expression comprises transcription of the nucleic acid molecule, for example into a translatable mRNA and translation into a polypeptide.

The nucleic acid molecules and/or vectors of the invention can be designed for introduction into cells by e.g. chemical based methods (polyethylenimine, calcium phosphate, liposomes, DEAE-dextrane, nucleofection), non chemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for delivery of the nucleic acid molecules into targeted cell population. Additionally, baculoviral systems can also be used as vector in eukaryotic expression system for the nucleic acid molecules of the invention. In one embodiment, the nucleic acid molecules and/or vectors of the invention are designed for transformation of chemical competent E. coli by calcium phosphate and/or for transient transfection of HEK293 and CHO by polyethylenimine- or lipofectamine-transfection.

Non-limiting examples of vectors include pQE-12, the pUC-series, pBluescript (Stratagene), the pET-series of expression vectors (Novagen) or pCRTOPO (Invitrogen), lambda gt11, pJOE, the pBBR1-MCS series, pJB861, pBSMuL, pBC2, pUCPKS, pTACT1, pTRE, pCAL-n-EK, pESP-1, pOP13CAT, the E-027 pCAG Kosak-Cherry (L45a) vector system, pREP (Invitrogen), pCEP4 (Invitrogen), pMClneo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, Okayama-Berg cDNA expression vector pcDVl (Pharmacia), pRc/CMV, pcDNA1, pcDNA3 (Invitrogen), pcDNA3.1, pSPORT1 (GIBCO BRL), pGEMHE (Promega), pLXIN, pSIR (Clontech), pIRES-EGFP (Clontech), pEAK-10 (Edge Biosystems) pTriEx-Hygro (Novagen) and pCINeo (Promega). A preferred vector is the pET30 vector. This vector has also been used in the appended examples.

Further it is envisaged herein that the nucleic acid molecule or vectors as described herein are transfected into a host cell.

Accordingly, the present invention further relates to a host cell comprising a nucleic acid, a vector, or an expression vector as described herein above.

The host cell can be any prokaryotic or eukaryotic cell. The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis.

Suitable bacterial expression hosts comprise e.g. strains derived from JM83, W3110, KS272, TG1, K12, BL21 (such as BL21(DE3), BL21(DE3)PlysS, BL21(DE3)RIL, BL21(DE3)PRARE) or Rosetta. In a preferred embodiment the bacterial expression host is E. coli BL21 Gold(DE3) as used in the appended examples.

The term “eukaryotic” is meant to include yeast, higher plant, insect and mammalian cells. Typical mammalian host cells include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells. Other suitable eukaryotic host cells include, without being limiting, chicken cells, such as e.g. DT40 cells, or yeasts such as Saccharomyces cerevisiae, Pichia pastoris, Schizosaccharomyces pombe and Kluyveromyces lactis. Insect cells suitable for expression are e.g. Drosophila S2, Drosophila Kc, Spodoptera Sf9 and Sf21 or Trichoplusia Hi5 cells. Suitable zebrafish cell lines include, without being limiting, ZFL, SID or ZF4.

The described vector(s) can either integrate into the genome of the host or can be maintained extrachromosomally. Once the vector has been incorporated into the appropriate host, the host is maintained under conditions suitable for high level expression of the nucleic acid molecules, and as desired, the collection and purification of the polypeptide of the invention may follow. Appropriate culture media and conditions for the above described host cells are known in the art.

The host cell described herein may express a methionyl aminopeptidase. A methionyl aminopeptidase is capable of removing the N-terminal methionine from a polypeptide. In other words the methionyl aminopeptidase removes the first amino acid from a polypeptide when the first amino acid is a methionine. A methionyl aminopeptidase may be able to remove the N-terminal methionine from a polypeptide of the present invention. A methionyl aminopeptidase may remove the methionine of an N-terminal MS or MT motif of a polypeptide of the present invention. A methionyl aminopeptidase used to remove the N-terminal serine or threonine of a polypeptide of the present invention may be E. coli MetAP (SEQ ID NO: 314).

The present invention further relates to a method for producing a polypeptide of the invention as described herein. The method may comprise cultivating the host cell as described herein above comprising the nucleic acid, the vector or the expression vector as described herein above under conditions conducive for production of the polypeptide and recovering said polypeptide from the cell culture and/or cells. The terms “recovering”, “purifying”, “collecting” and “isolating” are used interchangeably herein.

In the production methods of the present invention, the cells are cultivated in a nutrient medium suitable for production of the polypeptide using methods known in the art. For example, the cells may be cultivated by shake flask cultivation, and small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions. If the polypeptide is secreted into the nutrient medium, the polypeptide can be recovered directly from the medium. If the polypeptide is not secreted, it can be recovered from cell lysates. The cell lysate may be prepared by lysing the cells by ultra sonification or using a french press.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see for example Jansen (1989) Protein Purification, VCH Publishers, New York). The resulting polypeptide may be detected by methods known in the art (e.g., SDS-PAGE and Coomassie staining or Western Blotting)

A polypeptide of the present invention, preferably M. jannaschii Adriase (MJ_0548) can be cloned with a C-terminal His₆ (SEQ ID NO: 678)-tag (SEQ ID NO: 385) into a pET30 vector (SEQ ID NO: 516) and transformed in BL21 DE3 cells carrying the pACYC-RIL plasmid. The transformed cells can be grown at 25 °C in lysogeny broth (LB). The kanamycin concentration may be kept at 25 μg/ml and the chloramphenicol concentration at 12.5 μg/ml. Protein expression may be induced at an optical density of 0.4 at 600 nm with 500 μM isopropyl-β-D-thiogalactoside. After 16 h, cells can be harvested and all subsequent steps may be conducted at 7 °C. The cell pellet of His6 (SEQ ID NO: 678)-tagged constructs can be resuspended in 100 mM Tris-HCl pH 8.0, 10 mM Imidazole, 5 mM MgCl₂, 50 μg/ml DNAse (Applichem) and cOmplete protease inhibitor (Roche). Cells may be lysed by three french press passages at 16000 psi, and cleared from cell debris by ultracentrifugation at 100000 g for 45 min. The supernatant can then filtered using a membrane filters (Millipore) with a pore size of 0.22 μm.

The His₆ (SEQ ID NO: 678)-tagged protein can be purified via HisTrap HP columns (the columns may be obtained from GE Healthcare) using an Akta Pure FPLC (GE Healthcare) with Unicorn v5.1.0 software. The filtered supernatant can be applied to the equilibrated column (20 mM Tris-HCl pH 8.0, 250 mM NaCl, 20 mM imidazole) and washed with 10 additional column volumes of the same buffer. Bound proteins can then be eluted by gradually increasing the imidazole concentration up to 300 mM. The eluted fractions can be analyzed via SDS-PAGE and those containing the protein of interest at comparatively high purity may be pooled and used for subsequent purification steps. The protein can be concentrated using Amicon centrifugal filters with a 10 kDa molecular weight cut-off (Merck) to a concentration of 10 g/1. Finally, a maximum of 0.02 column volumes of the concentrated proteins may be applied to a Superdex 75 size-exclusion column (buffer A: 20 mM HEPES-NaOH pH 7.5, 100 mM NaCl, 50 mM KCl, 0.5 mM TCEP). Eluted fractions can be analyzed via SDS-PAGE, pooled and concentrated as described above. For long-term storage, the protein containing fractions may be supplemented with 15% glycerol, flash frozen in liquid nitrogen and stored at −80 °C.

Additional non-limiting examples of nucleotide sequences that might be fused to a tag for the production of polypeptides that have transpeptidase activity are SEq ID NOs: 312, 313, 389, 402 and 430. Said sequences also encode for polypeptides harboring an N-terminal methionine residue which has to be removed as described herein for the polypeptide to have transpeptidase activity according to the invention.

It is clear that all polypeptides, preferably SEQ ID NOs: 2-225 provided herein may be produced by the above described or other methods. The skilled person knows that the protein sequences have to be transformed in nucleotide sequences. Suitable tools are well known in the art, e.g. DNASTAR-Lasergene. It is of note that the start codon consisting of the nucleotides ATG may be added to induce translation of the polypeptide. Accordingly, the produced polypeptide may contain an N-terminal methionine residue. As mentioned herein the N-terminal methionine residue has to be removed to expose the catalytic serine or threonine residue at the N-terminus. A suitable measure may be to express the polypeptide in a host cell comprising a methionyl aminopeptidase as described herein.

The invention further relates to a method for producing a fusion polypeptide comprising contacting the polypeptide as defined herein with a first substrate polypeptide and a second substrate polypeptide, and reacting both substrate polypeptides.

Said method may comprise producing a fusion polypeptide.

The produced fusion polypeptide of said method may comprise a portion of the first substrate polypeptide and a portion of the second substrate polypeptide, or a portion of the first substrate polypeptide and the entire second polypeptide.

In the inventive method for producing a fusion polypeptide the first substrate polypeptide may comprise a DUF2121 recognition motif.

Note that all DUF2121 recognition motifs described herein are for illustrative purposes only and are in no way limiting besides the key motif X₁DPX₂A described herein. Also all SEQ ID NOs concerning DUF2121 recognition motifs are for illustrative purposes only and are in no way limiting. The skilled person is readily capable of identifying additional DUF2121 recognition motifs by the means and methods described herein.

In particular the DUF2121 recognition motif of the first substrate polypeptide may comprise and/or consist of an amino acid sequence selected from the group consisting of SEQ ID NOs: 308, 309, 310 and 311, preferably SEQ ID NOs: 310 and 311, most preferably SEQ ID NO: 311.

A further description of the first substrate polypeptide for the inventive polypeptide comprising an N-terminal DUF2121 domain as described herein is provided below.

Accordingly, in the inventive method for producing a fusion polypeptide the DUF2121 recognition motif of the first substrate polypeptide may comprise additional amino acids. Accordingly, the DUF2121 recognition motif of the first substrate polypeptide may comprise additionally at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids N-terminally of SEQ ID NOs: 308, 309, 310 and 311, respectively. Furthermore, the DUF2121 recognition motif of the first substrate polypeptide may comprise additionally at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids C-terminally of SEQ ID NOs: 308, 309, 310 and 311, respectively. Yet, also longer C-terminal additions are envisaged in context of the present invention for example as also illustrated in the experimental part. A DUF2121 recognition motif of the first substrate polypeptide additionally comprising at least 20 amino acids C-terminally of SEQ ID NOs: 308, 309, 310 and 311, respectively, may be particularly preferred.

Thus, in the inventive method for producing a fusion polypeptide the DUF2121 recognition motif of the first substrate polypeptide may comprise additionally at least 10, at least 15 or at least 20 amino acids C-terminally of SEQ ID NOs: 308, 309, 310 and 311, respectively.

The DUF2121 recognition motif of the first substrate polypeptide may comprise additionally at least 5 amino acids N-terminally and at least 10 amino acids C-terminally of SEQ ID NOs: 308, 309, 310 and 311, respectively.

The DUF2121 recognition motif of the first substrate polypeptide may comprise additionally at least 5 amino acids N-terminally and at least 15 amino acids C-terminally of SEQ ID NOs: 308, 309, 310 and 311, respectively.

The DUF2121 recognition motif of the first substrate polypeptide may comprise additionally at least 5 amino acids N-terminally and at least 20 amino acids C-terminally of SEQ ID NOs: 308, 309, 310 and 311, respectively.

The DUF2121 recognition motif of the first substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15 or 15 of any one of SEQ ID NOs: 315-366, 460-510 and 551-661 N-terminally, preferably directly N-terminally of SEQ ID NOs: 308, 309, 310 and 311, respectively.

The DUF2121 recognition motif of the first substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally, preferably directly C-terminally of SEQ ID NO: 308, 309, 310 and 311, respectively.

The DUF2121 recognition motifs may also be longer compared to the motifs described and provided above. Accordingly, the DUF2121 recognition motif of the first substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of SEQ ID NOs: 308, 309, 310 and 311, respectively.

It is evident that a sequence defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 may also comprise, for example, a sequence defined by positions 21 to 30 of any one of SEQ ID NOs: 315-366, 460-510 and 551-661. Thus, the DUF2121 recognition motif of the first substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally, preferably directly C-terminally of SEQ ID NO: 308, 309, 310 and 311, respectively, and/or may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of SEQ ID NOs: 308, 309, 310 and 311, respectively.

In a preferred embodiment of the inventive method for producing a fusion polypeptide the DUF2121 recognition motif of the first substrate polypeptide may consist of the sequence as defined in any one of SEQ ID NOs: 315-366, 460-510 and 551-661 or a sequence having or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

In the inventive method for producing a fusion polypeptide the first substrate polypeptide may have an N-terminal portion defined from the N-terminus of the first substrate polypeptide to the aspartate residue in position 2 of SEQ ID NOs: 308, 309, 310 and 311, respectively.

In a preferred embodiment of the inventive method for producing a fusion polypeptide the second substrate polypeptide may comprise a DUF2121 recognition motif.

In a more preferred embodiment of the inventive method for producing a fusion polypeptide the DUF2121 recognition motif of the second substrate polypeptide may comprise and/or consist of an amino acid sequence selected from the group consisting of SEQ ID NOs: 308, 309, 310 and 311, preferably SEQ ID NOs: 310 and 311, most preferably SEQ ID NO: 311.

A further description of the second substrate polypeptide for the inventive polypeptide comprising an N-terminal DUF2121 domain as described herein is provided below. Accordingly, in the inventive method for producing a fusion polypeptide the DUF2121 recognition motif of the second substrate polypeptide may comprise additional amino acids. Accordingly, the DUF 2121 recognition motif of the second substrate polypeptide may additionally comprise at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids N-terminally of said SEQ ID NOs: 308, 309, 310 and 311, respectively.

The DUF 2121 recognition motif of the second substrate polypeptide may comprise at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids C-terminally of said SEQ ID NOs: 308, 309, 310 and 311, respectively.

As also described above a DUF2121 recognition motif of the second substrate polypeptide additionally comprising at least 20 amino acids C-terminally of SEQ ID NOs: 308, 309, 310 and 311, respectively, may be particularly preferred.

Thus, in the inventive method for producing a fusion polypeptide the DUF2121 recognition motif of the second substrate polypeptide may comprise additionally at least 10, at least 15 or at least 20 amino acids C-terminally of SEQ ID NOs: 308, 309, 310 and 311, respectively.

The DUF2121 recognition motif of the second substrate polypeptide may comprise at least 5 amino acids N-terminally and at least 10 amino acids C-terminally of said SEQ ID NOs: 308, 309, 310 and 311, respectively.

The DUF2121 recognition motif of the second substrate polypeptide may comprise at least 5 amino acids N-terminally and at least 15 amino acids C-terminally of said SEQ ID NOs: 308, 309, 310 and 311, respectively.

The DUF2121 recognition motif of the second substrate polypeptide may comprise at least 5 amino acids N-terminally and at least 20 amino acids C-terminally of said SEQ ID NOs: 308, 309, 310 and 311, respectively.

The DUF 2121 recognition motif of the second substrate polypeptide may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15 or 15 of any one of SEQ ID NOs: 315-366, 460-510 and 551-661N-terminally, preferably directly N-terminally of said SEQ ID NOs: 308, 309, 310 and 311, respectively.

The DUF 2121 recognition motif of the second substrate polypeptide may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661C-terminally, preferably directly C-terminally of said SEQ ID NO: 308, 309, 310 and 311, respectively.

It is also envisaged that the DUF2121 recognition motifs are longer compared to the motifs described above. Accordingly, the DUF2121 recognition motif of the second substrate polypeptide may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of said SEQ ID NO: 308, 309, 310 and 311, respectively.

Thus, the DUF 2121 recognition motif of the second substrate polypeptide may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661C-terminally, preferably directly C-terminally of said SEQ ID NO: 308, 309, 310 and 311, respectively, and/or may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of said SEQ ID NO: 308, 309, 310 and 311, respectively.

In a preferred embodiment of the inventive method for producing a fusion polypeptide the DUF2121 recognition motif of the second substrate polypeptide may consist of the sequence as defined in any one of SEQ ID NOs: 315-366, 460-510 and 551-661 or a sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

In the inventive method for producing a fusion polypeptide the DUF2121 recognition sequence of the second substrate polypeptide may be identical with the DUF2121 recognition sequence of the first substrate polypeptide.

In the inventive method for producing a fusion polypeptide the second substrate polypeptide may have a C-terminal portion defined from the proline residue in position 3 of SEQ ID NOs: 308, 309, 310 and 311, respectively to the C-terminus of the second substrate polypeptide.

In the inventive method for producing a fusion polypeptide the first substrate polypeptide may have an N-terminal portion defined from the N-terminus of the first substrate polypeptide to the aspartate residue in position 2 of SEQ ID NO: 308, 309, 310 and 311, respectively, the second substrate polypeptide may have a C-terminal portion defined from the proline residue in position 3 of SEQ ID NOs: 308, 309, 310 and 311, respectively, to the C-terminus of the second substrate polypeptide and the produced fusion protein may comprise the N-terminal portion of the first substrate polypeptide and the C-terminal portion of the second substrate polypeptide C-terminally fused thereto.

In the inventive method for producing a fusion polypeptide the second substrate polypeptide may comprise a C-terminal portion of a DUF2121 recognition motif, said C-terminal portion of the DUF2121 recognition motif being positioned N-terminally of the second substrate polypeptide.

In the inventive method for producing a fusion polypeptide the C-terminal portion of the DUF2121 recognition motif may start with the amino acid sequence as defined in positions 3 to 5 of any one of SEQ ID NOs: 308, 309, 310 and 311, preferably SEQ ID NOs: 310 and 311, most preferably SEQ ID NO: 311.

In the inventive method for producing a fusion polypeptide the C-terminal portion of the DUF2121 recognition motif of the second substrate polypeptide may comprise additional amino acids. Accordingly, the C-terminal portion of the DUF2121 recognition motif may additionally comprise at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids C-terminally, preferably directly C-terminally of the N-terminal amino acids as defined by positions 3 to 5 of SEQ ID NOs: 308, 309, 310 and 311, respectively.

A C-terminal portion of the DUF2121 recognition motif additionally comprising at least 20 amino acids C-terminally, preferably directly C-terminally of the N-terminal amino acids as defined by positions 3 to 5 of SEQ ID NOs: 308, 309, 310 and 311, respectively, may be particularly preferred.

Accordingly, a C-terminal portion of the DUF2121 recognition motif may additionally comprise at least 10, at least 15 or at least 20 amino acids C-terminally, preferably directly C-terminally of the N-terminal amino acids as defined by positions 3 to 5 of SEQ ID NOs: 308, 309, 310 and 311.

The C-terminal portion of the DUF2121 recognition motif of the second substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally, preferably directly C-terminally of the N-terminal amino acids as defined by positions 3 to 5 of SEQ ID NOs: 308, 309, 310 and 311, respectively.

It is also envisaged that the C-terminal portion of the DUF2121 recognition motif is longer compared to the motifs described above. Accordingly, the C-terminal portion of the DUF2121 recognition motif of the second substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of the N-terminal amino acids as defined by positions 3 to 5 of SEQ ID NOs: 308, 309, 310 and 311, respectively.

Thus, the C-terminal portion of the DUF2121 recognition motif of the second substrate polypeptide may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally, preferably directly C-terminally of the N-terminal amino acids as defined by positions 3 to 5 of SEQ ID NOs: 308, 309, 310 and 311, respectively, and/or may additionally comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of the N-terminal amino acids as defined by positions 3 to 5 of SEQ ID NOs: 308, 309, 310 and 311, respectively.

The appended examples demonstrate that depending on the substrate 10, 15 or 20 preferably directly C-terminally of the N-terminal amino acids as defined by positions 3 to 5 of SEQ ID NOs: 308, 309, 310 and 311, respectively, may allow efficient ligations.

In the inventive method for producing a fusion polypeptide the C-terminal portion of the DUF 2121 recognition motif of the second substrate polypeptide may consist of the amino acid sequence as defined in positions 16 to 30 of any one of SEQ ID NOs: 315-366, 460-510 and 551-661 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

The C-terminal portion of the DUF 2121 recognition motif of the second substrate polypeptide may also consist of the amino acid sequence as defined in positions 16 to 35 of any one of SEQ ID NOs: 551-661 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

The C-terminal portion of the DUF 2121 recognition motif of the second substrate polypeptide may also consist of the amino acid sequence as defined in positions 16 to 40 of any one of SEQ ID NOs: 551-661 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence. In the inventive method for producing a fusion polypeptide the produced fusion polypeptide may comprise the complete second substrate polypeptide, preferably C-terminally when the C-terminal portion of the DUF2121 recognition motif being positioned N-terminally of the second substrate polypeptide

In the inventive method for producing a fusion polypeptide the first substrate polypeptide may have an N-terminal portion defined from the N-terminus of the first substrate polypeptide to the aspartate residue in position 2 of SEQ ID NO: 308, 309, 310 and 311, respectively, a C-terminal portion of the DUF2121 recognition motif as described herein above is positioned N-terminally of the second substrate polypeptide and the produced fusion polypeptide comprises the N-terminal portion of the first substrate polypeptide and the second substrate polypeptide C-terminally fused thereto.

In the inventive method for producing a fusion polypeptide the polypeptide of the present invention as described herein may be brought into contact with the first and the second substrate polypeptide as described herein simultaneously.

Accordingly, the polypeptide of the present invention may be brought into contact with the first substrate polypeptide as described herein and the second substrate polypeptide as described herein not simultaneously. Thus, in the inventive method for producing a fusion polypeptide of the present invention as described herein may be brought into contact with the first substrate polypeptide as described herein and the second substrate polypeptide as described herein may be only added after the first substrate polypeptide.

The polypeptide of the present invention can be attached to a solid carrier when contacted with the substrate polypeptides. Accordingly, in the inventive method for producing a fusion polypeptide the polypeptide of the present invention as described herein may be attached to a solid carrier and the solid carrier may be washed after addition of the first substrate polypeptide as described herein and before addition of the second substrate polypeptide as described herein.

The inventive method for producing a fusion polypeptide may be performed in vitro.

Although clear for the skilled person, it is noted that the fusion polypeptide produced by the inventive method can be collected. Thus, the inventive method for producing a fusion polypeptide may comprise collecting the produced fusion polypeptide. The terms “recovering”, “purifying”, “collecting” and “isolating” are used interchangeably herein. The produced polypeptide may be recovered by methods known in the art. For example, the polypeptide may be recovered from the reaction composition by conventional procedures including, but not limited to, centrifugation, filtration, extraction, spray-drying, evaporation, or precipitation. In another aspect the produced fusion polypeptide may be purified by chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see for example Jansen (1989) Protein Purification, VCH Publishers, New York). Non-limiting examples for affinity tags used in affinity chromatography are the His6 (SEQ ID NO: 678)-tag or the Strep-tag. The affinity tags bind to the corresponding affinity matrix. The affinity matrix may be a solid carrier comprising the structure having affinity for the affinity tag. Said structure may be Ni²⁺-NTA for the His-tag and streptavidin for the Strep-tag. Definition and examples for solid carriers are provided herein.

In one aspect the method for producing a fusion polypeptide in context of the present invention and collecting the produced fusion polypeptide may comprise
(i) incubating a first substrate polypeptide and a second substrate polypeptide each comprising an identical affinity tag in the portion not forming the desired fusion polypeptide with a polypeptide having transpeptidase activity comprising an affinity tag identical to the affinity tag of the substrate polypeptides
(ii) producing the fusion polypeptide
(iii) applying the reaction mixture to a corresponding affinity matrix
(iv) incubating the reaction mixture with said solid carrier allowing the affinity matrix to bind the affinity tags, and
(v) collecting the flow through comprising the desired produced fusion polypeptide.

It has to be pointed out that the affinity tags attached to the substrate polypeptides and to the polypeptide having transpeptidase may be not identical. The affinity matrices used have to be adapted accordingly.

The skilled is well aware how produced fusion polypeptides not comprising an affinity tag can be purified. For example, size-exclusion chromatography may be used if the produced fusion polypeptide differs substantially in size from the substrate polypeptide and the polypeptide having transpeptidase activity used. Furthermore, if the produced fusion polypeptide has a substantially different isoelectric point compared to the substrate polypeptides and the transpeptidase used the produced fusion polypeptide may be recovered via ion exchange chromatography. Further, it is clear that different purification strategies can be combined. For example, a transpeptidase containing an affinity tag may be removed from the reaction mixture and the residual reaction mixture may be applied to size-exclusion chromatography or ion exchange chromatography.

The invention also provides means and methods to generate and purify enzyme-substrate complexes comprising a protein of interest with the N-terminal portion of the DUF2121 recognition motif fused to the polypeptide of the present invention. The generation of these complexes may contain steps of
(i) obtaining a sample containing the protein of interest fused to the DUF2121 recognition motif.
(ii) immobilizing the polypeptide of the present invention on a solid carrier
(iii) incubating the sample containing the protein of interest fused to the DUF2121 recognition motif with the carrier-bound polypeptide of the present invention.
(iv) thereby producing a fusion polypeptide comprising the protein of interest with the N-terminal portion of the DUF2121 recognition motif fused to the polypeptide of the present invention
(v) washing the carrier with suitable buffers and thereby removing excess substrate containing the C-terminal portion of the DUF2121 recognition motif
(vi) optionally, removing the protein of interest with the N-terminal portion of the DUF2121 recognition motif fused to the polypeptide of the present invention from the solid carrier.

Suitable carriers are well known in the art. Non-limiting examples are polymers, a hydrogel, a microparticle, a nanoparticle, a sphere (e.g. a nano- or microsphere), beads (e.g. microbeads), quantum dots, prosthetics and a solid surface. In a preferred embodiment the carrier is a bead (e.g. a microbead), such as an agarose bead.

Such enzyme-substrate complexes may represent a ready-to-use reagent for producing a fusion polypeptide. In a preferred embodiment, a first protein of interest with the N-terminal portion of the DUF2121 recognition motif fused to the polypeptide of the present invention is brought in contact with a second protein of interest comprising the C-terminal portion of the DUF2121 recognition motif Thereby, a fusion polypeptide comprising the first protein of interest at the N-terminal end and the second protein of interest at the C-terminal end is generated.

The invention also provides means and methods to produce fusion polypeptides comprising non-proteinaceous moieties.

Accordingly, in the inventive method for producing a fusion polypeptide the portion of the first substrate polypeptide or the portion of the second substrate polypeptide forming part of the produced fusion polypeptide may comprise a non-proteinaceous moiety attached thereto so that the produced fusion polypeptide comprises said non-proteinaceous moiety. Furthermore, the portion of the first substrate polypeptide and the portion of the second substrate polypeptide forming part of the produced fusion polypeptide may comprise a non-proteinaceous moiety attached thereto so that the produced fusion polypeptide comprises said non-proteinaceous moieties. The non-proteinaceous moiety attached to the portion of the first substrate polypeptide forming part of the produced fusion polypeptide may be different or identical to the non-proteinaceous moiety attached to the portion of the second substrate polypeptide forming part of the produced fusion polypeptide. Accordingly, the produced fusion polypeptide may contain two different or two identical non-proteinaceous moieties.

The above mentioned non-proteinaceous moiety may be a fluorophore, a drug, a toxin, a carbohydrate, a lipid, a solid carrier, an oligonucleotide or a combination thereof. Non-limiting examples of said non-proteinaceous moieties are depicted in FIGS. 16 A, B and C.

Fluorophores are known in the art and are publicly and/or commercially available. Non-limiting examples are, e.g., fluorescein, FITC, Atto488 and Alexa488.

The term“drug” relates to medicinal or preventive agents. A drug may be a small molecule drug. Non-limiting examples of the small molecules are doxorubicin, calicheamicin, camptothecin, fumagillin, dexamethasone, geldanamycin, paclitaxel, docetaxel, irinotecan, cyclosporine, buprenorphine, naltrexone, naloxone, vindesine, vancomycin, risperidone, aripiprazole, palonosetron, granisetron, cytarabine, NX1838, leuprolide, goserelin, buserelin, octreotide, teduglutide, cilengitide, abarelix, enfuvirtide, ghrelin and derivatives, tubulysins and platin derivatives. The term “toxin” relates to agents which might have adverse effects on living organisms or cells.

Non-limiting examples of a solid carrier are described herein above.

The oligonucleotide may be a DNA, RNA or analogues of DNA or RNA made from nucleotide analogues.

It has to be pointed out that fluorophores are not limited to non-proteinaceous fluorophores but may also be fluorescent proteins. Non-limiting examples of fluorescent proteins are green fluorescent protein (GFP) or red fluorescent protein (RFP) or derivatives thereof.

Further it has to be noted that drugs that can be used in context of the present invention are not limited to small molecules but may also be or comprise biologically active peptides or proteins. Non-limiting examples for biologically active peptides or proteins are follicle-stimulating hormone, glucocerebrosidase, thymosin alpha 1, glucagon, somatostatin, adenosine deaminase, interleukin 11, hematide, leptin, interleukin-20, interleukin-22 receptor subunit alpha (IL-22ra), interleukin-22, hyaluronidase, fibroblast growth factor 18, fibroblast growth factor 21, glucagon-like peptide 1, osteoprotegerin, IL-18 binding protein, growth hormone releasing factor, soluble TACI receptor, thrombospondin-1, soluble VEGF receptor Flt-1, α-galactosidase A, myostatin antagonist, gastric inhibitory polypeptide, alpha-1 antitrypsin, IL-4 mutein, and the like.

In the method for producing a fusion polypeptide in context of the present invention the portion of the first substrate polypeptide or the portion of the second substrate polypeptide forming part of the produced fusion polypeptide may comprise an antibody, a domain or fragment thereof. It is envisaged herein to use the method of the present invention to create bispecific antibodies, hybrid antibodies or to couple other molecules like fluorophores or drugs to antibodies (FIG. 16 D).

An “antibody,” is used herein in the broadest sense, encompasses various antibody structures and can be any molecule that can specifically or selectively bind to a target protein. An antibody may include or be an antibody or a domain/fragment thereof, wherein the domain/fragment shows the substantially the same binding activity as the full-length antibody. Non-limiting examples are monoclonal antibodies, polyclonal antibodies, or multispecific antibodies (e.g., bispecific antibodies). Antibodies within the present invention may also be chimeric antibodies, recombinant antibodies, humanized antibodies or fully-human antibodies. Examples of antibody fragments include but are not limited to Fv, Fab, Fab′, Fab′ —SH, F(ab′)2.

Antibodies may also include multivalent molecules, multi-specific molecules (e.g., diabodies), fusion molecules, aptimers, avimers, or other naturally occurring or recombinantly created molecules. Illustrative antibodies useful in the present invention include antibody-like molecules. An antibody-like molecule is a molecule that can exhibit functions by binding to a target molecule (see for example Gill (2006) Curr Opin Biotechnol 17:653-658; Nygren (1997) Curr Opin Struct Biol 7:463-469; Hosse (2006) Protein Sci 15:14-27), and includes, for example, DARPins (WO 2002/020565), Affibody (WO 1995/001937), Avimer (WO 2004/044011; WO 2005/040229), Adnectin (WO 2002/032925) and fynomers (WO 2013/135588).

The present invention is also useful to fuse an enzyme to a polypeptide or to another enzyme. Accordingly, in the inventive method for producing a fusion polypeptide the portion of the first substrate polypeptide or the portion of the second substrate polypeptide forming part of the produced fusion polypeptide may comprise an enzyme attached thereto so that the produced fusion polypeptide comprises said enzyme. Furthermore, the portion of the first substrate polypeptide and the portion of the second substrate polypeptide forming part of the produced fusion polypeptide may comprise an enzyme attached thereto so that the produced fusion polypeptide comprises said enzymes. The enzyme attached to the portion of the first substrate polypeptide forming part of the produced fusion polypeptide may be different or identical to the enzyme attached to the portion of the second substrate polypeptide forming part of the produced fusion polypeptide. Accordingly, the produced fusion polypeptide may contain two different or two identical enzymes. In general, the term “enzyme” is used herein in the broadest sense and encompasses all macromolecules that are able to catalyze chemical reactions.

It is envisaged herein to use the method of the present invention to immobilize proteins on solid carriers. Accordingly, in the inventive method for producing a fusion polypeptide the portion of the first substrate polypeptide or the portion of the second substrate polypeptide forming part of the produced fusion polypeptide may comprise a protein and the portion of the other substrate polypeptide forming part of the produced fusion polypeptide may have a solid carrier attached thereto so that the produced fusion polypeptide comprises the protein immobilized on the solid carrier, preferably the protein may be an enzyme. It is understood by the skilled person that a solid carrier can contain several substrate polypeptides allowing to immobilize several protein molecules to the solid carrier. Accordingly, a solid carrier with several substrate polypeptides allows to immobilize different protein molecules to the solid carrier (FIG. 16 E).

It is also envisaged herein to use the method of the invention for covalent and/or geometrically defined attachment of proteins and/or protein complexes on surfaces/solid carriers for microscopy applications, e.g. electron microscopy, especially cryo-electron microscopy.

In the inventive method for producing a fusion polypeptide the first substrate polypeptide and the second substrate polypeptide may be isotopically labeled. Preferably, either the first or the second polypeptide may be isotopically labeled. Such segmentally labeled fusion polypeptides may be used in NMR experiments (FIG. 16 F).

The expression “isotopically labeled” is used herein in the broadest sense and may relate to non-radioactive (like [¹³C]carbon, [²H]deuterium, [¹⁵N]nitrogen) or radioactive labels (like [³H]hydrogen, [¹²⁵I]iodide or [¹²³I]iodide).

It was shown that strong immune reactions can be triggered by fusing an immunogenic structure to a virus like particle. However, genetically fusing an immunogenic structure of interest to the viral structural protein can lead to impaired virus like particle assembly. The present invention allows to circumvent said caveat. Thus, the present invention provides also means and methods to create highly immunogenic compounds. Accordingly, in the inventive method for producing a fusion polypeptide the portion of the first substrate polypeptide or the portion of the second substrate polypeptide forming part of the produced fusion polypeptide may be part of a virus-like particle and the portion of the other substrate polypeptide forming part of the produced fusion polypeptide may comprise an immunogenic structure.

Virus like particles are molecules that closely resemble viruses, but are non-infectious because they contain no viral genetic material. It is well known in the art how virus like particles can be produced (Zeltins (2013) Mol Biotechnol 53(1):92-107). The term “immunogenic structure” is used herein in the broadest sense and relates to all molecules that trigger any sort of immune response in a human or an animal.

The skilled person is well aware that the inventive method for producing a fusion polypeptide allows to fuse several different immunogenic structures to a virus like particle. The immunogenic structures may be an influenza antigen, a pox antigen, a SARS-CoV-2 antigen and a measles antigen (FIG. 16 G). The immunogenic compound depicted in FIG. 16 G may be used to vaccine an individuum against influenza, pox and measles simultaneously.

The present invention also provides means and methods to fuse polypeptides to membranes. In the inventive method for producing a fusion polypeptide the portion of the first substrate polypeptide or the portion of the second substrate polypeptide forming part of the produced fusion polypeptide may be comprised in a membrane, preferably a vesicle membrane. The substrate polypeptides may be anchored in the membrane for example by outer membrane protein A (FIG. 16 H).

It is clear for the skilled person that the inventive method for producing a fusion polypeptide may be performed with substrate polypeptides containing disulfide bonds. Accordingly, in the method for producing a fusion polypeptide in context of the present invention the first substrate polypeptide may comprise an intramolecular disulfide bond, preferably the first cysteine residue forming the disulfide bond is located N-terminally of the DUF2121 recognition sequence and the second cysteine residue forming the disulfide bond is located C-terminally of the DUF2121 recognition motif. FIG. 16 J depicts a potential reaction involving a substrate polypeptide containing a disulfide bond.

It can be useful that the fusion polypeptide produced by the method of the present invention contains an affinity tag. Accordingly, in the inventive method for producing a fusion polypeptide the portion of the first substrate polypeptide or the portion of the second substrate polypeptide forming part of the produced fusion polypeptide may comprise an affinity tag. In another aspect the portion of the first substrate polypeptide forming part of the produced fusion polypeptide may comprise a first affinity tag, and the portion of the second substrate polypeptide forming part of the produced fusion polypeptide may comprise a second affinity tag. Preferably the first and second affinity tags are different.

The skilled person is well aware how affinity chromatography can be used to purify the produced fusion polypeptides containing affinity tags. Examples for affinity tags and corresponding affinity matrices are described herein.

It is also envisaged that the polypeptide of the present invention and the method for producing a fusion polypeptide can be used in protein purification (FIG. 16 K). Specifically, the N-terminal portion of a protein of interest containing a DUF2121 recognition sequence as described herein may be purified.

Accordingly, said protein purification may comprise the steps of:
(i) immobilizing the polypeptide of the present invention to a column resin
(ii) contacting the polypeptide of the present invention immobilized to a column resin with the protein of interest containing a DUF2121 recognition motif as described herein
(iii) forming a covalent bond between the catalytic serine or threonine residue of the polypeptide of the present invention and the N-terminal portion of the protein of interest defined from the N-terminus of the protein of interest to the aspartate residue in position 2 of SEQ ID NO: 308, 309, 310 and 311, respectively
(iv) eluting the N-terminal portion of the protein of interest by applying to the column an elution polypeptide containing N-terminally the C-terminal portion of the DUF2121 recognition motif as described herein
(v) collecting the fusion polypeptide containing the N-terminal portion of the protein of interest and the elution polypeptide.

Further it is envisaged herein that catalytically inactive variants of the polypeptide of the present invention may be used in protein purification (FIG. 16 L). Specifically, proteins containing a DUF2121 recognition motif as described herein or the C-terminal portion of DUF2121 domain as described herein can be purified using the catalytically inactive variant of the polypeptide of the present invention. Said catalytically inactive variant may be a polypeptide of the present invention in which the catalytic serine or threonine residue is exchanged by another amino acid, preferably the catalytic residue is exchanged by alanine.

Accordingly, said protein purification using catalytically inactive variants of the polypeptide of the present invention may comprise the steps of:
(i) immobilizing the catalytically inactive polypeptide of the present invention to a column resin
(ii) contacting the polypeptide of the present invention immobilized to a column resin with the protein of interest containing a DUF2121 recognition motif as described herein or N-terminally a C-terminal portion of the DUF2121 recognition motif as described herein
(iii) forming a non-covalent interaction between the catalytically inactive polypeptide of the present invention and the protein of interest
(iv) eluting the protein of interest by applying to the column a polypeptide containing N-terminally the C-terminal portion of the DUF2121 recognition motif as described herein
(v) collecting the eluted protein of interest.

The skilled person is well aware which material can be used as column resin. Basically, material as described for the term “solid carrier” may be used as column resin.

It is further envisaged that the polypeptide of the present invention and the method for producing a fusion polypeptide of the present invention are used to obviate the need for antibodies for detection of proteins in Western Blotting (FIG. 16 M).

Accordingly, the said protein detection may contain the steps of
(i) transferring the protein to be detected containing N-terminally the C-terminal portion of the DUF2121 recognition motif as described herein to a membrane
(ii) incubating the membrane with the polypeptide of the present invention and a reporter polypeptide containing a DUF2121 recognition motif as described herein and a detectable marker N-terminally of the DUF2121 recognition motif
(iii) thereby producing a fusion polypeptide comprising the N-terminal portion of the reporter polypeptide comprising the detectable marker and the protein to be detected
(iv) detecting said fusion polypeptide via the detectable marker.

It also further envisaged that the polypeptide of the present invention and the method for producing a fusion polypeptide of the present invention are used to detect proteins in a complex mixture. The detection of said protein may contain the steps of
(i) obtaining a sample containing the protein of interest fused to the DUF2121 recognition motif Preferentially, the C-terminal portion of the DUF2121 recognition motif should be fused N-terminally of the protein of interest
(ii) incubating this sample with the polypeptide of the present invention and a reporter polypeptide containing a DUF2121 recognition motif as described herein and a detectable marker, preferentially N-terminally of the DUF2121 recognition motif
(iii) thereby producing a fusion polypeptide comprising the reporter polypeptide with the detectable marker and the protein to be detected
(iv) separating the proteins within the sample via SDS-PAGE. Optionally, the proteins can be transferred to a membrane in a second step
(v) detecting said fusion polypeptide in the gel or on the membrane via the detectable marker.

Furthermore, a second method is envisaged, in which the polypeptide of the present invention and the method for producing a fusion polypeptide of the present invention are used to detect proteins in a complex mixture. The detection of said protein may contain the steps of
(i) obtaining a sample containing the protein of interest fused to the DUF2121 recognition motif Preferentially, the C-terminal portion of the DUF2121 recognition motif should be fused N-terminally of the protein of interest
(ii) immobilizing the sample containing the protein of interest fused to the DUF2121 recognition motif on a microplate.
(iii) incubating said sample with the polypeptide of the present invention and a reporter polypeptide containing a DUF2121 recognition motif as described herein and a detectable marker, preferentially N-terminally of the DUF2121 recognition motif
(iv) thereby producing a fusion polypeptide comprising the reporter polypeptide with the detectable marker and the protein to be detected
(v) detecting said fusion polypeptide on the microplate via the detectable marker.

It also further envisaged that the polypeptide of the present invention and the method for producing a fusion polypeptide of the present invention are used to detect recombinant proteins containing a DUF2121 recognition motif. The skilled person is aware how to use routine methods to produce the desired recombinant protein with the DUF2121 recognition motif. Suitable techniques for the genetic introduction of the DUF2121 motif are well known in the art. Non-limiting examples include gene delivery through viruses, CaCl₂), liposomes, heat shock, electroporation or microinjection and gene editing using restriction enzymes, homologous recombination, CRISPR/Cas9, TALEN, Zinc finger and meganucleases. Specifically, the generation of cells capable of producing antibodies containing a DUF2121 recognition motif is envisaged. The skilled person is aware how these cells can be produced by routine methods. The skilled person is also aware how to use routine methods to select for cells capable of producing antibodies that contain a DUF2121 recognition motif and recognize the antigen of interest. The detection of such antigens may comprise steps of:
(i) incubating the antibodies bearing the DUF2121 recognition motif with the polypeptide of the present invention and a detectable maker bearing the DUF2121 recognition motif
(ii) thereby producing antibodies fused to the detectable maker
(iii) immobilizing the antigen of interest on a carrier. Non-limiting examples are microplates, PVDF or nitrocellulose blotting membranes.
(iv) bringing the antigen of interest in contact with antibodies bearing the DUF2121 recognition motif and the detectable maker
(v) detecting the antigen levels via the detectable maker

Depending on the methods used, a detectable marker may comprise a reporter enzyme, a fluorophore and/or a radioactive isotope. Suitable reporter enzymes and how to detect them are well known in the art. Non-limiting examples are alkaline phosphatase, horseradish peroxidase or luciferase enzymes. Suitable fluorophores and how to detect them are well known in the art. Non-limiting examples are Alexa Fluors, Bodipy dyes, Qdot probes, Fluorescein derivatives, fluorescent proteins, cyanine fluorophores or IRDyes, such as Alexa Fluor 750, Cy 7.5, Cy 5.5 or IRDye 800 fluorophores. Said fluorophores may be detected via fluorescence imaging. Suitable radioactive isotopes and how to detect them are well known in the art. Non-limiting examples are [³²P]phosphorus, [³³P]phosphorus [³⁵S]sulfur, [³H]hydrogen, [¹²⁵I]iodide, [¹²³I]iodide, or [¹³¹I]iodide.

It is also envisaged that the polypeptide of the present invention is used for production of a circular polypeptide (FIG. 16 I). Circular polypeptides are exceptionally useful in therapeutic applications, due to their increased stability (van 't Hof (2015) Biol Chem 396:283-93).

Accordingly, the present invention further relates to a method for producing a circular polypeptide comprising producing the circular polypeptide by bringing the polypeptide of the present invention into contact with a substrate polypeptide and reacting the substrate polypeptide.

Said method may further comprise producing a circular polypeptide.

In the inventive method for producing a circular polypeptide circularization may be generated between via the formation of a peptide bond between two residues of the substrate polypeptide.

The circularization of the substrate polypeptide may be generated by a peptide bond between residues of two DUF2121 recognition motifs.

Accordingly, in the inventive method for producing a circular polypeptide the substrate polypeptide may comprise two DUF2121 recognition motifs in a distance sufficient to allow circularization of the sequence.

In the inventive method for producing a circular polypeptide the circularization of the substrate polypeptide may be generated via the formation of a peptide bond between the proline residue of the first DUF2121 recognition motif in position 3 of SEQ ID NOs: 308, 309, 310 and 311, respectively, and the aspartate residue of the second DUF2121 recognition motif in position 2 of SEQ ID NOs: 308, 309, 310 and 311, respectively.

The circularization of the substrate polypeptide may be generated by a peptide bond between the N-terminal amino acid and an amino acid of an internal DUF2121 recognition motif.

In the inventive method for producing a circular polypeptide the substrate polypeptide may comprise at its N-terminus the C-terminal portion of the DUF2121 recognition motif, said C-terminal portion of the DUF2121 recognition motif starting with the amino acid residues as defined in positions 3 to 5 of any one of SEQ ID NOs: 308, 309, 310 and 311 and further a DUF2121 recognition motif comprising any one of SEQ ID NOs: 308, 309, 310 and 311 in a distance to the N-terminus sufficient to allow circularization

In the inventive method for producing a circular polypeptide the substrate polypeptide may comprise at its N-terminus the C-terminal portion of the DUF2121 recognition motif, said C-terminal portion of the DUF2121 recognition motif starting with the amino acid residues as defined in positions 3 to 5 of any one of SEQ ID NOs: 308, 309, 310 and 311 and further a DUF2121 recognition motif comprising any one of SEQ ID NOs: 308, 309, 310 and 311 in a distance to the N-terminus sufficient to allow circularization and the circularization of the substrate polypeptide may be generated via the formation of a peptide bond between the N-terminal amino acid and the aspartate residue of the DUF2121 recognition motif in position 2 of SEQ ID NOs: 308, 309, 310 and 311, respectively.

When the inventive method provided herein is used to produce a circular polypeptide the DUF2121 recognition motif may comprise additional amino acids.

Accordingly, in the inventive method for producing a circular polypeptide the DUF2121 recognition motif may comprise at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids N-terminally of SEQ ID NOs: 308, 309, 310 and 311, respectively.

In the inventive method for producing a circular polypeptide the DUF2121 recognition motif may comprise at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids C-terminally of SEQ ID NOs: 308, 309, 310 and 311, respectively.

As mentioned above a DUF2121 recognition motif comprising at least 20 amino acids C-terminally of SEQ ID NOs: 308, 309, 310 and 311, respectively, may be particularly preferred.

Thus, in the inventive method for producing a circular polypeptide the DUF2121 recognition motif may comprise at least 10, at least 15 or at least 20 amino acids C-terminally of SEQ ID NOs: 308, 309, 310 and 311, respectively.

In the inventive method for producing a circular polypeptide the DUF2121 recognition motif may comprise at least 5 amino acids N-terminally and at least 10 amino acids C-terminally of SEQ ID NOs: 308, 309, 310 and 311, respectively.

In the inventive method for producing a circular polypeptide the DUF2121 recognition motif may comprise at least 5 amino acids N-terminally and at least 15 amino acids C-terminally of SEQ ID NOs: 308, 309, 310 and 311, respectively.

In the inventive method for producing a circular polypeptide the DUF2121 recognition motif may comprise at least 5 amino acids N-terminally and at least 20 amino acids C-terminally of SEQ ID NOs: 308, 309, 310 and 311, respectively.

In the inventive method for producing a circular polypeptide the DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15 or 15 ofany one of SEQ ID NOs: 315-366, 460-510 and 551-661 N-terminally, preferably directly N-terminally of SEQ ID NOs: 308, 309, 310 and 311, respectively.

In the inventive method for producing a circular polypeptide DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661C-terminally, preferably directly C-terminally of SEQ ID NOs: 308, 309, 310 and 311, respectively.

It is also possible in the inventive method for producing a circular polypeptide that the DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of SEQ ID NOs: 308, 309, 310 and 311, respectively.

Thus, in the inventive method for producing a circular polypeptide DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661C-terminally, preferably directly C-terminally of SEQ ID NOs: 308, 309, 310 and 311, respectively, and/or may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of SEQ ID NOs: 308, 309, 310 and 311, respectively.

In the inventive method for producing a circular polypeptide the DUF2121 recognition motif may consist of the sequence as defined in any one of SEQ ID NOs: 315-366, 460-510 and 551-661 or a sequence having or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

In the inventive method for producing a circular polypeptide the C-terminal portion of the DUF2121 recognition motif may comprise at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids C-terminally, preferably directly C-terminally of the N-terminal amino acids as defined by positions 3 to 5 of SEQ ID NOs: 308, 309, 310 and 311, respectively.

A C-terminal portion of the DUF2121 recognition motif comprising at least 20 amino acids C-terminally, preferably directly C-terminally of the N-terminal amino acids as defined by positions 3 to 5 of SEQ ID NOs: 308, 309, 310 and 311, respectively, may be particularly preferred.

Thus, in the inventive method for producing a circular polypeptide the C-terminal portion of the DUF2121 recognition motif may comprise at least 10, at least 15 or at least 20 amino acids C-terminally, preferably directly C-terminally of the N-terminal amino acids as defined by positions 3 to 5 of SEQ ID NOs: 308, 309, 310 and 311, respectively.

In the inventive method for producing a circular polypeptide the C-terminal portion of the DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally, preferably directly C-terminally of the N-terminal amino acids as defined by positions 3 to 5 of SEQ ID NOs: 308, 309, 310 and 311, respectively.

It is also envisaged in the inventive method for producing a circular polypeptide that the C-terminal portion of the DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to31 ofany one of SEQ ID NOs: 551-661 C-terminally, preferably directly C-terminally of the N-terminal amino acids as defined by positions 3 to 5 of SEQ ID NOs: 308, 309, 310 and 311, respectively.

Thus, the inventive method for producing a circular polypeptide the C-terminal portion of the DUF2121 recognition motif may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally, preferably directly C-terminally of the N-terminal amino acids as defined by positions 3 to 5 of SEQ ID NOs: 308, 309, 310 and 311, respectively, and/or may comprise a sequence identical to or at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally, preferably directly C-terminally of the N-terminal amino acids as defined by positions 3 to 5 of SEQ ID NOs: 308, 309, 310 and 311, respectively.

In the inventive method for producing a circular polypeptide the C-terminal portion of the DUF2121 recognition motif may consist of the amino acid sequence as defined in positions 16 to 30 of any one of SEQ ID NOs: 315-366, 460-510 and 551-661 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

It is also envisaged in the inventive method for producing a circular polypeptide that the C-terminal portion of the DUF2121 recognition motif may consist of the amino acid sequence as defined in positions 16 to 35 of any one of SEQ ID NOs: 551-661 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

It is also envisaged in the inventive method for producing a circular polypeptide that the C-terminal portion of the DUF2121 recognition motif may consist of the amino acid sequence as defined in positions 16 to 40 of any one of SEQ ID NOs: 551-661 or an amino acid sequence having at least 20%, preferably at least 30%, even more preferably at least 40%, even more preferably at least 50%, even more preferably at least 60%, even more preferably at least 70%, even more preferably at least 80%, even more preferably at least 90%, even more preferably at least 95% and most preferably at least 99% sequence identity to said sequence.

The invention further relates to the fusion polypeptides obtained or obtainable by the method for producing a fusion polypeptide as described herein and to the circular polypeptide obtained or obtainable by the method for producing a circular polypeptide as described herein.

Of course, the invention also relates to the isolated fusion polypeptides obtained or obtainable by the method for producing a fusion polypeptide as described herein and to the isolated circular polypeptide obtained or obtainable by the method for producing a circular polypeptide as described herein.

The fusion polypeptides and the circular polypeptides produced by the methods of the present invention can be used for pharmaceutical or diagnostical purposes. Accordingly, the invention relates to the use of said fusion polypeptide or circular polypeptide in the prevention, treatment or amelioration of a disease. In other words, the invention relates to the use of said fusion polypeptide or circular polypeptide as a medicament. It is also envisaged that the fusion polypeptides or the circular polypeptides produced by the present invention form part of a composition. Said composition may comprise one or more of the fusion polypeptides or the circular polypeptide produced by the present invention. Said composition may be a pharmaceutical composition optionally further comprising a pharmaceutically acceptable carrier and/or diluent. Said composition may be used for pharmaceutical or diagnostical purposes. Accordingly, the invention relates to the use of a pharmaceutical composition comprising the fusion polypeptide or circular polypeptide produced by the invention in the prevention, treatment or amelioration of a disease. In other words the invention relates to the use of a pharmaceutical composition comprising the fusion polypeptide or circular polypeptide produced by the invention as a medicament. However, the fusion polypeptides produced by the methods of the present invention are also useful in certain other industrial areas, like in the food industry, the beverage industry, the cosmetic industry, the oil industry, the paper industry and the like.

Fusion polypeptides are interesting for pharmaceutical or diagnostical purposes because of their ability to extend protein and peptide drug lifetimes. By fusing biologically active proteins or peptides with a long half-life protein, the resulting fusion protein can have a significantly longer lifetime than that of the original drug. It is envisaged that the inventive polypeptide and the inventive method described herein may be used to create fusion proteins including but not limited to fusions of a pharmaceutically active protein/polypeptide with an antibody Fc fragment, with recombinant serum albumin, with transferrin, with carboxy-terminal peptide, with XTEN or elastin-like-peptide.

TABLE 1
Adriase proteins, their recognition motifs and organismal growth conditions.

| | | | | Secondary* | Secondary* | | | | |
|---|---|---|---|---|---|---|---|---|---|
| | | Adriase | Adriase | Adriase | Adriase | | | | |
| | | 15 × 10 | 15 × 20 | 15 × 10 | 15 × 20 | Max. | Optimal | | |
| | Adriase | recognition | recognition | recognition | recognition | growth | growth | Optimal | Additional |
| | SEQ | motif SEQ | motif SEQ | motif SEQ | motif SEQ | temperature | NaCl | growth | growth |
| Species | ID NO | ID NO | ID NO | ID NO | ID NO | [°C] | [mM] | pH | requirements |
| Candidatus | 99 | 324 | 551 | — | | n.d. | n.d. | n.d. | n.d. |
| Methanoperedens sp. | | | | | | | | | |
| BLZ2 | | | | | | | | | |
| Methanobacteriaceae | 215 | 364 | 552 | — | | n.d. | n.d. | n.d. | n.d. |
| archaeon 41_258 | | | | | | | | | |
| Methanobacteriales | 219 | 463 | 553 | — | | n.d. | n.d. | n.d. | n.d. |
| archaeon HGW- | | | | | | | | | |
| Methanobacteriales-1 | | | | | | | | | |
| Methanobacteriales | 195 | 485 | 554 | — | | n.d. | n.d. | n.d. | n.d. |
| archaeon HGW- | | | | | | | | | |
| Methanobacteriales-2 | | | | | | | | | |
| Methanobacterium | 211 | 474 | 555 | — | | n.d. | n.d. | n.d. | n.d. |
| arcticum | | | | | | | | | |
| Methanobacterium | 210 | 474 | 555 | 475 | 649 | 50 | 0.00 | 6.8-7.2 | n.d. |
| bryantii | | | | | | | | | |
| Methanobacterium | 217 | 496 | 556 | — | | 50 | n.d. | 6.3-6.8 | n.d. |
| congolense | | | | | | | | | |
| Methanobacterium | 199 | 469 | 557 | — | | 50 | n.d. | 6.9-7.4 | n.d. |
| formicicum | | | | | | | | | |
| Methanobacterium | 203 | 469 | 557 | — | | 50 | n.d. | 6.9-7.4 | n.d. |
| formicicum | | | | | | | | | |
| (GCF_001458655.1) | | | | | | | | | |
| Methanobacterium | 202 | 469 | 557 | — | | 50 | n.d. | 6.9-7.4 | n.d. |
| formicicum BRM9 | | | | | | | | | |
| (GCA_000762265.1) | | | | | | | | | |
| Methanobacterium | 201 | 469 | 557 | — | | 50 | n.d. | 6.9-7.4 | n.d. |
| formicicum BRM9 | | | | | | | | | |
| (GCF_000762265.1) | | | | | | | | | |
| Methanobacterium | 207 | 469 | 643 | — | | 50 | n.d. | 6.9-7.4 | n.d. |
| formicicum DSM 3637 | | | | | | | | | |
| Methanobacterium | 200 | 469 | 557 | — | | 50 | n.d. | 6.9-7.4 | n.d. |
| formicicum DSM1535 | | | | | | | | | |
| Methanobacterium | 204 | 469 | 557 | — | | 50 | n.d. | 6.9-7.4 | n.d. |
| formicicum MB9 | | | | | | | | | |
| Methanobacterium | 193 | 358 | 558 | — | | 41 | n.d. | 7 | acetate, yeast |
| lacus | | | | | | | | | extract, |
| | | | | | | | | | trypticase |
| Methanobacterium | 216 | 360 | 559 | — | | n.d. | n.d. | n.d. | n.d. |
| paludis | | | | | | | | | |
| Methanobacterium sp. | 209 | 492 | 560 | — | | n.d. | n.d. | n.d. | n.d. |
| Methanobacterium sp. | 218 | 472 | 561 | — | | n.d. | n.d. | n.d. | n.d. |
| BAmetb10 | | | | | | | | | |
| Methanobacterium sp. | 205 | 469 | 562 | — | | n.d. | n.d. | n.d. | n.d. |
| BAmetb5 | | | | | | | | | |
| Methanobacterium sp. | 212 | 464 | 563 | — | | n.d. | n.d. | n.d. | n.d. |
| BRmetb2 | | | | | | | | | |
| Methanobacterium sp. | 206 | 465 | 554 | 466 | 646 | n.d. | n.d. | n.d. | n.d. |
| Maddingley MBC34 | | | | | | | | | |
| Methanobacterium sp. | 198 | 469 | 564 | — | | n.d. | n.d. | n.d. | n.d. |
| MB1 | | | | | | | | | |
| (GCA_000499765.1) | | | | | | | | | |
| Methanobacterium sp. | 197 | 469 | 564 | — | | n.d. | n.d. | n.d. | n.d. |
| MB1 | | | | | | | | | |
| (GCF_000499765.1) | | | | | | | | | |
| Methanobacterium sp. | 194 | 465 | 565 | 467 | 647 | n.d. | n.d. | n.d. | n.d. |
| PtaB.Bin024 | | | | | | | | | |
| Methanobacterium sp. | 208 | 461 | 566 | 462 | 645 | n.d. | n.d. | n.d. | n.d. |
| PtaU1.Bin097 | | | | | | | | | |
| Methanobacterium sp. | 192 | 357 | 567 | — | | n.d. | n.d. | n.d. | n.d. |
| SMA-27 | | | | | | | | | |
| Methanobacterium | 196 | 465 | 554 | 468 | 648 | 40 | 0.00 | 7 | n.d. |
| subterraneum | | | | | | | | | |
| Methanobrevibacter | 184 | 365 | 568 | — | | 45 | n.d. | 7 | acetate, yeast |
| arboriphilus | | | | | | | | | extract, |
| | | | | | | | | | trypticase |
| Methanobrevibacter | 185 | 473 | 569 | — | | 45 | n.d. | 7 | acetate, yeast |
| arboriphilus DSM 1125 | | | | | | | | | extract, |
| | | | | | | | | | trypticase |
| Methanobrevibacter | 175 | 510 | 570 | — | | n.d. | n.d. | 7 | acetate, yeast |
| boviskoreani | | | | | | | | | extract, |
| | | | | | | | | | trypticase, |
| | | | | | | | | | coenzyme M, |
| | | | | | | | | | branched-chain |
| Methanobrevibacter | 171 | 359 | 571 | — | | 30 | 0.17 | 7.2 | acetate |
| curvatus | | | | | | | | | |
| Methanobrevibacter | 178 | 460 | 572 | — | | 33.5 | 0.86 | 7.6 | acetate |
| filiformis | | | | | | | | | |
| Methanobrevibacter | 189 | 479 | 573 | — | | n.d. | 0.05 | 7-7.2 | n.d. |
| gottschalkii | | | | | | | | | |
| Methanobrevibacter | 187 | 353 | 574 | — | | 43 | 1.4-1.9 | 9-9.5 | n.d. |
| millerae | | | | | | | | | |
| Methanobrevibacter | 172 | 361 | 575 | — | | 42 | 0.17 | 7 | acetate |
| olleyae | | | | | | | | | |
| Methanobrevibacter | 188 | 353 | 576 | — | | 39 | 0.09 | 6.7 | acetate |
| oralis | | | | | | | | | |
| Methanobrevibacter | 173 | 363 | 577 | — | | n.d. | 0.51 | 6.5 | n.d. |
| ruminantium | | | | | | | | | |
| Methanobrevibacter | 179 | 352 | 578 | — | | n.d. | 0.51 | 6.5 | n.d. |
| smithii | | | | | | | | | |
| Methanobrevibacter | 181 | 352 | 578 | — | | n.d. | 0.51 | 6.5 | n.d. |
| smithii ATCC 35061 | | | | | | | | | |
| Methanobrevibacter | 180 | 352 | 578 | — | | n.d. | 0.51 | 6.5 | n.d. |
| smithii CAG: 186 | | | | | | | | | |
| Methanobrevibacter | 182 | 352 | 578 | — | | n.d. | 0.51 | 6.5 | n.d. |
| smithii DSM 2374 | | | | | | | | | |
| Methanobrevibacter sp. | 177 | 362 | 579 | — | | n.d. | n.d. | n.d. | n.d. |
| 87.7 | | | | | | | | | |
| Methanobrevibacter sp. | 174 | 510 | 570 | — | | n.d. | n.d. | n.d. | n.d. |
| AbM4 | | | | | | | | | |
| Methanobrevibacter sp. | 183 | 365 | 580 | — | | n.d. | n.d. | n.d. | n.d. |
| NOE | | | | | | | | | |
| Methanobrevibacter sp. | 191 | 351 | 581 | 477 | 652 | n.d. | n.d. | n.d. | n.d. |
| YE315 | | | | | | | | | |
| Methanobrevibacter | 190 | 351 | 582 | 477 | 651 | n.d. | n.d. | 6 | rumen fluid, |
| thaueri | | | | | | | | | acetate, |
| | | | | | | | | | aminoacids |
| Methanobrevibacter | 186 | 354 | 583 | — | | n.d. | n.d. | 7.5-8 | Vitamins |
| woesei | | | | | | | | | |
| Methanobrevibacter | 176 | 362 | 584 | 476 | 650 | n.d. | 0.43 | 6.5 | n.d. |
| wolinii | | | | | | | | | |
| Methanocaldococcus | 160 | 505 | 585 | — | | n.d. | n.d. | n.d. | n.d. |
| bathoardescens | | | | | | | | | |
| Methanocaldococcus | 157 | 348 | 586 | — | | 92 | 0.50 | 6 | NaCl, Na2S, |
| fervens AG86 | | | | | | | | | |
| Methanocaldococcus | 154 | 343 | 587 | — | | 91 | 0.43 | 6.5 | n.d. |
| infernus ME | | | | | | | | | |
| Methanocaldococcus | 159 | 346 | 588 | — | | 85 | 0.43 | 6.5 | n.d. |
| jannaschii DSM 2661 | | | | | | | | | |
| Methanocaldococcus | 158 | 344 | 589 | — | | n.d. | n.d. | n.d. | n.d. |
| sp. FS406-22 | | | | | | | | | |
| Methanocaldococcus | 155 | 347 | 590 | — | | 90 | 0.00 | 7 | acetate |
| villosus KIN24-T80 | | | | | | | | | |
| Methanocaldococcus | 156 | 345 | 591 | — | | 89 | 0.00 | 6.8 | acetate |
| vulcanius M7 | | | | | | | | | |
| Methanococcales | 150 | 478 | 592 | — | | n.d. | n.d. | n.d. | n.d. |
| archaeon HHB | | | | | | | | | |
| Methanococcus | 147 | 340 | 593 | 504 | 660 | 50 | n.d. | 7 | acetate |
| aeolicus Nankai-3 | | | | | | | | | |
| Methanococcus | 165 | 341 | 594 | — | | 85 | 0.3-0.4 | 6.3-7.5 | n.d. |
| maripaludis | | | | | | | | | |
| Methanococcus | 162 | 480 | 595 | — | | 85 | 0.3-0.4 | 6.3-7.5 | n.d. |
| maripaludis C5 | | | | | | | | | |
| Methanococcus | 163 | 501 | 596 | 502 | 659 | 85 | 0.3-0.4 | 6.3-7.5 | n.d. |
| maripaludis C6 | | | | | | | | | |
| Methanococcus | 164 | 499 | 597 | 500 | 658 | 85 | 0.3-0.4 | 6.3-7.5 | n.d. |
| maripaludis C7 | | | | | | | | | |
| Methanococcus | 169 | 341 | 594 | — | | 85 | 0.3-0.4 | 6.3-7.5 | n.d. |
| maripaludis KA1 | | | | | | | | | |
| Methanococcus | 168 | 341 | 594 | — | | 85 | 0.3-0.4 | 6.3-7.5 | n.d. |
| maripaludis OS7 | | | | | | | | | |
| Methanococcus | 167 | 503 | 598 | — | | 85 | 0.3-0.4 | 6.3-7.5 | n.d. |
| maripaludis S2 | | | | | | | | | |
| Methanococcus | 166 | 341 | 594 | — | | 85 | 0.3-0.4 | 6.3-7.5 | n.d. |
| maripaludis X1 | | | | | | | | | |
| Methanococcus | 161 | 349 | 599 | — | | n.d. | n.d. | n.d. | n.d. |
| vannielii SB | | | | | | | | | |
| Methanococcus voltae | 146 | 339 | 600 | — | | 45 | 0.24-0.64 | 7-7.5 | n.d. |
| A3 | | | | | | | | | |
| Methanoculleus | 130 | 319 | 601 | — | | 50 | 0.00 | 7 | n.d. |
| bourgensis | | | | | | | | | |
| Methanoculleus | 129 | 319 | 601 | 494 | 657 | 50 | 0.00 | 7 | n.d. |
| bourgensis MAB1 | | | | | | | | | |
| Methanoculleus | 128 | 319 | 601 | — | | 50 | 0.00 | 7 | n.d. |
| bourgensis MS2 | | | | | | | | | |
| Methanoculleus | 138 | 482 | 602 | — | | 40 | 0.00 | 7 | acetate, yeast |
| chikugoensis | | | | | | | | | extract, peptone, |
| | | | | | | | | | heavy metal |
| | | | | | | | | | solution |
| Methanoculleus | 139 | 316 | 603 | — | | 45 | 0.3-0.5 | n.d. | acetate, yeast |
| horonobensis | | | | | | | | | extract |
| Methanoculleus | 136 | 317 | 604 | — | | 48 | 0.46 | 6.8-7.2 | yeast extract, |
| marisnigri JR1 | | | | | | | | | acetate, |
| Methanoculleus | 135 | 317 | 605 | — | | 50 | n.d. | 7.5-7.9 | acetate |
| sediminis | | | | | | | | | |
| Methanoculleus sp. | 131 | 495 | 606 | — | | n.d. | n.d. | n.d. | n.d. |
| MAB1 | | | | | | | | | |
| (GCA_900036045.1) | | | | | | | | | |
| Methanoculleus sp. | 137 | 317 | 605 | 484 | 653 | n.d. | n.d. | n.d. | n.d. |
| MH98A | | | | | | | | | |
| Methanoculleus sp. | 98 | 332 | 607 | 486 | 654 | n.d. | n.d. | n.d. | n.d. |
| SDB | | | | | | | | | |
| Methanoculleus | 96 | 488 | 608 | 489 | 656 | n.d. | n.d. | n.d. | n.d. |
| taiwanensis | | | | | | | | | |
| Methanoculleus | 127 | 318 | 609 | — | | 65 | 0.4-1.1 | 6-6.6 | acetate, |
| thermophilus | | | | | | | | | thiamine, |
| | | | | | | | | | riboflavin, |
| | | | | | | | | | vitamin B12, |
| | | | | | | | | | peptones |
| Methanofervidicoccus | 149 | 478 | 592 | — | | n.d. | n.d. | n.d. | n.d. |
| abyssi HHB | | | | | | | | | |
| Methanofollis | 97 | 315 | 610 | — | | 40 | 0.34 | 6.4-7.3 | acetate/ethanol, |
| ethanolicus | | | | | | | | | aminobenzoate, |
| | | | | | | | | | biotin, B12, |
| | | | | | | | | | tungsten |
| Methanolinea sp. SDB | 126 | 317 | 611 | — | | n.d. | n.d. | n.d. | n.d. |
| Methanolinea tarda | 142 | 320 | 612 | — | | 55 | n.d. | n.d. | n.d. |
| Methanomicrobiales | 134 | n.d. | — | — | | n.d. | n.d. | n.d. | n.d. |
| archaeon HGW- | | | | | | | | | |
| Methanomicrobiales2 | | | | | | | | | |
| Methanomicrobiales | 133 | n.d. | — | — | | n.d. | n.d. | n.d. | n.d. |
| archaeon HGW- | | | | | | | | | |
| Methanomicrobiales-6 | | | | | | | | | |
| Methanopyrus kandleri | 145 | 338 | 613 | — | | 110 | 0.50 | 7 | n.d. |
| AV19 | | | | | | | | | |
| Methanopyrus sp. | 144 | 338 | 613 | — | | n.d. | n.d. | n.d. | n.d. |
| KOL6 | | | | | | | | | |
| Methanoregulaceae | 143 | n.d. | — | — | | n.d. | n.d. | n.d. | n.d. |
| archaeon PtaB.Bin152 | | | | | | | | | |
| Methanoregulaceae | 141 | 481 | 614 | — | | n.d. | n.d. | n.d. | n.d. |
| archaeon PtaU1.Bin059 | | | | | | | | | |
| Methanoregulaceae | 140 | n.d. | — | — | | n.d. | n.d. | n.d. | n.d. |
| archaeon PtaU1.Bin066 | | | | | | | | | |
| Methanosaeta | 95 | 321 | 615 | — | | 45 | 0.6-2.5 | 6.5-7.4 | n.d. |
| harundinacea | | | | | | | | | |
| Methanosaeta sp. | 88 | n.d. | — | — | | n.d. | n.d. | n.d. | n.d. |
| ASM2 | | | | | | | | | |
| Methanosaeta sp. | 87 | n.d. | — | — | | n.d. | n.d. | n.d. | n.d. |
| ASO1 | | | | | | | | | |
| Methanosaeta sp. | 89 | n.d. | — | — | | n.d. | n.d. | n.d. | n.d. |
| NSM2 | | | | | | | | | |
| Methanosaeta sp. | 94 | 509 | 616 | — | | n.d. | n.d. | n.d. | n.d. |
| PtaB.Bin087 | | | | | | | | | |
| Methanosaeta sp. | 91 | 491 | 617 | — | | n.d. | n.d. | n.d. | n.d. |
| PtaU1.Bin112 | | | | | | | | | |
| Methanosaeta sp. SDB | 93 | n.d. | — | | | n.d. | n.d. | n.d. | n.d. |
| Methanosarcina | 104 | 335 | 618 | — | | 50 | 2-2.5 | 7.5 | n.d. |
| acetivorans C2A | | | | | | | | | |
| Methanosarcina | 118 | 329 | 619 | — | | n.d. | 0.5-2 | 6.5-7.5 | n.d. |
| barkeri CM1 | | | | | | | | | |
| Methanosarcina | 119 | 493 | 620 | — | | n.d. | 0.5-2 | 6.5-7.5 | n.d. |
| barkeri 3 | | | | | | | | | |
| Methanosarcina | 120 | 329 | 619 | — | | n.d. | 0.5-2 | 6.5-7.5 | n.d. |
| barkeri str. Fusaro | | | | | | | | | |
| Methanosarcina | 115 | 326 | 621 | — | | 50 | 0.15 | 7 | acetate |
| flavescens | | | | | | | | | |
| Methanosarcina | 102 | 336 | 622 | 487 | 655 | 42 | 0.00 | 7 | n.d. |
| horonobensis | | | | | | | | | |
| Methanosarcina | 103 | 337 | 623 | — | | 35 | 0.00 | 7 | acetate, yeast |
| lacustris Z-7289 | | | | | | | | | extract |
| Methanosarcina mazei | 106 | 328 | 624 | — | | 45 | 0.50 | 7.2 | n.d. |
| Methanosarcina mazei | 107 | 328 | 624 | — | | 45 | 0.50 | 7.2 | n.d. |
| C16 | | | | | | | | | |
| Methanosarcina mazei | 108 | 327 | 624 | — | | 45 | 0.50 | 7.2 | n.d. |
| Go1 | | | | | | | | | |
| Methanosarcina siciliae | 105 | 333 | 625 | — | | 50 | 0.48 | 8.2-9.2 | biotin, thiamine |
| Methanosarcina siciliae | 122 | 328 | 624 | — | | 50 | 0.48 | 8.2-9.2 | biotin, thiamine |
| C2 | | | | | | | | | |
| Methanosarcina sp. | 113 | 490 | 626 | — | | n.d. | n.d. | n.d. | n.d. |
| 1.H.A.2.2 | | | | | | | | | |
| Methanosarcina sp. | 111 | 331 | 627 | — | | n.d. | n.d. | n.d. | n.d. |
| 1.H.T.1A.1 | | | | | | | | | |
| Methanosarcina sp. | 112 | 490 | 626 | — | | n.d. | n.d. | n.d. | n.d. |
| 2.H.A.1B.4 | | | | | | | | | |
| Methanosarcina sp. | 110 | 331 | 627 | — | | n.d. | n.d. | n.d. | n.d. |
| 2.H.T.1A.3 | | | | | | | | | |
| Methanosarcina sp. | 101 | 334 | 628 | — | | n.d. | n.d. | n.d. | n.d. |
| Ant1 | | | | | | | | | |
| Methanosarcina sp. | 123 | 327 | 629 | — | | n.d. | n.d. | n.d. | n.d. |
| Kolksee | | | | | | | | | |
| (GCA_000969945.1) | | | | | | | | | |
| Methanosarcina sp. | 125 | 327 | 629 | — | | n.d. | n.d. | n.d. | n.d. |
| Kolksee | | | | | | | | | |
| (GCF_000969945.1) | | | | | | | | | |
| Methanosarcina sp. | 114 | 328 | 630 | — | | n.d. | n.d. | n.d. | n.d. |
| MSH10X1 | | | | | | | | | |
| Methanosarcina sp. | 100 | 330 | 631 | — | | n.d. | n.d. | n.d. | n.d. |
| MTP4 | | | | | | | | | |
| Methanosarcina sp. | 109 | 337 | 632 | — | | n.d. | n.d. | n.d. | n.d. |
| WWM596 | | | | | | | | | |
| Methanosarcina spelaei | 121 | 327 | 629 | — | | 54 | 0.35 | 6.5-6.6 | n.d. |
| Methanosarcina | 116 | 325 | 633 | — | | 60 | 0.2-0.25 | 7-7.2 | biotin |
| thermophila | | | | | | | | | |
| Methanosarcina | 117 | 325 | 633 | — | | 60 | 0.2-0.25 | 7-7.2 | biotin |
| thermophila CHTI-55 | | | | | | | | | |
| Methanosarcina | 124 | 327 | 629 | — | | 42 | 0.4-0.6 | 7-8.7 | biotin |
| vacuolata | | | | | | | | | |
| Methanothermobacter | 225 | 470 | 634 | — | | n.d. | n.d. | 7.7 | Rumen fluid, |
| defluvii | | | | | | | | | nutrient broth |
| Methanothermobacter | 220 | 356 | 635 | — | | 70 | 0.2-0.4 | 7 | n.d. |
| marburgensis | | | | | | | | | |
| Methanothermobacter | 222 | 470 | 634 | — | | n.d. | n.d. | n.d. | n.d. |
| sp. CaT2 | | | | | | | | | |
| Methanothermobacter | 224 | 470 | 634 | — | | n.d. | n.d. | n.d. | n.d. |
| sp. EMTCatA1 | | | | | | | | | |
| Methanothermobacter | 213 | 498 | 636 | — | | n.d. | n.d. | n.d. | n.d. |
| sp. MT-2 | | | | | | | | | |
| Methanothermobacter | 214 | 497 | 637 | — | | 80 | n.d. | 7-7.2 | yeast extract |
| tenebrarum | | | | | | | | | |
| Methanothermobacter | 223 | 470 | 634 | — | | 75 | 0.50 | 6 | NaCl, Na2S |
| thermautotrophicus | | | | | | | | | |
| Methanothermobacter | 221 | 356 | 635 | — | | 74 | n.d. | 8 | n.d. |
| wolfeii | | | | | | | | | |
| Methanothermococcus | 151 | 342 | 644 | — | | 75 | 0.34 | 7-7.4 | Ni, Fe, Co, Mg, |
| okinawensis IH1 | | | | | | | | | Ca, SeO4, CO2, |
| | | | | | | | | | 2methylbutyrate, |
| | | | | | | | | | propionate, |
| | | | | | | | | | isoleucine, |
| | | | | | | | | | leucine |
| Methanothermococcus | 148 | n.d. | — | — | | 70 | 0.00 | 6.5-7 | acetate, yeast |
| thermolithotrophicus | | | | | | | | | extract |
| Methanothermus | 170 | 355 | 638 | — | | 97 | n.d. | 7 | rumen fluid, |
| fervidus | | | | | | | | | yeast extract |
| Methanothrix | 86 | 322 | 639 | — | | n.d. | n.d. | n.d. | n.d. |
| soehngenii | | | | | | | | | |
| Methanothrix | 92 | 323 | 640 | — | | n.d. | n.d. | n.d. | n.d. |
| thermoacetophila PT | | | | | | | | | |
| Methanotorris | 153 | 506 | 641 | 507 | 661 | 83 | 0.00 | 7 | n.d. |
| formicicus Mc-S-70 | | | | | | | | | |
| Methanotorris igneus | 152 | 350 | 642 | — | | 91 | 0.00 | 6.8-7.5 | acetate, yeast |
| Kol 5 | | | | | | | | | extract, |
| | | | | | | | | | tungsten, |

*Alternative or secondary recognition motifs derive from additional MtrA paralogs in the respective organisms.

The present invention is also illustrated by the appended non-limiting Figures and Examples.

### BRIEF DESCRIPTION OF FIGURES

FIG. 1: Structure-based sequence alignment.

Shown is an alignment of the Methanocaldococcus jannaschii proteasome β subunit (SEQ ID NO: 435), M. jannaschii Adriase (SEQ ID NO: 159) and Methanosarcina mazei Adriase (SEQ ID NO: 108). Secondary structure elements are indicated by arrows (beta-sheets) and tubes (helices). Identical residues between two sequences are marked by asterisks. The alignment visualizes the distant relationship between proteasomal NTN (N-terminal nucleophile) and Adriase DUF2121 domains. Some Adriase variants, such as the one from M. jannaschii, contain an additional C-terminal six-stranded OB-like domain that is connected to the DUF2121 domain through a long helix.

FIG. 2: Adriase covalently modifies MtrA^{N}.

(A) SDS-PAGE of a pulldown experiment with His6 (SEQ ID NO: 678)-tagged M. mazei Adriase (SEQ ID NO: 380). While the control protein BSA is removed in flow-through (FT) and wash fractions (W₁₋₄), MtrA (SEQ ID NO: 390) co-elutes with Adriase and MtrA^{N}-Adriase conjugate (Elu; SEQ ID NO: 431). MtrA^{C} (SEQ ID NO: 432) is not detected, due to its small size.

(B-D) Liquid chromatography mass spectrometrical (LCMS) analysis of the Adriase (SEQ ID NO: 390) reaction with MtrA (SEQ ID NO: 380). The spectra show identified masses in the eluted fractions, which are interpreted as MtrA^{C} (SEQ ID NO: 432; theoretical mass: 6844.7 Da/detected mass: 6844.7 Da), MtrA (24445.9 Da/24442.7 Da), Adriase (22068.2 Da/22067.8 Da), MtrA^{N}-Adriase conjugate (SEQ ID NO 431; 39669.4 Da/39665.8 Da) and non-covalent MtrA-Adriase complex (46514.1 Da/46510.5 Da; disassembles during SDS-PAGE).

FIG. 3: Comparison of Adriase recognition motifs.

Shown is an alignment of the (K/R)DPGA (SEQ ID NO: 677) motif with the 15 preceding and the 10 following amino acids in a phylogenetically diverse set of MtrA proteins (SEQ ID NO 423 and 436-449). The sequence conservation is visualized above the alignment, with larger letters indicating higher conservation. All shown MtrA proteins co-occur with Adriase.

FIG. 4: The Adriase N-terminus forms an amide bond with MtrA aspartate.

Shown is an MS/MS spectrum of the fusion peptide resulting from amide bond formation between the M. mazei Adriase N-terminus (TLVIAFIGK (SEQ ID NO: 668) . . . ; SEQ ID NO: 108) and the MtrA KDPGA (SEQ ID NO: 311)-motif (SEQ ID NO: 328). The sample was digested with trypsin and free amino-groups dimethylated. The threonine amino group is not modified, suggesting its involvement in an amide bond.

FIG. 5: MtrA-Adriase forms a heterodimer, in which MtrA is bound via a short amino acid motif.

(A) M. jannaschii MtrA (SEQ ID NO: 420) and Adriase (SEQ ID NO: 386) proteins were loaded individually on a gel filtration column or as a 1:1 molar mixture. While Adriase and MtrA alone show a comparable elution behavior, the mixture of both elutes at a lower volume, indicating complex formation. This interpretation is supported by light scattering measurements (thick lines, plotted on the secondary Y-axis). The determined masses (table) closely resemble the theoretical monomeric masses for Adriase and MtrA alone and for the MtrA-Adriase heterodimer.

(B) The K_{D} for MtrA-Adriase (SEQ ID NO: 386 and 420) complex formation was determined via microscale thermophoresis in three independent measurements (chromatogram). Measurements for peptides (SEQ ID NO: 367-372) that mimic the MtrA binding site (table) show that the 15 amino acid peptide (0)KDPGA (SEQ ID NO: 311) (10) (SEQ ID NO: 372) is sufficient for this high affinity interaction.

FIG. 6: Adriase binds its recognition motif via beta-sheet interactions.

Shown is the crystal structure of M. jannaschii Adriase^{S1A} (SEQ ID NO: 386) in complex with (15)KDPGA (SEQ ID NO: 311) (10) (SEQ ID NO: 367). A dashed line indicates the modification site.

FIG. 7: Adriase modifications are reversible and allow the recombination of substrates.

(A) SDS-PAGE showing the time course of the M. jannaschii MtrA-Adriase reaction (SEQ ID NO: 385 and 420). Only a small fraction of MtrA is bound to Adriase at any time, suggesting the reversibility of the reaction.

(B) The same reaction in presence of a second Adriase substrate, Ubiquitin fused to KDPGA (SEQ ID NO: 311) (10) (SEQ ID NO: 392). Both MtrA and Ub N-termini can be linked covalently to Adriase (MtrA^{N}-Adriase, Ub^{N}-Adriase; Predicted SEQ ID NO: 433-434). In the reverse reaction, they can be recombined with each of the C-termini (MtrA^{N}-Ub^{C}, Ub^{N}-MtrA^{C} (SEQ ID NO: 427-428).

(C-D) LCMS analysis of the Adriase reaction shown in (B). The spectra show identified masses in the eluted fractions, which are interpreted as Ub-KDPGA (SEQ ID NO: 311) (10) (theoretical mass: 11194 Da), Ub^{N}-MtrA^{C} (13188 Da), MtrA (21571 Da) and MtrA^{N}-Ub^{C} (19577 Da). The exchange of the C-termini results in a determined mass shift of 1994 Da and 1995 Da, respectively (theoretical mass shift: 1994 Da). The method is not quantitative.

FIG. 8: Proposed mechanism for Adriase.

The shown Adriase mechanism is a combination of the first steps of two known proteasomal reactions: proteolysis and autolysis. A substrate, R₁-R₂ (left), is cleaved (middle) and the N-terminal fragment R₁ transferred on the Adriase Thr1/Ser1 N-terminus (right; see FIG. 3). The reaction is reversible and differs from proteolysis/autolysis by avoiding the irreversible hydrolysis step (bottom): hydrolysis products could not be identified in any of the shown mass spectrometrical analyses.

FIG. 9: Adriase efficiently ligates peptides with 5 residues N- and 10 residues C-terminal of the KDPGA (SEQ ID NO: 311) motif.

(A) SDS-PAGE analysis of Adriase (SEQ ID NO: 385) mediated peptide ligations with fluorophore-linked substrates (“F”), visualized by UV light. The substrate (F15)KDPGA(SEQ ID NO: 311) (10) (SEQ ID NO: 367) (lane 2-4) forms a covalent bond to Adriase ((F15)KD-Adriase; Predicted SEQ ID NO: 429), resulting in the release of non-fluorescent PGA(10) (not visible; SEQ ID NO: 374); In presence of PGA(17) (SEQ ID NO: 373), (F15)KDPGA(SEQ ID NO: 311) (10) is recombined to (F15)KDPGA(SEQ ID NO: 311) (17) (Predicted SEQ ID NO: 413). Substrates with C-terminal fluorophores (lane 5-13) also form covalent bonds to Adriase (non-fluorescent), resulting in the release of small quantities of PGA(10F) (Predicted SEQ ID NO: 414); In presence of PGA(17) (SEQ ID NO: 373), non-fluorescent ligation products (15/10/5)KDPGA(SEQ ID NO: 311) (17) (Predicted SEQ ID NOs: 415-417) and more PGA(10F) are formed. The migration of the peptides is dominated by the fluorophore and therefore not always proportional to their size.

(B) Michaelis-Menten plot for the recombination of (15)KDPGA(SEQ ID NO: 311) (10F) (SEQ ID NO: 418) with PGA(10) (SEQ ID NO: 374). The signal was obtained by quantification of fluorescent PGA(10F) (Predicted SEQ ID NO: 414) bands in polyacrylamide gels.

(C) Table summarizing ligation rates for various substrate combinations. Results presented in B and C were determined in single experiments.

FIG. 10: Sequence determinants governing Adriase activity.

(A) M. jannaschii MtrA (SEQ ID NO: 391) and Adriase^{ΔOBS1A} (SEQ ID NO: 132) proteins were loaded individually on a gel filtration column or as a 1:1 molar mixture. The elution profile of the mixture is identical to the combined profiles of its isolated components, indicating decreased affinity of Adriase^{ΔOBS1A} (SEQ ID NO: 132) for MtrA (SEQ ID NO: 391) compared to full length Adriase^{S1A} (SEQ ID NO: 386; FIG. 5A).

(B) A set of Adriase mutants (SEQ ID NO: 380, 384-388 and 450; see main text) were screened for ligase activity with M. jannaschii MtrA-derived (15)KDPGA(SEQ ID NO: 311) (10) (SEQ ID NO: 419) and PGA(10F) peptide (SEQ ID NO: 414) substrates. Fluorescent (F) substrates were subsequently detected via SDS-PAGE and UV exposure. A successful ligation produces (15)KDPGA(SEQ ID NO: 311) (10F) (Predicted SEQ ID NO: 418) and PGA(10) (not visible; Predicted SEQ ID NO: 374).

FIG. 11: Ligation rate and completeness can be controlled via substrate ratios.

(A) Higher primary substrate concentrations at constant secondary substrate levels increase Adriase ligation rates. Shown are apparent ligation rates, measured by the amount of ligated product in an early reaction phase.

(B) Higher secondary substrate concentrations at constant primary substrate levels increase Adriase ligation rates up to a ratio of 1:1. A higher ratio inhibits the reaction.

(C) The percentage of ligated primary substrate as a function of secondary substrate concentration. The determined data (dots) agree with the theoretical model (solid line, see main text). Results presented in this figure (A-C) were determined in single experiments.

FIG. 12: Time courses and Michaelis-Menten plot of the M. mazei Adriase reaction

(A) Time courses of Adriase catalyzed ligations with various substrate concentrations. The determined data (symbols) show that these reactions can be described by simple formulae (lines; see main text), allowing the determination of maximum reaction rates. Shown are data for 1.25 μM (squares/dotted line), 2.5 μM (diamonds/short dash line), 5 μM (triangles/medium dash line), 10 μM (circles/dash-dotted line), 20 μM (squares/long dash line) and 40 μM (triangles/solid line).

(B) Michaelis-Menten plot of the maximum reaction rates determined in (A). Highlighted are the approximated substrate concentration for half-maximal reaction speed and the maximum ligation rate. Results presented in this figure (A-B) were determined in single experiments.

FIG. 13: Adriase does not hydrolyze its substrates.

Shown are fluorescent peptides on an SDS-gel, visualized by UV fluorescence. The gel depicts the time course of a reaction with 15 μM (F10)KDPGA(SEQ ID NO: 311) (10) (SEQ ID NO: 456), 15 μM PGA(25) (SEQ ID NO: 457) and either 15 nM or 15 μM M. mazei Adriase (SEQ ID NO: 380). The products are shown in comparison to an experiment with 15 μM M. mazei Adriase and the hypothetical hydrolysis product (F10)KD (SEQ ID NO: 458) (Hydrolysis control). No band at the height of (F10)KD is observed in the first two experiments, neither at normal exposure levels (upper panel) nor with overexposure (lower panel), indicating that Adriase does not hydrolyze its substrates.

FIG. 14: An unmodified amino group at the Adriase active site serine/threonine is required for efficient ligations

Shown is an SDS-PAGE analysis of Adriase-mediated ligations of (15)KDPGA(SEQ ID NO: 311) (10) (SEQ ID NO: 419) and PGA(10F) (SEQ ID NO: 414) substrates, visualized by UV light. Unmodified Adriase (SEQ ID NO: 385) generates the reaction product (15)KDPGA(SEQ ID NO: 311) (10F) (SEQ ID NO: 418) at ~200× higher rates compared to N-terminally modified Adriase (SEQ ID NO: 450), highlighting the importance of a free amino group at the active site serine for efficient ligations.

FIG. 15: N-terminal Adriase modifications are subject to non-specific proteolysis

Liquid chromatography mass spectrometrical (LCMS) analysis of N-terminally modified Adriase (SEQ ID NO: 450). The spectra shows the most prominent masses, automatically assigned by the Bruker Compass DataAnalysis software. These are interpreted as N-terminally modified Adriase lacking the start-methionine (Δ1, SEQ ID NO: 511; theoretical mass: 35909.6 Da/detected mass: 35909.4 Da), the first 8 (Δ8, SEQ ID NO: 512; 34992.7 Da/34993.3 Da), the first 10 (Δ10, SEQ ID NO: 513; 34768.4 Da/34769.2 Da), the first 11 (Δ11, SEQ ID NO: 514; 34681.4 Da/34682.9 Da) or the first 21 (Δ21, SEQ ID NO: 515; 33746.3 Da/33745.7 Da) residues. The Δ21 truncation exposes the active site serine, rendering this variant catalytically active.

FIG. 16: Schematic representation of Adriase ligation strategies and their potential applications.

(A) When used on two (5)KDPGA(SEQ ID NO: 311) (10) substrates, Adriase catalyzes an equilibrium between the four possible (5)KD-PGA(10) combinations. The ratio of these products depends on the kinetic parameters for their reaction with Adriase and on substrate ratios. When these are similar, the reaction results in equimolar products.

(B) When used on one (5)KDPGA(SEQ ID NO: 311) (10) and one PGA(10) substrate, Adriase catalyzes an equilibrium between just two possible (5)KD-PGA(10) combinations, increasing yields for a given ligation product.

(C) The (5)KDPGA(SEQ ID NO: 311) (10) substrate can potentially be replaced by the reaction intermediate, (5)KD-Adriase.

(D) Adriase-mediated ligations are useful for engineering novel molecules that cannot be obtained by genetic or chemical means. For example, bispecific or hybrid antibodies (Alam (2017) Chembiochem 18:2217-2221) can be designed through rearrangement of the respective modular Fc, Fab and scFv regions. Similarly, proteins can be linked to non-proteinaceous compounds, such as imaging agents or bioactive molecules (Veggiani (2016) Proc Natl Acad Sci USA 113:1202-7).

(E) Adriase can be used to immobilize proteins on nanoparticles, increasing the efficiency of multienzyme pathways. Furthermore, it is envisaged herein to use Adriase for covalent and/or geometrically defined attachment of proteins and/or protein complexes on surfaces/solid carriers for microscopy applications, e.g. electron microscopy, especially cryo-electron microscopy.

(F) Adriase-mediated ligations enable segmental isotopic labeling, which is useful for structure determination via NMR (Liu (2017) Methods Mol Biol 1495:131-145).

(G) Adriase facilitates a variety of immunotherapeutic techniques. For example, a virus-like particle (VLP) fused to a variety of antigens is far more efficient in immunization than the individual antigens (Thrane (2016) J Nanobiotechnology 14:30). A VLP displaying the PGA(10) motif on its capsid proteins could present a versatile tool allowing for the simultaneous ligation of a selection of (5)KDPGA(SEQ ID NO: 311) (10)-linked antigens.

(H) Adriase allows anchoring of molecules on the cell surface or can target them to specific tissues or cell compartments. For example, proteins can be fused to membrane proteins and thereby be enriched in outer membrane vesicles, which increase their stability and are useful for drug delivery (Alves (2015) ACS Appl Mater Interfaces 7:24963-72).

(I) Adriase can be used to produce circular proteins, which are exceptionally useful in therapeutic applications, due to their increased stability (van 't Hof (2015) Biol Chem 396:283-93).

(J) Adriase can catalyze internal ligations in disulfide-bonded substrates, allowing the formation of non-canonical protein assemblies.

(K) Column resins with immobilized Adriase can be recycled and potentially obviate the need for subsequent purifications. They can be used to react with the Y-(5)KD moiety of a primary substrate. The ligation product can subsequently be eluted by addition of the secondary PGA(10)-Z substrate.

(L) The tight interaction between Adriase and its substrates can be used for affinity chromatography. A resin with immobilized inactive AdriaseS1A binds a KDPGA(SEQ ID NO: 311) (0)- and possibly also PGA(10)-tagged protein (shown) with submicromolar affinity. The purified protein can then be eluted with an excess of PGA(10) peptides. The resin is also useful for pulldown experiments.

(M) Adriase could potentially obviate the need for antibodies in western blots by ligating a reporter enzyme (e.g. alkaline phosphatase) bearing the Adriase recognition motif to PGA(10)-tagged proteins on the blot. These can subsequently be detected via established methods, e.g. with NBT/BCIP.

FIG. 17: The MtrA C-terminus is not required for interaction with Adriase

M. jannaschii MtrAi^{Δ174-245} (SEQ ID NO: 518) and Adriase (SEQ ID NO: 386) proteins were loaded individually on a gel filtration column or as a 1:1 molar mixture. The mixture of both elutes at a lower volume than Adriase and MtrA^{Δ174-245} alone, indicating complex formation. This interpretation is supported by light scattering measurements (thick lines, plotted on the secondary Y-axis). The determined masses (table) closely resemble the theoretical monomeric masses for Adriase and MtrA^{Δ174-245} alone and for the MtrA^{Δ174-245}-Adriase heterodimer. Thus, MtrA^{Δ174-245} behaves just like M. jannaschii MtrA^{Δ194-245} (SEQ ID NO: 420; FIG. 5A) in this respect, indicating that residues 174-245 are not required for an interaction with Adriase.

FIG. 18: The (0)KDPGA(SEQ ID NO: 311) (10) motif is sufficient for a high-affinity interaction with Adriase

The KD for Adriase (SEQ ID NO: 386) complex formation with M. jannaschii MtrA^{Δ174-245} (SEQ ID NO: 518) or MtrA-derived peptides (SEQ ID NO: 367-372) was determined via microscale thermophoresis in three independent measurements. The resulting chromatograms provide the basis for the KD values shown in FIG. 5B.

FIG. 19: The Adriase NTN domain is involved in binding of MtrA

M. jannaschii MtrA (SEQ ID NO:420) and M. jannaschii Adriase^{ΔNTN} (SEQ ID NO: 519) proteins were loaded individually on a gel filtration column or as a 1:1 molar mixture. The mixture of both elutes at the same volume as AdriaseΔNTN and MtrA alone, indicating that no complex is formed under these conditions. This interpretation is supported by light scattering measurements (thick lines, plotted on the secondary Y-axis; table).

FIG. 20: Adriase ligase reactivity is not dependent on the presence of the OB-like domain.

(a, b) SDS-polyacrylamide sample gels showing the time course of M. jannaschii Adriase and Adriase^{ΔOB}-catalyzed (SEQ ID NO: 385 and 387) ligations using M. jannaschii MtrA-derived His₆ (SEQ ID NO: 678)-Ub-(5)KDPGA (SEQ ID NO: 311) (10) (SEQ ID NO: 526) and PGA(15)-Ub-His6 (SEQ ID NO: 678) (SEQ ID NO: 527) substrates. The band intensities of educt and product bands in three such gels were quantified and used to visualize the ligation ratios in a plot (b). The observed ligation rates are similar for both full-length and Adriase and Adriase^{ΔOB}, indicating that the OB-like domain is not necessary for Adriase ligations.

FIG. 21: Kinetic analysis of an Adriase-catalyzed protein-protein ligation.

(a) SDS-polyacrylamide sample gels depicting the time course of the same ligation reaction at various substrate concentrations.

(b) Quantification of ligation reactions, as shown in (a).

(c) Michaelis-Menten plot with ligation rate coefficients based on the data fits in (b). The kinetic parameters (k_{cat} and [S]_{0.5}) are approximated and based on additional data (not shown) for higher substrate concentrations: 0.74±0.05 sec⁻¹ at 25 μM, 0.88±0.07 sec⁻¹ at 50 μM, and 0.90±0.14 sec⁻¹ at 100 μM substrate concentration.

FIG. 22: M. mazei Adriase does not hydrolyze protein substrates.

Polyacrylamide gel showing a test for M. mazei Adriase (SEQ ID NO: 380) hydrolase activity with Ub-(5)KDPGA (SEQ ID NO: 311) (15) (SEQ ID NO: 522) and PGA(10) (SEQ ID NO: 454) substrates. At an enzyme:substrate concentration of 1:1000 (1×), Adriase catalyzes ~50% product formation (i.e. Ub-(5)KDPGA (SEQ ID NO: 311) (10) (SEQ ID NO: 524)) in 30 minutes without forming the putative hydolysis product Ub-(5)KD (SEQ ID NO: 523). Even at 1000× increased enzyme concentrations and prolonged incubation times, no hydrolysis product can be detected.

FIG. 23: Adriase is a versatile protein ligase

Polyacrylamide gel showing the incubation of M. mazei Adriase (SEQ ID NO: 380) with various proteins bearing the Adriase recognition motif. A constant fraction of Ub-(5)KDPGA (SEQ ID NO: 311) (10) (SEQ ID NO: 524) forms a conjugate with Adriase (Ub-(5)KD-Adriase), suggesting a reversible reaction between the two (lanes 1-4). In place of PGA(10), PGA(10)-sdAb/CyP/GST (SEQ ID NO: 663-665) can be used as alternative substrates for the “reverse reaction”, resulting in an equilibrium of the respective educts and Ub-(5)KDPGA(SEQ ID NO: 311) (10)-sdAb/CyP/GST products (lanes 5-10).

FIG. 24: (5)KDPGA(SEQ ID NO: 311) (10) is a good ligation motif for sterically accessible substrates, whereas (5)KDPGA(SEQ ID NO: 311) (15) is more efficient for sterically demanding substrates.

(a, b) Time courses of M. mazei Adriase (SEQ ID NO: 380)-mediated ligations of various peptide or protein substrate pairs, visualized via SDS-PAGE and analyzed by band quantification (plots). Shown are sample gels, each representing three independent experiments. Substrates were used at 25 μM, in 400× molar excess over Adriase. For the ligation with Ub-(5)KDPGA(SEQ ID NO: 311) (15) and PGA(10)/PGA(15) peptides (SEQ ID NO: 454 and 528), an additional Strep-tag sequence (indicated by an asterisk) was added C-terminal of the Ub-(5)KDPGA(SEQ ID NO: 311) (15) substrate (SEQ ID NO: 522), so that the ligation results in a size shift. The data fits allow the determination of the rate coefficients depicted in tables. No Adriase activity was observed with (0)KDPGA(SEQ ID NO: 311) (10) or PGA(5) substrates (n.d.; SEQ ID NO: 531 and 529).

FIG. 25: Comparison of Adriase- and Sortase-mediated protein-protein ligations.

(a) SDS-polyacrylamide sample gels and derived plot showing the time course (triplicates) of comparable protein-protein ligations using M. mazei Adriase (SEQ ID NO: 380), S. aureus Sortase A (SrtA, SEQ ID NO: 662) or evolved SrtA pentamutant (SrtA5*, SEQ ID NO: 535). Although Adriase is used at a 400× lower concentration, it catalyzes the ligation at much higher rates and without side-products.

(b) The same experiments at 32× higher substrate concentrations (i.e. 100 μM). Although these conditions are much more suitable for the Sortase reaction, Adriase shows ~4000× higher ligase activity compared to non-optimized (SrtA) and ~40× higher ligase activity compared to optimized (SrtA5*) Sortase enzymes

FIG. 26: Adriase shows high substrate specificity in complex solutions

Polyacrylamide gel showing the ligation of two recombinantly expressed substrates (Strep-Ub-(5)KDPGA(SEQ ID NO: 311) (10)-His6 (SEQ ID NO: 678) and PGA(15)-Ub (SEQ ID NO: 538 and 539) in cell lysates and the single step purification of the respective ligation product (Strep-Ub-(5)KDPGA(SEQ ID NO: 311) (15)-Ub) using a Ni-NTA column in series with a Streptavidin column.

### EXAMPLE 1: DESIGN, CLONING AND PURIFICATION OF RELEVANT GENES

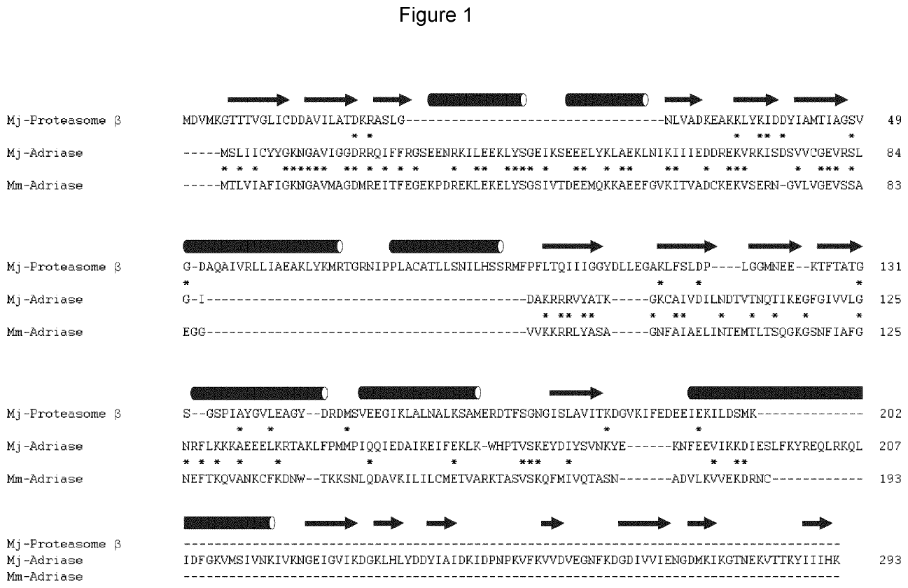

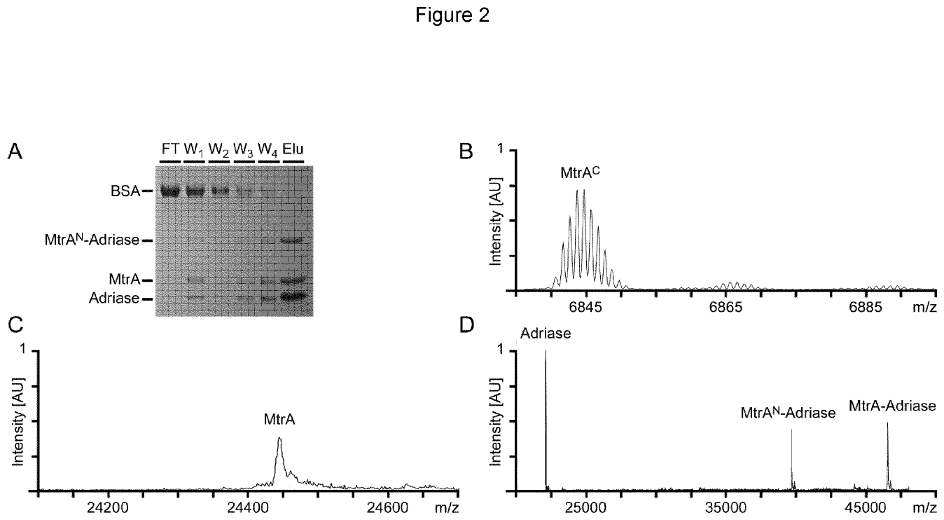

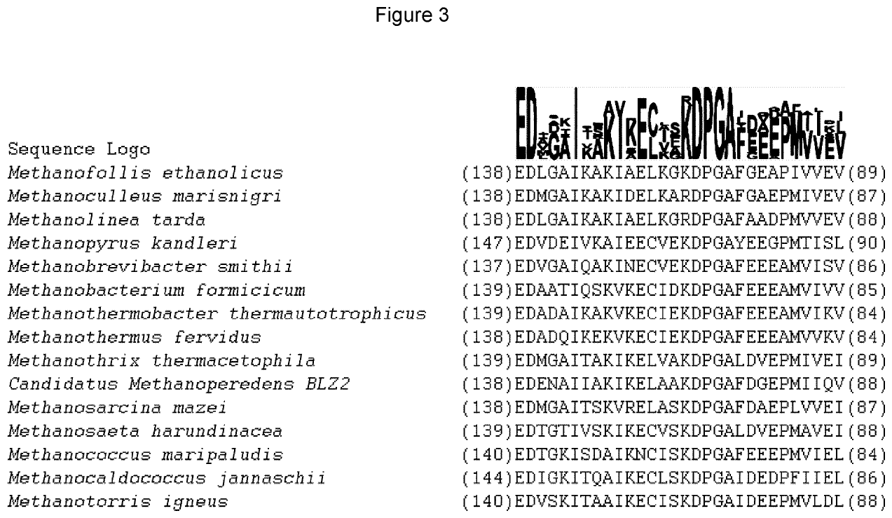

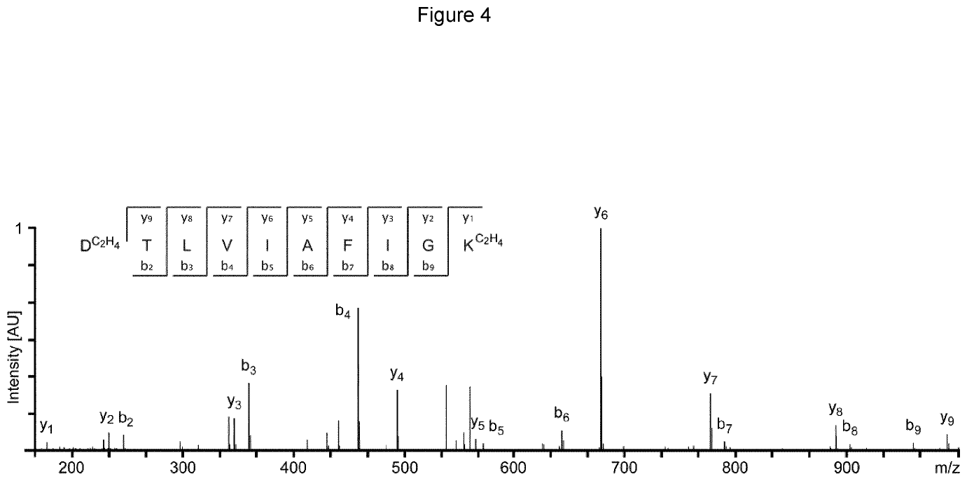

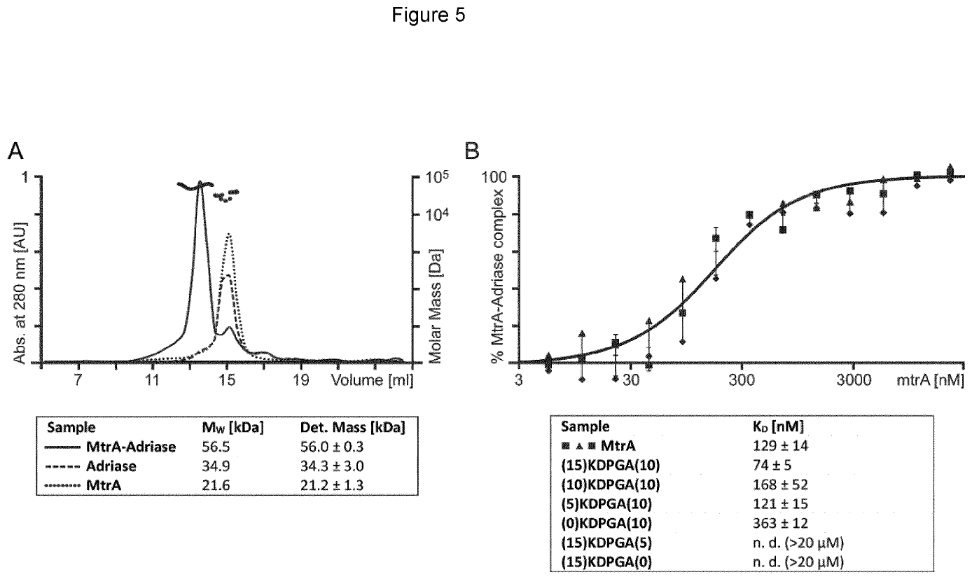

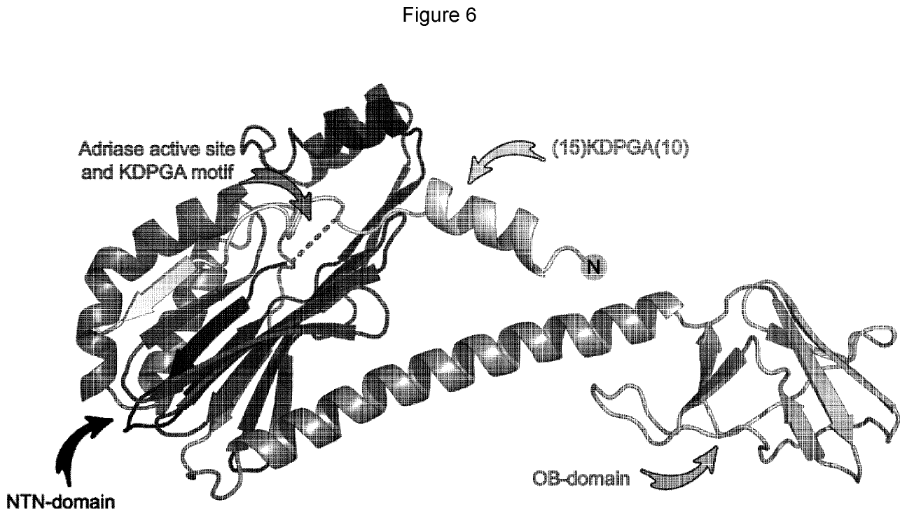

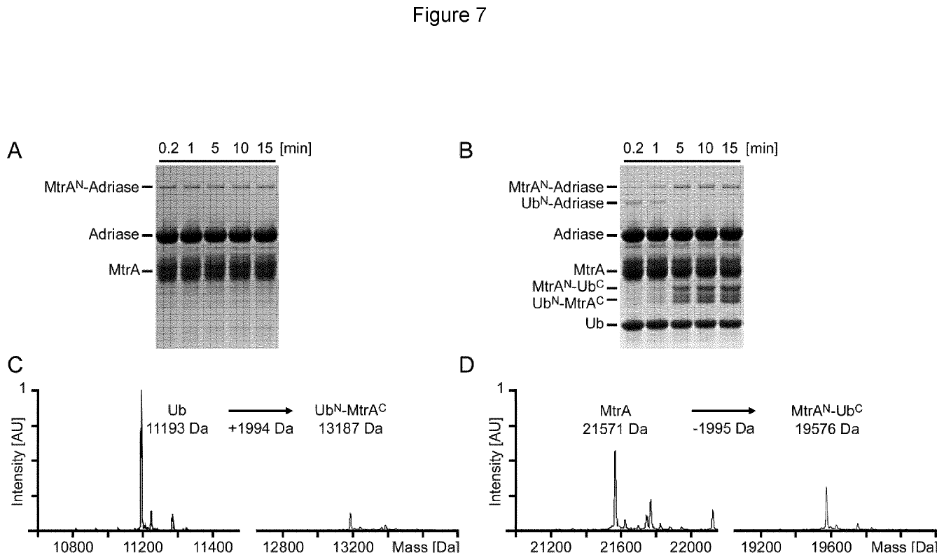

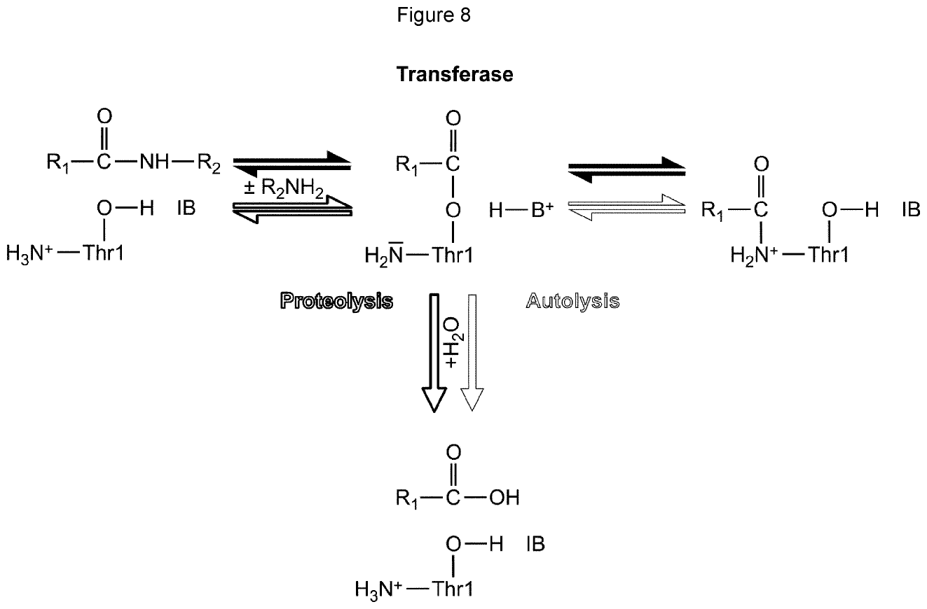

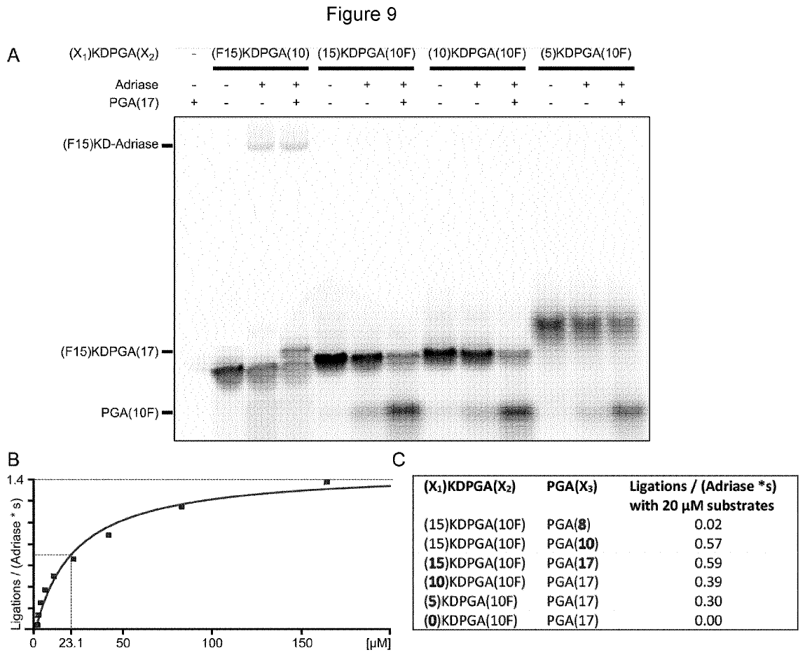

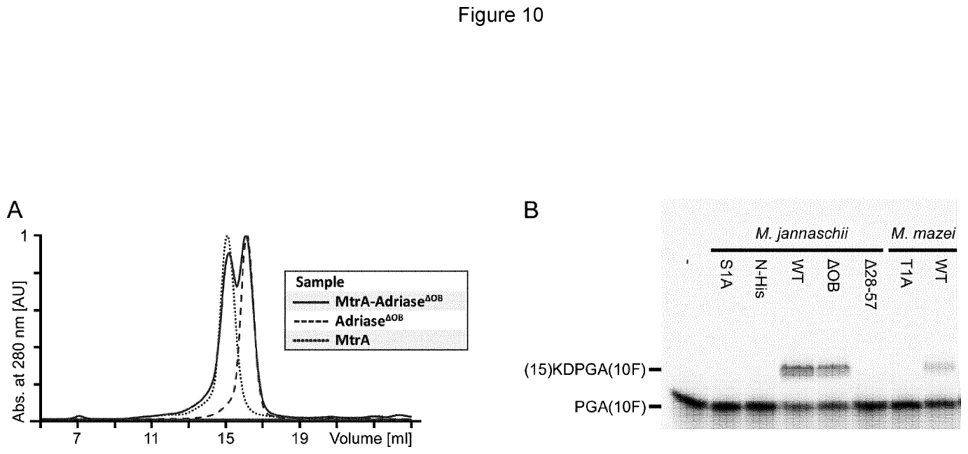

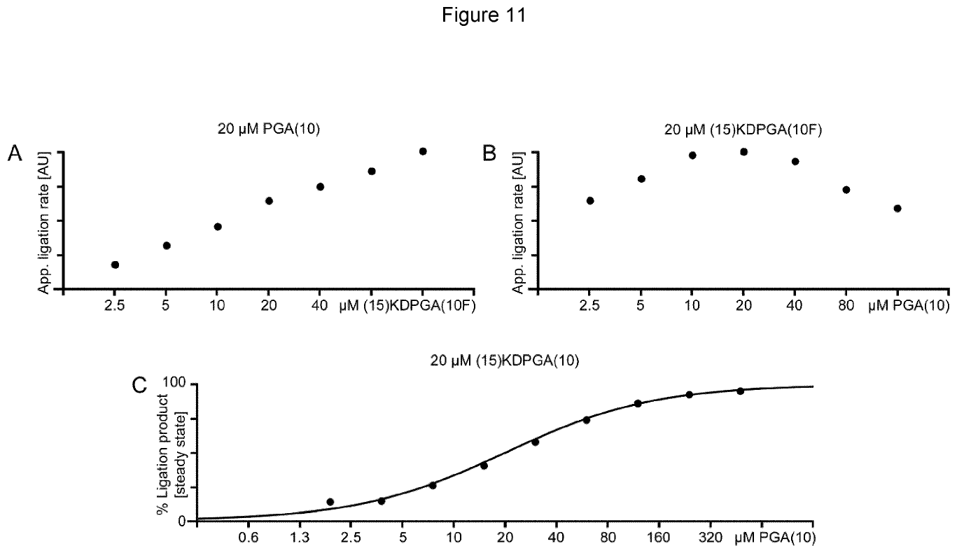

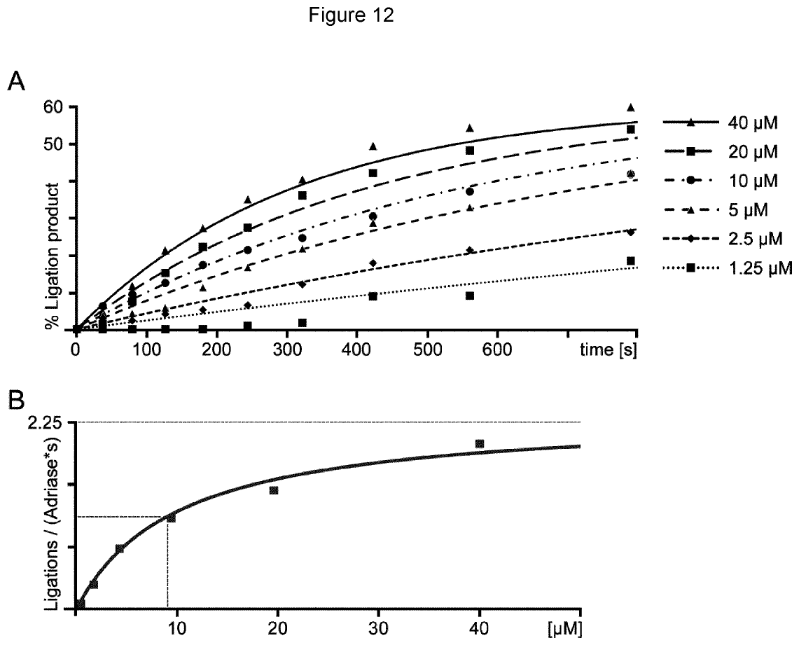

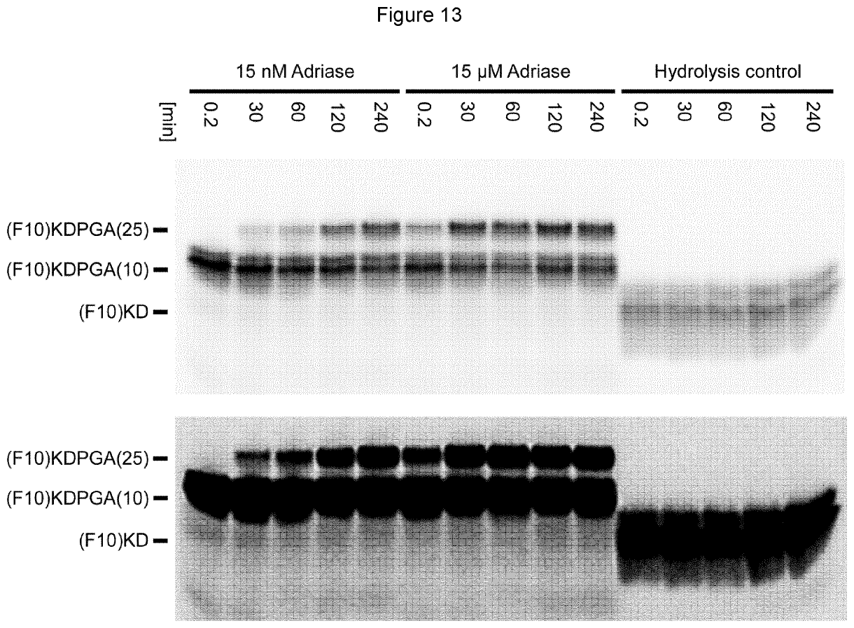

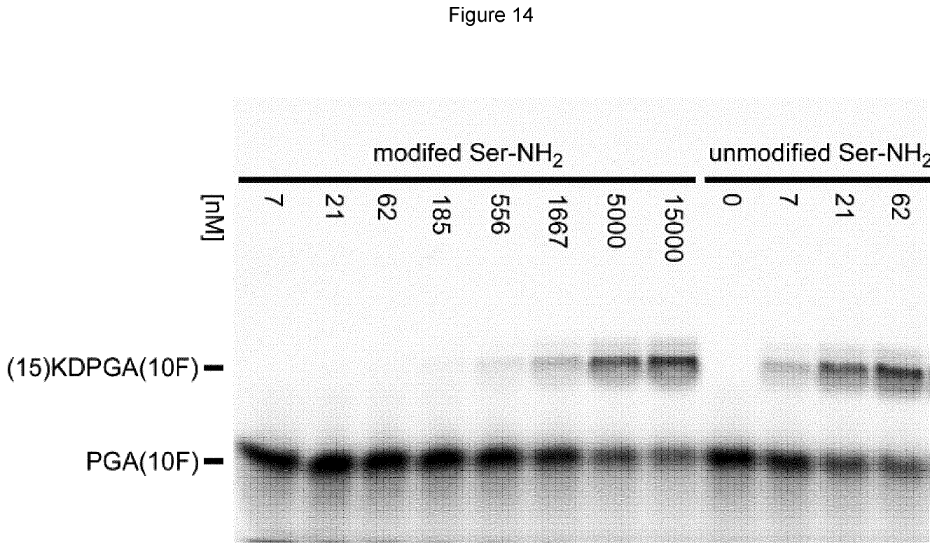

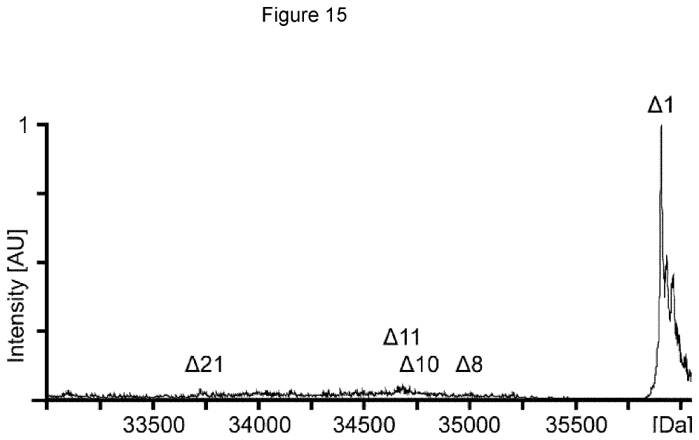

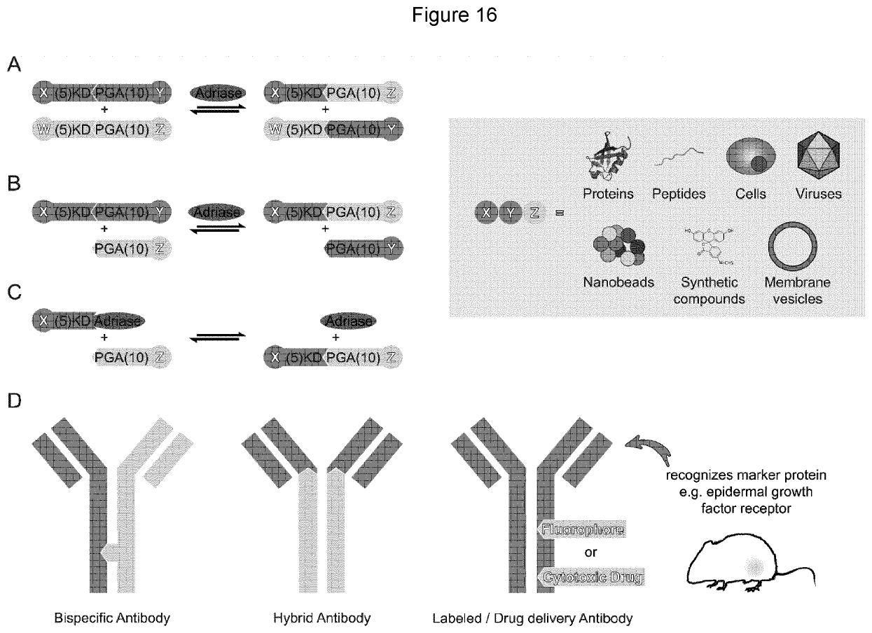

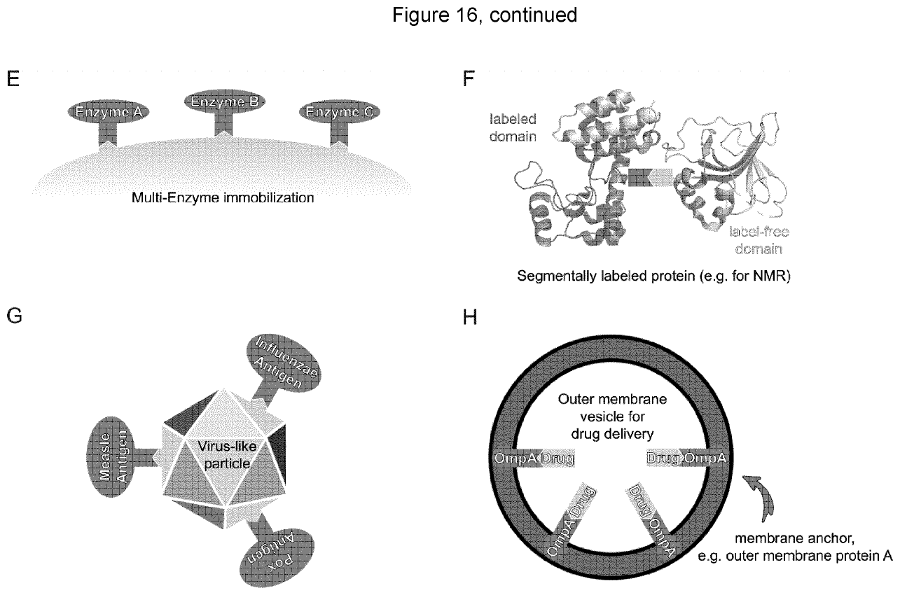

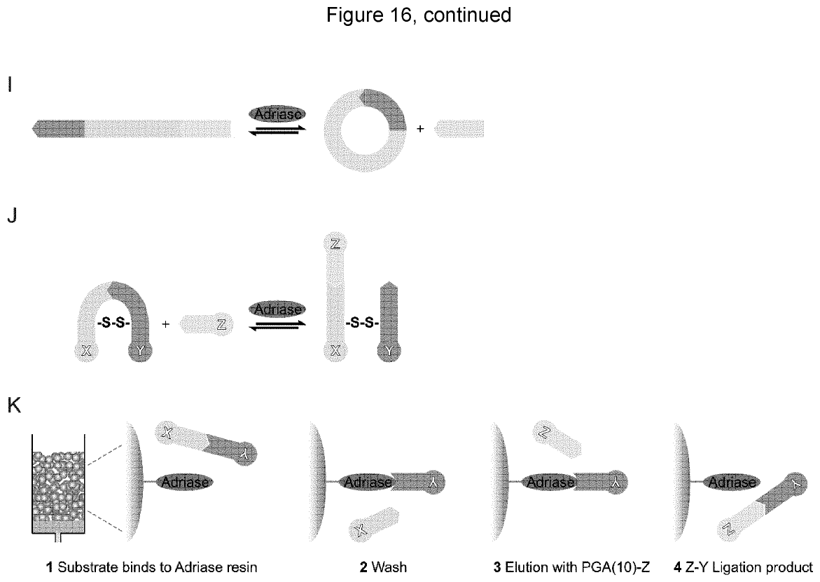

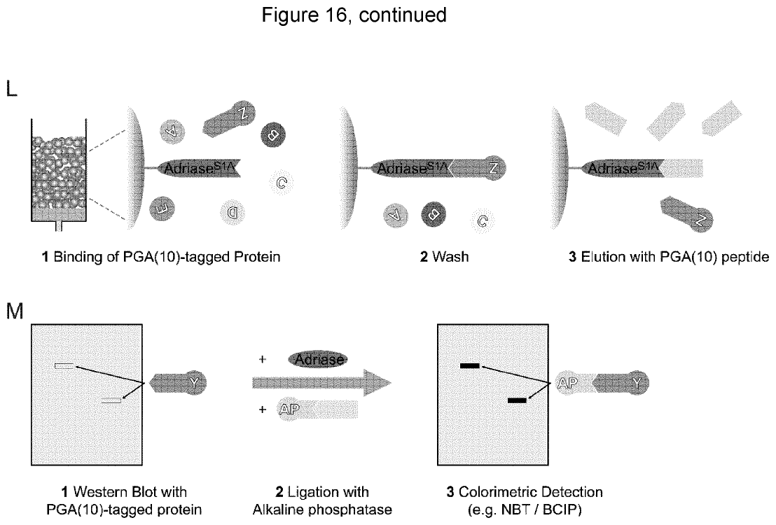

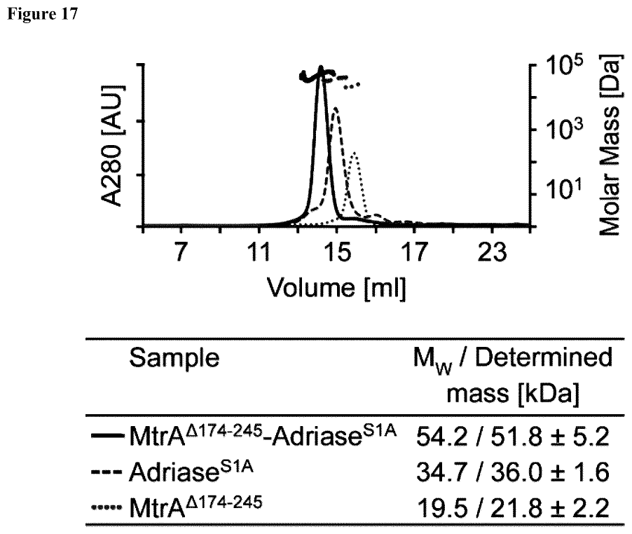

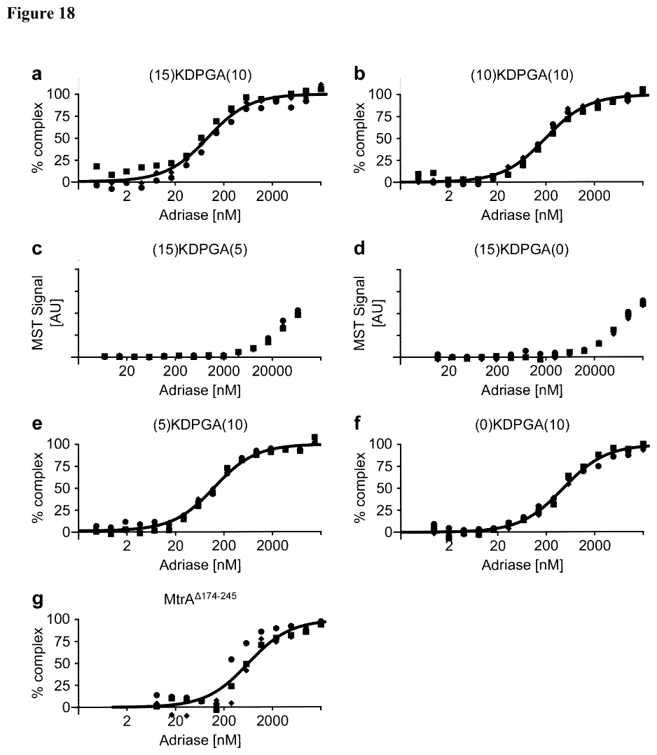

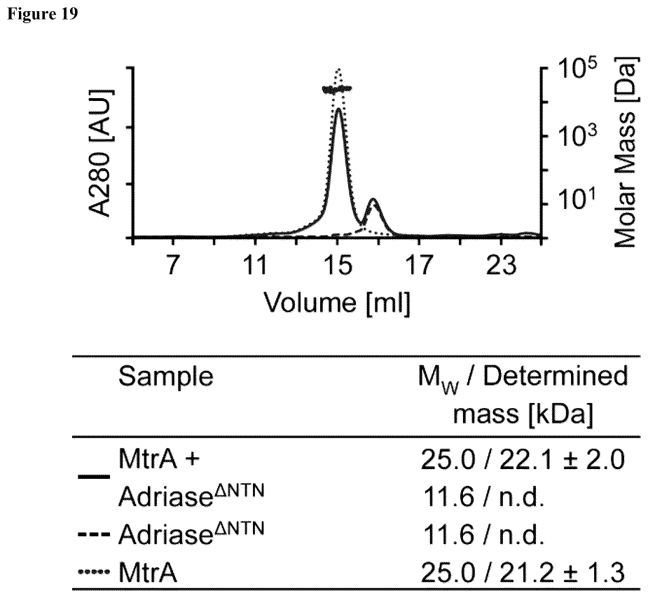

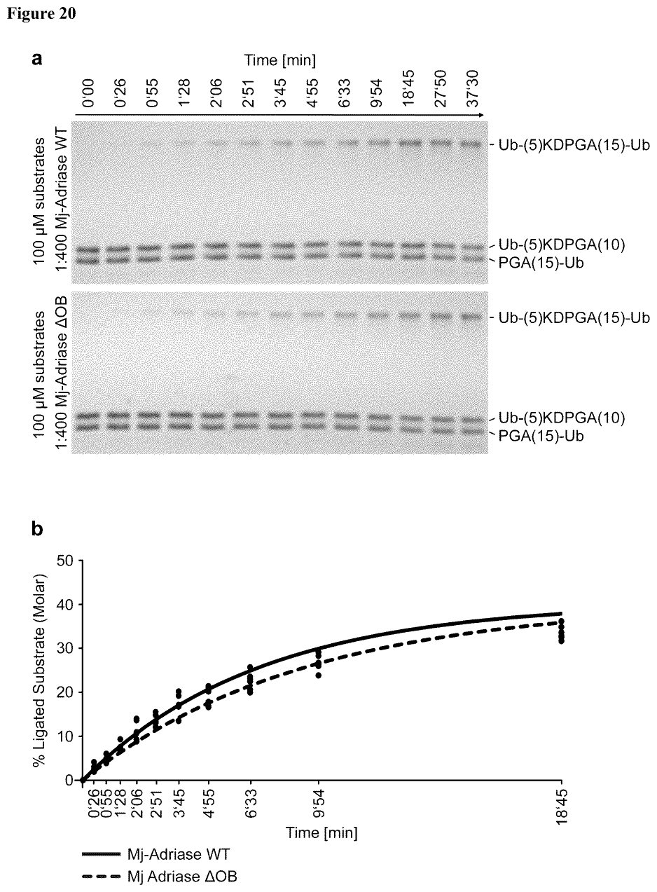

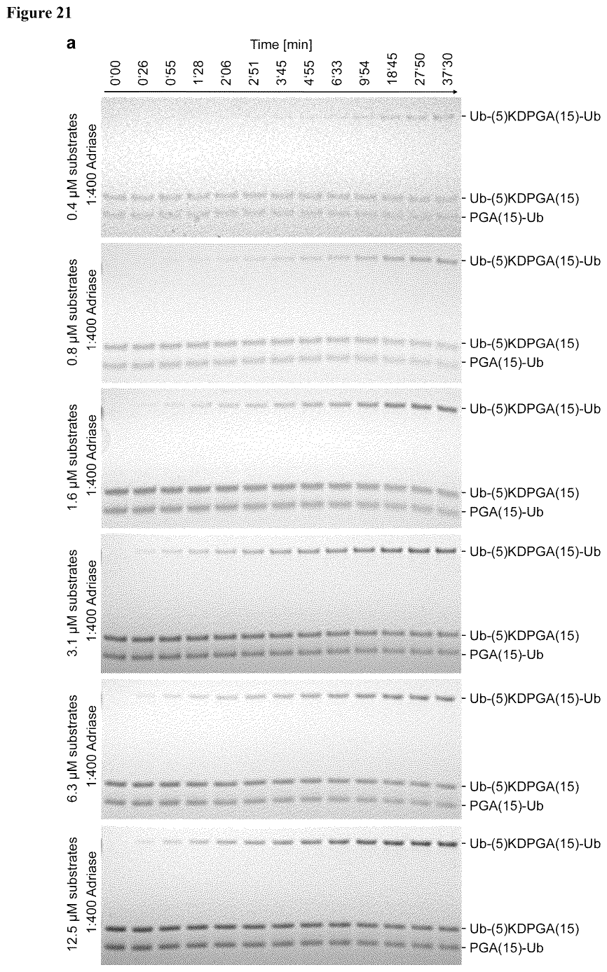

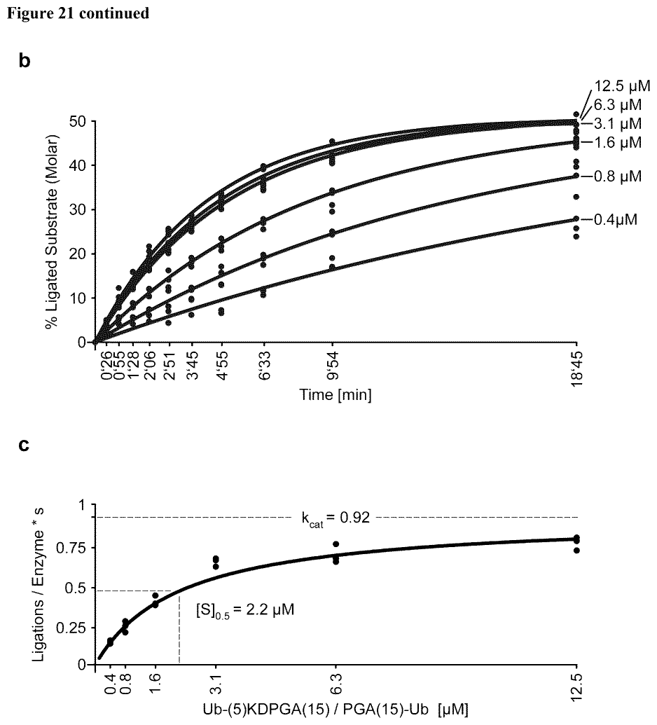

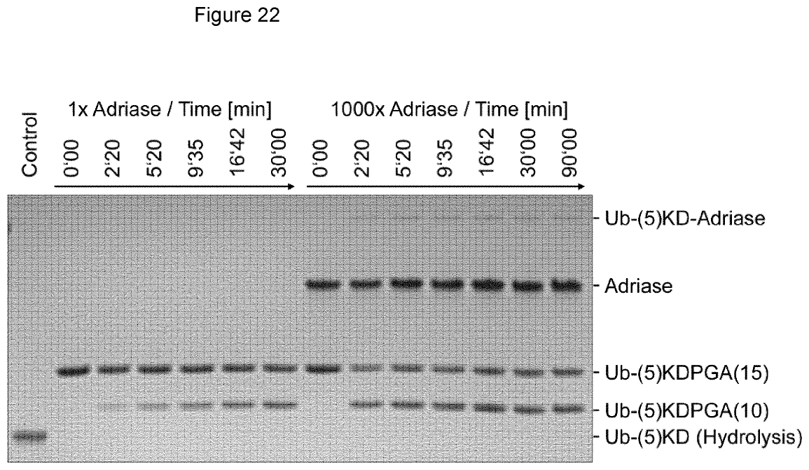

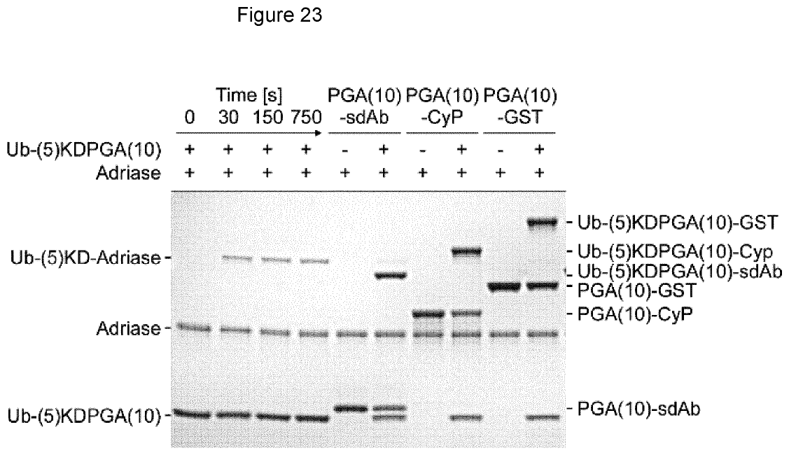

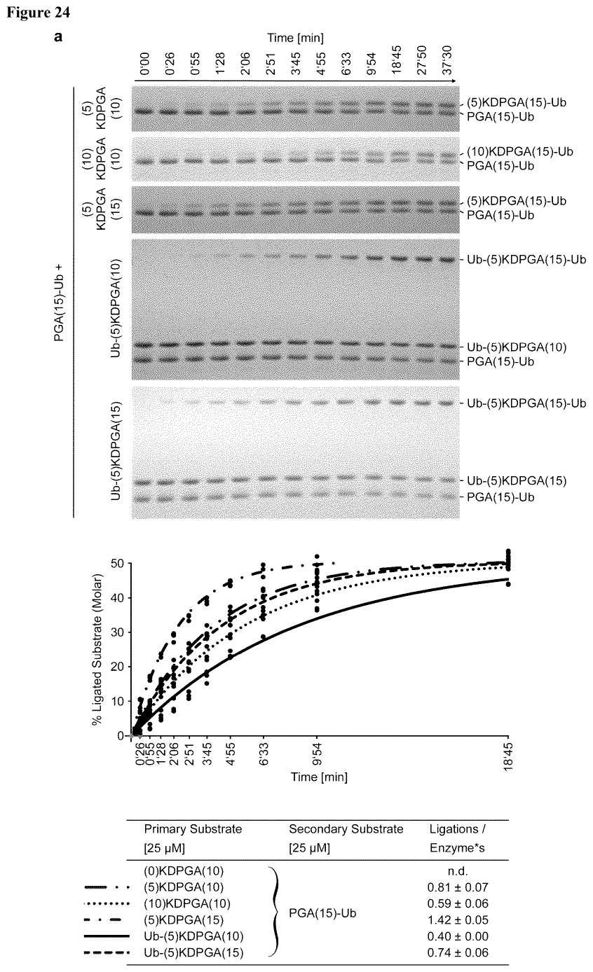

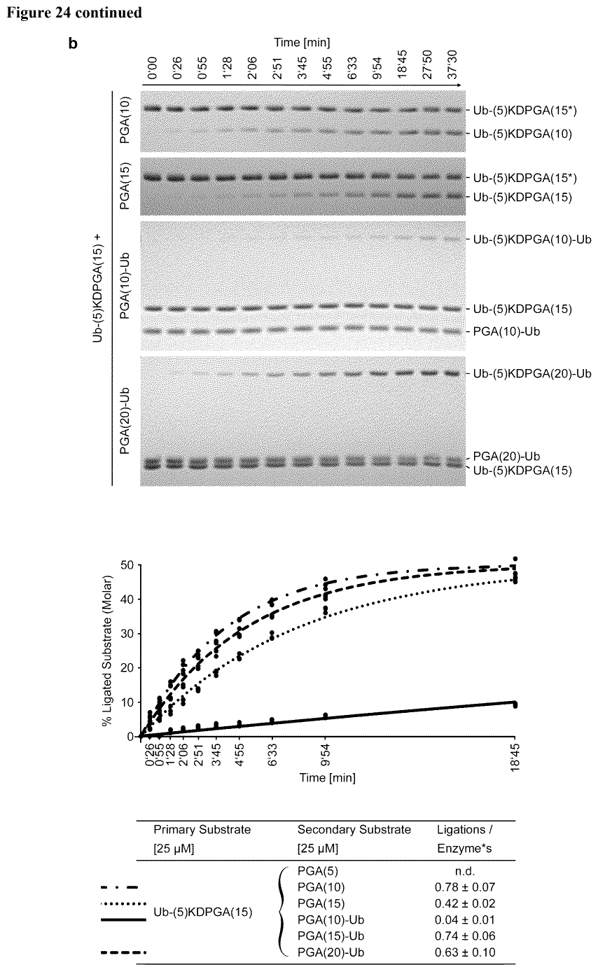

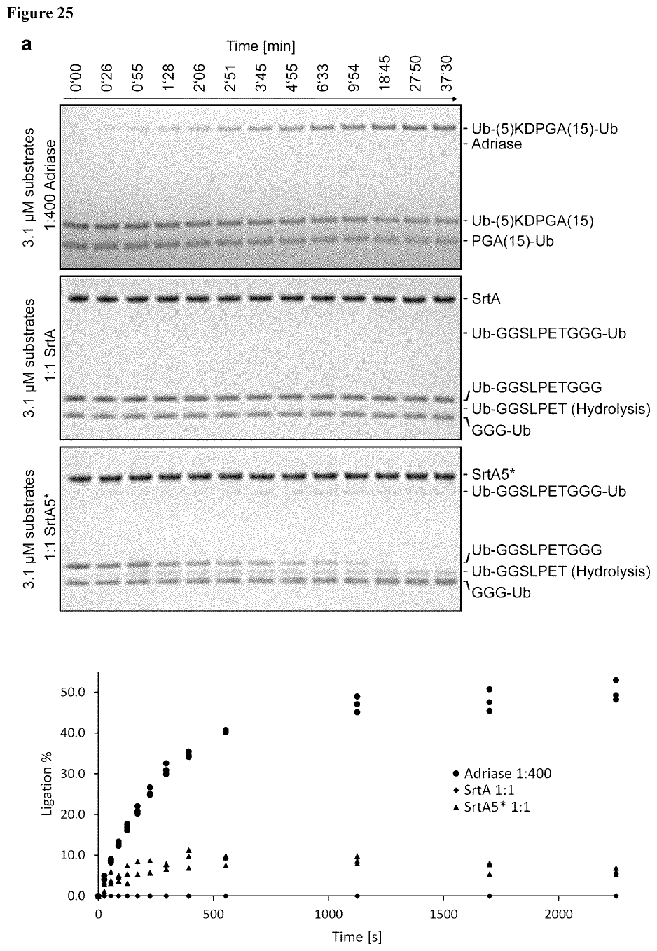

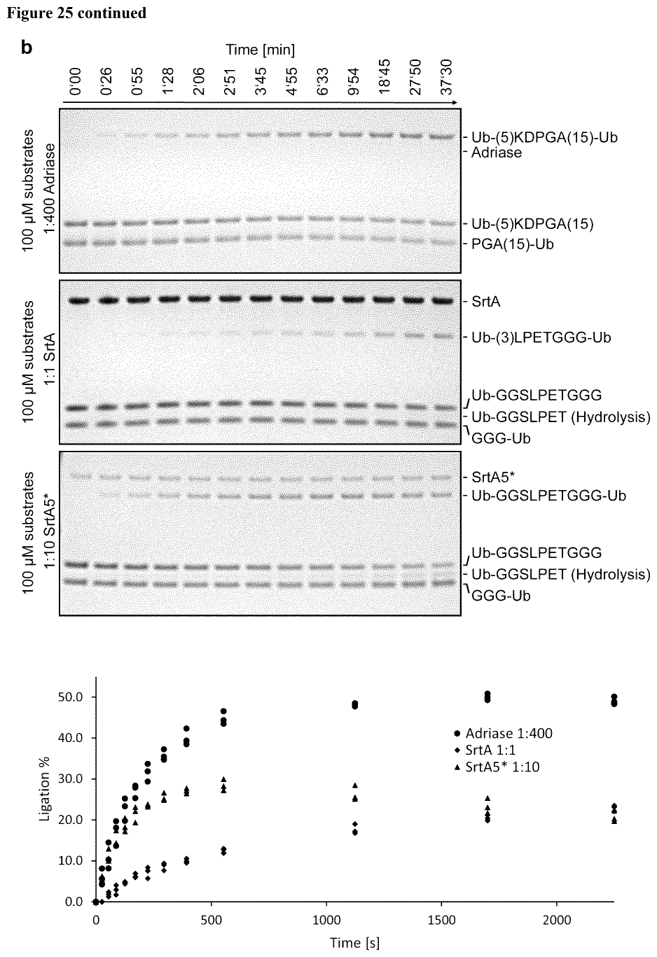

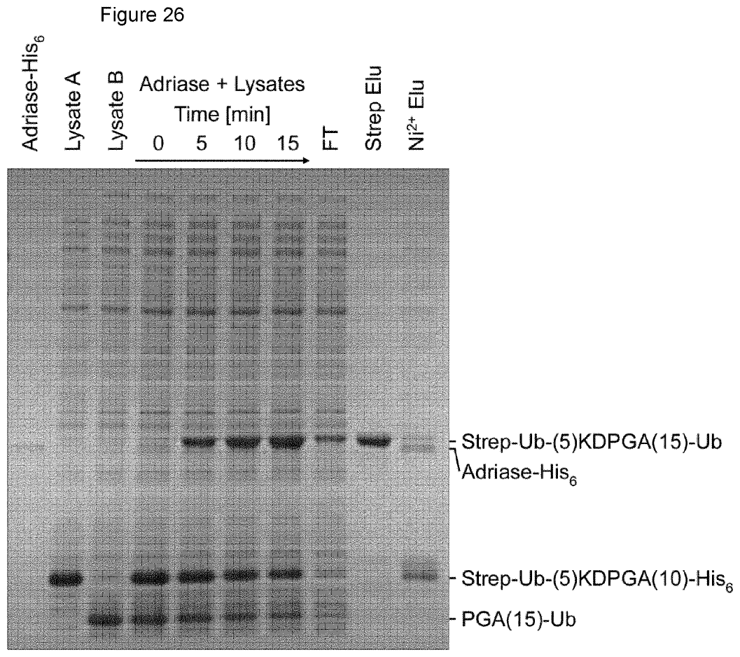

As will be shown in the following examples, Adriase is not only highly divergent in sequence, structure and substrate specificity from the proteasome, but also assumes an entirely different catalytic mechanism and function. Despite these differences, it was envisaged in context of the present invention that in analogy to the proteasome, a conserved serine or threonine residue may act as catalytic residue upon removal of the preceding amino acids (FIG. 1). In the proteasome, this is achieved through autocatalytic cleavage of the propeptide upon complex assembly. Because Adriase neither forms such a complex (FIGS. 5A and 6) nor appears to possess hydrolytic activity, it was further envisaged that a methionine aminopeptidase might be used for its activation by removing the start-methionine preceding the conserved serine or threonine residue. Thus, in the following, all Adriase constructs were produced without further N-terminal modifications (such as purification tags) in methionine aminopeptidase encoding strains. The so produced proteins indeed lack the start-methionine (FIG. 2D) and instead have a free amino group at the active site, a prerequisite for efficient catalysis (FIG. 10B).

For the following experiments, Adriase genes MM_2909 (SEQ ID NO: 376) and MJ_0548 (SEQ ID NO: 378) as well the MtrA genes MM_1543 (SEQ ID NO: 377) and MJ_0851 (SEQ ID NO: 379) originating from Methanocaldococcus jannaschii and Methanosarcina mazei were amplified via PCR from genomic DNA of said archaea (DSM3647 and DSM2661 (DSMZ)) and cloned into pET30 vectors for recombinant protein expression in Escherichia coli BL21 DE3 cells (Stratagene) with the help of the rare codon plasmid pACYC-RIL. An exception presented N-terminally modified M. jannaschii MtrA (SEQ ID NO: 450), which was cloned into pET28b vector instead. Through choice of appropriate PCR primers (SEQ ID NO: 394-401, 403-412, 451-452 and 540-542), the following variations were produced: M. mazei Adriase (MM 2909; SEQ ID NO:376) with a sequence encoding a C-terminal His6 (SEQ ID NO: 678)-(SEQ ID NO: 380)), Strep-(SEQ ID NO: 381)), Myc-(SEQ ID NO: 382) or HA-tag (SEQ ID NO:383) and as active site mutant AdriaseT1A with C-terminal His6 (SEQ ID NO: 678)-tag (SEQ ID NO: 384); M. jannaschii Adriase (MJ 0548) was cloned with a C-terminal His6 (SEQ ID NO: 678)-tag (SEQ ID NO: 385), as active site mutant Adriase^{S1A} with C-terminal His₆ (SEQ ID NO: 678)-tag (SEQ ID NO: 386), without OB-like domain (A203-293, C-terminal His₆ (SEQ ID NO: 678)-tag; SEQ ID NO: 387), as active site mutant without OB-domain (Adriase^{ΔOBS1A}, C-terminal His₆ (SEQ ID NO: 678)-tag; SEQ ID NO: 132), without NTN domain (SEQ ID NO: 519), without insertion element (Δ28-57, C-terminal His₆ (SEQ ID NO: 678)) (SEQ ID NO: 388) and with N-terminal His6 (SEQ ID NO: 678)-tag (N-His₆ (SEQ ID NO: 678), SEQ ID NO: 450); Truncated MtrA constructs, M. mazei MtrA^{Δ219-240} (MM_1543; SEQ ID NO: 377) with N-terminal Strep-tag (SEQ ID NO: 390), M. jannaschii MtrA^{Δ225-245} (MJ_0851; SEQ ID NO: 379) with N-terminal His₆ (SEQ ID NO: 678)-tag (SEQ ID NO: 391) and M. jannaschii MtrA^{Δ174-245} with N-terminal His₆ (SEQ ID NO: 678)-tag (SEQ ID NO: 519), were also generated. The Ub-KDPGA(SEQ ID NO: 311) (10) construct (SEQ ID NO: 392) was produced by replacing the C-terminus (amino acids 82-87 of SEQ ID NO: 422) of precursor C. subterraneum ubiquitin (Csub_C1474, synthesized by Eurofins; SEQ ID NO: 393) with the M. jannaschii MtrA KDPGA(SEQ ID NO: 311) (10) motif (amino acids 159-173; SEQ ID NO: 423) and by introducing an N-terminal His₆ (SEQ ID NO: 678)-tag (a summary of the templates and primers used can be found in Table 2). In a similar manner, N-terminally His-tagged Ubiquitin was modified C-terminally with the M. mazei MtrA-derived sequences (5)KDPGA(SEQ ID NO: 311) (15), (5)KDPGA(SEQ ID NO: 311) (15)-Strep, (5)KDPGA(SEQ ID NO: 311) (10), (5)KD and with the M. jannaschii MtrA derived sequences (5)KDPGA(SEQ ID NO: 311) (10) (SEQ ID NO: 520, 522-524, 526). C-terminally His-tagged Ubiquitin was modified N-terminally with a start-methionine and the M. mazei MtrA-derived sequence PGA(10), PGA(15), PGA(20) or the M. jannaschii MtrA derived sequence PGA(15) (SEQ ID NO: 521, 525, 527 and 530). Furthermore, Ubiquitin gene variants with an a C-terminal GGSLPETGGGHHHHHH (SEQ ID NO: 669) tag (SEQ ID NO: 536), with an N-terminal His₆ (SEQ ID NO: 678)-tag, TEV cleavage site and GG modification (SEQ ID NO: 537) or with N-terminal Strep- and C-terminal M. mazei MtrA derived (5)KDPGA(SEQ ID NO: 311) (10)-His modification (SEQ ID NO: 538) were generated. Camelid α-ricin single-domain antibody (sdAb), Cyclophilin (CyP) or Glutathione-S-Transferase (GST) sequences were fused to an N-terminal M. mazei PGA(10) sequence and a C-terminal His₆ (SEQ ID NO: 678)-tag (SEQ ID NO: 663-665). Finally, Sortase A (SrtA; SEQ ID NO: 662) and the Sortase A pentamutant (SrtA5*; SEQ ID NO: 535) were cloned as published by Chen et al. (Chen (2011) loc. cit.) without the N-terminal membrane anchor (residues 1-59) and with an N-(SrtA) or C-terminal (SrtA5*) His₆ (SEQ ID NO: 678)-tag. Amongst the above constructs, SEQ ID NO: 522, 526, 527, 535-539 and 662-665 were synthesized by Biocat. SEQ ID NO: 380-388, 521, 525, 527, 530, 539 were cloned with a start-methionine, that was removed through expression in a methionine aminopeptidase encoding strain.

TABLE 2
Primer and template DNA sequences for the generation of protein constructs in this work

| | | Template | fwd | rvs |
|---|---|---|---|---|
| SEQ | | DNA | Primer | Primer |
| ID | | SEQ | SEQ | SEQ |
| NO | Name | ID NO | ID NO | ID NO |
| 380 | M. mazei Adriase with C-terminal His-tag | 376 | 394 | 396 |
| 381 | M. mazei Adriase with C-terminal Strep-tag | 376 | 394 | 397 |
| 382 | M. mazei Adriase with C-terminal Myc-tag | 376 | 394 | 398 |
| 383 | M. mazei Adriase with C-terminal HA-tag | 376 | 394 | 399 |
| 384 | M. mazei Adriase T1A mutant with C-terminal His-tag | 376 | 395 | 396 |
| 385 | M. jannaschii Adriase with C-terminal His-tag | 378 | 400 | 403 |
| 386 | M. jannaschii Adriase S1A mutant with | 378 | 401 | 403 |
| | C-terminal His-tag | | | |
| 387 | M. jannaschii Adriase without OB, with | 378 | 400 | 404 |
| | C-terminal His-tag | | | |
| 132 | M. jannaschii Adriase S1A without OB, | 378 | 401 | 404 |
| | with C-terminal His-tag | | | |
| 388 | M. jannaschii Adriase Δ28-57, C-terminal His6 | 378 | 400/405 | 403/406 |
| 390 | M. mazei MtrAΔ219-240 with N-terminal Strep-tag | 377 | 407 | 408 |
| 391 | M. jannaschii MtrAΔ225-245 with N-terminal His6-tag | 379 | 409 | 410 |
| 392 | M. jannaschii Ub-KDPGA(10) | 393 | 411 | 412 |
| 450 | M. jannaschii Adriase with N-terminal His6-tag | 378 | 451 | 452 |
| 518 | M. jannaschii MtrAΔ174-240 with N-terminal His6-tag | 379 | 409 | 540 |
| 519 | M. jannaschii Adriase without NTN, with | 378 | 541 | 542 |
| | C-terminal His-tag | | | |
| 520 | M. mazei Ub-(5)KDPGA(15) with N-terminal His-tag | 393 | 543 | 545 |
| 521 | M. mazei PGA(15)-Ub with C-terminal His-tag | 393 | 549 | 544 |
| 522 | M. mazei Ub-(5)KDPGA(15) with N-terminal | synthesized | | |
| | His- and C-terminal Strep-tag | by Biocat | | |
| 523 | M. mazei Ub-(5)KD with N-terminal His-tag | 393 | 543 | 547 |
| 524 | M. mazei Ub-(5)KDPGA(10) with N-terminal His-tag | 393 | 543 | 546 |
| 525 | M. mazei PGA(10)-Ub with C-terminal His-tag | 393 | 548 | 544 |
| 526 | M. jannaschii Ub-(5)KDPGA(10) with | synthesized | | |
| | N-terminal His-tag | by Biocat | | |
| 527 | M. jannaschii PGA(15) with C-terminal His-tag | synthesized | | |
| | | by Biocat | | |
| 530 | M. mazei PGA(20)-Ub with C-terminal His-tag | 393 | 550 | 544 |
| 535 | SrtA5* delta 1-59 with C-terminal His-tag | synthesized | | |
| | | by Biocat | | |
| 536 | Ub-GGSLPETGGG | synthesized | | |
| | | by Biocat | | |
| 537 | GGG-Ub | synthesized | | |
| | | by Biocat | | |
| 538 | M. mazei Ub-(5)KDPGA(10) with N-terminal | synthesized | | |
| | Strep and C-terminal His-tag | by Biocat | | |
| 539 | M. mazei PGA(15)-Ub | synthesized | | |
| | | by Biocat | | |
| 662 | His₆-SrtA^{Δ1-59} | synthesized | | |
| | | by Biocat | | |
| 663 | M. mazei PGA(10)-sdAb | synthesized | | |
| | | by Biocat | | |
| 664 | M. mazei PGA(10)-CyP | synthesized | | |
| | | by Biocat | | |
| 665 | M. mazei PGA(10)-GST | synthesized | | |
| | | by Biocat | | |

In general, PCR was performed with Q5 polymerase (NEB) according to the manufacturer's instructions, except for the use of just 0.2 μM of each primer. The PCR fragments were then visualized on 1% agarose gels with Stain G (Serva, used 1:30000). In case of a successful amplification, the PCR products were purified with the PCR purification kit (Qiagen), digested with NdeI/XhoI Fast-Digest enzymes (Thermo) and purified yet another time with the PCR purification kit (Qiagen). pET30 vectors were digested and purified in the same manner, except for the addition of alkaline phosphatase (FastAP, Thermo). Ligations were then performed with 100 ng vector and a threefold molar excess of PCR insert in a 20 μl reaction with T4 Ligase (NEB) at 16 °C. After 1 h, the ligations were used for transformation of chemically competent Top10 cells (Thermo) and selected for pET30 plasmid on agar plates with 50 μg/ml kanamycin (Roth) at 37 °C. After 16 h, resistant colonies were cultivated in LB supplemented with 50 μg/ml kanamycin and used for plasmid isolation with the QIAprep Spin Miniprep Kit (Qiagen). The insertion of the PCR product of interest was then tested by Sanger sequencing using BigDye Terminator v3.1 (Thermo). In case of a successful insertion, the respective plasmids were used for transformation of BL21 DE3 cells (Stratagene) containing the rare codon plasmid pACYC-RIL.

The transformed cells were grown at 25 °C. in M9 minimal medium supplemented with 50 μg/ml Se-Met, Leu, Ile, Phe, Thr, Lys and Val for Se-Met labeling, or in lysogeny broth (LB) for all other purposes. The kanamycin concentration was kept at 25 μg/ml and the chloramphenicol concentration at 12.5 μg/ml. Protein expression was induced at an optical density of 0.4 at 600 nm with 500 μM isopropyl-β-D-thiogalactoside. After 16 h, cells were harvested and all subsequent steps conducted at 7 °C, unless stated otherwise. The cell pellet of His₆ (SEQ ID NO: 678)-tagged constructs was resuspended in 100 mM Tris-HCl pH 8.0, 10 mM Imidazole, 5 mM MgCl₂, 50 μg/ml DNAse (Applichem) and cOmplete protease inhibitor (Roche) and the pellet of all other constructs in 100 mM Tris-HCl pH 8.0, 10 mM TCEP, 5 mM MgCl₂, 50 μg/ml DNAse and cOmplete protease inhibitor. Cells were lysed by three french press passages at 16000 psi, and cleared from cell debris by ultracentrifugation at 100000 g for 45 min. The supernatant was then filtered using membrane filters (Millipore) with a pore size of 0.22 μm.

His₆ (SEQ ID NO: 678)-tagged proteins were purified via HisTrap HP columns (all columns obtained from GE Healthcare) using an Akta Pure FPLC (GE Healthcare) with Unicorn v5.1.0 software. The filtrated supernatant was applied to the equilibrated column (20 mM Tris-HCl pH 8.0, 250 mM NaCl, 20 mM imidazole) and washed with 10 additional column volumes of the same buffer. Bound proteins were then eluted by gradually increasing the imidazole concentration up to 300 mM. The eluted fractions were analyzed via SDS-PAGE and those containing the protein of interest at comparatively high purity pooled and used for subsequent purification steps.

Strep-tagged constructs were purified in the same manner, except for the use of Streptavidin HP columns, a buffer containing 20 mM HEPES-NaOH pH 7.5 and 250 mM NaCl, and a gradient ranging from 0-2.5 mM desthiobiotin.

Next, the His₆ (SEQ ID NO: 678)-tag of the GGG-Ub substrate (SEQ ID NO: 537) was removed using TEV protease (resulting in GGG-Ub (SEQ ID NO: 666)) and the His₆ (SEQ ID NO: 678)-tag of the N-terminally tagged Adriase variant (SEQ ID NO: 451) was removed using Thrombin (resulting in SEQ ID NO: 667). TEV protease was purchased from Sigma and used at weight ratio of 1:10 for 24 h at room temperature in a dialysis tube (buffer exchange to 50 mM Tris pH 8, 0.5 mM DTT, 0.1 mM EDTA). Thrombin protease was purchased from Calbiochem and used at ratio of 10 units per mg of protein for 24 h at room temperature in a dialysis tube (buffer exchange to 50 mM Tris pH 8, 10 mM CaCl₂). Both digests (i.e. GGG-Ub and N-terminally modified Adriase) were then applied a second time to an equilibrated Ni-NTA column (20 mM Tris-HCl pH 8.0, 250 mM NaCl, 20 mM imidazole) and the processed proteins collected in the flow-through.

After these initial purification steps, thermostable M. jannaschii and C. subterraneum proteins were incubated for 10 min at 80 °C and denatured protein removed via centrifugation. The supernatant was then filtered using a membrane filters (Millipore) with a pore size of 0.22 μm and used for subsequent purification steps.

Next, all proteins were concentrated using Amicon centrifugal filters with a 10 kDa molecular weight cut-off (Merck) to a concentration of 10 g/1. An exception presented the MtrA constructs, which were concentrated to 3 g/l (M. mazei MtrA^{Δ219-240} with N-terminal Strep-tag (SEQ ID NO: 390)) and 8.5 g/l (M. jannaschii MtrA^{Δ225-245} with N-terminal His₆ (SEQ ID NO: 678)-tag (SEQ ID NO: 391)), respectively.

Finally, a maximum of 0.02 column volumes of the concentrated proteins were applied to a Superdex 75 size-exclusion column (buffer A: 20 mM HEPES-NaOH pH 7.5, 100 mM NaCl, 50 mM KCl, 0.5 mM TCEP). Eluted fractions were analyzed via SDS-PAGE, pooled and concentrated as described above. For long-term storage, the protein containing fractions were supplemented with 15% glycerol, flash frozen in liquid nitrogen and stored at −80 °C.

The identity and purity of the purified proteins was confirmed via SDS-PAGE (all constructs) and a variety of other methods, including mass spectrometry, light scattering and X-ray crystallography, as described in the following. While these methods confirmed the expected sequence of all other constructs, LC-MS (Liquid chromatography-mass spectrometry) revealed the mass of an MtrA^{Δ194-245} (SEQ ID NO: 420) truncation (FIG. 7D) for the M. jannaschii MtrA^{Δ225-245} (SEQ ID NO: 391) construct. This suggested proteolysis by endogenous E. coli proteases, a common phenomenon when purifying proteins with long unstructured terminal regions, such as the MtrA C-terminal linker between catalytic domain and membrane anchor. Because the obtained MtrA^{Δ194-245} (SEQ ID NO: 420) showed high stability and contained the catalytic domain with the crucial Adriase recognition motif, it was used in the following experiments.

### EXAMPLE 2: ADRIASE INTERACTS WITH METHYLTRANSFERASE A (MTRA)

To elucidate the so far unknown function of Adriase proteins, a pulldown experiment was conducted, in which M. mazei Adriase was coupled to magnetic beads via C-terminal Strep-(Experiment 2.1; SEQ ID NO:381), Myc-(Experiment 2.2; SEQ ID NO:382) or HA-tags (Experiment 2.3; SEQ ID NO:383) and incubated with whole cell extract from M. mazei. After several washes, bound proteins were eluted and analyzed via mass spectrometry.

Specifically, M. mazei Adriase fused to Strep- Myc- or HA-tags was recombinantly expressed in E. coli BL21 (DE3) containing the pACYC-RIL plasmid. Cells were grown at 25 °C in 50 ml LB medium, supplemented with 25 μg/ml kanamycin and 12.5 μg/ml chloramphenicol. Protein expression was induced at an optical density of 0.4 at 600 nm with 500 μM isopropyl-β-D-thiogalactoside. After 16 h, cells were harvested and all subsequent steps conducted at 7 °C, unless stated otherwise. The cell pellet of was resuspended in 20 mM MOPS-NaOH pH 7.1, 150 mM NaCl, 100 mM KCl, lysed by three french press passages at 16000 psi, and cleared from cell debris by ultracentrifugation at 100000 g for 45 min. The supernatant was then filtered using a membrane filters (Millipore) with a pore size of 0.22 μm and supplemented with 0.15% NP40. For binding the tagged Adriase proteins to beads, Magnetic Streptavidin-(87.5 μl; Experiment 1), anti-Myc (Thermo, 175 μl Experiment 2) or anti-HA magnetic beads (Thermo; 175 μl; Experiment 3) were incubated for 1 h at room temperature with the E. coli extracts, containing Strep-, Myc- or HA-tagged Adriase, respectively. Afterwards, the beads were washed five times with 1 ml of buffer B (20 mM MOPS-NaOH pH 7.1, 150 mM NaCl, 100 mM KCl, 0.15% NP40).

The M. mazei cell extract for the pulldown experiments was produced by cultivating M. mazei cells in anaerobic medium according to the recommendations of the DSMZ (German Collection of Microorganisms and Cell Cultures). 20 g of stationary phase cells were harvested by centrifugation, resuspended in 50 ml 20 mM MOPS-NaOH pH 7.1, 150 mM NaCl, 100 mM KCl, lysed by three french press passages and cleared from insoluble fractions by ultracentrifugation at 100000 g for 45 min. The supernatant was then filtered using a membrane filters (Millipore) with a pore size of 0.22 μm and supplemented with 0.15% NP40.

For the pulldown experiments, the produced Adriase beads were incubated with 15 ml of the M. mazei extract for 1 h at room temperature. After two washes with buffer B, bound proteins were eluted with 40 μl buffer B supplemented with 10 μM desthiobiotin or 2 mg/ml HA- or Myc-peptides, respectively. Subsequently, mass spectrometrical analysis of the final eluate was conducted.

For the mass spectrometrical analysis, bound proteins were separated via SDS-PAGE, following an in-gel tryptic digest (13 ng/μl trypsin, 20 mM ammonium bicarbonate; Borchert (Borchert (2010) Genome Res 20:837-46)). LC-MS/MS analysis was performed on a Proxeon Easy nano-LC (Thermo) coupled to an LTQ OrbitrapElite mass spectrometer (Thermo). The data were processed using MaxQuant v1.6.4 (Cox (2008) Nat Biotechnol 26:1367-72) and spectra were searched against the Uniprot M. mazei Gol proteome (UniProt Proteome ID: UP000000595).

As illustrated in Table 3, methyltransferase A (MtrA; Seq ID NO: 423) was detected at high intensities (arbitrary units) in all three experiments. This protein is present in almost all Adriase organisms, indicating an interaction of biological significance. MtrA is part of the membrane-bound MtrA-MtrH complex and acts in the hydrogenotrophic methanogenesis pathway (Wagner (2016) Sci Rep 6:28226). For other subunits of the MtrA-MtrH complex, a significantly lower signal was determined indicating that Adriase interacts specifically and directly with MtrA.

TABLE 3
Adriase interacts with MtrA.

| | | Intensity | Intensity | Intensity |
|---|---|---|---|---|
| Rank by | Detected | (Experiment | (Experiment | (Experiment |
| Intensity | Protein | 2.1) | 2.2) | 2.3) |
| 1 | Adriase | 6.4E+09 | 6.8E+09 | 2.1E+10 |
| 2 | MtrA | 3.2E+07 | 4.9E+08 | 4.1E+09 |
| 26 | MtrH | 6.0E+07 | 6.5E+07 | 2.0E+08 |
| 263 | MtrG | 8.6E+05 | 1.2E+06 | 1.5E+07 |
| 325 | MtrB | 1.4E+06 | 3.2E+06 | 6.2E+06 |
| 373 | MtrE | 2.1E+06 | 2.2E+06 | 3.8E+06 |
| 661 | MtrF | n.d. | n.d. | 4.3E+05 |

### EXAMPLE 3: ACTIVATED ADRIASE FORMS A COVALENT BOND WITH THE N-TERMINAL MTRA (MTRAN-ADRIASE) FRAGMENT

To study the interaction between Adriase and MtrA as found in Example 2 in more detail, a second pulldown experiment was performed using purified M. mazei Adriase and a purified MtrA variant (SEQ ID NO: 390) lacking the C-terminal membrane anchor.

Specifically, 50 μg His6 (SEQ ID NO: 678)-tagged M. mazei Adriase (SEQ ID NO: 380), 50 μg Strep-tagged M. mazei MtrA^{Δ219-240} (SEQ ID NO:390) and 50 μg BSA were incubated with 100 μl 50% (v/v) Protino Ni-NTA beads (Machery Nagel) in buffer C (20 mM Tris-HCl pH 8, 250 mM NaCl) for 5 min at room temperature. Unbound proteins were removed by centrifugation at 100 g for 1 min. After four wash steps, bound proteins were eluted with buffer C supplemented with 500 mM imidazole and the fractions analyzed via SDS-PAGE.

This analysis (FIG. 2A) did not only confirm the interaction between MtrA and Adriase by presence of both in the final eluate, but surprisingly also revealed the formation of a slower migrating reaction product. To characterize this product, Adriase and MtrA were incubated and subsequently subjected to LC-MS (Liquid Chromatography-Mass Spectrometry) analysis.

Specifically, 0.5 g/l C-terminally His₆ (SEQ ID NO: 678)-tagged M. mazei Adriase (SEQ ID NO:380) was incubated with 0.5 g/l Strep-tagged M. mazei MtrA^{Δ219-240} (SEQ ID NO:390) in buffer A (20 mM HEPES-NaOH pH 7.5, 100 mM NaCl, 50 mM KCl, 0.5 mM TCEP) over night at 4 °C. Desalted samples were subjected to a Phenomenex Aeris Widepore 3.6 μm C4 200 Å (100×2.1 mm) column, eluted with a 30-80% H₂O/acetonitrile gradient over 15 min in the presence of 0.05% trifluoroacetic acid and analyzed with a Bruker Daltonik microTOF. Data processing was performed with Bruker Compass DataAnalysis 4.2 and the m/z deconvoluted with the MaxEnt module to obtain the protein mass.

The mass spectrometrical analysis of the reaction identified masses for MtrA and for activated Adriase without N-terminal methionine, a prerequisite for catalytic activity (see Example 1; FIG. 2C-D). Moreover, it revealed a C-terminal MtrA fragment (MtrA^{C}, corresponding to positions 166 to 229 of SEQ ID NO: 390; FIG. 2B) and the corresponding MtrA^{N}-Adriase conjugate (SEQ ID NO: 431; FIG. 2D). This conjugate corresponds to the slower migrating reaction product observed in the pulldown analysis (FIG. 2A). Its mass is 18 Da lighter than the combined mass of its components (FIG. 2C-D), indicating that it is a covalent protein adduct formed by condensation. Thus, this experiment confirmed the interaction between Adriase and MtrA and revealed the formation of a covalent conjugate between the two proteins, which migrates slower in SDS-PAGE analysis.

### EXAMPLE 4: THE ADRIASE N-TERMINUS FORMS AN AMIDE BOND WITH MTRA ASPARTATE

From the protein masses determined in Example 2 (FIG. 2B-D), the MtrA modification site can be inferred: It is the position at which MtrA is processed to Adriase-MtrA^{N} and MtrA^{C}, specifically the bond between aspartate and proline within a highly conserved KDPGA (SEQ ID NO: 311) motif (FIG. 3; R is used instead of K in some MtrA homologs (SEQ ID NO: 310-311)). As the Adriase catalytic center has been hypothesized to involve a conserved serine or threonine residue at its activated N-terminus (see Example 1), the postulated conjugation of MtrA^{N}(KD . . . ) with the threonine of the M. mazei Adriase N-terminus in its active form (TLVIAFIGK (SEQ ID NO:668) . . . ; see positions 1 to 9 of SEQ ID NO: 380) should result in the fusion peptide [ . . . ]KDTLVIAFIGK[ . . . ](SEQ ID NO: 425).

Based on these considerations, a re-analysis of the MS data of Example 2 was performed. Because this analysis involved a trypsin digest, a tryptic peptide with the sequence DTLVIAFIGK (SEQ ID NO:426) was expected. Indeed, this fragment was as abundant as the unmodified M. mazei Adriase N-terminus, while the respective unmodified MtrA fragment was not identified (Table 4). These results show that, despite mechanistic differences in the activation of their catalytic center, both Adriase and proteasome utilize an N-terminal serine or threonine for their diverse functions.

TABLE 4
Adriase modifies MtrA.

| | | # Identi- | # Identi- | # Identi- |
|---|---|---|---|---|
| | Corres- | fications | fications | fications |
| | ponding | (Experiment | (Experiment | (Experiment |
| Peptide | Protein | 2.1) | 2.2) | 2.3) |
| TLVIAFIGK | Adriase | 23 | 26 | 26 |
| DPGAFDADPLV | MtrA | 0 | 0 | 0 |
| VEISEEGEEEEE | | | | |
| GGVVR | | | | |
| DTLVIAFIGK | MtrA^{N}- | 5 | 28 | 39 |
| | Adriase | | | |

While these results confirm that a covalent bond between the Adriase active site threonine and MtrA aspartate is formed by condensation, the nature of this bond remained enigmatic. In analogy to the first step of proteasomal hydrolysis (Huber (2016) Nat Commun 7:10900), it appeared possible that an ester bond is formed involving the threonine hydroxyl group and the aspartate carbonyl group. However, such a bond would be labile and accordingly, is hydrolyzed in the second step of proteasomal hydrolysis. This is not observed in case of Adriase and so, it appeared possible that the aspartate carbonyl group is subsequently transferred to the threonine amino group, forming a stable, regular peptide bond.

To discriminate between these two scenarios—hydroxyl ester or peptide bond —, a dimethyl labeling experiment (Jhan (2017) Anal Chem 89:4255-4263) was conducted, a method that modifies all free amino groups. For this purpose, the MtrA^{N}-Adriase conjugate band was excised from the SDS-gel shown in FIG. 2A (“Elu”). Following an in-gel tryptic digest (13 ng/μl trypsin, 20 mM ammonium bicarbonate; Borchert (2010) loc. cit.), extracted protein fragments were desalted with C18 StageTips (Rappsilber (2007) Nat Protoc 2:1896-906) and dimethylated (0.16% CH₂O, 22 mM NaBH₃CN, 100 mM TEAB; (Boersema (2009) Nat Protoc 4:484-94)) with an incorporation rate of 91.3%. LC-MS/MS analysis on a Proxeon Easy nano-LC (Thermo) coupled to an LTQ OrbitrapElite mass spectrometer (Thermo). The data were processed using MaxQuant v.1.6.4 (Cox (2008) loc. cit.) spectra searched against a custom peptide database and the Uniprot M. mazei Gol proteome.

This analysis showed that dimethyl modifications in the fusion peptide DTLVIAFIGK (SEQ ID NO: 426) were found only at the newly generated aspartate N-terminus and the lysine residue (FIG. 4). By contrast, a methylation of the Adriase threonine, as it would be observed in case of a hydroxyl ester, is not detected. This indicates that its amino group is engaged in a regular amide bond with the MtrA aspartate.

To further substantiate these results, peptides from the MtrA^{N}-Adriase conjugate gel band were compared quantitatively with peptides from gel bands corresponding to MtrA or Adriase proteins alone. For this purpose, MtrA and Adriase gel bands were excised from the SDS-gel shown in FIG. 2A (“Elu”) and processed just like the MtrA^{N}-Adriase conjugate (see above): Following an in-gel tryptic digest (13 ng/μl trypsin, 20 mM ammonium bicarbonate; Borchert (2010) loc. cit.), extracted protein fragments were desalted with C18 StageTips (Rappsilber (2007) loc. cit.) and dimethylated (0.16% CH₂O, 22 mM NaBH₃CN, 100 mM TEAB; (Boersema (2009) loc. cit.)) with an incorporation rate of 89-92%. For each of the samples, reagents with different isotopes were used in the labeling procedure: CH₂O/NaBH₃CN were used for the MtrA^{N}-Adriase conjugate gel band, resulting in a (CH₃)₂ modification of primary amines (light label); CD₂O/NaBH₃CN were used for the MtrA gel band, resulting in a (CHD₂)₂ modification of primary amines (medium label); ¹³CD₂O/NaBD₃CN were used for the Adriase gel band, resulting in a (¹³CD₃)₂ modification of primary amines (heavy label). The samples were then combined and LC-MS/MS analysis on a Proxeon Easy nano-LC (Thermo) coupled to an LTQ OrbitrapElite mass spectrometer (Thermo). The data were processed using MaxQuant v.1.6.4 (Cox (2008) loc. cit.) spectra searched against a custom peptide database and the Uniprot M. mazei Gol proteome.

The results of this quantitative analysis (Table 5) support the formation of the MtrA^{N}-Adriase conjugate: Peptide fragments corresponding to MtrA^{N} (residues 1-154 (SEQ ID NO: 517)) and Adriase (SEQ ID NO: 107) are abundant in the to MtrA^{N}-Adriase gel band, while fragments corresponding to MtrA^{C} (residues 155-218 (SEQ ID NO: 424)) are only detected at very low levels. Furthermore, just like in the first experiment, no methylation is observed at the threonine within the DTLVIAFIGK (SEQ ID NO: 426) fusion peptide, indicating that its amino group is engaged in a regular amide bond with the MtrA aspartate.

TABLE 5
Adriase forms a covalent conjugate with MtrA^{N} (residues 1-154).

| | | Intensity L | | |
|---|---|---|---|---|
| | Sequence | (MtrA^{N}- | Intensity M | Intensity H |
| Fragment | (* = dimethyl label) | Adriase) | (MtrA) | (Adriase) |
| MtrA 125-146 | *FQEQVQVVNLLDT | 496 | 1137 | 0 |
| | EDMGAITSK* | | | |
| MtrA 154-191 | *DPGAFDADPLVVEI | 0 | 100 | 0 |
| | SEEGEEEEEGGVVRP | | | |
| | VSGEIAVLR | | | |
| MtrA 201-209 | *MMDIGNLNK* | 53 | 10000 | 1 |
| MtrA 149-154 + | *ELASKDTLVIAFIG | 48 | 0 | 0 |
| Adriase 1-9 (amide) | K | | | |
| MtrA 149-154 + | *ELASKD*TLVIAFIG | 0 | 0 | 0 |
| Adriase 1-9 (ester) | K* | | | |
| MtrA D154 + | *DTLVIAFIGK* | 1975 | 0 | 0 |
| Adriase 1-9 (amide) | | | | |
| MtrA D154 + | *D*TLVIAFIGK* | 0 | 0 | 0 |
| Adriase 1-9 (ester) | | | | |
| Adriase 10-19 | *NGAVMAGDMR | 407 | 0 | 1723 |
| Adriase 1-9 | *TLVIAFIGK* | 85 | 1 | 1639 |

Detected protein fragments in excised polyacrylamide gel bands corresponding to Adriase (H), MtrA (M) or an MtrA^{N}-Adriase conjugate (L; see FIG. 2A). The samples were digested with trypsin and dimethylated at primary amine groups (indicated by asterisks), using different isotopes (H = Heavy; M = Medium; L = Light). Note, that the relative intensities (normalized to 10000) for a given peptide reflect quantitative differences between the samples. The band corresponding to MtrA^{N}-Adriase also contains small amounts of unconjugated MtrA and Adriase proteins, possibly due to the reversibility of the reaction.

Accordingly, the generated data show that Adriase can form a covalent conjugate with MtrA^{N}, by forming a peptide bond between the N-terminal threonine/serine of the active Adriase and the aspartate residue in the conserved KDPGA (SEQ ID NO: 311) motif within the MtrA protein.

### EXAMPLE 5: A SHORT RECOGNITION MOTIF IS SUFFICIENT FOR THE INTERACTION WITH ADRIASE

In order to further study conjugate formation between MtrA and Adriase, static light scattering (SEC-MALS) experiments were performed using proteins derived from M. jannaschii, a hyperthermophilic organism known for its stable proteins. These results were then further substantiated with MST (Microscale thermophoresis) measurements and a crystal structure of Adriase with a bound substrate.

For light scattering experiments, 50 μl of the catalytically inactive M. jannaschii Adriase^{S1A} mutant (SEQ ID NO: 386) at 200 μM, 50 μl M. jannaschii MtrA^{Δ194-245} (SEQ ID NO: 420) at 200 μM or a 1:1 molar mixture of the same were injected on a Superdex S200 10/300 GL gel size-exclusion column (20 mM HEPES-NaOH pH 7.5, 50 mM NaCl, 100 mM KCl) coupled to a miniDAWN Tristar Laser photometer (Wyatt) and a RI-2031 differential refractometer (JASCO). Data analysis was carried out with ASTRA v7.3.0.18 software (Wyatt).

The results depicted in FIG. 5A show that Adriase and MtrA alone display a comparable elution behavior, whereas the mixture of both elutes at a lower volume, indicating complex formation. This interpretation is supported by light scattering measurements (thick lines, plotted on the secondary Y-axis in FIG. 5A). The determined masses (Table in FIG. 5A) closely resemble the theoretical monomeric masses for Adriase and MtrA alone and for a complex formed by one Adriase and one MtrA molecule. Hence, this experiment demonstrated the monomeric nature of both proteins and that MtrA and inactive Adriase form a heterodimer.

To further investigate, which regions in MtrA are required for this interaction, a similar experiment using a shorter MtrA version, M. jannaschii MtrA^{Δ174-245} was conducted. Specifically, 50 μl of the catalytically inactive M. jannaschii Adriase^{S1A} mutant (SEQ ID NO: 386) at 200 μM, 50 μl M. jannaschii MtrA^{Δ174-245} (SEQ ID NO: 518) at 200 μM or a 1:1 molar mixture of the same were injected on a Superdex S200 10/300 GL gel size-exclusion column (20 mM HEPES-NaOH pH 7.5, 50 mM NaCl, 100 mM KCl) coupled to a miniDAWN Tristar Laser photometer (Wyatt) and a RI-2031 differential refractometer (JASCO). Data analysis was carried out with ASTRA v7.3.0.18 software (Wyatt).

The results depicted in FIG. 17 show the formation of a Adriase:Mtr^{Δ174-245} heterodimer, just like in the above experiment (FIG. 5A). Accordingly, residues within the truncated C-terminal MtrA element are not necessary for the Adriase-MtrA interaction.

To substantiate these results, the dissociation constant (K_{D}) for this interaction was determined via MST (Microscale thermophoresis). Specifically, M. jannaschii MtrA^{Δ194-245} (SEQ ID NO: 420) or M. jannaschii MtrA^{Δ174-245} (SEQ ID NO: 518) were fluorescently labeled using the NT-647-NHS kit (Nanotemper). Next, a serial 1:1 dilution of the catalytically inactive M. jannaschii Adriase^{S1A} mutant (SEQ ID NO:386) ranging from 90 μM to 2.7 nM was prepared and mixed with 50 nM labeled M. jannaschii MtrA^{Δ194-245} or 50 nM labeled M. jannaschii MtrA^{Δ174-245} (20 mM HEPES-NaOH pH 7.5, 150 mM NaCl, 50 mM KCl, 0.5 mM TCEP, 0.05% NP40, 0.1 g/l BSA). MST measurements were performed with a Monolith NT.115 (Nanotemper), using various MST power and laser intensity settings to test the general validity of the obtained data. The final results were obtained in three independent experiments and measured at a temperature of 25 °C, using MST power 80% and laser intensity 40%. The binding curve shown in FIGS. 5B and 18G were fitted to the data, using the NT Analysis 1.5.41 software (Nanotemper).

In a second set of MST experiments, a more detailed analysis of the binding motif (FIG. 18A-F and table in FIG. 5B) was performed using synthetic peptides (Genscript) based on the M. jannaschii MtrA sequence. These peptides contained the KDPGA (SEQ ID NO: 311) motif plus up to 15 N- and 10 C-terminal residues and were linked to the fluorophore fluorescein-5-isothiocyante (FITC) either N-terminally via aminohexanoic acid (Ahx) or C-terminally via an extra lysine (SEQ ID NOs: 367-372; Table 6). For the MST measurements, 10 nM of these peptides were mixed with a serial dilution of M. jannaschii Adriase^{S1A} and the experiment was otherwise performed as described for the M. jannaschii MtrA^{Δ194-245}-Adriase^{S1A} and the M. jannaschii MtrA^{Δ174-245}-Adriase^{S1A} interactions, above.

TABLE 6
Fluorophore-coupled peptides used for MST analysis

| | | SEQ |
|---|---|---|
| | | ID |
| Peptide | Sequence | NO |
| (10)KDPGA(SEQ | ITQAIKECLSKDPGAIDEDPFIIELK-FITC | 370 |
| ID NO: 311) | | |
| (10) | | |
| (5)KDPGA (SEQ | KECLSKDPGAIDEDPFIIELK-FITC | 371 |
| ID NO: 311) | | |
| (10) | | |
| (0)KDPGA(SEQ | KDPGAIDEDPFIIELK-FITC | 372 |
| ID NO: 311) | | |
| (10) | | |
| (15)KDPGA(SEQ | FITC-Ahx- | 367 |
| ID NO: 311) | EDIGKITQAIKECLSKDPGAIDEDPFIIEL | |
| (10) | | |
| (15)KDPGA(SEQ | FITC-Ahx-EDIGKITQAIKECLSKDPGAIDEDP | 368 |
| ID NO: 311) | | |
| (5) | | |
| (15)KDPGA(SEQ | FITC-Ahx-EDIGKITQAIKECLSKDPGA | 369 |
| ID NO: 311) | | |
| (0) | | |

The results of these experiments as depicted in FIG. 18 and the table of FIG. 5B show that Adriase binds the 20 amino acid motif (5)KDPGA(SEQ ID NO: 311) (10) as tightly as M. jannaschii MtrA^{Δ194-245} and that the 15 amino acid motif (0)KDPGA(SEQ ID NO: 311) (10) is still bound with sub-micromolar affinity. Accordingly, the data shows that a short recognition motif is sufficient for Adriase binding, even when presented as isolated peptide.

To support these conclusions, crystal structures of Adriase and of a complex between catalytically inactive Adriase and the (15)KDPGA(SEQ ID NO: 311) (10) peptide were determined.

For this purpose, N-terminally modified Adriase (SEQ ID NO: 450) was purified and processed (yielding SEQ ID NO: 667) as described in Example 1, except for the use of a different gel filtration buffer (20 mM HEPES-NaOH pH 7.5, 150 mM NaCl, 0.5 mM TCEP) and a final concentration of 15 g/1. Crystals were obtained in “sitting drops” by mixing 15 g/l protein with an equal volume of crystallization buffer (100 mM HEPES-NaOH pH 7.5, 70% MPD). Crystals were flash frozen in liquid nitrogen and data collected at 100K at beamline X10SA of the Swiss Light Source (Villigen, Switzerland), using a MarCCD 225 mm CCD detector.

As the obtained data alone allowed no structure solution, the above experiment was repeated with a Se-Met labeled version of the above protein (SEQ ID NO: 667), which crystallized with a concentration of 6 g/l under the same buffer conditions. Crystals were flash frozen in liquid nitrogen and data collected at 100K at beamline X₁₀SA of the Swiss Light Source (Villigen, Switzerland), using a MarCCD 225 mm CCD detector. All data were indexed, integrated and scaled using XDS (Kabsch (2010) Acta Crystallogr D Biol Crystallogr 66:125-32). After heavy atom localization and density modification with SHELX (Sheldrick (2008) Acta Crystallogr A 64:112-22), substructure refinement with SHARP (Vonrhein (2008) Mehods Mol Biol 364:215-30), density modification with Solomon (Abrahams (1996) Acta Cryst D52:30-42) and secondary structure recognition with ARP/WARP (Perrakis (1999) Nat Struct Biol 6:458-63), most of the structure could be traced and built by Buccaneer (Cowtan (2006) Acta Crystallogr D Biol Crystallogr 62:1002-11). The data was refined against the higher-resolution native data (see above) and completed by cyclic manual modeling with Coot (Emsley (2004) Acta Crystallogr D Biol Crystallogr 60:2126-32) and refinement with REFMAC (Murshudov (1999) Acta Crystallogr D Biol Crystallogr 55:247-55).

The so obtained Adriase structure could in the following be used to solve the structure of a M. jannaschii Adriase^{S1A}-(15)KDPGA(SEQ ID NO: 311) (10) complex. The respective crystals were obtained and measured in a similar fashion, except that they grew by mixing an equimolar solution of protein and peptide (SEQ ID NO: 386 and 367; 10.5 g/l in 20 mM HEPES-NaOH pH 7.5, 50 mM NaCl, 0.5 mM TCEP) with crystallization buffer (100 mM MES-NaOH pH 6.0, 200 mM NaCl, 20% PEG2000). Prior to loop-mounting and flash-cooling in liquid nitrogen, crystals were briefly transferred to a droplet of crystallization buffer supplemented with 20% glycerol for cryoprotection. Diffraction experiments were performed at 100K and a wavelength of 1 Å at beamline X10SA, using a Pilatus 6M-F hybrid pixel photon counting detector. Data were indexed, integrated and scaled using XDS, yielding a dataset in space group P2₁2₁2₁ with a resolution cutoff at 3.05 Å. The complex structure was solved by molecular replacement with MOLREP (Vagin (2000) Acta Crystallogr D Biol Crystallogr 56:1622-4) using the above described structure of SeMet-labeled M. jannaschii Adriase as a search model and subsequent refinement with Coot and REFMAC.

The obtained crystal structure of the (15)KDPGA(SEQ ID NO: 311) (10)-Adriase^{S1A} complex (FIG. 6) shows that the helix preceding the KDPGA (SEQ ID NO: 311) motif is not crucial for the interaction, while the KDPGA (SEQ ID NO: 311) residues and the ten amino acid residues following the motif are bound via beta-sheet interactions. This result supports the conclusion, that a small amino acid motif, such as (5)KDPGA(SEQ ID NO: 311) (10), is sufficient for a high affinity interaction with Adriase.

### EXAMPLE 6: ADRIASE MODIFICATIONS ARE REVERSIBLE AND ALLOW THE RECOMBINATION OF SUBSTRATES VIA THE ADRIASE RECOGNITION MOTIF

To test the kinetics MtrA^{N}-Adriase conjugate formation, Adriase (SEQ ID NO: 385) and MtrA from M. jannaschii (SEQ ID NO: 420) were recombinantly expressed, purified and subjected to a time course experiment analyzing the reaction between Adriase and MtrA over time. Specifically, 14 μM of M. jannaschii Adriase (SEQ ID NO: 385) and M. jannaschii MtrA^{Δ194-245} (SEQ ID NO:420) were mixed in buffer A (20 mM HEPES-NaOH pH 7.5, 100 mM NaCl, 50 mM KCl, 0.5 mM TCEP) at room temperature. The reaction was stopped by addition of 2% SDS at the time points indicated in FIG. 7A and the samples analyzed via SDS-PAGE.

Surprisingly, a nearly constant fraction of MtrA^{N} was conjugated to Adriase that did not change significantly over time (FIG. 7A). This finding suggests that the reaction of Adriase and MtrA is reversible, resulting in an equilibrium between unmodified and modified MtrA. In the reverse Adriase reaction, MtrA^{C} would react with MtrA^{N}-Adriase, yielding unmodified MtrA^{NC} and Adriase.

To test hypothesis, the above experiment was repeated in the presence of a second, artificial Adriase substrate, namely ubiquitin (Ub)C-terminally fused to the Adriase recognition motif KDPGA(SEQ ID NO: 311) (10) (i.e. KDPGA (SEQ ID NO: 311) and the ten following amino acids (SEQ ID NO: 392)). Specifically, 14 μM of M. jannaschii Adriase (SEQ ID NO: 385), M. jannaschii MtrA^{Δ194-245} (SEQ ID NO:420) and Ub-KDPGA(SEQ ID NO: 311) (10) were mixed in buffer A (20 mM HEPES-NaOH pH 7.5, 100 mM NaCl, 50 mM KCl, 0.5 mM TCEP) at room temperature. The reaction was stopped by addition of 2% SDS at the time points indicated in FIG. 7B and the samples analyzed via SDS-PAGE.

As predicted, Adriase reacts with both substrates to form MtrA^{N}-Adriase (SEQ ID NO: 434) and Ub^{N}-Adriase (Predicted SEQ ID NO: 433) and remove the respective C-terminal fragments (MtrA^{C}, Ub^{C}; FIG. 7B). In the reverse reaction, the C-terminal fragments then react with both Adriase conjugates (MtrAN-Adriase, UbN-Adriase), producing the fusion proteins MtrA^{N}-Ub^{C} (SEQ ID NO: 427) and Ub^{N}-MtrA^{C} (SEQ ID NO: 428), respectively (FIG. 7B-D).

To verify these findings, the observed recombination was also analyzed via LCMS. Specifically, 0.5 g/l of M. jannaschii Adriase (SEQ ID NO:385), 0.5 g/l M. jannaschii MtrA Δ194-245 (SEQ ID NO: 420) and 0.5 g/l Ub-KPDGA(10) (SEQ ID NO: 392) were incubated overnight at room temperature in the same buffer (20 mM HEPES-NaOH pH 7.5, 100 mM NaCl, 50 mM KCl, 0.5 mM TCEP). Desalted samples were subjected to a Phenomenex Aeris Widepore 3.6 μm C4 200 Å (100×2.1 mm) column, eluted with a 30-80% H2O/acetonitrile gradient over 15 min in the presence of 0.05% trifluoroacetic acid and analyzed with a Bruker Daltonik microTOF. Data processing was performed with Bruker Compass DataAnalysis 4.2 and the m/z deconvoluted with the MaxEnt module to obtain the protein mass.

The observed spectra (FIG. 7C-D) confirm the Adriase-catalyzed recombination of MtrA and Ub-KDPGA(SEQ ID NO: 311) (10) via the Adriase recognition motif, resulting in the “chimeric” fusion proteins MtrA^{N}-Ub^{C} (SEQ ID NO: 427) and Ub^{N}-MtrA^{C} (SEQ ID NO: 428). Accordingly, the formation of the covalent peptide bond between Adriase and MtrAN is indeed reversible, enabling the post-translational recombination and/or ligation of two substrates. This shows that the KDPGA(SEQ ID NO: 311) (10) motif is sufficient for Adriase to act on a given substrate protein such as ubiquitin.

Based on the above experiments, a catalytic mechanism for the Adriase reaction can be deduced (FIG. 8). In brief, active Adriase bearing an N-terminal serine or threonine residue reacts with a substrate protein having the conserved KDPGA (SEQ ID NO: 311) recognition motif, cleaves the same between the aspartate (D) and proline (P) residues and forms a new peptide bond between the aspartate and its N-terminus. The reaction releases the C-terminal portion of the substrate protein bearing the PGA sequence as N-terminus. This process is reversible so that either the original C-terminal portion or a different molecule with the PGA sequence can react with the Adriase-substrate^{N} conjugate to restore the substrate protein. Thus, Adriase has peptide recombinase or transpeptidase activity allowing post-translational fusion of protein portions.

Chemically, the proposed catalytic mechanism of the Adriase recombination is a completely unexpected combination of two known proteasomal reactions, hydrolysis and autolysis (Huber (2016) loc. cit.). As depicted in FIG. 8, the reversible Adriase reaction is proposed to differ from proteolysis/autolysis only by avoiding the irreversible hydrolysis step (bottom). Hydrolysis products could not be identified in any of the shown mass spectrometrical analyses of the Adriase reaction (see also Example 11).

### EXAMPLE 7: KINETICS OF THE M. JANNASCHII ADRIASE REACTION

Example 6 showed that Adriase can recombine two proteins via the (X₁)KDPGA(X₂) (SEQ ID NO: 681) motif, by exchanging the respective PGA(X₂) fragments (FIG. 16A). Considering the proposed mechanism (FIG. 8), the same reaction using (X₁)KDPGA(X₂) (SEQ ID NO: 681) as primary and PGA(X₃) as secondary substrate should further promote the formation of the fusion peptide, because more Adriase is available and the number of possible reactions is decreased (FIG. 16B).

To test this assumption, the Adriase reaction was performed with (X₁)KDPGA(X₂) (SEQ ID NO: 681) peptides bearing a C-terminal fluorophore (SEQ ID NOs: 370-371 and 418; see also Table 6) and unmodified PGA(X₃) synthetic peptides (SEQ ID NOs: 373-375; see Table 7) as model substrates. The recombination reaction is expected to result in the formation of a (X₁)KDPGA(X₃) (SEQ ID NO: 681) fusion peptide, releasing the respective C-terminal peptides PGA(X₂) (SEQ ID NOs: 414) with the C-terminal fluorescent label. The latter can be visualized in order to track the progress of the reaction.

TABLE 7
Secondary substrates used for ligation rate
analysis.

| Peptide | Sequence | SEQ ID NO |
|---|---|---|
| PGA(17) | PGAIDEDPFIIELEGGKGGG | 373 |
| PGA(10) | PGAIDEDPFIIEL | 374 |
| PGA(8) | PGAIDEDPFI | 375 |

Specifically, 100 nM M. jannaschii Adriase were added to optimized Adriase buffer (20 mM MES-NaOH pH 5.8, 150 mM NaCl, 100 mM KCl, 5 mM TCEP-HCl), incubated with or without 60 μM fluorophore-coupled primary (SEQ ID NOs: 370-371 and 418; see Table 6) and 100 μM non-fluorescent secondary substrates (SEQ ID NO: 373-375; see Table 7) in the combinations as indicated in FIG. 9A. The reaction was performed for 8 min at 85 °C and stopped by addition of 2% SDS. Samples were then separated on 12% polyacrylamide gels (Thermo) and fluorescent products visualized by UV light.

FIG. 9A shows that the substrate (F15)KDPGA(SEQ ID NO: 311) (10) with SEQ ID NO: 367 (lane 2-4) forms a covalent bond to Adriase ((F15)KD-Adriase; SEQ ID NO: 429), resulting in the release of non-fluorescent PGA(10) with SEQ ID NO: 374 (not visible due to the lack of a fluorescent label); In presence of PGA(17) (SEQ ID NO: 373), (F15)KDPGA(SEQ ID NO: 311) (10) (SEQ ID NO: 367) is recombined to (F15)KDPGA(SEQ ID NO: 311) (17) (SEQ ID NO: 413). Substrates with C-terminal fluorophores (lane 5-13) also form covalent bonds to Adriase (non-fluorescent), resulting in the release of small quantities of PGA(10F) (SEQ ID NO: 414); In presence of PGA(17) (SEQ ID NO: 373), non-fluorescent ligation products (15/10/5)KDPGA(SEQ ID NO: 311) (17) (SEQ ID NOs: 415-417, respectively) and more PGA(10F) are formed.

These experiments show that PGA(X) can be used as secondary substrate, supporting the proposed reaction mechanism (FIG. 8). They also show that fluorescent peptides are useful to assay the characteristics of Adriase-mediated ligations. In the following, they are used to determine Adriase ligation rates (FIG. 9B).

For this purpose, ligations were performed and visualized as described above, except for the use of just 6 nM Adriase and the increasing concentrations of (15)KDPGA(SEQ ID NO: 311) (10F) (SEQ ID NO: 418) and PGA(10) (SEQ ID NO: 374) peptides (2.5/5/10/20/40/80/160 μM of each peptide of the peptide pairs). The band intensity of the respectively fluorescently labeled PGA(10F)-peptide (see SEQ ID NO: 414), which is released by the ligation reaction was quantified using ImageJ v1.50i and subtracted from background signal in control reactions without PGA(10) peptide.

The determined values are shown in FIG. 9B. While it is unclear, whether the assayed Adriase reaction follows classical Michaelis-Menten kinetics, the determined ligation rates are well described by the Michaelis-Menten equation (black line in FIG. 9B). Thus, the Michaelis-Menten model as implemented in SigmaPlot v12.3 was used to approximate the maximum rate of ~1.4 ligations per enzyme and second. The half maximal reaction speed is observed at a substrate concentration of 23 μM each. Thus, thanks to its high affinity and reaction rate, nanomolar concentrations of Adriase efficiently catalyze ligations within minutes, even at low substrate concentrations, making Adriase an attractive choice for a wide range of applications.

To determine how the reaction is influenced by recognition motif characteristics, ligation rates for other substrates were determined in the same manner, except for the use of 20 μM of the indicated substrates (see also Tables 6 and 7) and varying concentrations of Adriase (6/30/150/750 nM). The results (FIG. 9C) show that M. jannaschii Adriase efficiently ligates (X₁)KDPGA(X₂) (SEQ ID NO: 681) and PGA(X₃) peptides with X₁≥5 and X₂=X₃≥10. In combination with the above experiments (FIGS. 9A and B), they provide the means to design substrates for efficient Adriase-mediated ligations.

### EXAMPLE 8: SEQUENCE DETERMINANTS GOVERNING ADRIASE ACTIVITY

To understand the role of Adriase sequence characteristics, experiments with a set of mutants were conducted. First, the function of the OB-like domain, which is found in a subset of Adriase proteins (SEQ ID NOs: 144 to 225), including the here studied M. jannaschii Adriase, was studied. For this purpose, size exclusion chromatography using 50 μl 200 μM M. jannaschii Adriase^{ΔOB} (SEQ ID NO: 132), M. jannaschii MtrA^{Δ194-245} (SEQ ID NO:420) or a 1:1 molar mixture of the Adriase-MtrA pair was analyzed using a Superdex S200 10/300 GL gel size-exclusion column (20 mM HEPES-NaOH pH 7.5, 50 mM NaCl, 100 mM KCl) coupled to a miniDAWN Tristar Laser photometer (Wyatt).

The results of these analyses (FIG. 10A) show that the elution profile of the mixture is identical to the combined profiles of its isolated components. Consequently, Adriase^{ΔOB} has a lower affinity for MtrA^{Δ194-245} compared to the full-length enzyme (FIG. 5A).

To investigate, whether the OB fold alone is sufficient to bind Adriase in the above experimental set-up, we repeated the same analysis using M. jannaschii Adriase^{ΔNTN} (SEQ ID NO: 519). Specifically, size exclusion chromatography using 50 μl 200 μM M. jannaschii Adriase^{ΔNTN}(SEQ ID NO: 519), M. jannaschii MtrA^{Δ194-245} (SEQ ID NO:420) or a 1:1 molar mixture of the Adriase-MtrA pair was analyzed using a Superdex S200 10/300 GL gel size-exclusion column (20 mM HEPES-NaOH pH 7.5, 50 mM NaCl, 100 mM KCl) coupled to a miniDAWN Tristar Laser photometer (Wyatt).

Just like in the above assay, the elution profile of the mixture is identical to the combined profiles of its isolated components (FIG. 19). Consequently, both NTN and OB domain contribute to the high-affinity interaction between M. jannaschii Adriase and MtrA (FIG. 5A).

In a second set of experiments, the ligase activity of Adriase variants was tested with (15)KDPGA(SEQ ID NO: 311) (10) (SEQ ID NO: 419) and PGA(10F) (SEQ ID NO:414) substrates. These included M. jannaschii Adriase^{ΔOB} (SEQ ID NO: 387), a variant with a deletion of an insertion that distinguishes Adriase from proteasome subunits (M. jannaschii Adriase^{Δ28-57}. SEQ ID NO: 388; see also FIG. 1), an active site mutant (M. jannaschii Adriase^{S1A}; SEQ ID NO: 386), and a variant that lacked a free amino group at the active site serine (N-His, SEQ ID NO: 450). Furthermore, M. mazei Adriase (SEQ ID NO: 380) was tested for ligase activity with the same M. jannaschii peptide substrates to assess whether variety within the recognition motif is tolerated. The read out for Adriase activity was the detection of fluorescently labeled ligation product (10)KDPGA(SEQ ID NO: 311) (10F) (SEQ ID NO: 418).

Specifically, 100 nM of the M. jannaschii Adriase variants (SEQ ID NO: 385-388 and 450) or the M. mazei Adriase (SEQ ID NO: 380) were incubated with or without 15 μM (15)KDPGA(10) (SEQ ID NO: 419) and 15 μM fluorophore-coupled PGA(10F) (SEQ ID NO: 374) substrates. The reaction was performed in optimized M. jannaschii Adriase buffer (20 mM MES-NaOH pH 5.8, 150 mM NaCl, 100 mM KCl, 5 mM TCEP-HCl) for 8 min at 85 °C when M. jannaschii derived Adriase variants were used and at 50 °C in optimized M. mazei Adriase buffer (50 mM acetic acid, 50 mM MES, 50 mM HEPES, 100 mM NaCl, 50 mM KCl, 5 mM TCEP, pH 7.0) when M. mazei Adriase was used and stopped by addition of 2% SDS. Samples were then separated on 12% polyacrylamide gels (Thermo) and fluorescent products visualized by UV light.

The results as depicted in FIG. 10B show that Adriase^{ΔOB} is still catalytically active because it catalyzed the ligation of (10)KDPGA(SEQ ID NO: 311) (10F). Hence, while the OB-like domain increases affinity for MtrA, it is not required for ligations via the Adriase recognition motif. By contrast, deletion of the insertion that distinguishes Adriase from proteasome subunits (Δ28-57, see FIG. 1) abolishes ligase activity. Likewise, no ligation product is observed for an active site mutant (S1A) and the N-terminally modified Adriase version (N-His), suggesting that both the serine hydroxyl and the unmodified serine amino group are required for efficient ligations (FIG. 8). Interestingly, also M. mazei Adriase showed activity when incubated with the M. jannaschii derived peptides indicating that sequence variability in the regions upstream and downstream of the conserved KDPGA (SEQ ID NO: 311) (SEQ ID NO: 315-366, 460-510 and 551-661) motif of the recognition motif is tolerated by the enzyme. The corresponding MtrA proteins of both organisms share only 47% and 40% sequence identity in the 15 residues upstream or 10 residues downstream of the KDPGA (SEQ ID NO: 311) motif. Consequently, a given Adriase enzyme may be cross-functional with Adriase recognition motifs derived from other organisms.

To investigate the effect of the OB domain on Adriase ligation kinetics in more detail, the time course of such a ligation was recorded for both full length Adriase and Adriase^{ΔOB}

Specifically, in three independent experiments, 100 μM M. jannaschii MtrA-derived His6 (SEQ ID NO: 678)-Ub-(5)KDPGA(SEQ ID NO: 311) (10) (SEQ ID NO: 526) and PGA(15)-Ub-His₆ (SEQ ID NO: 678) (SEQ ID NO: 527) substrates were incubated with 0.0025 molar equivalents of full-length M. jannaschii Adriase (SEQ ID NO: 385) or Adriase^{ΔOB} (SEQ ID NO: 387) in optimized M. jannaschii Adriase buffer (20 mM MES-NaOH pH 5.8, 150 mM NaCl, 100 mM KCl, 5 mM TCEP-HCl) at 85 °C. At various time points (0 s, 28 s, 55 s, 88 s, 126 s, 171 s, 225 s, 295 s, 393 s, 554 s, 1125 s, 1670 s and 2250 s), an aliquot of the reaction was mixed with 2% SDS. Samples were then separated on 12% polyacrylamide gels (Thermo) and stained with Coomassie blue (FIG. 20A). The band intensities of educt and product bands were quantified using ImageJ v1.52a, assuming that all ubiquitin molecules bind the coomassie dye in a similar manner. The results were then used to plot the time courses for each experiment (FIG. 20B).

The results (FIG. 20) show that both Adriase and the shortened version Adriase^{ΔOB} catalyze the ligation at similar rates, suggesting that, although it may assist in binding MtrA, the OB domain does not greatly affect the ligation of other protein substrates bearing the Adriase recognition motif.

### EXAMPLE 9: LIGATION RATE AND COMPLETENESS CAN BE CONTROLLED VIA SUBSTRATE RATIOS

The so far presented results suggest a reversible ordered ping-pong mechanism for Adriase ligations, in which both primary (X₁)KDPGA(X₂) (SEQ ID NO: 681) and secondary PGA(X₃) substrates bind at the same site (FIG. 6). In a first step, the primary substrate modifies the Adriase catalytic Ser/Thr and PGA(X₂) is released (FIG. 8). This process should be accelerated by high primary substrate concentrations but inhibited by high secondary substrate concentrations, as the latter cannot be utilized upon binding to the unmodified enzyme. In a second step, the modified Adriase enzyme reacts with the secondary substrate—a process that should by accelerated by high secondary substrate concentrations. Consequently, the ratio between primary and secondary substrates is an important reaction parameter.

To study this parameter in more detail, 6 nM M. jannaschii Adriase (SEQ ID NO: 385) in optimized buffer (20 mM MES-NaOH pH 5.8, 150 mM NaCl, 100 mM KCl, 5 mM TCEP-HCl) was incubated with various substrate ratios: In a first experiment, the 20 μM secondary PGA(10) (SEQ ID NO: 374) substrate and varying concentrations (2.5-160 μM) of primary (15)KDPGA(SEQ ID NO: 311) (10F) (SEQ ID NO: 418) were used (FIG. 11A); In a second experiment, 20 μM (15)KDPGA(SEQ ID NO: 311) (10F) and varying concentrations (2.5-160 μM) of PGA(10) were used (FIG. 11B). The reactions were performed for 7 min at 85 °C and stopped by addition of 2% SDS. Samples were then separated on 12% polyacrylamide gels (Thermo) and fluorescent products visualized by UV light. The band intensity of the respective fluorescently labeled PGA(IOF) peptides (SEQ IDs NO: 414), which were released by the ligation reactions were quantified using ImageJ v1.50i and subtracted from background signal in control reactions without PGA(10) peptides.

Interestingly, while the ligation rate is generally higher at higher primary substrate concentrations (FIG. 11 A), high secondary substrate concentrations appear to inhibit the reaction (FIG. 11B). Instead, for the highest ligation rates, both substrates should be used at equimolar ratio. Nevertheless, different substrate ratios may find use where complete ligation of one reaction partner is desired, for example tagging a protein with a fluorophore. In these cases, product formation could be driven by using excess concentrations of the fluorophore. If Adriase binds both substrates equally well, the effect of their ratio on the proportion of ligated protein at the equilibrium should be described by the following formula:

$$Substrate ratio = Ligation product ratio + \frac{Ligation product {ratio}^{2}}{1 - Ligation product ratio}$$

To test this assumption, the above experiment, which analyzed ligation rates at the start of the reaction, was performed with much higher Adriase concentrations. This way, product quantities at the reaction equilibrium could be studied. Specifically, 0.5 μM of M. jannaschii Adriase (SEQ ID NO: 385) in optimized buffer (20 mM MES-NaOH pH 5.8, 150 mM NaCl, 100 mM KCl, 5 mM TCEP-HCl) were incubated with 30 μM (15)KDPGA(SEQ ID NO: 311) (10F) (SEQ ID NO: 418) and varying concentrations (1.875-480 μM) of PGA(10) (SEQ ID NO: 374). The reactions were performed for 10 min at 85 °C and stopped by addition of 2% SDS. Samples were then separated on 12% polyacrylamide gels (Thermo) and fluorescent products visualized by UV light. The band intensity of the respective fluorescently labeled PGA(10F) peptides (SEQ IDs NO: 414), which were released by the ligation reactions were quantified using ImageJ v1.50i and subtracted from background signal in control reactions without PGA(10) peptides.

The results (FIG. 11C) show that the above equation (solid line) can be used to estimate the amount of ligated product and thus serve as a guideline when designing a ligation experiment in which Adriase binds both substrates equally well. In this case, for instance 90% of a given protein can be ligated to a substrate added in nine fold excess:

$$9 = 0.9 + \frac{0 . {9}^{2}}{1 - 0.9}$$

### EXAMPLE 10: TIME COURSE OF THE ADRIASE REACTION

Because Adriase reactions are reversible, the observed product formation proceeds fastest at the beginning of the reaction and then gradually slows down as the equilibrium is approximated. At a given time (t), the observed reaction rate can be described by the following formula:

$$observed rate at t = maximum rate \times 1 - ( \frac{Product concentration at t}{Maximum product concentration} ) ,$$

where the “maximum product concentration” is the concentration at the equilibrium (i.e. usually 50% when using equimolar substrates). The amount of ligation product can be calculated by integrating these rates over time:

$$Ligated product = {\int }_{t \_ start}^{t \_ end} observed rate at t * dt$$

Both formula can be combined to:

$$Ligated product = {\int }_{t \_ start}^{t \_ end} [ maximum ∘ rate ] ∘ * ( 1 - \frac{Product ∘ concentration ∘ at ∘ t}{Maximum ∘ product ∘ concentration} ) * dt$$

Using these formulae, the time course of an Adriase reaction with known maximum ligation rate can be predicted. Conversely, the maximum ligation rate can be determined by recording the time course. To test these assumptions, the time course of Adriase ligations was recorded for various concentrations and compared to the above models.

Specifically, different concentrations (1.25 μM, 2.5 μM, 5 μM, 10 μM, 20 μM or 40 μM) of (5)KDPGA(SEQ ID NO: 311) (10F) (SEQ ID NO: 453) and PGA(10) (SEQ ID NO: 454; synthesized by Genscript) substrates were incubated with 0.001 molar equivalents of M. mazei Adriase (SEQ ID NO: 380) in reaction buffer (50 mM acetic acid, 50 mM MES, 50 mM HEPES, 100 mM NaCl, 50 mM KCl, 5 mM TCEP, pH 7.0) at 50 °C. At various time points (0 s, 37 s, 79 s, 126 s, 180 s, 244 s, 322 s, 423 s, 561 s and 791 s), an aliquot of the reaction was mixed with 2% SDS. Samples were then separated on 12% polyacrylamide gels (Thermo) and fluorescent products visualized by UV light. The band intensity of the respective fluorescently labeled PGA(10F) peptides (SEQ ID NO: 455), which were released by the ligation reactions were quantified using ImageJ v1.50i and subtracted from background signal in control reactions without PGA(10) peptides.

As depicted in FIG. 12A, the above models fit the determined data well and allow the determination of the maximum ligation rate at a given substrate concentration. Using the Michaelis-Menten model as implemented in SigmaPlot v12.3 (FIG. 12B), these data can be used to approximate a maximum rate of ~2.25 ligations per enzyme and second. The half maximal reaction speed is observed at a substrate concentration of ~9 μM each. Thus, M. mazei Adriase displays similar but slightly more favorable characteristics compared to M. jannaschii Adriase (FIG. 9). In light of the high degree of sequence diversity between both variants (35% sequence identity), these results suggest that the findings presented so far hold true for a wide range of very different Adriase proteins.

To investigate, how the above peptide-peptide ligation rates compare to protein-protein ligations, experiments using the protein substrates Ub-(5)KDPGA(SEQ ID NO: 311) (15) (SEQ ID NO: 520) and PGA(15)-Ub (SEQ ID NO: 521) were conducted.

Specifically, different concentrations (0.39 μM, 0.78 μM, 1.56 μM, 3.13 μM, 6.25 μM, 12.5, 25, 50 or 100 μM; three independent experiments per concentration) of Ub-(5)KDPGA(SEQ ID NO: 311) (15) (SEQ ID NO: 520) and PGA(15)-Ub (SEQ ID NO: 521) substrates were incubated with 0.0025 molar equivalents of M. mazei Adriase (SEQ ID NO: 380) in reaction buffer (50 mM acetic acid, 50 mM MES, 50 mM HEPES, 100 mM NaCl, 50 mM KCl, 5 mM TCEP, pH 7.0) at 50 °C. At various time points (0 s, 28 s, 55 s, 88 s, 126 s, 171 s, 225 s, 295 s, 393 s, 554 s, 1125 s, 1670 s and 2250 s), an aliquot of the reaction was mixed with 2% SDS. Samples were then separated on 12% polyacrylamide gels (Thermo) and stained with Coomassie blue (FIG. 21A). The band intensities of educt and product bands were quantified using ImageJ v1.52a, assuming that all ubiquitin molecules bind the coomassie dye in a similar manner. The results were used to plot the time courses for each experiment (FIG. 21B). Using the above described formula

$$Ligated product = {\int }_{t \_ start}^{t \_ end} [ maximum ∘ rate ] ∘ * ( 1 - \frac{Product ∘ concentration ∘ at ∘ t}{Maximum ∘ product ∘ concentration} ) * dt ,$$

the data fit for each time course allowed the determination of the maximum rate parameter for each concentration. These maximum rates were then used in a Michaelis-Menten plot (FIG. 21C) to approximate kinetic measures.

According to the resulting Michaelis-Menten plot (FIG. 21C), M. mazei Adriase catalyzes the above protein-protein ligation at a maximum rate of 0.92 ligations per enzyme and second; the half-maximum rate is observed at a substrate concentration of 2.2 μM each. These values are overall comparable with the parameters determined for the peptide-peptide ligation (FIG. 12) and show that Adriase is capable of catalyzing protein-protein ligations at high rates, even with low substrate concentrations.

### EXAMPLE 11: M. MAZEI ADRIASE DOES NOT HYDROLYZE ITS SUBSTRATES

Although protein ligase enzymes are generally rare in nature, Adriase shares this functionality with a few other representatives, such as Sortase (Pishesha (2018) Annu Rev Cell Dev Biol 34:163-188) or Butelase (Nguyen (2014) Nat Chem Biol 10:732-8). All known protein ligases, however, use thio- or hydroxylesters as reaction intermediates that are prone to hydrolysis. The irreversible nature of this side-reaction necessitates timely removal of the protein ligase. Adriase is thought also to use a hydroxylester as a reaction intermediate (FIG. 8, Step 1), which is, however, subsequently stabilized via amide bond formation (FIG. 8, Step 2). To check, whether the Adriase intermediate is subject of a hydrolysis side reaction, the Adriase reaction was analyzed at high concentrations for several hours. The results were then compared to the predicted hydrolysis product (F10)KD, which would be formed upon hydrolysis of the (F10)KD-Adriase intermediate.

Specifically, 15 μM (F10)KDPGA(SEQ ID NO: 311) (10) (SEQ ID NO: 456) and 15 μM PGA(25) (SEQ ID NO: 457) were incubated with either 15 nM or 15 μM M. mazei Adriase (SEQ ID NO: 380) in optimized buffer (50 mM acetic acid, 50 mM MES, 50 mM HEPES, 100 mM NaCl, 50 mM KCl, 5 mM TCEP, pH 7.0) at 37 °C. In a control reaction, the predicted hydrolysis product, (F10)KD (SEQ ID NO: 458) was incubated under the same conditions with 15 μM Adriase (Hydrolysis control). The lower incubation temperature compared to example 10 was chosen to avoid denaturation of the enzyme. After 12 s, 0.5 h, 1 h, 2 h and 4 h, aliquots were removed and mixed with 2% SDS to stop the reaction. Samples were then separated on 12% polyacrylamide gels (Thermo) and fluorescent products visualized by UV light.

The resulting gel (FIG. 13) shows the generation of the ligation product (F10)KDPGA(SEQ ID NO: 311) (25) (SEQ ID NO: 459). The experiment with just 15 nM Adriase shows that this product is formed over the entire 4 h period, suggesting that the enzyme does not denaturate under these conditions. A band at the height of the hypothetical hydrolysis product, (F10)KD, is not observed. This is true even for 1000× increased Adriase concentrations (15 μM) and when the gel is overexposed for maximum sensitivity (lower panel in FIG. 13). Moreover, the amount of ligation product (F10)KDPGA(SEQ ID NO: 311) (25) does not decrease over time, as it would be expected if its reversible generation competed with an irreversible hydrolysis side reaction. Together, these results show that M. mazei Adriase does not hydrolyze its substrates and hence does not have to be removed after ligations to avoid product loss.

To investigate, whether this characteristic also holds true with protein substrates, the above experiment was repeated with Ub-(5)KDPGA(SEQ ID NO: 311) (15)-Strep substrate (SEQ ID NO: 522). Specifically, 25 μM Ub-(5)KDPGA(SEQ ID NO: 311) (15)-Strep and 25 μM PGA(10) (SEQ ID NO: 454) were incubated with either 25 nM or 25 μM M. mazei Adriase (SEQ ID NO: 380) in optimized buffer (50 mM acetic acid, 50 mM MES, 50 mM HEPES, 100 mM NaCl, 50 mM KCl, 5 mM TCEP, pH 7.0) at 50 °C. After 140 s, 320 s, 575 s, 1002 s, 30 min or 90 min, aliquots were removed and mixed with 2% SDS to stop the reaction. Samples were then separated on 12% polyacrylamide gels (Thermo) and their migration behaviour compared to that of the putative hydrolysis product Ub-(5)KD (SEQ ID NO: 523) and to that of the educts (0 s).

The resulting gel (FIG. 22) shows the time-dependent formation of the reaction product Ub-(5)KDPGA(SEQ ID NO: 311) (10) (SEQ ID NO: 524) in the sample with low Adriase concentrations (25 nM/1×), while no band at the height of the putative hydrolysis product Ub-(5)KD is visible. Even at prolonged incubation times (90 min) and 1000× increased Adriase concentrations, the amount of ligation product remains constant at ~50% and no hydrolysis product is visible. In agreement with the first experiment, this result show that Adriase does not possess hydrolase activity, neither towards peptides, nor towards proteins.

### EXAMPLE 12: A GENERAL TEST TO EVALUATE ADRIASE LIGATION EFFICIENCY

The results presented in example 10 suggest that all Adriase proteins share similar characteristics despite being encoded by very divergent sequences. It is therefore possible to suggest a general test to evaluate the efficiency of a given Adriase variant.

Step 1: Design of primary and secondary substrates. Suitable substrates are for instance (15)KDPGA(SEQ ID NO: 311) (10) and PGA(10F) sequences derived from the respective Adriase recognition motif (SEQ ID NO: 315-366, 460-510 and 551-661). The fluorophore FITC (F; Fluorescein-5-isothiocyanate) can be linked to the amino group of an extra lysine at the C-terminus. Synthesis of these compounds is offered by various companies; the ones used in the above experiments were produced by Genscript.

Step 2: Set-up of the ligation reactions. As a starting point, 15 μM primary substrate, 15 μM secondary substrate and various Adriase concentrations, ranging from 0 μM (control) to 15 μM, should be used. The reaction should be performed for 8 min, preferably at physiological conditions (see appended Table 1 for known optimal growth conditions of Adriase organisms).

Step 3: Visualization of the reactions. Ligations of the above substrates can be monitored by UV exposure of SDS gels. For the above experiments, 7.5 μl of the above reactions (Step 2) were mixed with 2.5 μl sample buffer (200 mM Tris-HCl pH 6.8, 8% SDS, 0.4% bromophenol blue, 40% glycerol) and applied them to 12% Bis-Tris gels (Thermo) with MES running buffer (50 mM MES, 50 mM Tris, 0.1% SDS, 1 mM EDTA, pH 7.3). After running the gels according to the manufacturer's instructions, they were imaged using a Vilber Lourmat Fusion SL instrument and the UV fluorescence autoexposure option within the FusionCapt Advance SL2 Xpress software. If subsequent quantification of the reaction products is desired, it is important to avoid overexposure. If educts and products cannot be discriminated by their SDS-PAGE migration behavior, other methods, such as size-exclusion chromatography or mass spectroscopy, may be employed.

Step 4: Interpretation of the results. In case of a successful ligation, a specific product (i.e. (15)KDPGA(SEQ ID NO: 311) (10F) with the suggested substrates) can be observed on the gel. The formation of this product can then be quantified using a variety of densitometric tools, such as ImageJ v1.50i. Densitometry is a well-established technique (Gassmann (2009) Electrophoresis 30:1845-55) (Tan (2008): Opt. Commun. 281:3013-3017), allowing the evaluation of Adriase ligation efficiencies. It relies on the quantification of pixel grayscales (0-255) in the individual gel lanes. When these are plotted (signal vs location), fluorescent peptide bands show as peaks. After subtraction from background signals, the integral of these peaks is proportional to the respective peptide quantity.

### EXAMPLE 13: AN UNMODIFIED AMINO GROUP AT THE ADRIASE ACTIVE SITE SER/THR IS REQUIRED FOR EFFICIENT LIGATIONS

To exemplify the above procedure (Example 12), Adriase variants with and without N-terminal modification were analysed. In example 9, ligase activity was only observed for Adriase variants with exposed amino group at the active site, highlighting its significance in the reaction mechanism (FIG. 8). To study the role of this group in more detail, a re-analysis of N-terminally modified Adriase at far higher concentrations was performed.

Specifically, 15 μM of (15)KDPGA(SEQ ID NO: 311) (10) (SEQ ID NO: 419) and 15 μM of fluorophore-coupled PGA(10F) (SEQ ID NO: 374) substrates were incubated optimized M. jannaschii Adriase buffer (20 mM MES-NaOH pH 5.8, 150 mM NaCl, 100 mM KCl, 5 mM TCEP-HCl) for 8 min at 80 °C with either various Adriase concentrations. The assay was performed with either 7 nM, 21 nM, 62 nM, 185 nM, 556 nM, 1666 nM, 5000 nM or 15000 nM N-terminally modified Adriase (N-His, SEQ ID NO: 450) or 0 nM, 7 nM, 21 nM or 62 nM unmodified Adriase (SEQ ID NO: 385) and stopped by addition of 2% SDS. Samples were then separated on 12% polyacrylamide gels (Thermo) and fluorescent products visualized by UV light.

The resulting SDS-gel (FIG. 14) visualizes the generation of the fluorescent reaction product (15)KDPGA(SEQ ID NO: 311) (10F) (SEQ ID NO: 418), accompanied by a decrease of PGA(10F) substrate. While no activity for N-terminally modified Adriase is observed at low concentrations (see also FIG. 10), this variant retains a ~200× decreased activity that only becomes apparent at high concentrations. This residual activity was surprising, as an exposed amino group at the active site was considered essential for catalysis (FIG. 8). To investigate this phenomenon, an LCMS analysis was performed.

Specifically, 0.5 g/l N-terminally modified M. jannaschii Adriase (SEQ ID NO: 450) was desalted and subjected to a Phenomenex Aeris Widepore 3.6 μm C4 200 Å (100×2.1 mm) column, eluted with a 30-80% H₂O/acetonitrile gradient over 15 min in the presence of 0.05% trifluoroacetic acid and analyzed with a Bruker Daltonik microTOF. Data processing was performed with Bruker Compass DataAnalysis 4.2 and the m/z deconvoluted with the MaxEnt module to obtain the protein mass.

The analysis (FIG. 15) reveals the expected mass for N-terminally modified Adriase without the start-methionine in the main peak (Δ1; SEQ ID NO: 511). In addition, considerably smaller peaks for the same protein, lacking the first 8 (Δ8; SEQ ID NO: 512), 10 (Δ10; SEQ ID NO: 513), 11 (Δ11; SEQ ID NO: 514) or 21 (Δ21; SEQ ID NO: 515) residues were automatically assigned by the Bruker Compass DataAnalysis software. This pattern suggests a small degree of non-specific degradation at the unstructured N-terminal modification, a problem frequently faced in recombinant protein expression and purification (Ryan (2013) Curr Protoc Protein Sci Chapter 5:Unit5 25). It also provides an explanation for the observed ligase activity of the sample as the A21 truncation removes the N-terminal modification and exposes the amino group of the active site serine at position 22. In agreement with the ~200× decreased ligase activity compared to unmodified Adriase (see above), the A21 truncation accounts only for a small fraction of the sample. Consequently, evidence suggests that N-terminal modifications can be subject to proteolytic degradation but inactivate the enzyme as long as they persist and that an exposed serine/threonine residue is indeed required for catalytic activity.

### EXAMPLE 14: GENERAL APPLICABILITY OF ADRIASE AS A PROTEIN-PROTEIN LIGASE

To investigate, whether Adriase can act as a general ligase for any protein substrate bearing the Adriase ligation motif, further ligation experiments with other unrelated protein substrates were conducted.

Specifically, 0.9 μM M. mazei Adriase (SEQ ID NO: 380) were added to 3.7 μM Ub-(5)KDPGA(SEQ ID NO: 311) (10) (SEQ ID NO: 524) and/or 3.7 μM PGA(10)-sdAb (single-domain antibody; SEQ ID NO: 663), PGA(10)-CyP (Cyclophilin; SEQ ID NO: 664) or PGA(10)-GST (Glutathione-S-Transferase; SEQ ID NO: 665) as indicated (FIG. 23). The reaction was conducted reaction buffer (50 mM acetic acid, 50 mM MES, 50 mM HEPES, 100 mM NaCl, 50 mM KCl, 5 mM TCEP, pH 7.0) at 37 °C and stopped either after the indicated time (FIG. 23, lanes 1-4) or after 10 min (lanes 5-10) by addition of 2% SDS, following SDS-PAGE analysis.

The resulting SDS gel (FIG. 23) shows that Adriase efficiently ligates all three protein pairs, suggesting that Adriase can generally ligate any two proteins bearing the Adriase ligation motif.

### EXAMPLE 15: ANALYSIS OF THE ADRIASE LIGATION MOTIF

To study which ligation motifs are processed most efficiently, a systematic analysis of sequence determinants N- and C-terminal of the MtrA-derived (X₁)KDGPA(X₂) (SEQ ID NO: 679)/PGA(X₃) motif was conducted.

Specifically, in three independent experiments, 25 μM primary and 25 μM secondary substrates were incubated with 0.0025 molar equivalents of M. mazei Adriase (SEQ ID NO: 380) in reaction buffer (50 mM acetic acid, 50 mM MES, 50 mM HEPES, 100 mM NaCl, 50 mM KCl, 5 mM TCEP, pH 7.0) at 50 °C. In a first set of experiments, different primary substrates—(0)KDPGA(SEQ ID NO: 311) (10), (5)KDPGA(SEQ ID NO: 311) (10), (10)KDPGA(SEQ ID NO: 311) (10), (5)KDPGA(SEQ ID NO: 311) (15), Ub-(5)KDPGA(SEQ ID NO: 311) (10) and Ub-(5)KDPGA(SEQ ID NO: 311) (15) (SEQ ID NO: 520, 524, 531-534; Table 8)—were combined with the same secondary substrate, PGA(15)-Ub (SEQ ID NO: 521); In a second set of experiments, different secondary substrates—PGA(5), PGA(10)-Ub, PGA(15)-Ub and PGA(20)-Ub (SEQ ID NO: 521, 525, 529-530)—were combined with the same primary substrate, Ub-(5)KDPGA(SEQ ID NO: 311) (15) (SEQ ID NO: 520). Similarly, PGA(10) and PGA(15) (SEQ ID NO: 454 and 528) were combined with an analogous substrate, Ub-(5)KDPGA(SEQ ID NO: 311) (15)-Strep (SEQ ID NO: 522). At various time points (0 s, 28 s, 55 s, 88 s, 126 s, 171 s, 225 s, 295 s, 393 s, 554 s, 1125 s, 1670 s and 2250 s), an aliquot of the reaction was mixed with 2% SDS. Samples were then separated on 12% polyacrylamide gels (Thermo) and stained with Coomassie blue. The band intensities of educt and product bands were quantified using ImageJ v1.52a, assuming that all ubiquitin molecules bind the coomassie dye in a similar manner. The results were used to plot the time courses for each experiment. Using the above described formula

$$Ligated product = {\int }_{t \_ start}^{t \_ end} [ maximum ∘ rate ] ∘ * ( 1 - \frac{Product ∘ concentration ∘ at ∘ t}{Maximum ∘ product ∘ concentration} ) * dt ,$$

the data fit for each time course allowed the determination of the maximum rate parameter in each experiment.

The results (FIG. 24) show that 5 residues N-terminal of KDPGA (SEQ ID NO: 311) and 10 residues C-terminal of KDPGA (SEQ ID NO: 311)/PGA allow efficient ligations in most cases, but that sterically demanding protein-protein ligations are much faster with 15 residues C-terminal of PGA.

TABLE 8
Peptides used for ligation motif analysis
(synthesized by Genscript)

| | | SEQ |
|---|---|---|
| | | ID |
| Peptide | Sequence | NO |
| (0)KDPGA(SEQ | KDPGAFDADPLVVEI | 531 |
| ID NO: 311) | | |
| (10) | | |
| (5)KDPGA(SEQ | RELASKDPGAFDADPLVVEI | 532 |
| (10)KDPGA(SEQ | ITSKVRELASKDPGAFDADPLVVEI | 533 |
| ID NO: 311) | | |
| (10) | | |
| (5)KDPGA(SEQ | RELASKDPGAFDADPLVVEISEEGE | 534 |
| ID NO: 311) | | |
| (15) | | |
| PGA(5) | PGAFDADP | 529 |
| PGA(10) | PGAFDADPLVVEI | 454 |
| PGA(15) | PGAFDADPLVVEISEEGE | 528 |

### EXAMPLE 16: COMPARISON WITH SORTASE

The most widely used enzyme ligase is Sortase A, which has proven a powerful and reliable tool in numerous remarkable applications. Sortase A has been extensively optimized and state of the art in many labs is currently the Sortase A pentamutant (SrtA5*), which has been reported to show up to 120× increased rates compared to the wild type enzyme (Chen (2011) loc. cit.). Analogous to Adriase, Sortase A ligates two sequences bearing the LPET-G (SEQ ID NO: 682) motif and an N-terminal glycine, respectively, though additional linker sequences are usually introduced to avoid steric hindrances and to increase reactivity (Heck (2014) loc. cit.). In addition, Sortase also catalyzes the irreversible hydrolysis of substrates and products featuring this motif at a lower rate (K_{cat Ligation}/K_{cat Hydrolysis}≈3.3 (Frankel (2005) loc. cit.) and the maximum amount of product can therefore only be obtained by monitoring the ligation/hydrolysis ratio and by stopping the reaction at just the right time.

To evaluate the applicability of Adriase, its ligase efficiency was compared with that of SrtA and SrtA5*. Specifically, in three independent experiments, the time courses of a M. mazei Adriase (SEQ ID NO: 380) catalyzed ligation of Ub-(5)KDPGA(SEQ ID NO: 311) (15) (SEQ ID NO: 520) and PGA(15)-Ub (SEQ ID NO 521) as well as the time courses of a SrtA5* (SEQ ID NO: 535) or SrtA (SEQ ID NO: 662) catalyzed ligation of Ub-GGSLPETGGGMHHHHH (SEQ ID NO: 669) (SEQ ID NO: 536) and GGG-Ub (SEQ ID NO: 666) were recorded. The Adriase assays were conducted with 3.13 μM and 100 μM substrate concentration and 0.0025 molar equivalents Adriase (SEQ ID NO: 380) in reaction buffer (50 mM acetic acid, 50 mM MES, 50 mM HEPES, 100 mM NaCl, 50 mM KCl, 5 mM TCEP, pH 7.0) at 50 °C. The SrtA5* and SrtA assays were conducted with 3.13 μM and 100 μM substrate concentration and either 1 or 0.1 molar equivalents SrtA5* or SrtA (SEQ ID NO: 535 and 662) in Sortase buffer (50 mM Tris-HCl pH 7.5, 150 mM NaCl, 10 mM CaCl₂)) at 37 °C. At various time points (0 s, 28 s, 55 s, 88 s, 126 s, 171 s, 225 s, 295 s, 393 s, 554 s, 1125 s, 1670 s and 2250 s), an aliquot of the reaction was mixed with 2% SDS. Samples were then separated on 12% polyacrylamide gels (Thermo) and stained with Coomassie blue. The band intensities of educt and product bands were quantified using ImageJ v1.52a, assuming that all ubiquitin molecules bind the coomassie dye in a similar manner. The results were used to plot the time courses for each experiment.

The results (FIG. 25) show that, at low substrate concentrations (3.13 μM each), SrtA and SrtA5* display only spurious ligase activity, even at an enzyme:substrate ratio of 1:1. By contrast, Adriase ligates ~50% (on a molar basis) of the substrates in an analogous assay at an enzyme:substrate ratio of only 1:400. At high substrate ratios (100 μM), SrtA and SrtA5* show more favorable characteristics. Yet, even under those conditions, Adriase shows >4000× higher ligase activity than non-optimized SrtA and >40× increased ligase activity compared to optimized SrtA5*. Furthermore, we observed increased ligation yields in Adriase reactions (~50% compared to ~30%), which we attribute to the apparent absence hydrolysis side-reactions. These side-reactions are particularly pronounced in case of SrtA5*, likely due to its low affinity for the secondary (GGG-) substrate (K_{M LPETG} (SEQ ID NO: 682)=170 μM; K_{M GGG}=4700 μM (Chen (2011) loc. cit.). These results are comparable with Sortase ligations of other protein substrates (Levary (2011) PLoS One 6:e18342; Li (2020) JBC 295:2664-2675; Heck (2014) loc. cit.) and demonstrate, why the secondary substrate is often added in 10× excess (Antos (2016) loc. cit.). Hence, Adriase is advantageous compared to Sortase enzymes, as it combines substantially higher substrate affinities, reaction rates and ligation yields without catalyzing detectable side reactions.

### EXAMPLE 17: ADRIASE CATALYZES SPECIFIC LIGATIONS IN COMPLEX SOLUTIONS

To test whether Adriase is also specific in more complex solutions, two independently expressed protein substrates were ligated within their respective cell lysate and subsequently purified the ligation products in a single step using a Ni-NTA column in series with a streptavidin column.

Specifically, Strep-Ub-(5)KDPGA(SEQ ID NO: 311) (10)-His6 (SEQ ID NO: 678) (SEQ ID NO: 538) and PGA(15)-Ub (SEQ ID NO: 539) without affinity tag were recombinantly expressed in E. coli. Transformed cells carrying the respective plasmids (see Example 1) were grown in at 25 °C in 2 L lysogeny broth (LB) and protein expression was induced at an optical density of 0.4 at 600 nm with 500 μM isopropyl-β-D-thiogalactoside. After 16 h, cells were harvested and all subsequent steps conducted at 7 °C, unless stated otherwise. The cell pellet of constructs was resuspended in buffer (50 mM acetic acid, 50 mM MES, 50 mM HEPES, 100 mM NaCl, 50 mM KCl, 10 mM Imidazole, 5 mM MgCl₂, 50 μg/ml DNAse (Applichem) and cOmplete protease inhibitor (Roche) pH 7.0), lysed by three french press passages at 16000 psi, and cleared from cell debris by ultracentrifugation at 100000 g for 45 min. The supernatant was then filtered using membrane filters (Millipore) with a pore size of 0.22 μm. For the ligation, equal volumes of each cell lysate were mixed with 0.09 g/l M. mazei Adriase-His₆ (SEQ ID NO: 678) (SEQ ID NO: 380), which corresponds to a molar enzyme:substrate ratio of roughly 1:30. After incubation for 15 min at 37 °C, the pH was adjusted to 8.0 and the mixture applied on a HisTrap FF Ni²⁺-NTA column in series with a HiTrap streptavidin column (GE). Following rigorous washing (20 mM Tris, 250 mM NaCl, pH 8), the reaction product was eluted with desthiobiotin (20 mM Tris, 250 mM NaCl, 2.5 mM desthiobiotin pH 8). In a second step, His₆ (SEQ ID NO: 678)-tagged educts were eluted with desthiobiotin and imidazole desthiobiotin (20 mM Tris, 250 mM NaCl, 2.5 mM desthiobiotin, 250 mM imidazole pH 8).

The results (FIG. 26) show that only one protein species, Strep-Ub-(5)KDPGA(SEQ ID NO: 311) (10), could be eluted with desthiobiotin, indicating that no other proteins in the lysate reacted with the Strep-Ub-(5)KD-Adriase intermediate. In accordance with the nanomolar affinity interaction between Adriase and its conserved recognition motif (FIGS. 5 and 18), this observation suggests that Adriase ligations are highly specific and applicable even in complex solutions. Moreover, this experiment highlights the feasibility of Adriase-mediated ligations for the large-scale generation and single-step purification of a given ligation product in short time and with minimal amounts of enzyme.

An overview of potential applications is shown in FIG. 16.

1. A method for producing a polypeptide comprising an N-terminal DUF2121 domain having an N-terminal serine or threonine residue, the method comprising:
a) cultivating a host cell comprising a nucleic acid encoding the polypeptide; and
b) recovering the polypeptide from the cell culture and/or the cells.

2. A method for producing a fusion polypeptide comprising contacting a polypeptide comprising an N-terminal DUF2121 domain having an N-terminal serine or threonine residue with a first substrate polypeptide and a second substrate polypeptide, and reacting both substrate polypeptides.

3. The method of claim 2, wherein the method further comprises producing a fusion polypeptide.

4. The method of claim 2, wherein the produced fusion polypeptide comprises:
(i) a portion of the first substrate polypeptide and a portion of the second substrate polypeptide; or
(ii) a portion of the first substrate polypeptide and the entire second polypeptide.

5. The method of claim 2, wherein the first substrate polypeptide comprises a DUF2121 recognition motif.

6. The method of claim 5, wherein the DUF 2121 recognition motif of the first substrate polypeptide comprises an amino acid sequence selected from the group consisting of SEQ ID NOs: 308, 309, 310 and 311, preferably SEQ ID NOs: 310 and 311, most preferably SEQ ID NO: 311.

7. The method of claim 5, wherein the DUF 2121 recognition motif of the first substrate polypeptide comprises at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 and most preferably at least 15 amino acids N-terminally of said SEQ ID NOs: 308, 309, 310 and 311, respectively.

8. The method of claim 5, wherein the DUF2121 recognition motif of the first substrate polypeptide comprises at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10, even more preferably at least 15 and even more preferably at least 20 amino acids C-terminally of said SEQ ID NOs: 308, 309, 310 and 311, respectively.

9. The method of claim 8, wherein the DUF2121 recognition motif of the first substrate polypeptide comprises at least 10, at least 15 or least 20 amino acids C-terminally of said SEQ ID NOs: 308, 309, 310 and 311, respectively.

10. The method of claim 5, wherein the DUF2121 recognition motif of the first substrate polypeptide comprises at least 5 amino acids N-terminally and at least 10 amino acids C-terminally of said SEQ ID NOs: 308, 309, 310 and 311, respectively.

11. The method of claim 5, wherein the DUF2121 recognition motif of the first substrate polypeptide comprises a sequence identical to or at least 60% identical to a sequence as defined by position(s) 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15 or 15 of any one of SEQ ID NOs: 315-366, 460-510 and 551-661 N-terminally of said SEQ ID NOs: 308, 309, 310 and 311, respectively.

12. The method of claim 5, wherein the DUF2121 recognition motif of the first substrate polypeptide comprises a sequence identical to or at least 60% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally of said SEQ ID NO: 308, 309, 310 and 311, respectively; and/or wherein the DUF2121 recognition motif of the first substrate polypeptide comprises a sequence identical to or at least 60% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally of said SEQ ID NO: 308, 309, 310 and 311, respectively.

13. The method of claim 5, wherein the DUF2121 recognition motif of the first substrate polypeptide consist of the sequence as defined in any one of SEQ ID NOs: 315-366, 460-510 and 551-661 or a sequence having at least 60% sequence identity to said sequence.

14. The method of claim 6, wherein the first substrate polypeptide has an N-terminal portion defined from the N-terminus of the first substrate polypeptide to the aspartate residue in position 2 of SEQ ID NO: 308, 309, 310 and 311, respectively.

15. The method of claim 5, wherein the second substrate polypeptide comprises a DUF2121 recognition motif.

16. The method of claim 15, wherein the DUF2121 recognition motif of the second substrate polypeptide comprises an amino acid sequence selected from the group consisting of SEQ ID NOs: 308, 309, 310 and 311, preferably SEQ ID NOs: 310 and 311, most preferably SEQ ID NO: 311.

17. The method of claim 15, wherein the DUF2121 recognition motif of the second substrate polypeptide comprises an amino acid sequence selected from the group consisting of SEQ ID NOs: 308, 309, 310 and 311.

18. The method of claim 15, wherein the DUF2121 recognition sequence of the second substrate polypeptide is identical with the DUF2121 recognition sequence of the first substrate polypeptide.

19. The method of claim 16, wherein the second substrate polypeptide has a C-terminal portion defined from the proline residue in position 3 of SEQ ID NOs: 308, 309, 310 and 311, respectively to the C-terminus of the second substrate polypeptide.

20. The method of claim 5, wherein the first substrate polypeptide has an N-terminal portion defined from the N-terminus of the first substrate polypeptide to the aspartate residue in position 2 of SEQ ID NO: 308, 309, 310 and 311, respectively and the second substrate polypeptide has a C-terminal portion defined from the proline residue in position 3 of SEQ ID NOs: 308, 309, 310 and 311, respectively to the C-terminus of the second substrate polypeptide, wherein the produced fusion protein comprises the N-terminal portion of the first substrate polypeptide and the C-terminal portion of the second substrate polypeptide C-terminally fused thereto.

21. The method of claim 5, wherein the second substrate polypeptide comprises a C-terminal portion of a DUF2121 recognition motif, said C-terminal portion of the DUF2121 recognition motif being positioned N-terminally of the second substrate polypeptide.

22. The method of claim 21, wherein the C-terminal portion of the DUF2121 recognition motif starts with the amino acid sequence as defined in positions 3 to 5 of any one of SEQ ID NOs: 308, 309, 310 and 311, preferably SEQ ID NOs: 310 and 311, most preferably SEQ ID NO: 311.

23. The method of claim 22, wherein the C-terminal portion of the DUF2121 recognition motif of the second substrate polypeptide comprises at least 1, preferably at least 2, even more preferably at least 3, even more preferably at least 4, even more preferably at least 5, even more preferably at least 6, even more preferably at least 7, even more preferably at least 8, even more preferably at least 9, even more preferably at least 10 even more preferably at least 15 and even more preferably 20 amino acids C-terminally of the N-terminal amino acids as defined by positions 3 to 5 of SEQ ID NOs: 308, 309, 310 and 311, respectively.

24. The method of claim 23, wherein the C-terminal portion of the DUF2121 recognition motif of the second substrate polypeptide comprises at least 10, at least 15 or at least 20 amino acids C-terminally of the N-terminal amino acids as defined by positions 3 to 5 of SEQ ID NOs: 308, 309, 310 and 311, respectively.

25. The method of claim 21, wherein the C-terminal portion of the DUF2121 recognition motif of the second substrate polypeptide comprises a sequence identical to or at least 60% identical to a sequence as defined by position(s) 21 to 30, 21 to 29, 21 to 28, 21 to 27, 21 to 26, 21 to 25, 21 to 24, 21 to 23, 21 to 22 or 21 of any one of SEQ ID NOs: 315-366, 460-510 and 551-661 C-terminally of the N-terminal amino acids as defined by positions 3 to 5 of SEQ ID NOs: 308, 309, 310 and 311, respectively; and/or wherein the C-terminal portion of DUF2121 recognition motif of the second substrate polypeptide comprises a sequence identical to or at least 60% identical to a sequence as defined by position(s) 21 to 40, 21 to 39, 21 to 38, 21 to 37, 21 to 36, 21 to 35, 21 to 34, 21 to 33, 21 to 32, 21 to 31 of any one of SEQ ID NOs: 551-661 C-terminally of said SEQ ID NO: 308, 309, 310 and 311, respectively.

26. The method of claim 21, wherein the C-terminal portion of the DUF2121 recognition motif of the second substrate polypeptide consist of the amino acid sequence as defined in positions 16 to 30 of any one of SEQ ID NOs: 315-366, 460-510 and 551-661 or an amino acid sequence having at least 60% sequence identity to said sequence, wherein the C-terminal portion of the DUF2121 recognition motif of the second substrate polypeptide consist of the amino acid sequence as defined in positions 16 to 35 of any one of SEQ ID NOs: 551-661 or an amino acid sequence having at least 60% sequence identity to said sequence or wherein the C-terminal portion of the DUF2121 recognition motif of the second substrate polypeptide consist of the amino acid sequence as defined in positions 16 to 40 of any one of SEQ ID NOs: 551-661 or an amino acid sequence having at least 60% sequence identity to said sequence.

27. The method of claim 21, wherein the produced fusion polypeptide comprises the second substrate polypeptide, preferably C-terminally.

28. The method of claim 2, wherein the first substrate polypeptide has an N-terminal portion defined from the N-terminus of the first substrate polypeptide to the aspartate residue in position 2 of SEQ ID NO: 308, 309, 310 and 311, respectively, wherein the produced fusion polypeptide comprises the N-terminal portion of the first substrate polypeptide and the second substrate polypeptide C-terminally fused thereto.

29. The method of claim 2, wherein the polypeptide comprising an N-terminal DUF2121 domain having an N-terminal serine or threonine residue is brought into contact with the first and the second substrate polypeptide simultaneously.

30. The method of claim 2, wherein the polypeptide comprising an N-terminal DUF2121 domain having an N-terminal serine or threonine residue is brought into contact with the first substrate polypeptide and wherein the second substrate polypeptide is only added after the first substrate polypeptide.

31. The method of claim 30, wherein the polypeptide comprising an N-terminal DUF2121 domain having an N-terminal serine or threonine residue is attached to a solid carrier and wherein the method further comprises washing the solid carrier after adding the first substrate polypeptide and before adding the second substrate polypeptide.

32. The method of claim 2, wherein the method is an in vitro method.

33. The method of claim 2, wherein the method further comprises collecting the produced fusion polypeptide.

34. The method of claim 2, wherein at least the portion of the first substrate polypeptide or the portion of the second substrate polypeptide forming part of the produced fusion polypeptide comprises a non-proteinaceous moiety attached thereto so that the produced fusion polypeptide comprises said non-proteinaceous moiety.

35. The method of claim 34, wherein the non-proteinaceous moiety is selected from the group consisting of a fluorophore, a drug, a toxin, a carbohydrate, a lipid, a solid carrier and an oligonucleotide.

36. The method of claim 2, wherein at least the portion of the first substrate polypeptide or the portion of the second substrate polypeptide forming part of the produced fusion polypeptide comprises an antibody, a domain or fragment thereof.

37. The method of claim 2, wherein at least the portion of the first substrate polypeptide or the portion of the second substrate polypeptide forming part of the produced fusion polypeptide comprises an enzyme.

38. The method of claim 2, wherein the portion of the first substrate polypeptide or the portion of the second substrate polypeptide forming part of the produced fusion polypeptide comprises a protein and wherein the portion of the other substrate polypeptide forming part of the produced fusion polypeptide has a solid carrier attached thereto, wherein the produced fusion polypeptide comprises the protein immobilized on the solid carrier, preferably wherein the protein is an enzyme.

39. The method of claim 2, wherein the first substrate polypeptide and/or the second substrate polypeptide is/are isotopically labeled, preferably wherein either the first or the second polypeptide is isotopically labeled.

40. The method of claim 2, wherein the portion of the first substrate polypeptide or the portion of the second substrate polypeptide forming part of the produced fusion polypeptide is part of a virus-like particle and wherein the portion of the other substrate polypeptide forming part of the produced fusion polypeptide comprises an immunogenic structure.

41. The method of claim 2, wherein the portion of the first substrate polypeptide or the portion of the second substrate polypeptide forming part of the produced fusion polypeptide is comprised in a membrane, preferably a vesicle membrane.

42. The method of claim 2, wherein the first substrate polypeptide comprises an intramolecular disulfide bond, preferably wherein the first cysteine residue forming the disulfide bond is located N-terminally of the DUF2121 recognition sequence and the second cysteine residue forming the disulfide bond is located C-terminally of the DUF2121 recognition motif.

43. The method of claim 2, wherein at least the portion of the first substrate polypeptide or the portion of the second substrate polypeptide forming part of the produced fusion polypeptide comprise an affinity tag.

44. The method of claim 2, wherein the portion of the first substrate polypeptide forming part of the produced fusion polypeptide comprises a first affinity tag, and wherein the portion of the second substrate polypeptide forming part of the produced fusion polypeptide comprises a second affinity tag, preferably wherein the first and second affinity tags are different.

45. A method for producing a circular polypeptide, comprising producing the circular polypeptide by bringing the polypeptide of claim 1 into contact with a substrate polypeptide and reacting the substrate polypeptide.

46. The method of claim 45, wherein the method further comprises producing a circular polypeptide.

47. The method of claim 45, wherein the circularization is generated via the formation of a peptide bond between two residues of the substrate polypeptide.

48. The method of claim 45, wherein the substrate polypeptide comprises two DUF2121 recognition motifs in a distance sufficient to allow circularization of the sequence.

49. The method of claim 48, wherein for the substrate polypeptide the circularization is generated via the formation of a peptide bond between the proline residue of the first DUF2121 recognition motif in position 3 of SEQ ID NOs: 308, 309, 310 and 311, respectively, and the aspartate residue of the second DUF2121 recognition motif in position 2 of SEQ ID NOs: 308, 309, 310 and 311, respectively.

50. The method of claim 45, wherein the substrate polypeptide comprises at its N-terminus the C-terminal portion of the DUF2121 recognition motif, said C-terminal portion of the DUF2121 recognition motif starting with the amino acid residues as defined in positions 3 to 5 of any one of SEQ ID NOs: 308, 309, 310 and 311 and further a DUF2121 recognition motif comprising any one of SEQ ID NOs: 308, 309, 310 and 311 in a distance to the N-terminus sufficient to allow circularization.

51. The method of claim 50, wherein for the substrate polypeptide the circularization is generated via the formation of a peptide bond between the N-terminal amino acid and the aspartate residue of the DUF2121 recognition motif in position 2 of SEQ ID NO:308, 309, 310 and 311, respectively.

52. The method of claim 48, wherein the DUF2121 recognition motif(s) of the substrate polypeptide is/are a DUF2121 recognition motif comprises at least 1 amino acid N-terminally of said SEQ ID NOs: 308, 309, 310 and 311, respectively.

53. The method of claim 50, wherein the C-terminal portion of the DUF2121 recognition sequence of the substrate polypeptide comprises at least 1 amino acids C-terminally of the N-terminal amino acids as defined by positions 3 to 5 of SEQ ID NOs: 308, 309, 310 and 311, respectively.


SEQUENCE LISTING (682 sequences)

SEQ ID NO: 1 - protein, 95 aa, synthetic construct
  source 1..95: note = >OB_consensus
LEKAKEIQMASKIITEGEVGKVVNIDGNMLEVKLNKDVEALDTNWKLAKPGENVLMFIDDPGEVKIGDKVVIENENLCIKRNKSPLKCDVILCKL

SEQ ID NO: 2 - protein, 196 aa, synthetic construct
  source 1..196: note = >DUF2121_consensus
SLVIAYVGKKGAVIAGDKREILFRGDKEKREKLEEELYSGKIKTDEELLKRAEELGVKIKIRDDREKVRKRGDVLVGEVTEIGAETRRRRIYATKGAYAIVEIEGSEITKKSGGGSGIIVFGNKITKEIANKEIRKAWKKKGSLKDAVRIIELIMEEAARTPSVSKEYDLLQTTEKDLEETIEEDIKELGKYRLKM

SEQ ID NO: 3 - protein, 295 aa, synthetic construct
  source 1..295: note = >NTN+OB_consensus
SLIIAYVGKKGAVIAGDKREILFRGDKEKREKLEEELYSGKIKTDEELLKRAEELGVTLKISDDREKVRKRGGVLVGEVSEIGAGETRRRRIYATTGGYAIVEIEGSEITNKSGGGSGIIVFGNKITKEIANKEIKKYWKKKGSLKDAGRIIELIMEEAARTPSVSKEYDLLQTTKEKDLEETIEEDIKELEKYRLKELLKQAEEIQMASKIITEGEVGKVSNIKGNMLEVKLNKDVEALDTNWKLLAKPGEKVLMFIDEPGDVKIGDKVVIENENLCIKRNKSPLNCDIILCKS

SEQ ID NO: 4 - protein, 203 aa, Methanococcus voltae
  source 1..203: note = >WP_013180795.1 DUF2121
SVIIGYYGNNGSAIAGDKRNILFRGSEANREKLESLLYNGKLKNDKELKEKAEEYDVKVHITDSQDKIKKLDGCLMGEVKTISNQSKRKRMYLSKNNCALVEIIDDDMTKKEIKKGSGIIIFGNKHLKQLVQAELQKNVNIVKSGDILKIEKLFKDILSNIDTPSSSNDVDSYSNFKYENLEKVISQELSNLVDYRNGLKNQI

SEQ ID NO: 5 - protein, 204 aa, Methanococcus aeolicus
  source 1..204: note = >WP_011973890.1 DUF2121
SIIVGYYGANGAVVGGDKRNIIFRGNAEQREELEKLLYSGNIKTDEEFKKIADEYGIKVYIEDKQLKVREVNGVLVGEVKSIGTDSQRRRMYLSTGNCAIIDILNDTITKKSSNVGHGLILFGNRYLKEIVHNELKKDAPNFKNMPIEKVKNIIENAIKRCDGPTVSEEIILLHTKDKHNNFKELIKKDINDLKEYRNGLRQQM

SEQ ID NO: 6 - protein, 204 aa, Methanothermococcus thermolithotrophicus
  source 1..204: note = >WP_018153627.1 DUF2121
SVIIGYYGKNGAVIAGDRRNILFRGDPKKREELEKKLYSGELKSDEELIRAAKDLGINIHIDDEKIKVRKIEDVLVGEVSSIGAESKRRRMYITKGKCAIIDILNDTITNKSIREGSSIVVFGNKYIKNLVQQELVKYKNKILKMSILEIKNLFEEILKNCNNPTVSKKYDVFYTVNCSKNFEETIENDIKDLFNYRNELNKKM

SEQ ID NO: 7 - protein, 205 aa, unidentified
  source 1..205: note = >GBF37010.1 DUF2121 Methanococcales archaeon HHB
SIVIGYYGNNGAVIGGDKRNIIFRGDPKKREELEKKLYSGKIKDEEELKKVAEELGVKVYIEDERVKVKKINNTLVGEVRSIGADSRRRRMYLTKGMCVIVDILNDDVIGKSVKRGNGVIIFGNKHLKEIARKELRKYMYDFTRMGIGEVKDVIEGVLKRCCRGPTSSKDFDILYIDEGEKDLEKVIEEDLEELRRYREELRQKM

SEQ ID NO: 8 - protein, 205 aa, unidentified; identical to SEQ ID NO: 7
  source 1..205: note = >WP_131007839.1 DUF2121 Methanococcales archaeon HHB

SEQ ID NO: 9 - protein, 204 aa, Methanothermococcus okinawensis
  source 1..204: note = >WP_013866916.1 DUF2121
SIVIGYYGNNGAVVAGDRRNIMFRGNPEKRAELEKDLYCGKIKNEEELKNRAEELGVKIFIEDERTKVKKIGDVLVGEVKSIGADSKRRKMYLTKGNCAIVDILNDTITNKSIKNGSSIIIFGNKYLKDIVQKELKKYMNNFGKMDILDVKNTIENALKKCDGPTLSPELDILHTNKKVFNLEEIIEKDLNDLKEYRNDLKQKM

SEQ ID NO: 10 - protein, 205 aa, Methanotorris igneus
  source 1..205: note = >WP_013799607.1 DUF2121
SLIIGYYGRNGAVLGGDKRNIMFRGDIEKRKKLEEMLYNGEIKDDEELLKKAEEFGIKLHIEDNRKKVRKIGDDVLVGEVKSIGIDSERRRVYLTKGKCIIVDVFNDEIRNKWTKTGSGVVVFGNRYVKKIAEEEFRKAGYRLTMMSLEEIRDLIENIFKKCNTPSVSKGYDIIYTTKECKNLEEVVKKDLDALFEYRETLRKKM

SEQ ID NO: 11 - protein, 205 aa, Methanotorris formicicus
  source 1..205: note = >WP_007043881.1 DUF2121
SVIIGYYGKNGAVLGGDKRRIMFRGDVEKRKMLEKLLYNGEIKNDEELLKKAEELGIKLHIEDGKKKVRKIGNDVLVGEVKTIGIDSKRRRVYLTKGKCMIVDVFNDEIKNKLIKTGSGVVVFGNKYVKKIAEDEFKKIGHRLTMMSLEEIKDIIENIFKKYDTPSVSKEYIVVYTTKECKNLEEVVKKDLDELFEYRETLRKKM

SEQ ID NO: 12 - protein, 205 aa, Methanocaldococcus infernus
  source 1..205: note = >WP_013100627.1 DUF2121
SLILCYFGNNGAIILGDRREMFFRGNEEKRKELEELLYSGEIKSEEELRKKAEELGVKIIIEDKRRKVWKIKNVVVGEVRSLGLDAKRRRVYATKRKAKILEILNDQIIGEKNLEGFSIIIFSNKFIKEEINKYLKEYYRVLPMKRIDEVGEIFKEIYKKVSWHPTLSEEFDVEKTDKEEEDFEEVIEEDVKKLFEYREELKKKL

SEQ ID NO: 13 - protein, 205 aa, Methanocaldococcus villosus
  source 1..205: note = >WP_004589885.1 DUF2121
SLVICYYGNNGAVMAGDRRQMFFKGPEEKRKILEEKLYSGEIQNEEDLYKLADDLGVKVIIEDNREKVRKIGNTLVGEVRSIGLEAKRRRVYATKGKCMILEILGDVITDRMLKNGAGLIVFGNRYLKNKAEKILKNVAKDFPKMDIDEVGKVIKDVFERFKEHPTISREYDIYVTKNIDINFEKTVEEDINKLFKYREDIRKKM

SEQ ID NO: 14 - protein, 206 aa, Methanocaldococcus vulcanius
  source 1..206: note = >WP_015733029.1 DUF2121
TLVICYYGKNGAVIGGDRRQIFFRGNEEKRKLLEEKLYSGEIKTEEELYKLAEELGVKIIIEDDREKVRKISDTVVCGEVRSLGVDAKRRRVYATKGKCAIIDILNDTITKEIIKEGFGLVIFGNKFLKNRAEEELRNKAKLFPMMPIQQIESEIREIFEKLKWHPTVSKEYDIYSVNKYEKNFEEVIKKDIGDLFEYREKLRQQL

SEQ ID NO: 15 - protein, 206 aa, Methanocaldococcus fervens
  source 1..206: note = >WP_015791349.1 DUF2121
SLIICYYGKNGAVIGGDKRQIFFRGNEEKRKILEEKLYNGEIKSEEELYKLADELNIKIIIEDNREKVRKISDSVVVGEVRSLGIEAKRRRVYATKGKCAIVDILNDTVTNEVIKEGFGIVVLGNRFLKKKAEEELKSSAKLFPMMPLEQIENVIRDIFEKLNWNPTISKEYDIYSVDKYEKNFEEVVRKDIESLFKYRDELRKQL

SEQ ID NO: 16 - protein, 206 aa, Methanocaldococcus sp.
  source 1..206: note = >WP_012979926.1 DUF2121; note = Methanocaldococcus sp. FS40622
SLIICYYGKNGAVIGGDRRQIFFRGEEQKRKILEEKLYSGEITSEDELYNLARELNIKIIIEDDREKVRKISDSVVVGEVRSLGIDARRRRVYATKGKCAIIDILNDTVTNQMIKEGFGVVVLGNRFLKKKAEEELKRTAKLFPMMPLQQIEDAIKEIFEKLKWHPTISKEYDIYSVNKYEKDFEEVIKKDIESLFKYREQLRKQL

SEQ ID NO: 17 - protein, 206 aa, Methanocaldococcus jannaschii
  source 1..206: note = >WP_010870052.1 DUF2121
SLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQL

SEQ ID NO: 18 - protein, 206 aa, Methanocaldococcus sp.
  source 1..206: note = >WP_048201244.1 DUF2121; note = Methanocaldococcus bathoardescens
SLIICYYGKNGAVIGGDRRQIFFRGNEENRKILEEKLYSGEIKSEEELYKLAKELDIKIIIEDNREKVRKISDSVVVGEVRSLGIDAKRRRVYATKGKCAIVDILNDTVTNQTIKEGFGIVVLGNRFLKKSAEEELKRTAKLFPMMPLEQIEDTIREIFEKLKWNPTISKEYDIYSINKYEKNFEDVVKKDIESLFKYREELRKQL

SEQ ID NO: 19 - protein, 204 aa, Methanococcus vannielii
  source 1..204: note = >WP_012066013.1 DUF2121
SVIIGYYGKNGVVIAGDKRTILFNGEEKKREMLEELLYSGKIKNDDDLSEKSAEFSVKVHILDDQKKIKQLENTLIGEVRTIGKDSKRKKMYLSKDICVILDIENDNIVKKSVKTGTGIVVFGNKYVKKIVELELKKYLSEISKMDVLKIRTIFEKILVGIENPTLSKGYDFLYCQNAEKDIESIIKKDLNDLREYREELSIKM

SEQ ID NO: 20 - protein, 204 aa, Methanococcus maripaludis
  source 1..204: note = >WP_011869407.1 DUF2121
SVIIGYYGKNGAVIAGDSRNLLFKGIKSNREKLEELLYSGKIRTDEELMKKASEFDVNVYINDSEEKVRILGNLLCGEIKSIGKDSKRRRMYLSKEKCAVLDIENDQITNKSVKTGSGIVVFGNKYIKSLVESEIKKYIKDILKMSVKEIKNLFEKILEKTENATLSGKFEYYFVETYENNFEMAVEDDLNELFEYRKELSLKM

SEQ ID NO: 21 - protein, 204 aa, Methanococcus maripaludis
  source 1..204: note = >WP_012193694.1 DUF2121
SVIIGYYGKNGAVIAGDRRNLLFNGIESNRKKLEELLYSGKIRTDEELMKKASEFDVNVYINDSEEKVRSLGTLLCGEVKSIGKDSKRRRMYLSREKCAVIDIENDRITNKSVKTGSGIVVFGNKYIKNLVESEIKKYIKNILKMSAEEIKNLFVKILENTKNATLSGNFEYHFVETYENNFEKVIEDDLNELFDYRKELSIKM

SEQ ID NO: 22 - protein, 204 aa, Methanococcus maripaludis
  source 1..204: note = >WP_011977398.1 DUF2121
SVVIGYYGKNGAVIAGDKRNLLFNGIESNREKLEEIMYSGQLRTDEELLKKASEFDVKIHINDTREKVKKFGNVLSGEVLSIGRESKRRRMYLTKEKCVILDIENDQITNKSVKNGSGIVVFGNRYIKNLVESEIKKHVQKILKMSAKEISALFEKILKNIENATLSNEFEYYFVETYENNLEKVIENDLNDLFNYRKELSLKM

SEQ ID NO: 23 - protein, 204 aa, Methanococcus maripaludis
  source 1..204: note = >WP_104837987.1 DUF2121
SVVIGYYGKNGAVIAGDKRNLLFNGIESNREKLEEVLYSGEIKSDEELFKKASEFDVTVHINDTREKVKSLGNLLSGEVLSIGKDSKRRRMYLTKEKCAIIDIENDQITNKSVKTGSGIVVFGNRHIKHFVESEIKKHIQKLLKMNAREIRDLFEKILKKIENATLSDTFEYYFVEAGEPEFEKAVNDDLDDLFNYRHDLSIKM

SEQ ID NO: 24 - protein, 204 aa, Methanococcus maripaludis
  source 1..204: note = >WP_013998520.1 DUF2121
SVVIGYYGKNGAVIAGDKRNLLFNGIESNRAKLEKVLYSGEIKTDEELFKKASEFDVTVHINDTREKVKSLGNLLSGEVVSIGKDSKRRRMYLTKEKCAIIDIENDQITNKSVKTGSGIVVFGNRYIKHFVESEIKKHVQKLLKMSAREIRDLFEKILKNIENATLSDNFEYYVVEAGEPEFEKAVNKDLDDLFNYRHDLSIKM

SEQ ID NO: 25 - protein, 204 aa, Methanococcus maripaludis
  source 1..204: note = >WP_011169965.1 DUF2121
SVVIGYYGKNGAVIAGDKRNLLFNGIESNRAKLEEVLYSGEIKNDEELFKKASEFEVTVHINDTREKVKSLGNLLSGEVVSIGKDSKRRRMYLTKEKCAIIDIENDQITNKSVKTGSGIVVFGNRYVKHFVESEIKKHVQKLLKMSAREIRDLFEKILKNIENATLSDTFEYYVVEAGEPEFEKAVNKDLDDLFNYRHDLSIKM

SEQ ID NO: 26 - protein, 204 aa, Methanococcus maripaludis
  source 1..204: note = >WP_119720453.1 DUF2121
SVVIGYYGKNGAVIAGDKRNLLFNGIESNRAKLEEVLYSGEIKTDEELFKKASEFDVTVHINDTREKVKSLGNLLSGEVVSIGKDSKRRRMYLTKEKCAIIDIENDRITNKSVKTGSGIVVFGNRYIKHFVESEIKKHVQKLLKMSAREIRDLFEKILKNIENATLSDTFEYYVVETGEPEFEKAVNKDLDDLFNYRHDLSIKM

SEQ ID NO: 27 - protein, 204 aa, Methanococcus maripaludis
  source 1..204: note = >BAP60138.1 DUF2121; note = Methanococcus maripaludis KA1
SVVIGYYGKNGAVIAGDKRNLLFNGIESNRAKLEKVLYSGEIKTDEELFKKASEFDVTVHINDTREKVKSLGNLLSGEVVSIGKDSKRRRMYLTKEKCAIIDIENDRITNKSVKTGSGIVVFGNRYIKHFVESEIKKHVQKLLKMSAREIRDLFEKILKNIENATLSDTFEYYVVETGEPEFEKAVNKDLDDLFNYRHDLSIKM

SEQ ID NO: 28 - protein, 213 aa, Methanopyrus sp.
  source 1..213: note = Methanopyrus sp. KOL6; note = >WP_088335494.1 DUF2121
TLVLAYAGTDGALVAGDHRILVARMDEEKMREVEEKLYSGEIRTEEELESFLKDLEVEGGYFEFHDDRKKVWKVNDEVVAGEVGVRSAKGVRRRRVYATPGAHAIIELEGEKVLSKNFGGPALIVEGPKVVKELVIEFVDSELGGKPDLESLRNALDDIFEYVSSNTVLVSSEYDAFEVKGRVDHLARIHLQKAIDEDIERLRKYRRRLVEEM

SEQ ID NO: 29 - protein, 213 aa, Methanopyrus kandleri
  source 1..213: note = >AAM01502.1 DUF2121; note = Methanopyrus kandleri AV19
TLVLAYAGTDGALVAGDRRTLVARMDEEKMRKVEEKLYSGEIRTEEELESFLKDLDVEDGYFEFHDDRKKVWKVNDEVVAGEVGVRSAKGVRRRRVYATPGAHAIVELEGEKVLSKNFGGPALIVEGPKVVKELVIEFVNSELGGKPDLESLRNALDDLFEYVSSGTILVSSEYDAYEVKGKADPLARARLQKAIDEDIERLREHRRRLAEEM

SEQ ID NO: 30 - protein, 210 aa, Methanothermus fervidus
  source 1..210: note = >WP_013413908.1 DUF2121
SLILAYVGKKGCVIAGDKRKIAYFGDKSSRKILEEKLYTGKIKTLDELFEEAKKLGVSINITDNAKKVREKGHVAIGEVGIKATHSAKRRRLYATTNCYEIIELEGSKIVKRKKGNSALIVFGNRITKKISNEILKKEFNPKMSLQEIANLFKKIIKKASELTPTINKSCDIVIKNPILNKEMAEKILEMSISEDIKNLRKWRKSLKDKM

SEQ ID NO: 31 - protein, 210 aa, Methanobrevibacter curvatus
  source 1..210: note = >WP_067092528.1 DUF2121
SLIIAYVGKKGCVMVADKRRISYFGDKEDIESLEDKLYSGDLENDEELKKTANLLNVSLKISDKESKIRSLESSLVAEVSTKSTHKAMRKRIYATTNAYQIIELEGSNISNLDKGDSSIIIFGNKITKSLANDFLSKEFKADVSLKYMGDIFTRILSKIHLKTPSIGNEFDVLVKTPKFSKEEAQKYLDDLVKVDIKLLVKFRDKLTQDL

SEQ ID NO: 32 - protein, 210 aa, Methanobrevibacter olleyae
  source 1..210: note = >WP_067146985.1 DUF2121
SLIIAYVGNKGCVMVGDKRRIAYFGSKEERELLEQEIYSGDISSDEDLYARAEELEISLKISDDACKVKSLENVAVGEVSSRGAMETKRKRIYGTTNGFQIVELTGSEIVNVKRGESSIIVFGNKITKSLANDMLKNKWKPSFSLKYMGEIFGQIIEDISTKTPSLGTKYDVVIQQNNLSKTKVQDYLDEVIERDINLLAKFRNKLKEDL

SEQ ID NO: 33 - protein, 210 aa, Methanobrevibacter ruminantium
  source 1..210: note = >WP_012956362.1 DUF2121
SLIIAYVGKKGCVMAADKRRIAYFGLKEQREILEQEIYSGEIHNDEELYARAEELDVNLKVTDNACKVKSIENVAIGEVSSRGTMETKRKRIYGTSNGFKIIELSGSHVLNSKRGVSSIIVFGNKVTKSLANKMIQERWKPSFSLKYMGEIFGEILKDIAEITPSVGKDYDVIIEKNNLAKSQVQDYIDEVAERDVKVLAKFRAQLREQL

SEQ ID NO: 34 - protein, 211 aa, Methanobrevibacter sp.
  source 1..211: note = >WP_016358763.1 DUF2121; note = Methanobrevibacter sp. AbM4
SIIIAYIGRKGCVMASDKRRIAYFGDKDNREKLEEELYSGNIRNDEELYKEAAKLGIDLKISDDAKKIKSIGNAVMGEVSSKTTREVRRRRIYGTTNGYQIIELIGSELKNKETGSSGIIIFGNKIAKQMANSLIKQRWKSSISLKYTGDIFEEIIKEIAEKTPSVGKNVDVIIGKDSKLTKTTAQKYLDETETRDIKVLEKYRQKLAEDL

SEQ ID NO: 35 - protein, 211 aa, Methanobrevibacter sp.
  source 1..211: note = >WP_040681637.1 DUF2121; note = Methanobrevibacter boviskoreani
SIIIAYIGRKGCVMASDKRRIAYFGDKDNREKLEEELYSGNIRNDEELYKEAAKLGIDLKISDDAKKIKSIGNAVMGEVSSKTTREVRRRRIYGTTNGYQIIELIGSELKNKETGSSGIIIFGNKIAKQMANSLIKQRWKSSISLKYTGDIFEEIIKEIAEKTPSVGKNIDVIIGKDSKLTKTTAQKYLDETETRDIKVLEKYRQKLAEDL

SEQ ID NO: 36 - protein, 211 aa, Methanobrevibacter wolinii
  source 1..211: note = >WP_042707893.1 DUF2121
SVIIAYIGKKGCVMASDKRRIAYFGDKDEREKLEEDLYTGKIRNDEELYNEAKKRQITLKISDDVNKIKKIENALMGEVSTKTTMEIRRKRIYGTTNGYQIVELLGSETQNKERGSSGIIIFGNKIAKSMANSLIKERWKSSLSLKYIGDIFEEVIKEIASKTPSVGKNVDVLYSKDNTLNKTSAQEYLDKTEERDNKLLEKYRQKLTEDL

SEQ ID NO: 37 - protein, 211 aa, Methanobrevibacter sp.
  source 1..211: note = >WP_088539534.1 DUF2121; note = Methanobrevibacter sp. 87.7
SVIIAYIGKKGCVMASDKRRIAYFGDKEEREKLEEDLYSGKIRNDEELYNEAAKRQITLKISDDVNKIKKIETVLMGEVSTKTTMEIRRKRIYGTSNGYQIVELLGSEVQNKERGNSGIIIFGNKIAKSIANSLIKERWKSNLSLKYIGDIFEDIIKEIASKTPSVGKNVEVLYTKNNNLNKVTAQEYLDKTEEIDNKLLEKYRQKLSEEL

SEQ ID NO: 38 - protein, 210 aa, Methanobrevibacter filiformis
  source 1..210: note = >WP_066972212.1 DUF2121
SLIIAYVGKKGCVIASDKRKIAYFGDKENIDKLEKELYNGNIKGDDDLYDTASKLDVSLKISEDGIKLRSFDKINVGEVSSKSTTETNRKRIYGTTNGYQIVELSGSKIVHTEKGNNALIIFGNKRTKTLANDLISKKWKSNFSLKYMGDIFLRILEEISAKTPSIGKAYDLNIINHKFTKEEANEYLDYFIEKDIQVLGKFRNQLKSEL

SEQ ID NO: 39 - protein, 210 aa, Methanobrevibacter smithii
  source 1..210: note = >WP_019266968.1 DUF2121
SLIIAYIGKKGCVMASDKRRIGYFGNKEQLNALESELYSGKIKNDDEFKKKADEYGIAIKLTEDATKITTVGNCVRGEVTTKKAFETYRKRIYGTTMGYQIVELSGSETVSREAGEKAIIVFGNKFAKQEAEKLINKKWKPSLSLKYMGDIFEEILTEISRKTPTIGNEFDVLIKQPKFNKSEAQRHLNVSIDKDIKVLSKFRQKLQEDL

SEQ ID NO: 40 - protein, 210 aa, Methanobrevibacter smithii
  source 1..210: note = >WP_019262821.1 DUF2121
SLIIAYIGKKGCVMASDKRRIGYFGNKEQLNALESELYSGKIKTDEEFKKKADEYGISIKLTEDATKITTVGNCVRGEVTTKKVFETYRKRIYGTTMGYQIVELSGSETVSREAGEKAIIVFGNKFAKQEAEKLINKKWKPSLSLKYMGDIFEEILTEISRKTPTIGNTFDVLIKQPKFNKSEAQRHLNVSIDKDIKVLSKFRQKLQEDL

SEQ ID NO: 41 - protein, 210 aa, Methanobrevibacter sp.; identical to SEQ ID NO: 40
  source 1..210: note = >WP_004035387.1 DUF2121

SEQ ID NO: 42 - protein, 210 aa, Methanobrevibacter smithii
  source 1..210: note = >WP_004033346.1 DUF2121
SLIIAYIGKKGCVMVSDKRRIGYFGNKEQLNALESELYSGKIKTDEEFKKKADEYGISIKLTEDATKITTVGNCVRGEVTTKKVFETYRKRIYGTTMGYQIVELSGSETVSREAGEKAIIVFGNKFAKQEAEKLINKKWKPSLSLKYMGDIFEEILTEISRKTPTIGNTFDVLIKQPKFNKSEAQRHLNVSIDKDIKVLSKFRQKLQEDL

SEQ ID NO: 43 - protein, 210 aa, Methanobrevibacter sp.
  source 1..210: note = >RBQ22740.1 DUF2121; note = Methanobrevibacter sp. NOE
SLIIAYVGKKGCVMASDKRRIAYFGEKDNREELEEELYSGSIANDDDLKKRSAELDITLKISDDANKIRYIEDVIVGEVSSRSTHETRRKRIYGTSNGYQLIEMLGSEITHSEKGESAIILFGNKVSKSLANDLISKKWKPRLSLKYMGEIFEDVLSEVSSKTPSLGKKYDVMIVQPKFNEKKAQSYLDETIKRDVQLLGKWREKLKNDL

SEQ ID NO: 44 - protein, 210 aa, Methanobrevibacter sp.
  source 1..210: note = >WP_042704220.1 DUF2121; note = Methanobrevibacter arboriphilus
SLIIAYVGKKGCVMASDKRRIAYFGDKENREKLEEEIYSGAIKNDEDLKKRSDELDITLKISDDANKIRYIENVIVGEVSSRTTFETRRKRIYGTTYGYQIIEMLGSDITHSEKGENAIILFGNKISKAMANDLISKKWKSSFSLKYMGEIFESVLEEVASKTPSLGKKCDVMIVHPELDKKSSQKYLDETIKRDVQLLGKWREKLKNDL

SEQ ID NO: 45 - protein, 210 aa, Methanobrevibacter sp.
  source 1..210: note = >WP_080459963.1 DUF2121; note = Methanobrevibacter arboriphilus
SLIIAYVGKKGCVMASDKRRIAYFGDKENREKLEEEIYSGSIKNDEDLKKRSDELNITLKISDDANKIRYIENVIVGEVSSRTTFETRRKRIYGTTYGYQIIEMLGSDITHSEKGENAIILFGNKISKAMANDLISKKWKSSFSLKYMGEIFESVLEEVASKTPSLGKKCDVMIVHPELDKKSSQEYLDETIRRDVQLLGKWREKLKSDL

SEQ ID NO: 46 - protein, 210 aa, Methanobrevibacter woesei
  source 1..210: note = >WP_116669733.1 DUF2121
SLIIAYVGKKGCVMASDKRKIAYFGNKENLEALESELYNGEIATDEELYKRAKDFDISIKISDDGNKIDTVGNTVMGEVASKGAFETKRRRIYGTTNGYQIIEIIGSEIVSRSAGEKAIIIFGNNFAKKEAEALISKKWKSTLSLKYMGDIFKEIINEVSQKTPTLGDKFDVLIQQPKFTADEAQKYLNETIDQDVKVLAKIRQKLQEDL

SEQ ID NO: 47 - protein, 208 aa, Methanobrevibacter millerae
  source 1..208: note = >WP_058739601.1 DUF2121
SLIIAYVGKKGCVMASDKRRIAYFGNNRQALEDELYDGSITTDEELYQRSKELDVPIKISDDADKIRVVGNTVRGEVSTKGTHETKRKRIYGTTNGYQIVELIGSETKSRQAGEKGIILFGNDFAKKQAETLIQRRWKASHSLRLMGDIFEEILSDIAQKTPTIGKEFDVLIQQPKFTASEAQKHLNITIDADIKVLTKYRQQLTEEM

SEQ ID NO: 48 - protein, 210 aa, Methanobrevibacter oralis
  source 1..210: note = >WP_042694031.1 DUF2121
SLIIAYIGKKGCVMASDKRKIGYFGNKENLEILENELYNAVIRDDDEFLKRAEELGISIKITDDANKISIVENTIRGEVSTKGTFETKRKRIYGTTNGYQIVELLGSEVSSRQAGKSGIIIFGNKFAKQKAETLINRYWKSSQSLKYMGEIFGNILEEISKKTPSIGSQFDILVKSPNFDEMKAQKHLDVTIDHDIKVLTKFRQKLTEEL

SEQ ID NO: 49 - protein, 210 aa, Methanobrevibacter sp.
  source 1..210: note = >WP_069573863.1 DUF2121
SLIIAYIGKKGCVMAADKRRIGYFGDKKNLDLLEEELYEGSIGNDEEFLARASELGISIKITDDANKLKTVGNCIRGEVSTKGTLETKRRRIYGTTNGYQIIELLGSDTKSRNAGENGIVIFGNNFAKQLAHNLISKKLKGSKSLRYMGEVFEDILKEVSSKTPTVGKNIDVLMQQPDFNKTEAQKHLNLTIDHDIKVLTKFRQELTEQI

SEQ ID NO: 50 - protein, 210 aa, Methanobrevibacter thaueri
  source 1..210: note = >WP_116591264.1 DUF2121
SLIIAYVGKKGCVMASDKRKIGYFGDKENLKKLEDELYSGKITADEDFLKRAKELGISIKITDDASKLKIIGNTIRGEVSTKGTFETRRRRIYGTTSGYQIIELLGSDTQSRKAGKNGVIIFGNDYAKRMAETFIKREWKSSQSLRYIGEIFQGIIEDVASKTPTVGKNVDVMIQHPEFNEREAQKHLDITIDHDIKVLTKFRQELTEQI

SEQ ID NO: 51 - protein, 210 aa, Methanobrevibacter sp.
  source 1..210: note = >WP_067043438.1 DUF2121; note = Methanobrevibacter sp. YE315
SLIIAYIGKKGCVMASDKRKIGYFGDKQNLKTLEEELYSGAIDNDEDFLNRASELGISIKITDDANKLKVVGNTIRGEVSTKGTFETRRRRIYGTTNGYQIIELLGSETESRKAGEKGIVIFGNVYAKQLAEPLIKRKVKASQSLRYMGEVFESILAEVASKTPTVGDKFDVLMQQPVFSVSEAQKHLNITIDNDIKVLTKFRQELTEQL

SEQ ID NO: 52 - protein, 210 aa, Methanobacterium sp.
  source 1..210: note = >WP_048190775.1 DUF2121; note = Methanobacterium sp. SMA27
SLIITYIGSKGCVMAGDKRRIGFFGPEGPRENLEEKLYSGSINTKEELLKKADELGINIKISDDADKIHEIGDVVVGEVRSKTVYETKRKRIYATTGAYNIIELLGSDLKTVKSGGSSIIVFGNKYTKELAEKTIKRYWKSKLNLVTIRKIFESVMEEVAIETPTVSPEYDVMMKYPSIDTKEAKELLRVTILQDVKDLEEYRTQLKENL

SEQ ID NO: 53 - protein, 210 aa, Methanobacterium sp.
  source 1..210: note = Methanobacterium lacus; note = >WP_013644693.1 DUF2121
SLIVTYIGSKGCIMAGDKRRIGFYGSPEARETLEDELYAGKIKTEEDLIKKAEEQGINLKISDDTTKIREMDNVLVGEVISRATHETKRKRIYAISGAYSMVELTGSNIDTVKSGGRSIIVYGNKYTQELAEKTIKKYWKPKMKFVDVERIMELVMSEVSENTPSVSKSFDLMIKYNSMDQKLAKEVIRTTVLEDVKNLEAFRNQLREEN

SEQ ID NO: 54 - protein, 210 aa, Methanobacterium sp.
  source 1..210: note = >OPX57417.1 DUF2121; note = Methanobacterium sp. PtaB.Bin024
SLIITYIGSKGCVMAGDKRSIGFLGDKNQREILEEELYSGKIKTKDQLTKRAHQLDINLKITDESEKVRKLDKVLVGEVKIRATHETRRKRIYATTNGYHQVELVGSEIKNVKSGKSSIVIFGNKLTKEITGKRLKKHWKSKISLNEVGEIFQKVMEDVAQVTPSVSPQYDLSIIHPQMDHKQAMELLRTTIISDVKELEKWREQLREDM

SEQ ID NO: 55 - protein, 210 aa, unidentified
  source 1..210: note = Methanobacteriales archaeon HGW Methanobacteriales2; note = >PKL73782.1 DUF2121
SLIMTYIGSKGCVMAADKRSIGFLGDKNQRELLEEDLYSGKIDSDEKLLKRAKELDINLKISDQGEKIRNLGKVLVGEVKIRATHETKRKRIYATTNGYHQVELTGSEIQNVKSGQSSIVIFGNKITKEIAGKSLKKHFKSKISLNEVGKIFQKVMEDVAEATPSVSSEHDIFIIHPQMDHKQAMELLRTTIISDVKELEKWRNKLKEDM

SEQ ID NO: 56 - protein, 210 aa, Methanobacterium sp.
  source 1..210: note = >WP_100906514.1 DUF2121
SLIMTYIGSKGCVMAADKRSIGFLGDKNQRELLEEDLYSGKIDSDEKLLKRARELDINLKISDQGEKIRNLGKVLVGEVKIRATHETKRKRIYTTTNGYHQVELTGSEIQNVKSGQSSIVIFGNKITKEIAGKSLKKHFKSKISLNEVGKIFQKVMEDVAEATPSVSSEHDIFIIHPQMDHKQAMELLRTTIISDVKELEKWRNKLKEDM

SEQ ID NO: 57 - protein, 210 aa, Methanobacterium sp.
  source 1..210: note = Methanobacterium sp. MB1; note = >WP_039376705.1 DUF2121
SLIITYVGSKGCVMAGDKRSIGFLGDKEQREVLEEEMYSGKIKTTEELLQRAEQLDINLKITDDVEKVRKIGDVLVGEVKLRTTTETKRKRIYGTSGGYHEVELSGSQIKNAKSGQSSIVVFGNKFTKEIANKHLQKYWKSQTSIKEIAKIFQRIMEDVAQSTPSVSPQFDIYTVHPQLNHKQAMEILRTTIVKDVKELEKWREELKTQM

SEQ ID NO: 58 - protein, 210 aa, Methanobacterium sp.; identical to SEQ ID NO: 57
  source 1..210: note = >CDG65302.1 DUF2121; note = Methanobacterium sp. MB1

SEQ ID NO: 59 - protein, 210 aa, Methanobacterium formicicum
  source 1..210: note = >WP_048073463.1 DUF2121
SLIITYVGSKGCVMAGDKRSIGFLGDKEQREVLEEEMYRGKIKTTEELLQRAGQLDINLKITDSVEKVRNLGDVLVGEVKLRTTTETRRKRIYGTSSGYHQVELSGSEIKNVKSGQSSIVVFGNKLTKEITNKHLQKYWKSQTSLNEVSKIFQRIMEDVAQSTPSVSSQFDIYMVHPRLDHKQAMELLRTTIIEDVKELEKWREELKTQM

SEQ ID NO: 60 - protein, 210 aa, Methanobacterium formicicum; identical to SEQ ID NO: 59
  source 1..210: note = >CEA14429.1 DUF2121

SEQ ID NO: 61 - protein, 210 aa, Methanobacterium formicicum
  source 1..210: note = >WP_048086016.1 DUF2121
SLIITYVGSKGCVMAGDKRSIGFLGDREQREVLEEEMYSGKIKTTEELLQRASQLDINLKITDSVEKVRNLGDVLVGEVKLRTTTETRRKRIYGTSSGYHQVELSGSEIKNVKSGQSSIVVFGNKLTKEIANKHLQKYWKSQTSLNEVSKIFQRIMEDVAQSTPSVSSQFDIYMVHPRLDHKQAMELLRTTIIKDVKELEKWREELKTQM

SEQ ID NO: 62 - protein, 210 aa, Methanobacterium formicicum; identical to SEQ ID NO: 61
  source 1..210: note = >AIS32222.1 DUF2121

SEQ ID NO: 63 - protein, 210 aa, Methanobacterium formicicum
  source 1..210: note = >WP_060537561.1 DUF2121
SLIITYVGSKGCVMAGDKRSIGFLGDREQREVLEEEMYSGKIKTTEELLQRASQLDINLKITDSVEKVRNLGDVLVGEVKLRTTTETRRKRIYGTSSGYHQVELSGSEIKNVKSGQSSIVVFGNKLTKEIANKHLQKYWKSQTSLNEVSKIFQRIMEDVAQSTPSVSSQFDIYMVHPRLDHKQAMELLRTTIIEDVKELEKWREELKTQM

SEQ ID NO: 64 - protein, 210 aa, Methanobacterium formicicum; identical to SEQ ID NO: 63
  source 1..210: note = >CEL24543.1 DUF2121

SEQ ID NO: 65 - protein, 210 aa, Methanobacterium sp.; identical to SEQ ID NO: 63
  source 1..210: note = >AXV39335.1 DUF2121; note = Methanobacterium sp. BAmetb5

SEQ ID NO: 66 - protein, 210 aa, Methanobacterium sp.
  source 1..210: note = Methanobacterium sp. Maddingley MBC34; note = >EKQ53249.1 DUF2121
SLIITYVGKKGCVMAGDKRSIGFLGDRKQRELLEEELYAGKIKTTEDLHKRAEQLDINLKITDNVEKVRNLGEVLVGEVKLRTTLETRRKRIYGTSSGYHQVELTGSEIRKVQSGQSSIVVFGNKITKEIANKHLKKYWKSKTSLDEVGKIFRRIMEDVAQATPSVSQQYDIFIIHPQLNHKQAMELLRTTIVGDVKELEKWREKLKQEM

SEQ ID NO: 67 - protein, 210 aa, Methanobacterium formicicum
  source 1..210: note = >WP_004031535.1 DUF2121
SLIITYVGSKGCVMAGDKRSIGFLGDKEQRELLEEEMYTGKIKTTEELHKRAKELEINLKITDNVEKVRNLGEVLVGEVKLRTTLETRRKRIYGTSSGYHQVELTGSEIKNVKSGQSSIVIFGNKITKEIANKHLKKHWKSKTSLNEVGKIFRNIMEDVAQLTPTVSSQYDIFTVHPQLDHKQAMELLRTTMIADVKQLQKWREDLKQEM

SEQ ID NO: 68 - protein, 210 aa, Methanobacterium sp.
  source 1..210: note = >OPY22699.1 DUF2121; note = Methanobacterium sp. PtaU1.Bin097
SLIICYTGSNGSVIIGDKRRIGFFGNEKKRELLEEEFYSGSIKTQEALLKRAEELGITLKITDDAEKVREIGDVITGEVKTTTPFETKRKRIYATTGSYSLVELSGSTIKNMEGGTTSIVVFGNKYTKEIANKAIQDNWKKKVSLKDVGEIFEKAMDEVSRQTPSVSPNYDLIIKHPSLNKKQARELLRTTILQDVKELEKWREELKEQM

SEQ ID NO: 69 - protein, 210 aa, Methanobacterium sp.
  source 1..210: note = >TMS41273.1 DUF2121
SLIICYTGSNGSVIIGDKRRIGFLGNEKKRKLLEEELYSLSIKSQDELLKRAAELEITLKISDDAEKVRELGDVVVGEVKNTTPFETKRKRIYATTGSYNLVELTGSSIEKMDGGTTSIVVFGNKSTKKIANQVIEENWKKKLSLKDVAGIFKKAMEEVSRQTPSVSQDYDIIVKNPELNKNQAREILRTTIIQDVKDLEKWRSQLKEQM

SEQ ID NO: 70 - protein, 210 aa, Methanobacterium sp.
  source 1..210: note = >WP_069583131.1 DUF2121
SLIMSYIGSNGCVMAGDKRRIGFFGDKEKREKLEEYLYSGAIQTDEELVKVAKEQEVTLKISDDARKIRSIADVVVGEVRFKTPFETKRKRIYGTTNGYIITELSGSDIKKVENGASSIVVFGNKVTKQMANEIIKKHWKSKASLENVESVFKKVMDEISGKTPSVSKDYDIIIKHSGMDKREAQKHLREILIKDVKELQKWREGLRQQL

SEQ ID NO: 71 - protein, 210 aa, Methanobacterium sp.
  source 1..210: note = >WP_048080675.1 DUF2121
SLIMSYIGSNGCVMAGDKRRIGFFGDKEKREKLEEYLYSGAIQTDEELVKVAKEQEVTLKISDDARKIRSIDDVVVGEVRFKTPFETKRKRIYGTTNGYIITELSGSDIKKIENGASSIVVFGNKVTKQMANEIIKKHWKSKASLENVESVFKRVMDEISTKTPSVSKEYDIITKHSGLDKKEAQKHLREILIKDVKELQKWREGLRQQL

SEQ ID NO: 72 - protein, 210 aa, Methanobacterium sp.
  source 1..210: note = >AXV36996.1 DUF2121; note = Methanobacterium sp. BRmetb2
SLIMTYIGSHGCVMIGDKRRIAFFGDKELIQKLEEELYSGKIKNDEELLERSKSLDVTIKVVDDAKKVRSLGDVVVGEVRFKTTFETRRKRIYATTNGYDIVELLGSKIDKIQKGESSIIVFGNKMTKKIANDTIRENWKPKTNLKDIGELFKVIMEEIALLTPSISKDYDFFVKSKNMNNNEAQKLLRETIVRDVKLLEKWRNKLKDDL

SEQ ID NO: 73 - protein, 210 aa, Methanothermobacter sp.
  source 1..210: note = Methanothermobacter sp. MT2; note = >BAW31310.1 DUF2121
SLIITYVSSKGCVIVGDKRKIAYFGDRENRKKLEKELYEGKIKNEKELRKRAEELGITLNIRDDAKKVRSIGDVVIGEVSLKTPFEAKRRRIYATTNAYQIVELVGSNITKLEKGESSIIIFGNKITKELTGKLLKKRWKKKTNLKEVSKLLEEVLEEVAKKTPTISQEHDKFIKTPRLDKRKAHKLLRETIVRDVKVLQKWRATLKMEL

SEQ ID NO: 74 - protein, 210 aa, Methanothermobacter sp.
  source 1..210: note = >WP_112094313.1 DUF2121; note = Methanothermobacter tenebrarum
SLIITYTSSKGCVMIGDKRKIAYLGSPQKRRELERELYNGDIKTEEELRDRAEEFGIRLNIRDDARKVRSIGDVVVGEVSLKTPFEAKRRRIYATTGAYQIIELIGSNITKFEKGESSIVIFGNKITKKLASSLLKKYWKKKTSLKEVSKLLAKIVEEVAAKTPTISPEYDLYIKTPRLDKRKAHRILRETILRDVKTLQKWRAKLQKEL

SEQ ID NO: 75 - protein, 210 aa, unidentified
  source 1..210: note = Methanobacteriaceae archaeon 41_258; note = >KUK01501.1 DUF2121
SLIITYISSKGCVIIGDKRKIAYLGSAQNRRELERELYNGNIKTEEELRSRAEQLGITLNITDDAKKVRSIGDVVVGEVSLKTPFEAKRRRIYATTGAYQIIELIGSNITKFEKGESSIVIFGNKITKKLASKLLKKHWKKKTSLEGISKLLKKILEDVATKTPTISREYDMYIKTPRLDKRKAHKLLRETILRDVKTLQKWRAKLQKEL

SEQ ID NO: 76 - protein, 210 aa, Methanobacterium sp.
  source 1..210: note = Methanobacterium paludis; note = >WP_013825588.1 DUF2121
SLIITYIGSKGCVMIADKRRIGFFGEEKTREKLEEELYSGAIKTDEELLKKASSLGITLKITDDAEKIREIGDIVVGEVRFKTPFETKRKRIYGTTGAYNVVELLGSEIKRIKTGESSIVVFGNKVTKEIANKYIQNHWKKKTNLKDIGEIFKAAMEEVAAKTPSVSQKYDLFIKHADIDKKEAKELLRTTIITDVKDLKKWREDLTDEM

SEQ ID NO: 77 - protein, 210 aa, Methanobacterium congolense
  source 1..210: note = >WP_071906792.1 DUF2121
SLIITYVGSKGCVMAGDKRRIGFFGDEANREKLEEKLYSGEIRTDEELLKRASELEITLKITDDAEKVREIDDVAVGEVRFKTPFKTKRKRIYGTTGAYNIVELLGSDIKTIKGGESSIVVFGNKMTKEIANKTIKDNWKNKTSLQDIAKLFSKVMEDVARQTPSVSQEHDVIIKHPSMEYKEAKELLRTTIIQDVKDLTEWRNSLKEEL

SEQ ID NO: 78 - protein, 210 aa, Methanobacterium sp.
  source 1..210: note = >RJS49378.1 DUF2121
SLIITYIGAQGCVMAGDKRRIAFFGDKSNREILEEELYSGKIENESQLKKRASELEITLKITDDAVKLRSLGKVVVGEVSSKSSFETKRRRIYATTNGFQIIELTGSNITKMDKGESSIIVFGNKFTKKTASKILKKYWKSKTTLKEIETIFQKVLHEVSEKTPSVGSSCDLFIKTPALDKKESQKILRETVVRDVKLLQKWRQKLKKDL

SEQ ID NO: 79 - protein, 210 aa, unidentified
  source 1..210: note = >PKL67848.1 DUF2121; note = Methanobacteriales archaeon HGW Methanobacteriales1
SLIMSYIGSKGCVISADKRRIAYFGDKSNREQLEEELYAGAIINDKELQARALDLGITLKISDDVCKVRSVGEVVVGEVSVKSPFETKRRRIYGTTNGYQIVELSGSDIAKMEKGEISIVVFGNKKTKEVANEIIKKRWENKTSLKGIADIFKEVMVEVAKVSPSISSENDIFIKHPKLDKREAQKLLRETILRDVKLLKKWREKLQNDL

SEQ ID NO: 80 - protein, 210 aa, Methanothermobacter marburgensis
  source 1..210: note = >WP_013295962.1 DUF2121
SLIITYISTRGCVIAGDKRRIAYFGDKSKREKLEEELYSGKIKSDDELHRRASELGVSIKVTDDTAKVRSLGDVVVGEISQKTPFETKRRRIYATTGAYQIIDLTGSRITSMEKGETAIIVFGNKVAKELTNRFLKKRWKTKTTLKEVENLFRELMDYVSSRTPSVGSKYDIFMKSPSLDKRSAHKLLSDTIVRDVRLLQKWRAKLKQEM

SEQ ID NO: 81 - protein, 210 aa, Methanothermobacter wolfeii
  source 1..210: note = >WP_074359097.1 DUF2121
SLIVTYISTRGCVIIGDKRRIAYFGDRSAREKLEEELYSGRIKSDEELQRRASELGLNIKVTDDACKVRSIGDVVVGEISQKTPFETKRRRIYATTGAYQIIELTGSRITSMEKGDTAIIVFGNRIAKEITNGFLKKRWKTRTTLKEVSELLTELMEHVSSRTPSVGSKYDLFMKSPALDKKSAHRILSETIVRDVKVLQKWRAKLRQEM

SEQ ID NO: 82 - protein, 210 aa, Methanothermobacter sp.
  source 1..210: note = Methanothermobacter sp. CaT2; note = >WP_048175474.1 DUF2121
SLIITYISSKGCVIAGDKRRIAYFGDKSSREVLEEELYTGKIKSDEELQRRASELGVNIKVTDDTCKVRSLGDVVVGEISQKTPFETRRRRIYATTGAYQIIELTGSKITSMEKGDTAIIVFGNKIAKEITNRFLKKRWKTKTSLKDVADLFRELMDHVSSQTPSVGSEYDLFIKSPSLDKKSAHKLLSDTIVRDVRLLQKWRAKLKQEM

SEQ ID NO: 83 - protein, 210 aa, Methanothermobacter thermautotrophicus
  source 1..210: note = >WP_010876390.1 DUF2121
SLIITYISSKGCVIAGDKRRIAYFGDKSSREVLEEELYTGKIKSDEELQRRASELGVNIKVTDDTCKVRSLGDVVVGEISQKTPFETRRRRIYATTGAYQIIELTGSRITSMEKGDTAIIVFGNKIAKEITNRFLKKRWKTKTSLKDVADLFRELMDHVSSQTPSVGSEYDLFIKSPSLDKKSAHRLLSDTIVRDVRLLQKWRAKLKQEM

SEQ ID NO: 84 - protein, 210 aa, Methanothermobacter sp.; identical to SEQ ID NO: 82
  source 1..210: note = >BAZ98793.1 DUF2121; note = Methanothermobacter sp. EMTCatA1

SEQ ID NO: 85 - protein, 210 aa, Methanothermobacter defluvii; identical to SEQ ID NO: 83
  source 1..210: note = >WP_115892198.1 DUF2121

SEQ ID NO: 86 - protein, 197 aa, Methanothrix sp.
  source 1..197: note = Methanothrix concilii; note = Methanothrix concilii Adriase; note = >WP_013718183.1
SLVVALTKSKEAVIGGDRRSITFLGSWPELEEELYCGRISDDEALLAKAMEIGAILQVTDGRDKVWRRGDLLVGEVTEITPQLERRRRIYLAPGSQLQVDITGKEVRIRDRGAMGCIIYGNRFTQQIAVSIVVQADGRVDEALIRKIFQEAAERTASVSQEHVILKSERMLSNPQKALEKALEEDCQENGWKLCDPL

SEQ ID NO: 87 - protein, 209 aa, unidentified
  source 1..209: note = Methanosaeta sp.; note = Methanosaeta sp. ASO1; note = >OYV11463.1 Methanosaeta sp. ASO1 Adriase
SLVIALAVSRQALIAADRRAIVFLGSCRKLEEELYSGQIKSDLELEARADALGAALHISDSREKIWRRGDVLVGEVTELSALLQRRRRIYLAPGASLQVEIRSGEDDGMEPGTVGGEARITAWGGVGCTIFGNRFTQKLAAEEIALAGAKVDRSLIERIFQKAGERTASVSRQHTILSSSLIKADARADLLAALQEDCKERGWRLCGPQ

SEQ ID NO: 88 - protein, 209 aa, unidentified
  source 1..209: note = Methanosaeta sp.; note = Methanosaeta sp. ASM2; note = >OYV11309.1 Methanosaeta sp. ASM2 Adriase
SLVIALAVSRQALIAADRRAIVFLGSCRKLEEELYSGQIKSDLELEARADALGAALHISDSREKIWKRGDVLVGEVTELSALLQRRRRIYLAPGASLQVEIRSGEDDGLEPGTVGGEARITAWGGVGCTIFGNRFTQKLAAEEIAIAGAKVDRSLIESIFQKAGERTASVSRQHTILSSSLIKADARADLLAALQEDCKESGWRLCGPQ

SEQ ID NO: 89 - protein, 209 aa, unidentified
  source 1..209: note = Methanosaeta sp. NSM2; note = Methanosaeta sp.; note = >OYV11827.1 Methanosaeta sp. NSM2 Adriase
SLVIALAVSRQALIAADRRAIVFLGSCRKLEEELYSGQIKSDLELEARADALGAALHISDSREKIWKRGDVLVGEVTELSALLQRRRRIYLAPGASLQVEIRSGEDDGLEPGTVGGEARITAWGGVGCTIFGNRFTQKLAAEEIAIAGAKVDRSLIERIFQKAGERTASVSRQHTILSSSLIKADARADLLAALQEDCKESGWRLCGPQ

SEQ ID NO: 90 - protein, 200 aa, Methanothrix sp.
  source 1..200: note = >RQW79829.1 Methanothrix sp. Adriase
SLVVALAGSREAVIGGDRRSISFLGSCAKLEEELYSGQIKNDEELLARAREHEATLQVSDGREKVWKTGEVLVGEVTEISERLQRRRRIYLVPGAHLVVDITGSEAKISSTGKVGCVVFGNRITQRLAAEGAAKAKGRLNEAVIESILVDVADKTPSVSREHSVLRTDARPSNPDAALMGALQADCRKNGWRLCGPCAPQ

SEQ ID NO: 91 - protein, 225 aa, unidentified
  source 1..225: note = Methanosaeta sp. PtaU1.Bin112; note = Methanosaeta sp.; note = >OPY53627.1 Methanosaeta sp. PtaU1.Bin112 Adriase
SLVIALAASREAVIGADRRAITFLGSCPRLEEELYSGQIKDDQQLLDRAGELGAVLQITSSKEKVWRRGDLLVGEVTEISALLSRRRRIYLTPGASLQVDITSDGLEELADAGACKEAKKEAPDRASDKAHGEARIRSWNGVGCTVFGNLFTQKLAYDEVGRAGGRVNEPLVKSILAKAGERTASVSRESTVLRSDIKQPDPKAALLRALEADCKENGWRLCAPQ

SEQ ID NO: 92 - protein, 191 aa, Methanothrix thermoacetophila
  source 1..191: note = >ABK15022.1 Methanothrix thermoacetophila PT Adriase; note = Methanothrix thermoacetophila PT
SLVIAFSGREAVIAGDRRSIAFAGDCRLLEEELYSGRIHDDVELLRRAGELSATIYISDGREKVWRDGDVLVGEVTEVSASSSKRRRVYVTSGAYLIVDVVDGNARISGRGGASLIILGNRHTRDAAGKALQGLGKISADAIARVMDEVSAGTATMSREHTLLKITAHHKDPASILMQTFSRDCERMGWRL

SEQ ID NO: 93 - protein, 199 aa, unidentified
  source 1..199: note = Methanosaeta sp.; note = >KQC14876.1 Methanosaeta sp. SDB Adriase; note = Methanosaeta sp. SDB
SLVVGFAGKKRAILGGDRRSIVFFGGAEALEEELYDGKIRTDEELKKRAEELGSVIQIANGREKVWKRNDGKVLVGEVTELSASSQRRRRVYLTPGGYILVEISGKRAEVSKKGKSTLIILGNKFTRELAYRRLGSAGKPPEISSMKALFEEARRATATISPDYTLLSSDEICSNPEAVLLQALREDCEERGWELFGLA

SEQ ID NO: 94 - protein, 196 aa, unidentified
  source 1..196: note = >OPX80929.1 Methanosaeta sp. PtaB.Bin087 Adriase; note = Methanosaeta sp.; note = Methanosaeta sp. PtaB.Bin087
SLVVGFAGKKRAILAGDKRRIAFFGRAERLEEELYGGEIRSSEDLERRAVEFGSKIAITDGREKVWKRGGVQVGEVTELSAERQRRRRVYLVPGGYLLVEVDGQKAEISKRGASTLIILGNRFTRDFAFKRLGSGMPPDEASLRSLFEEVGRLTASVSPDYTILISDSVHPDPEAVLLRALKEDCEEGGWELSGPA

SEQ ID NO: 95 - protein, 196 aa, unidentified
  source 1..196: note = Methanosaeta harundinacea; note = >WP_014586755.1 Methanosaeta harundinacea Adriase
SLVVGFAGKKRAIVAGDKRSIAFFGRAEELEEELYGGEIRSREDLQRRCEELGSKVAIADGREKVWDRGGVLAGEVTEISAERERRRRIYLVPGGYLLVEVDGKRAEISKRGASTLIILGNRFTRDFAFKRLGSGRPPDEASIRALFEEAGRATASVSPDYTVLTSSSIHRDPEAALLRALREDCEEGGWELCGLA

SEQ ID NO: 96 - protein, 200 aa, Methanoculleus sp.
  source 1..200: note = Methanoculleus taiwanensis; note = >WP_128693800.1 Methanoculleus taiwanensis Adriase
SLVIAFIGARGAVMAGDMREILFSGDDSCRDRLEAELYDGRIVTDEALARRAQEIGIGVYVRDTKNKVAERSGILVGEVGETEKGVVRSRRLYASAGRYAIADFEDGCMTVVSQGTGSTFIVLGNAIAKQVTNRCIRDTWKGGTPRDAVRTIVLAMETAARTTASVSERYVLVQTPASRDLRPLLDEDARRGEERSNSAE

SEQ ID NO: 97 - protein, 207 aa, Methanofollis ethanolicus
  source 1..207: note = >WP_067053047.1 Methanofollis ethanolicus Adriase
SLVIAFVGSKSAVMAGDMREIFFWGDETAVQALEKELYGGGLVADNDLYRRAEALGVGVVVRDGREKVREQDGVLVGEVRETVGGTVRARRLYATAGRYAIAEFEGSCGRLTGKGTGSTFVVLGNRAAQAIALASIREGWKDGGTEDAVRVIILVMKTAAAATASVSKRFVLIRTEAGADLDTLLARDLGEGEVAMKNTSGIFRKNA

SEQ ID NO: 98 - protein, 193 aa, Methanoculleus sp.
  source 1..193: note = Methanoculleus sp. SDB; note = >KQC03708.1 Methanoculleus sp. SDB Adriase
TLVMAFVGKNGAAVAGDMREILFRGDEARRRELESELYDGRIVTDADLEKRAAEIGVRISVRDDKSKVIRRDDGILTGEVSKFERGTVSRRRIYAVAGQYAIADFEGGRMVIVQKKEGSAFVVLGNDITKKIANEIIRKNWKNGGFEDAVRVIVLAMEEAGRRTASVSRAFSIEQTDINVNISAIVDSEAEGS

SEQ ID NO: 99 - protein, 207 aa, unidentified
  source 1..207: note = Candidatus Methanoperedens sp. BLZ2; note = >WP_097297421.1 Candidatus Methanoperedens sp. BLZ2 Adriase; note = Candidatus Methanoperedens sp.
SLIIAFVGGTEAAIVADMREITFQGDKVLRERLERKLYRGAIATDIELEKMAEEIGVKIKIRDNKIKVMQRYGALVGEVVSVEDGFVRKRRLYATAGSYMIAETRDSEVKVTNKGGSAFIVLGNNITKEIANKCIRENWKNGGFHEAIKVLILSMEAAAKVSASVSKKFIIIQTSSNVNLSEVIEQDKIGRKNLPWCSSVYAFGGYD

SEQ ID NO: 100 - protein, 192 aa, Methanosarcina sp.
  source 1..192: note = >WP_048178734.1 Methanosarcina sp. MTP4 Adriase; note = Methanosarcina sp. MTP4
SLVIAFIGKNGAVMAGDMREITFEGEKENREKLEKDLYSGAIVSDAELEKKAEELGVKITVRDDKNKLEERDGVLVGEVSSVEGGVVRKRRLYVSAGNYAIAEIEGSEIRVTAQGKGSNFIAFGNDTTKAIANKCFKDNWKKGNLQLAVKIIMLAMETAARKTASVSKKYMLLQTASNVDVSGVVEKDLSEP

SEQ ID NO: 101 - protein, 192 aa, Methanosarcina sp.
  source 1..192: note = >OEU41885.1Methanosarcina sp. Ant1 Adriase; note = Methanosarcina sp. Ant1
SLVIAFIGKNGAVMAGDMREISFQGEKPDREKLEKELYSGALVTDDELTKKAEELKVKINVTDFKNKVSERDGILIGEVSSFEDGVIKKRRLYASAGNYAIAELRNSKITLTSHGKGSNFIAFGNEFTKQTANKCFQDNWTKKSNFQDAVKILKLCMETAARKTASISKQFVLVQTVSNNDVLNVVEKDRQG

SEQ ID NO: 102 - protein, 192 aa, Methanosarcina horonobensis
  source 1..192: note = >WP_048140017.1 Methanosarcina horonobensis Adriase
TLVIAFIGKNGAVMAGDLREITFEGEKPDREKLEKELYNGAIVTDEDLAKKAEECGVKIAVTDCKEKVSERDGILVGEVSSVEGGVVKKRRLYAAAGNYAIAELRDSELTLTSQGKGSNFIAFGNEFTKQVANKCFKDHWMKKSTFQDAVKILMLCMETASKATASVSKQFYLIQTTSNVDVLKVVEKDRQG

SEQ ID NO: 103 - protein, 196 aa, Methanosarcina lacustris
  source 1..196: note = >WP_048125541.1 Methanosarcina lacustris Adriase
TLVIAFIGKNGAVMAGDLREITFDGEKPDMEKLEKELYSGTIVTDEELTKKAEEFGVKIMVTDCKSKVSERNGILVGEVSSVEGSAVKKRRLYASAGNYAIAELRDSELTMTSQGKGSNFIAFGNEFTKQIANKCFTDNWTKKSKLQDAVKILMLCMETAARETASVSKQFVIMQTASNVDVLKVVEKDRKNCIKC

SEQ ID NO: 104 - protein, 192 aa, Methanosarcina acetivorans
  source 1..192: note = >AAM05704.1 Methanosarcina acetivorans C2A Adriase; note = Methanosarcina acetivorans C2A
TLVIAFIGKNGAVMAGDMREITFEGEKPDREKLEKELYNGAIVTDEELARKAEEAGVKITVTDCKNKVSERNGILVGEVSSVERGIVKKRRLYASAGSYAIAELRDSELTLTSQGKGSNFIAFGNEFTKQVANKCFKDNWTKKSKLQDAVKVLMLCMETAAKKTASVSKQFVIVQTSSNVDVLKLVEKDGGT

SEQ ID NO: 105 - protein, 192 aa, Methanosarcina siciliae
  source 1..192: note = >WP_048172501.1 Methanosarcina siciliae Adriase
TLVIAFIGKNGAIMAGDMREITFEGEKPDREKLEKELYNGTIVTDEELARKAEEAGVKITVTDCKNKVSERNGILVGEVSSVEGGIVKKRRLYASAGAYAIAELRDLELTLISQGKSSNFIAFGNEFTKQVANKYFKDNWTKKSKLQDAVKILMLCMETAAKKTASVSKQFVIVQTSSNADVLKLVEKDRKS

SEQ ID NO: 106 - protein, 192 aa, Methanosarcina mazei
  source 1..192: note = >WP_048039088.1 Methanosarcina mazei Adriase
TLVIAFIGKNGAVMAGDMREITFEGEKPDREKLEKELYSGSIVTDEEMQKKAEEFGVKITVADCKEKVSERNGVLVGEVSSVEGGVVKKRRLYASAGNFAIAELTNTEMTLTSQGKGSNFIAFGNEFTKQVANKCFKDNWTKKSNLQDAVKILILCMETVARKTASVSKQFMIVQTASNADVLKVVEKDRHC

SEQ ID NO: 107 - protein, 192 aa, Methanosarcina mazei
  source 1..192: note = >WP_048036387.1 Methanosarcina mazei Adriase
TLVIAFIGKNGAVMAGDMREITFEGEKPDREKLEKELYSGSIVTDEKMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNC

SEQ ID NO: 108 - protein, 192 aa, Methanosarcina sp.
  source 1..192: note = >WP_011034811.1 Methanosarcina Adriase
TLVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNC

SEQ ID NO: 109 - protein, 192 aa, Methanosarcina sp.
  source 1..192: note = >WP_048127718.1 Methanosarcina Adriase
TLVIAFIGKNGAVMTGDMREIIFEGDSSQRQKLEKELYSGAIVTDEELEKKAEELGIKITVTDCKSKVSERNSILIGEVSSVEGGIVKKRRLYASAGNYAIAELRDSELTLTSQGKGSNFIAFGNEFTKQVANKCFKDNWTKKSNLQDAVKILMLCMETAAKKTASVSKQFVIMQTASNVDVLKIVEKDRNS

SEQ ID NO: 110 - protein, 192 aa, Methanosarcina sp.
  source 1..192: note = >WP_048138687.1 Methanosarcina Adriase
TLVIAFIGKNGAVMAGDMREITFEGERPDREKLEKELYSGAIVTDEELAKKAEELGVKITVTDCKSKVSERNGILVGEVSSVEGGVVKKRRLYASAGNYAIAELRDSELTLTSQGKGSNFIAFGNEFTKQIANKCFKDNWTKKSNLQDAVKILMLCMETAAKKTASVSKQFVIVQTSSNVDVLKVVEKDRKS

SEQ ID NO: 111 - protein, 192 aa, Methanosarcina sp.
  source 1..192: note = Methanosarcina sp. 1.H.T.1A.1; note = >WP_048136381.1 Methanosarcina sp. 1.H.T.1A.1 Adriase
TLVIAFVGKNGAVMAGDMREITFEGEKSDREKLEKELYSGEIVIDEELAKKAEEFGVKITVTDCKSKVSERNGILVGEVSSVEGGVVKKRRLYASAGNYAIAELRDSELTLTSQGKGSNFIAFGNEFTKQVANKCFKDNWTKKSNLQDAVKILMLCMETAAKKTASVSKQFVIVQTASNVDVLKIVEKDRKG

SEQ ID NO: 112 - protein, 192 aa, Methanosarcina sp.
  source 1..192: note = Methanosarcina sp. 2.H.A.1B.4; note = >WP_048170135.1 Methanosarcina sp. 2.H.A.1B.4 Adriase
TLVIAFVGKNGAVMAGDMREITFEGEKSDREKLEKELYSGEIVTDEELAKKAEEFRVKITVTDCKSKVSERNGILVGEVSSVEGGVVKKRRLYASAGNYAIAELRDSELTLSSQGKGSNFIAFGNEFTKQVANKCFKDNWTKKSNLQDAVKILMLCMETAAKKTASVSKQFVIVQTASNVDVLKIVEKDRKG

SEQ ID NO: 113 - protein, 192 aa, Methanosarcina sp.
  source 1..192: note = >WP_048161572.1 Methanosarcina sp. 1.H.A.2.2 Adriase; note = Methanosarcina sp. 1.H.A.2.2
TLVIAFVGKNGAVMAGDMREITFEGEKSDREKLEKELYSGEIVTDEELAKKAEEFGVKITVTDCKSKVSERNGILVGEVSSVEGGIVKKRRLYASAGNYAIAELRDSELTLTSQGKGSNFIAFGNEFTKQVANKCFKDNWTKKSNLQDAVKILMLCMETAAKKTASVSKQFVIVQTASNVDVLKIVEKDRKG

SEQ ID NO: 114 - protein, 192 aa, Methanosarcina sp.
  source 1..192: note = Methanosarcina sp. MSH10X1; note = >WP_128507564.1 Methanosarcina sp. MSH10X1 Adriase
TLVIAFIGKNGAVMTGDLREITFEGDKPDREKLEKELYSGAIATDGELAEKAREFGVGITVTDCKSKISERNGVLAGEVSSIEGGVVKKRRLYASAGNYAIAELIDFELTLTSHAKGSTLIVLGNEFTKQVANKCFKDNWTKKSTFQDAVKILMLCMETAARKTASVSQQFYLIQTMSSVDVLKAVEEDRKG

SEQ ID NO: 115 - protein, 194 aa, Methanosarcina sp.
  source 1..194: note = Methanosarcina flavescens; note = >WP_054300058.1 Methanosarcina flavescens Adriase
TLVIAFIGKNGAVMTGDLREITFEGDKQDREKLEKELYSGAIVTDDELAEKARKFGVGITVTDCKSKISEKNGVLVGEVSSIEGGVIKKRKLYASAGNYAIAELRDSELTLTSHAKGSTLIVLGNEFTKQIANKCFKDNWTKKSTFQDAVKILMLCMETAARKTASVSQQFYLIQTTSNVDVLKAVEMDKTQKA

SEQ ID NO: 116 - protein, 194 aa, Methanosarcina thermophila
  source 1..194: note = >WP_048168365.1 Methanosarcina thermophila Adriase
TLVIAFIGKNGAVMTGDLREITFEGDKQDREKLEKELYSGAIVTDDELAEKAREFGVSITVKDCKSKIFERDGVLIGEVSSIEGGVVKKRRLYASAGNYAIAEIRDSELTLTSHAKGSTLIVLGNEFTKQIANKCFKDNWTKKSTFQDAVKILMLCMETAARKTASVSQQFYLIQTTSNVDVLKAMEIDKTQEA

SEQ ID NO: 117 - protein, 194 aa, Methanosarcina thermophila; identical to SEQ ID NO: 116
  source 1..194: note = Methanosarcina thermophila TM1; note = >AKB12469.1 Methanosarcina thermophila TM1 Adriase

SEQ ID NO: 118 - protein, 192 aa, Methanosarcina barkeri
  source 1..192: note = >WP_048117266.1 Methanosarcina barkeri Adriase
TLVIAFIGKNGAVMTGDLREITFDGDKSNREKLEKELYNGTIVTDDELAKKAREFGVGITVMDCKSKISERDGILIGEVSSIVCGVIKKRRLYASAGNYAIAEFKDSEITLTSHAKGSNLIVLGNDFTKQVANKCFKDNWTKKSTFQDAVKILMLCMETAARKTASVSKQFYLIQTTSNVDVLKIVDKDRNN

SEQ ID NO: 119 - protein, 192 aa, Methanosarcina barkeri
  source 1..192: note = >WP_048106651.1 Methanosarcina barkeri Adriase
TLVIAFIGKNGAVMTGDLREITFEGEKPNREKLEKELYNGTIVTDNELANKAREFGIVITVTDCKNKISERDGVLIGEVSSIEDGVTKKRRLYTSAGNYAIAELRDSEITLTSHAKGSNLIVLGNDFTKQVANKCFKDNWTKKSTFQDAVKILILCMETAARKTASVSKQFFLIQTTSSVDVLKIVEKDRNS

SEQ ID NO: 120 - protein, 192 aa, Methanosarcina barkeri
  source 1..192: note = >WP_011308259.1 Methanosarcina barkeri Adriase
TLVIAFIGKNGAVMTGDLREITFEGEKPNREKLEKELYNGTIVTDDELAKKARAFGVGITVTDCKSKISEREGILIGEVSSIEGGVTKKRRLYASAGNYAIAELKDSKITLISHAKGSNLIILGNDFTKQVANKCFKDNWTKKSTFQDAIKILMLCMETAARKTASVSKQFFLIQTTSNVDVLKIVEKDRNN

SEQ ID NO: 121 - protein, 192 aa, Methanosarcina sp.
  source 1..192: note = Methanosarcina spelaei; note = >WP_095644589.1 Methanosarcina spelaei Adriase
TLVIAFIGKNGAVMTGDLREITFEGEKQNRDKLEKELYNGTIVTDDELANKAREFGIGITVTDCKSKISERDGVLIGEVSSIEGGVTKKRRLYASAGNYAIAELRNSEITLTSHAKGSNLIVLGNDFTKQVANKCFKDNWTKKSTFQDAVKILILCIETAARKTASVSKQFFLIQTASNVDVLKIVEKDRNS

SEQ ID NO: 122 - protein, 192 aa, Methanosarcina vacuolata
  source 1..192: note = >AKB44029.1 Methanosarcina vacuolata Z761 Adriase; note = Methanosarcina vacuolata Z761
TLVIAFIGKNGAVMTGDLREITFEGEKQNREKLEKDLYNGTIVTDDELAKKAREFGVGITVTDCKSKISERDGILIGEVSSIEGGVTKKRRLYASAGNHAIAEIRDSEITLTSHTKGSNLIVLGNDFTKQVANKCFKDNWTKKSTFQDAIKILILCMETAARKTASVSKQFFLIQTTSNVDVLKIVEKDRNN

SEQ ID NO: 123 - protein, 192 aa, Methanosarcina sp.
  source 1..192: note = Methanosarcina sp. Kolksee; note = >AKB47506.1 Methanosarcina sp. Kolksee Adriase
TLVIAFIGKNGAVMTGDLREITFEGEKQNREKLEKDLYNGTIVTDDELAKKAREFGVGITVTDCKSKISEREGILIGEVSSIEGGVTKKRRLYASAGNHAIAEIRDSEITLTSHTKGSNLIVLGNDFTKQVANKCFKDNWTKKSTFQDAIKILILCMETAARKTASVSKQFFLIQTTSNVDVLKIVEKDRNN

SEQ ID NO: 124 - protein, 192 aa, Methanosarcina vacuolata; identical to SEQ ID NO: 122
  source 1..192: note = >WP_048123851.1 Methanosarcina vacuolata Adriase

SEQ ID NO: 125 - protein, 192 aa, Methanosarcina sp.; identical to SEQ ID NO: 123
  source 1..192: note = >WP_048158264.1 Methanosarcina sp. Kolksee Adriase; note = Methanosarcina sp. Kolksee

SEQ ID NO: 126 - protein, 189 aa, Methanolinea sp.
  source 1..189: note = >KQC10780.1 Methanolinea sp. SDB Adriase; note = Methanolinea sp. SDB
SLVICFSGKKGAIIAGDTREILFGGDDDAVSALEEELYSGAITTEGDLQERARSLGVTIVIRDGREKVSRRGDILIGEVGERERDRVRKRRVYATAGSYALAEVHNSRIISAERRAGAGFVVLGNEMTKRIANQCIRDYWKAGTLDDAVRVIVMAMETASRVTPTVSKQYSLLQTRGRGDLLALLEGEK

SEQ ID NO: 127 - protein, 191 aa, Methanoculleus thermophilus
  source 1..191: note = >WP_066953911.1 Methanoculleus thermophilus Adriase
TLVIAFVGKHGAVMAGDMREIAFQGDDSCIEELECELYSGRITSDSELNTRADEVGVTIQVRDDKAKVSQQDGVLIGEVTESDGPAVRRKRLYATTGKFAIAEVTHSQLKVTERGGASNFVVLGNEITKRIANQCIQGAWEGGSVPDAIRLIMLIMQIAASVTASVSRTFILVHTDLTGNLDDAISQGLRV

SEQ ID NO: 128 - protein, 191 aa, Methanoculleus bourgensis
  source 1..191: note = >WP_014868173.1 Methanoculleus bourgensis Adriase
TLVIAFIGKQGAVMAGDMREIAFFGDDSCIEELERELYNGLIASDDELKGRASEIGVTIRVRDDKAKVSQRDGVLIGEVTETDGSAVAKKRLYATRGSYTIAEVIDSRLRVTRRGSASNFVVLGNNITKQIANQCIQGAWEGGTISDAIRLIMLTMQIAASVTASVSRTFILVHTDLAANLGSAIEQDSRG

SEQ ID NO: 129 - protein, 191 aa, Methanoculleus bourgensis
  source 1..191: note = >SAI88311.1 Methanoculleus bourgensis Adriase
TLVIAFIGKQGAVMAGDMREIAFLGDDSCIEELERELYNGLIASDDELKERASEIGVTIRVRDDKAKVLQRDGVLIGEVTETDGLAVARKRLYVTKGSYVIAEVIDSRLRVTRRGSASNFVVLGNNITKQIANQCIQGAWEGGTISDAIRLIMLTMQIAASVTASVSRTFILVHTELAANLSSAIEQDSRG

SEQ ID NO: 130 - protein, 191 aa, Methanoculleus bourgensis; identical to SEQ ID NO: 129
  source 1..191: note = >WP_082864992.1 Methanoculleus bourgensis Adriase

SEQ ID NO: 131 - protein, 191 aa, Methanoculleus sp.
  source 1..191: note = >CVK34221.1 Methanoculleus sp. MAB1 Adriase; note = Methanoculleus sp. MAB1
TLVIAFIGKQGAVMAGDMREIAFLGDNACIEELERELYNGLIASDDELKERASEIGVTIRVRDDKAKVSQRDGVLIGEVTETDGLAVARKRLYVTKGSYVIAEVIDSRLRVTRRGSASNFVVLGNNITKQIANQCIQGAWEGGTISDAIRLIMLTMQIAASVTASVSRTFILVHTDLAANLGSAIEQDSRG

SEQ ID NO: 132 - protein, 210 aa, Methanocaldococcus jannaschii
  source 1..210: note = M. jannaschii Adriase OB S1A-GS-His6
MALIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQGSHHHHHH

SEQ ID NO: 133 - protein, 192 aa, unidentified
  source 1..192: note = Methanomicrobiales archaeon HGWMethanomicrobiales6; note = >PKL55839.1 Methanomicrobiales archaeon HGWMethanomicrobiales6 Adriase
TLVIAFIGNQGAVMAGDMREIAFGGDDSRIEDLERELYSGSITSDTDLAERADEMGVTIRVRDDKAKVSQQDGVLVGEVTETEGAAVAKRRLYATTGSYAIAEVLDSRLRVMQRGRASNFVVLGNDITKKIANQCIQGMWEGGTIQDALRLLMLTMQIAASATASVSRTFILVHTDRAANLADAIDRDSHRE

SEQ ID NO: 134 - protein, 191 aa, unidentified
  source 1..191: note = >PKL61271.1 Methanomicrobiales archaeon HGWMethanomicrobiales2 Adriase; note = Methanomicrobiales archaeon HGWMethanomicrobiales2
TLVIAFIGRQATVMAGDMREIAFEGDDSCIEELEREIYSGSITSDTDLAARADEIGVTIRVRDDKAKVSQQDGVLIGEVTETEGPVTRRKRLYATAGSYAIAEAIDSRLRVTQRGRASNFVVLGNEVTKRIANQCIQGMWEGGTIQDAMRLLMLTMQITASVTASVSRTFILVHTDRAANLADTINRDLRK

SEQ ID NO: 135 - protein, 196 aa, Methanoculleus sp.
  source 1..196: note = Methanoculleus sediminis; note = >WP_048184178.1 Methanoculleus sediminis Adriase
TLVIAFIGKQGAVMAGDMREIAFGGDDASIEDLERELYDGSIASDSDLAQRAGEIGVTIRVRDDKAKVSQRDGVLVGEVTETEGAKTSRKRLYATKGSYVIAEIVDSRLRVMQKGQASNFVVLGNDITKRIANQCIQGMWEGGTIQDALRLIMLTMQIAASVTASVSRTFVLVHTDSAADITTAIATDSRKEQNPD

SEQ ID NO: 136 - protein, 196 aa, Methanoculleus marisnigri
  source 1..196: note = >WP_011844100.1 Methanoculleus marisnigri Adriase
TLVIAFIGKQGAVMAGDMREIAFGGDDSSIEDLERELYGGSIASDSDLAKRADEIGVTIQLRDDKTKVSQRDGVLVGEVTETEGAKTSRKRLYATKGSYVIAEIVDSRLRVMQKGQASNFVVLGNDTTKEVANQCIQGMWEGGTIQDALRLIMLTMQIAASVTASVSRTFVLVHTDIAADVPAAIANDSRKEQNPD

SEQ ID NO: 137 - protein, 196 aa, Methanoculleus sp.
  source 1..196: note = >WP_048113726.1 Methanoculleus sp. MH98A Adriase; note = Methanoculleus sp. MH98A
TLVIAFIGKQGAVMAGDMREIAFGGDDSSIEDLERELYGGAIVSDSDLAKRADEIGVTIQLRDDKTKVSQRDGTLVGEVTETEGANISRKRLYATKGSYVIAEIVDSRLRVMQKGRASNFVVLGNDITKRVANQCIQGMWEGGTIQDALRLIMLTMQIAASVTASVSRTFVLVHTDTAADIPAAIANDSRKEQNPD

SEQ ID NO: 138 - protein, 196 aa, Methanoculleus chikugoensis
  source 1..196: note = >WP_074370403.1 Methanoculleus chikugoensis Adriase
TLVIAFIGEQGAVMAGDMREIAFGGDDSLIEELERELYGGSIASDRDLAERADAIGVTVRVRDDKAKVSQRDGVLVGEVTETEGVKTSRRRLYATKGRYAIADVVDSRLRVTQRGRASNFVVLGNDITKEVANQCIRGMWEGGTIQDALRLIMLTMQIAASVTASVSRTFVLVHTDIAADISGAIANDSRKGQNPD

SEQ ID NO: 139 - protein, 196 aa, Methanoculleus sp.
  source 1..196: note = >WP_067073447.1 Methanoculleus horonobensis Adriase; note = Methanoculleus horonobensis
TLVIAFIGKQGAVMAGDMREIAFGGDDSLIEELERELYDGSIASDRDLAERADAIGVAIRVRDDKAKVSQRDGMLVGEVTETEGAKTSRKRLYATKGNYAIAEVVESRLQVTQKGRASNFVVLGNEITKRVADQCIQGMWEGGTIRDALRLIMLTMQIAASVTTSVSRTFVLVHTDTMADISGAIANDARKEQKPD

SEQ ID NO: 140 - protein, 189 aa, unidentified
  source 1..189: note = Methanoregulaceae archaeon PtaU1.Bin066; note = >OPY42232.1 Methanoregulaceae archaeon PtaU1.Bin066 Adriase
SLVMAFSGTYSAVIAGDQREILFRGEASCIALLESELYSNRLCTEEQLHARAAELDVSITIRDDKCKVWEQDGILIGEVTETDGPVIRKRRICVSCGRYAIFEKSGESWSLLSRGEGSHFLVLGNEATKGIAHQCIREHWKPGGGTLEDAVKVLILSMQEAARRTASVSRTFSILQTRTKADVPCSLGM

SEQ ID NO: 141 - protein, 189 aa, unidentified
  source 1..189: note = >OPY42561.1 Methanoregulaceae archaeon PtaU1.Bin059 Adriase; note = Methanoregulaceae archaeon PtaU1.Bin059
SLVIAFCGKERAFMAGDRREIFFSGNDSATGRLEEELYSGEIWDEEGLFRRAGKLGVRLFVHDGKCKVTERNGVLVGEVSESEGEQKRTRRLLVTAGTYAISEIREGKEVEVARGQASRFIVLGNRVMQEVANWCIRSCWKENGGSPADALRVILVTMKEAAAATPSVSRGYTLVQTTRCGAPETGPGG

SEQ ID NO: 142 - protein, 185 aa, Methanolinea tarda
  source 1..185: note = >WP_007313979.1 Methanolinea tarda Adriase
SLVIAFCGRDRSFMAGDRREIVFRGDDLRTRTLEDELYSGRIRSRDALHERARELGIDLWIHDDRCKAWEEDGILIGQVNERDGLSTRSRRLCVSCGNYVITEVLEGIKREVTRGCASRFIVLGNKTTQEIAHRCIRSHWSDTGGTPADALRIIILAMEEAASLTPSVSGTYTLVQTTAKTVLPP

SEQ ID NO: 143 - protein, 219 aa, unidentified
  source 1..219: note = Methanoregulaceae archaeon PtaB.Bin152; note = >OPX76491.1 Methanoregulaceae archaeon PtaB.Bin152 Adriase
SLVIAFCGRDRSFMAGDRREIVFRGDDLRTRTLEDELYSGRIRSRDALHERARELGIDLWIHDDRCKAWEEDGILIGQVSERDGLSTRSRRLCVSCGNYVITEVQEGIKREVTRGCASRFIVLGNKTTQEIAHRCIRSHWSDTGGTPADALRIIILAMEEAASLTPSVSGTYTLVQTTAKTVLPPQGKTQRSLVFPIFFLSFAIPGGQCDTEGRGVRLP

SEQ ID NO: 144 - protein, 321 aa, Methanopyrus sp.
  source 1..321: note = Methanopyrus sp. KOL6; note = >WP_088335494.1 Methanopyrus sp. KOL6 Adriase
TLVLAYAGTDGALVAGDHRILVARMDEEKMREVEEKLYSGEIRTEEELESFLKDLEVEGGYFEFHDDRKKVWKVNDEVVAGEVGVRSAKGVRRRRVYATPGAHAIIELEGEKVLSKNFGGPALIVEGPKVVKELVIEFVDSELGGKPDLESLRNALDDIFEYVSSNTVLVSSEYDAFEVKGRVDHLARIHLQKAIDEDIERLRKYRRRLVEEMLKHIREGYDILKEGVVGEVVEVGTEEEGEGVEDVPPERRIVVRLVEDVDARHMGDVVAGPGEEIVMAVEGDPEKVEPGDTVVVKDGIMKIDGKDLPVVTGYSICRTRR

SEQ ID NO: 145 - protein, 321 aa, Methanopyrus kandleri
  source 1..321: note = >AAM01502.1 Methanopyrus kandleri AV19 Adriase; note = Methanopyrus kandleri AV19
TLVLAYAGTDGALVAGDRRTLVARMDEEKMRKVEEKLYSGEIRTEEELESFLKDLDVEDGYFEFHDDRKKVWKVNDEVVAGEVGVRSAKGVRRRRVYATPGAHAIVELEGEKVLSKNFGGPALIVEGPKVVKELVIEFVNSELGGKPDLESLRNALDDLFEYVSSGTILVSSEYDAYEVKGKADPLARARLQKAIDEDIERLREHRRRLAEEMLKHIREGYDILKEGVVGEVVEVGTEEEGKGVEDVPPERRIVVRLAEDVDARHMGDVVAGPGEEIVMAVEGNPEEVEPGDRVVVKDGVMKIDGKDLPVITGYSICRTRR

SEQ ID NO: 146 - protein, 289 aa, Methanococcus voltae
  source 1..289: note = >WP_013180795.1 Methanococcus voltae Adriase
SVIIGYYGNNGSAIAGDKRNILFRGSEANREKLESLLYNGKLKNDKELKEKAEEYDVKVHITDSQDKIKKLDGCLMGEVKTISNQSKRKRMYLSKNNCALVEIIDDDMTKKEIKKGSGIIIFGNKHLKQLVQAELQKNVNIVKSGDILKIEKLFKDILSNIDTPSSSNDVDSYSNFKYENLEKVISQELSNLVDYRNGLKNQIIKVQKLMLIEKKIATKGEIGYVNEGKLKLYDKYLAIDRICENPNLYYEIDITGDVKEGDIILIDENNLKVKNKDVIVSVNKIICNR

SEQ ID NO: 147 - protein, 290 aa, Methanococcus aeolicus
  source 1..290: note = >WP_011973890.1 Methanococcus aeolicus Adriase
SIIVGYYGANGAVVGGDKRNIIFRGNAEQREELEKLLYSGNIKTDEEFKKIADEYGIKVYIEDKQLKVREVNGVLVGEVKSIGTDSQRRRMYLSTGNCAIIDILNDTITKKSSNVGHGLILFGNRYLKEIVHNELKKDAPNFKNMPIEKVKNIIENAIKRCDGPTVSEEIILLHTKDKHNNFKELIKKDINDLKEYRNGLRQQMIDIKKTMLIAEKIEINGEVGYIKNGKLVLDENHLAIDTICSNPNLYNEIEIKGEYEEGDIIYIDNGELKVKGKDSSVCVDKIICKA

SEQ ID NO: 148 - protein, 290 aa, Methanothermococcus thermolithotrophicus
  source 1..290: note = >WP_018153627.1 Methanothermococcus thermolithotrophicus Adriase
SVIIGYYGKNGAVIAGDRRNILFRGDPKKREELEKKLYSGELKSDEELIRAAKDLGINIHIDDEKIKVRKIEDVLVGEVSSIGAESKRRRMYITKGKCAIIDILNDTITNKSIREGSSIVVFGNKYIKNLVQQELVKYKNKILKMSILEIKNLFEEILKNCNNPTVSKKYDVFYTVNCSKNFEETIENDIKDLFNYRNELNKKMFEFGKIMTIVNKIANEGEIGTIKNGKLVLNDDHIAIDKVCPNPKLFTEIEVEGNFEDGDIVVIENGNMKIKGKNENITTKYIVCKK

SEQ ID NO: 149 - protein, 291 aa, unidentified
  source 1..291: note = >GBF37010.1 Methanococcales archaeon HHB Adriase
SIVIGYYGNNGAVIGGDKRNIIFRGDPKKREELEKKLYSGKIKDEEELKKVAEELGVKVYIEDERVKVKKINNTLVGEVRSIGADSRRRRMYLTKGMCVIVDILNDDVIGKSVKRGNGVIIFGNKHLKEIARKELRKYMYDFTRMGIGEVKDVIEGVLKRCCRGPTSSKDFDILYIDEGEKDLEKVIEEDLEELRRYREELRQKMIDFRKVLAIVDKIENNGEVGIIKDGKLVLNEDHLAIDKICPNPRQFREIEIEGDVEDGDIVVIENGTLKVKGKDAILKTNYIICRK

SEQ ID NO: 150 - protein, 291 aa, unidentified; identical to SEQ ID NO: 149
  source 1..291: note = >WP_131007839.1 Methanococcales archaeon HHB Adriase

SEQ ID NO: 151 - protein, 290 aa, Methanothermococcus okinawensis
  source 1..290: note = >WP_013866916.1 Methanothermococcus okinawensis Adriase
SIVIGYYGNNGAVVAGDRRNIMFRGNPEKRAELEKDLYCGKIKNEEELKNRAEELGVKIFIEDERTKVKKIGDVLVGEVKSIGADSKRRKMYLTKGNCAIVDILNDTITNKSIKNGSSIIIFGNKYLKDIVQKELKKYMNNFGKMDILDVKNTIENALKKCDGPTLSPELDILHTNKKVFNLEEIIEKDLNDLKEYRNDLKQKMIDFKKVMIIADKIENNGEVGIIKNGKLVLDDNHIAIDKVCPNPKLFNEIEIEGDVEDGDVVLIEDGSLKIKGKDIPLAINHIICKK

SEQ ID NO: 152 - protein, 291 aa, Methanotorris igneus
  source 1..291: note = >WP_013799607.1 Methanotorris igneus Adriase
SLIIGYYGRNGAVLGGDKRNIMFRGDIEKRKKLEEMLYNGEIKDDEELLKKAEEFGIKLHIEDNRKKVRKIGDDVLVGEVKSIGIDSERRRVYLTKGKCIIVDVFNDEIRNKWTKTGSGVVVFGNRYVKKIAEEEFRKAGYRLTMMSLEEIRDLIENIFKKCNTPSVSKGYDIIYTTKECKNLEEVVKKDLDALFEYRETLRKKMIDFGKVMTIVNKIVKNGEIGVIKNGKLVLYDKYIAIDKICPNPKMFNEIEVKGDVEDGDVIVIENGEMKVKGKNTPVFVDYTICEK

SEQ ID NO: 153 - protein, 291 aa, Methanotorris formicicus
  source 1..291: note = >WP_007043881.1 Methanotorris formicicus Adriase
SVIIGYYGKNGAVLGGDKRRIMFRGDVEKRKMLEKLLYNGEIKNDEELLKKAEELGIKLHIEDGKKKVRKIGNDVLVGEVKTIGIDSKRRRVYLTKGKCMIVDVFNDEIKNKLIKTGSGVVVFGNKYVKKIAEDEFKKIGHRLTMMSLEEIKDIIENIFKKYDTPSVSKEYIVVYTTKECKNLEEVVKKDLDELFEYRETLRKKMIDFSKVMTIVNKIVKNGEVGVVKNGKLVLYDKYIAIDKICPNPNIFNDIEIRGDVEEGDVVVIENGEMKIKGKNTPVFVDHIICEK

SEQ ID NO: 154 - protein, 291 aa, Methanocaldococcus infernus
  source 1..291: note = >WP_013100627.1 Methanocaldococcus infernus Adriase
SLILCYFGNNGAIILGDRREMFFRGNEEKRKELEELLYSGEIKSEEELRKKAEELGVKIIIEDKRRKVWKIKNVVVGEVRSLGLDAKRRRVYATKRKAKILEILNDQIIGEKNLEGFSIIIFSNKFIKEEINKYLKEYYRVLPMKRIDEVGEIFKEIYKKVSWHPTLSEEFDVEKTDKEEEDFEEVIEEDVKKLFEYREELKKKLVDFGKVMDIVNRIVKNGEIGEVKNGKLHLFDDYIAVDSIKPNPKTYKVIDIEGAEDGDIIVIENGEMKVKGKDKKVNTNYIIIKSW

SEQ ID NO: 155 - protein, 291 aa, Methanocaldococcus villosus
  source 1..291: note = >WP_004589885.1 Methanocaldococcus villosus Adriase
SLVICYYGNNGAVMAGDRRQMFFKGPEEKRKILEEKLYSGEIQNEEDLYKLADDLGVKVIIEDNREKVRKIGNTLVGEVRSIGLEAKRRRVYATKGKCMILEILGDVITDRMLKNGAGLIVFGNRYLKNKAEKILKNVAKDFPKMDIDEVGKVIKDVFERFKEHPTISREYDIYVTKNIDINFEKTVEEDINKLFKYREDIRKKMIDFGKVMSIVNKIVKNGEVGVIKEGKLHLYDQYIAIDKISPNFKTFRIIDVKGDVEDGDIVVIENGDMKIKNKGIKVMTDYIICYK

SEQ ID NO: 156 - protein, 292 aa, Methanocaldococcus vulcanius
  source 1..292: note = >WP_015733029.1 Methanocaldococcus vulcanius Adriase
TLVICYYGKNGAVIGGDRRQIFFRGNEEKRKLLEEKLYSGEIKTEEELYKLAEELGVKIIIEDDREKVRKISDTVVCGEVRSLGVDAKRRRVYATKGKCAIIDILNDTITKEIIKEGFGLVIFGNKFLKNRAEEELRNKAKLFPMMPIQQIESEIREIFEKLKWHPTVSKEYDIYSVNKYEKNFEEVIKKDIGDLFEYREKLRQQLIDFGKVMSIVNKIVKNGEIGVIKDGKLHLYDEYIAVDKIDPNPNTFKIIEVKGNFKDGDVIVIENGDMKVKGTDERVETDYIIISK

SEQ ID NO: 157 - protein, 291 aa, Methanocaldococcus fervens
  source 1..291: note = >WP_015791349.1 Methanocaldococcus fervens Adriase
SLIICYYGKNGAVIGGDKRQIFFRGNEEKRKILEEKLYNGEIKSEEELYKLADELNIKIIIEDNREKVRKISDSVVVGEVRSLGIEAKRRRVYATKGKCAIVDILNDTVTNEVIKEGFGIVVLGNRFLKKKAEEELKSSAKLFPMMPLEQIENVIRDIFEKLNWNPTISKEYDIYSVDKYEKNFEEVVRKDIESLFKYRDELRKQLIEFGKVMTIINKIVKNGEIGVIKDGKLHLYDNYIAIDKIDPPNVFKVVEVEGNFKDGDVVVIENGEMKIKGTNEKIMTKYIICHK

SEQ ID NO: 158 - protein, 292 aa, Methanocaldococcus sp.
  source 1..292: note = Methanocaldococcus sp. FS40622; note = >WP_012979926.1 Methanocaldococcus sp. FS40622 Adriase
SLIICYYGKNGAVIGGDRRQIFFRGEEQKRKILEEKLYSGEITSEDELYNLARELNIKIIIEDDREKVRKISDSVVVGEVRSLGIDARRRRVYATKGKCAIIDILNDTVTNQMIKEGFGVVVLGNRFLKKKAEEELKRTAKLFPMMPLQQIEDAIKEIFEKLKWHPTISKEYDIYSVNKYEKDFEEVIKKDIESLFKYREQLRKQLIDFGKVMSIVNKIVKNGEIGVIKDGKLHLYDNYIAIDKIDPNPKVFKVVEVEGNFKDGDIVVIENGDMKIKGTNEKLTTNYIIIHK

SEQ ID NO: 159 - protein, 292 aa, Methanocaldococcus jannaschii
  source 1..292: note = >WP_010870052.1 Methanocaldococcus jannaschii Adriase
SLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 160 - protein, 292 aa, Methanocaldococcus sp.
  source 1..292: note = >WP_048201244.1 Methanocaldococcus bathoardescens Adriase; note = Methanocaldococcus bathoardescens
SLIICYYGKNGAVIGGDRRQIFFRGNEENRKILEEKLYSGEIKSEEELYKLAKELDIKIIIEDNREKVRKISDSVVVGEVRSLGIDAKRRRVYATKGKCAIVDILNDTVTNQTIKEGFGIVVLGNRFLKKSAEEELKRTAKLFPMMPLEQIEDTIREIFEKLKWNPTISKEYDIYSINKYEKNFEDVVKKDIESLFKYREELRKQLINFGKIMTIVNKIVKNGEIGVIKDGKLHLYDNYVAIDKIDPNPNVFKVVEVEGNFKDGDIIVIENGNMKIKGTDERVTTKYIICHK

SEQ ID NO: 161 - protein, 290 aa, Methanococcus vannielii
  source 1..290: note = >WP_012066013.1 Methanococcus vannielii Adriase
SVIIGYYGKNGVVIAGDKRTILFNGEEKKREMLEELLYSGKIKNDDDLSEKSAEFSVKVHILDDQKKIKQLENTLIGEVRTIGKDSKRKKMYLSKDICVILDIENDNIVKKSVKTGTGIVVFGNKYVKKIVELELKKYLSEISKMDVLKIRTIFEKILVGIENPTLSKGYDFLYCQNAEKDIESIIKKDLNDLREYREELSIKMAEMQKLMTIVNKIAKSGEIGIIKNGSLVLNESFLAIDKICLNPEIYSEIDINGNFKEGDLIEIQNGELTVKGTNNYVSVQKIICNR

SEQ ID NO: 162 - protein, 290 aa, Methanococcus maripaludis
  source 1..290: note = >WP_011869407.1 Methanococcus maripaludis Adriase
SVIIGYYGKNGAVIAGDSRNLLFKGIKSNREKLEELLYSGKIRTDEELMKKASEFDVNVYINDSEEKVRILGNLLCGEIKSIGKDSKRRRMYLSKEKCAVLDIENDQITNKSVKTGSGIVVFGNKYIKSLVESEIKKYIKDILKMSVKEIKNLFEKILEKTENATLSGKFEYYFVETYENNFEMAVEDDLNELFEYRKELSLKMVEMQKVMMIADKIVKIGDVGKIENGNLVLYDDFLAIDKICPEPTIYCEIEITGEFIEGDVITIDNESLKVKRTGNPVVVEKIICKK

SEQ ID NO: 163 - protein, 290 aa, Methanococcus maripaludis
  source 1..290: note = WP_012193694.1 Methanococcus maripaludis Adriase
SVIIGYYGKNGAVIAGDRRNLLFNGIESNRKKLEELLYSGKIRTDEELMKKASEFDVNVYINDSEEKVRSLGTLLCGEVKSIGKDSKRRRMYLSREKCAVIDIENDRITNKSVKTGSGIVVFGNKYIKNLVESEIKKYIKNILKMSAEEIKNLFVKILENTKNATLSGNFEYHFVETYENNFEKVIEDDLNELFDYRKELSIKMIEMQKVMMIADKIVKIGDVGKIENGSLVLYDEFLAIDRICPEPTLYCEIGITGEFIEGDVITIDSESLKVKRTGNPAVVEKIICRK

SEQ ID NO: 164 - protein, 290 aa, Methanococcus maripaludis
  source 1..290: note = >WP_011977398.1 Methanococcus maripaludis Adriase
SVVIGYYGKNGAVIAGDKRNLLFNGIESNREKLEEIMYSGQLRTDEELLKKASEFDVKIHINDTREKVKKFGNVLSGEVLSIGRESKRRRMYLTKEKCVILDIENDQITNKSVKNGSGIVVFGNRYIKNLVESEIKKHVQKILKMSAKEISALFEKILKNIENATLSNEFEYYFVETYENNLEKVIENDLNDLFNYRKELSLKMVEMQKVMMIADKIVKIGDVGEIENGNLVLYDEFLAIDKICPEPTIYCEIGITGEFAEGDIITIDNESLKVKRTGSPVVVEKIICRK

SEQ ID NO: 165 - protein, 290 aa, Methanococcus maripaludis
  source 1..290: note = >WP_104837987.1 Methanococcus maripaludis Adriase
SVVIGYYGKNGAVIAGDKRNLLFNGIESNREKLEEVLYSGEIKSDEELFKKASEFDVTVHINDTREKVKSLGNLLSGEVLSIGKDSKRRRMYLTKEKCAIIDIENDQITNKSVKTGSGIVVFGNRHIKHFVESEIKKHIQKLLKMNAREIRDLFEKILKKIENATLSDTFEYYFVEAGEPEFEKAVNDDLDDLFNYRHDLSIKMAEMQILTMIAEKIVKIGDVGIIKNGTLVLYDEFLAINKICPEPEIYSEIEIKGEFIEGDVITIDNESLKVKRTGNPVVVQKIICKK

SEQ ID NO: 166 - protein, 290 aa, Methanococcus maripaludis
  source 1..290: note = WP_013998520.1 Methanococcus maripaludis Adriase
SVVIGYYGKNGAVIAGDKRNLLFNGIESNRAKLEKVLYSGEIKTDEELFKKASEFDVTVHINDTREKVKSLGNLLSGEVVSIGKDSKRRRMYLTKEKCAIIDIENDQITNKSVKTGSGIVVFGNRYIKHFVESEIKKHVQKLLKMSAREIRDLFEKILKNIENATLSDNFEYYVVEAGEPEFEKAVNKDLDDLFNYRHDLSIKMAEMQILTMIAEKIVKIGDVGVIKNGTLVLYDEFLAINKICPEPEIYSEIEITGEFIEGDIITIDNESLKVKRTGTPVVVQKIICKK

SEQ ID NO: 167 - protein, 290 aa, Methanococcus maripaludis
  source 1..290: note = >WP_011169965.1 Methanococcus maripaludis Adriase
SVVIGYYGKNGAVIAGDKRNLLFNGIESNRAKLEEVLYSGEIKNDEELFKKASEFEVTVHINDTREKVKSLGNLLSGEVVSIGKDSKRRRMYLTKEKCAIIDIENDQITNKSVKTGSGIVVFGNRYVKHFVESEIKKHVQKLLKMSAREIRDLFEKILKNIENATLSDTFEYYVVEAGEPEFEKAVNKDLDDLFNYRHDLSIKMAEMQILTMIAEKIVKIGDVGVIKNGTLVLYDEFLAINKICPEPEIYSEIEITGEFIEGDIITIDNESLKVKRTGSPVAVQKIICKK

SEQ ID NO: 168 - protein, 290 aa, Methanococcus maripaludis
  source 1..290: note = >WP_119720453.1 Methanococcus maripaludis Adriase
SVVIGYYGKNGAVIAGDKRNLLFNGIESNRAKLEEVLYSGEIKTDEELFKKASEFDVTVHINDTREKVKSLGNLLSGEVVSIGKDSKRRRMYLTKEKCAIIDIENDRITNKSVKTGSGIVVFGNRYIKHFVESEIKKHVQKLLKMSAREIRDLFEKILKNIENATLSDTFEYYVVETGEPEFEKAVNKDLDDLFNYRHDLSIKMAEMQILTMIAEKIVKIGDVGVIKNGTLVLYDEFLAINKICPEPEIYSEIEITGEFIEGDIITIDNESLKVKRTGSPVAVQKIICKK

SEQ ID NO: 169 - protein, 290 aa, Methanococcus maripaludis
  source 1..290: note = Methanococcus maripaludis KA1; note = >BAP60138.1 Methanococcus maripaludis KA1 Adriase
SVVIGYYGKNGAVIAGDKRNLLFNGIESNRAKLEKVLYSGEIKTDEELFKKASEFDVTVHINDTREKVKSLGNLLSGEVVSIGKDSKRRRMYLTKEKCAIIDIENDRITNKSVKTGSGIVVFGNRYIKHFVESEIKKHVQKLLKMSAREIRDLFEKILKNIENATLSDTFEYYVVETGEPEFEKAVNKDLDDLFNYRHDLSIKMAEMQILTMIAEKIVKIGDVGVIKNGTLVLYDEFLAINKICPEPEIYSEIEITGEFIEGDIITIDNESLKVKRTGSPVAVQKIICKK

SEQ ID NO: 170 - protein, 309 aa, Methanothermus fervidus
  source 1..309: note = >WP_013413908.1 Methanothermus fervidus Adriase
SLILAYVGKKGCVIAGDKRKIAYFGDKSSRKILEEKLYTGKIKTLDELFEEAKKLGVSINITDNAKKVREKGHVAIGEVGIKATHSAKRRRLYATTNCYEIIELEGSKIVKRKKGNSALIVFGNRITKKISNEILKKEFNPKMSLQEIANLFKKIIKKASELTPTINKSCDIVIKNPILNKEMAEKILEMSISEDIKNLRKWRKSLKDKMIKAAKEITLASKIITEGDVGEIVDIKDGKIMVKLNQDVCAYDLNWEKVANPGEIVTMTADNPSEIKVGDKVVIENEELYVERCKEPLQCEVILTYCNRG

SEQ ID NO: 171 - protein, 318 aa, Methanobrevibacter curvatus
  source 1..318: note = >WP_067092528.1 Methanobrevibacter curvatus Adriase
SLIIAYVGKKGCVMVADKRRISYFGDKEDIESLEDKLYSGDLENDEELKKTANLLNVSLKISDKESKIRSLESSLVAEVSTKSTHKAMRKRIYATTNAYQIIELEGSNISNLDKGDSSIIIFGNKITKSLANDFLSKEFKADVSLKYMGDIFTRILSKIHLKTPSIGNEFDVLVKTPKFSKEEAQKYLDDLVKVDIKLLVKFRDKLTQDLFDKSEEIKIASKIINEGYIGVVSKIKNNFIYVKLNYNVQAYNTRWKLLVKPNEEALMFLGEDSIKTHNSNNTQENIVKLGDKILIENEIIVIERNKTPLNCDIILTSV

SEQ ID NO: 172 - protein, 307 aa, Methanobrevibacter olleyae
  source 1..307: note = >WP_067146985.1 Methanobrevibacter olleyae Adriase
SLIIAYVGNKGCVMVGDKRRIAYFGSKEERELLEQEIYSGDISSDEDLYARAEELEISLKISDDACKVKSLENVAVGEVSSRGAMETKRKRIYGTTNGFQIVELTGSEIVNVKRGESSIIVFGNKITKSLANDMLKNKWKPSFSLKYMGEIFGQIIEDISTKTPSLGTKYDVVIQQNNLSKTKVQDYLDEVIERDINLLAKFRNKLKEDLLKQNETIKLASTIIDEGPIGIVDSIDENLIQVKLNPDVRAFDINWKLLAKPGENVIMFVEGEDAVSLKDQVVIENEALCIKGNKANLKCDIILCHLE

SEQ ID NO: 173 - protein, 307 aa, Methanobrevibacter ruminantium
  source 1..307: note = >WP_012956362.1 Methanobrevibacter ruminantium Adriase
SLIIAYVGKKGCVMAADKRRIAYFGLKEQREILEQEIYSGEIHNDEELYARAEELDVNLKVTDNACKVKSIENVAIGEVSSRGTMETKRKRIYGTSNGFKIIELSGSHVLNSKRGVSSIIVFGNKVTKSLANKMIQERWKPSFSLKYMGEIFGEILKDIAEITPSVGKDYDVIIEKNNLAKSQVQDYIDEVAERDVKVLAKFRAQLREQLMEQNENIKLASTIIEEGSVGEIVIIDDTLIEVKLNPDVRAFDVNWKVLAGPGENVLMVVDDSEGLDKGDEIVIEDEVLCVKKNKANLKCDIILCHLE

SEQ ID NO: 174 - protein, 319 aa, Methanobrevibacter sp.
  source 1..319: note = WP_016358763.1 Methanobrevibacter sp. AbM4 Adriase; note = Methanobrevibacter sp. AbM4
SIIIAYIGRKGCVMASDKRRIAYFGDKDNREKLEEELYSGNIRNDEELYKEAAKLGIDLKISDDAKKIKSIGNAVMGEVSSKTTREVRRRRIYGTTNGYQIIELIGSELKNKETGSSGIIIFGNKIAKQMANSLIKQRWKSSISLKYTGDIFEEIIKEIAEKTPSVGKNVDVIIGKDSKLTKTTAQKYLDETETRDIKVLEKYRQKLAEDLEEQRESIEVANKIINKGTIGYISKIEGKMLNVKLTDKVNAYDTNLKKVAGSNEEVIMFLDKDTEGNEIKEADVSSYAKVNDKVVIKNENLCIDKNNIPLNCGIILCNE

SEQ ID NO: 175 - protein, 319 aa, Methanobrevibacter sp.
  source 1..319: note = >WP_040681637.1 Methanobrevibacter boviskoreani Adriase; note = Methanobrevibacter boviskoreani
SIIIAYIGRKGCVMASDKRRIAYFGDKDNREKLEEELYSGNIRNDEELYKEAAKLGIDLKISDDAKKIKSIGNAVMGEVSSKTTREVRRRRIYGTTNGYQIIELIGSELKNKETGSSGIIIFGNKIAKQMANSLIKQRWKSSISLKYTGDIFEEIIKEIAEKTPSVGKNIDVIIGKDSKLTKTTAQKYLDETETRDIKVLEKYRQKLAEDLEEQRESIEVANKIINKGTIGYISKIEGKMLNVKLTDKVNAYDTNLKKVAGSNEEVIMFLDKDTEGNEIKEADVSSYAKVNDKVVIKNENLCIDKNNIPLNCGIILCNE

SEQ ID NO: 176 - protein, 319 aa, Methanobrevibacter wolinii
  source 1..319: note = >WP_042707893.1 Methanobrevibacter wolinii Adriase
SVIIAYIGKKGCVMASDKRRIAYFGDKDEREKLEEDLYTGKIRNDEELYNEAKKRQITLKISDDVNKIKKIENALMGEVSTKTTMEIRRKRIYGTTNGYQIVELLGSETQNKERGSSGIIIFGNKIAKSMANSLIKERWKSSLSLKYIGDIFEEVIKEIASKTPSVGKNVDVLYSKDNTLNKTSAQEYLDKTEERDNKLLEKYRQKLTEDLIKQQKSVELAGKIVTKGDVGFVESNEGKILKVKLAKDVQAFDMDWKVLAKPGEEIIMFLDKDNNGNEIKDTEVSNYVQKFDKVVIKDENLCLEKNNIKLNCNIILCNT

SEQ ID NO: 177 - protein, 319 aa, Methanobrevibacter sp.
  source 1..319: note = >WP_088539534.1 Methanobrevibacter sp. 87.7 Adriase; note = Methanobrevibacter sp. 87.7
SVIIAYIGKKGCVMASDKRRIAYFGDKEEREKLEEDLYSGKIRNDEELYNEAAKRQITLKISDDVNKIKKIETVLMGEVSTKTTMEIRRKRIYGTSNGYQIVELLGSEVQNKERGNSGIIIFGNKIAKSIANSLIKERWKSNLSLKYIGDIFEDIIKEIASKTPSVGKNVEVLYTKNNNLNKVTAQEYLDKTEEIDNKLLEKYRQKLSEELLSQQKSIEFASKIITKGDVGFVNSYEGKILKIKLADNVQAFDMDWKVLAKPGEEIIMFIDKDNNGNEIKDIEVSNYVQKFDKVVVKDENLCLDKNNIKLNCDIILCNT

SEQ ID NO: 178 - protein, 306 aa, Methanobrevibacter filiformis
  source 1..306: note = >WP_066972212.1 Methanobrevibacter filiformis Adriase
SLIIAYVGKKGCVIASDKRKIAYFGDKENIDKLEKELYNGNIKGDDDLYDTASKLDVSLKISEDGIKLRSFDKINVGEVSSKSTTETNRKRIYGTTNGYQIVELSGSKIVHTEKGNNALIIFGNKRTKTLANDLISKKWKSNFSLKYMGDIFLRILEEISAKTPSIGKAYDLNIINHKFTKEEANEYLDYFIEKDIQVLGKFRNQLKSELLEQSEAIQMATKIINQGFVGKIANIENNMLEVQLNKNVQGFDHNWKLLVKPGEKAFMFAPENIDVKIGDKIVIQNETLCVEQNNVQLSCNIILCHL

SEQ ID NO: 179 - protein, 305 aa, Methanobrevibacter smithii
  source 1..305: note = >WP_019266968.1 Methanobrevibacter smithii Adriase
SLIIAYIGKKGCVMASDKRRIGYFGNKEQLNALESELYSGKIKNDDEFKKKADEYGIAIKLTEDATKITTVGNCVRGEVTTKKAFETYRKRIYGTTMGYQIVELSGSETVSREAGEKAIIVFGNKFAKQEAEKLINKKWKPSLSLKYMGDIFEEILTEISRKTPTIGNEFDVLIKQPKFNKSEAQRHLNVSIDKDIKVLSKFRQKLQEDLIQKNKEIALADKIIDKGDIGEVVSVDGKMLHVKLNSKTQAFDGNWKQIAKPNETVFMFSDNGDAELGDKVVIQNEKLCLKKDKSNLKCDIILCGL

SEQ ID NO: 180 - protein, 305 aa, Methanobrevibacter smithii
  source 1..305: note = >WP_019262821.1 Methanobrevibacter smithii Adriase
SLIIAYIGKKGCVMASDKRRIGYFGNKEQLNALESELYSGKIKTDEEFKKKADEYGISIKLTEDATKITTVGNCVRGEVTTKKVFETYRKRIYGTTMGYQIVELSGSETVSREAGEKAIIVFGNKFAKQEAEKLINKKWKPSLSLKYMGDIFEEILTEISRKTPTIGNTFDVLIKQPKFNKSEAQRHLNVSIDKDIKVLSKFRQKLQEDLIQKNKEIALADKIINKGDVGEVVSVDGKMLHVKLNSKTQAFDGNWKQIAKPNETVFMFSDHNHVELGDKVVIQDEKLCLKKDKSNLKCNIILCSL

SEQ ID NO: 181 - protein, 305 aa, Methanobrevibacter sp.
  source 1..305: note = >WP_004035387.1 Methanobrevibacter Adriase
SLIIAYIGKKGCVMASDKRRIGYFGNKEQLNALESELYSGKIKTDEEFKKKADEYGISIKLTEDATKITTVGNCVRGEVTTKKVFETYRKRIYGTTMGYQIVELSGSETVSREAGEKAIIVFGNKFAKQEAEKLINKKWKPSLSLKYMGDIFEEILTEISRKTPTIGNTFDVLIKQPKFNKSEAQRHLNVSIDKDIKVLSKFRQKLQEDLIQKNKEIALADKIINKGDVGEVVSVDGKMLHVKLNSKTQAFDGNWKQIAKPNETVFMFSDHNHVELGDKVVIQDEKLCLKKDKSNLKCDIILCSL

SEQ ID NO: 182 - protein, 305 aa, Methanobrevibacter smithii
  source 1..305: note = >WP_004033346.1 Methanobrevibacter smithii Adriase
SLIIAYIGKKGCVMVSDKRRIGYFGNKEQLNALESELYSGKIKTDEEFKKKADEYGISIKLTEDATKITTVGNCVRGEVTTKKVFETYRKRIYGTTMGYQIVELSGSETVSREAGEKAIIVFGNKFAKQEAEKLINKKWKPSLSLKYMGDIFEEILTEISRKTPTIGNTFDVLIKQPKFNKSEAQRHLNVSIDKDIKVLSKFRQKLQEDLIQKNKEIALADKIINKGDVGEVVSVDGKMLHVKLNSKTQAFDGNWKQIAKPNETVFMFSDHNHVELGDKVVIQDEKLCLKKDKSNLKCDIILCSL

SEQ ID NO: 183 - protein, 305 aa, Methanobrevibacter sp.
  source 1..305: note = >RBQ22740.1 Methanobrevibacter sp. NOE Adriase; note = Methanobrevibacter sp. NOE
SLIIAYVGKKGCVMASDKRRIAYFGEKDNREELEEELYSGSIANDDDLKKRSAELDITLKISDDANKIRYIEDVIVGEVSSRSTHETRRKRIYGTSNGYQLIEMLGSEITHSEKGESAIILFGNKVSKSLANDLISKKWKPRLSLKYMGEIFEDVLSEVSSKTPSLGKKYDVMIVQPKFNEKKAQSYLDETIKRDVQLLGKWREKLKNDLLEQNETIQLASKIIDEGDIGLVSSADGSILQVTLNDDVQAYDTNWKQLAKPGELVVMISDEKEVEVGDEIVIENETLCIKRNKANLKCDIILCDL

SEQ ID NO: 184 - protein, 305 aa, Methanobrevibacter sp.
  source 1..305: note = Methanobrevibacter arboriphilus; note = >WP_042704220.1 Methanobrevibacter arboriphilus Adriase
SLIIAYVGKKGCVMASDKRRIAYFGDKENREKLEEEIYSGAIKNDEDLKKRSDELDITLKISDDANKIRYIENVIVGEVSSRTTFETRRKRIYGTTYGYQIIEMLGSDITHSEKGENAIILFGNKISKAMANDLISKKWKSSFSLKYMGEIFESVLEEVASKTPSLGKKCDVMIVHPELDKKSSQKYLDETIKRDVQLLGKWREKLKNDLLEQNETVQLAAKIINEGDIGLVSNTDGPILQVTLNKDVKAYDTNWKQLAKPGEVVVMISNQGDVEIGDEVVIENEILCIKRNKSSLKCDIILCDI

SEQ ID NO: 185 - protein, 305 aa, Methanobrevibacter sp.
  source 1..305: note = Methanobrevibacter arboriphilus; note = >WP_080459963.1 Methanobrevibacter arboriphilus Adriase
SLIIAYVGKKGCVMASDKRRIAYFGDKENREKLEEEIYSGSIKNDEDLKKRSDELNITLKISDDANKIRYIENVIVGEVSSRTTFETRRKRIYGTTYGYQIIEMLGSDITHSEKGENAIILFGNKISKAMANDLISKKWKSSFSLKYMGEIFESVLEEVASKTPSLGKKCDVMIVHPELDKKSSQEYLDETIRRDVQLLGKWREKLKSDLLEQNETIQLATKIINEGDIGVVSTTDGPILQVTLNKDVQAYDTNWKQLAKPGEIVVMISNPGDVEIGDEVVIENEILCIKRNKSHLKCDIILCDT

SEQ ID NO: 186 - protein, 305 aa, Methanobrevibacter woesei
  source 1..305: note = >WP_116669733.1 Methanobrevibacter woesei Adriase
SLIIAYVGKKGCVMASDKRKIAYFGNKENLEALESELYNGEIATDEELYKRAKDFDISIKISDDGNKIDTVGNTVMGEVASKGAFETKRRRIYGTTNGYQIIEIIGSEIVSRSAGEKAIIIFGNNFAKKEAEALISKKWKSTLSLKYMGDIFKEIINEVSQKTPTLGDKFDVLIQQPKFTADEAQKYLNETIDQDVKVLAKIRQKLQEDLIEKSREIELASKIINEGDIGEVSSIEGNMLQVTLNDKTQAFDNNWRQLAKPKGKVIMFCETDDVKIGDKVVIENENLCLEKNKSNLSCNIILCNL

SEQ ID NO: 187 - protein, 303 aa, Methanobrevibacter millerae
  source 1..303: note = >WP_058739601.1 Methanobrevibacter millerae Adriase
SLIIAYVGKKGCVMASDKRRIAYFGNNRQALEDELYDGSITTDEELYQRSKELDVPIKISDDADKIRVVGNTVRGEVSTKGTHETKRKRIYGTTNGYQIVELIGSETKSRQAGEKGIILFGNDFAKKQAETLIQRRWKASHSLRLMGDIFEEILSDIAQKTPTIGKEFDVLIQQPKFTASEAQKHLNITIDADIKVLTKYRQQLTEEMIQKTREIELANKIIDDGPIGKVESIDGKMVEVKLNDKTQAFNFNWKPLAGPNGNVIMFADTDDIKIGDKVIIKDEVLCLKKNSSPLSCNIILCSL

SEQ ID NO: 188 - protein, 305 aa, Methanobrevibacter oralis
  source 1..305: note = >WP_042694031.1 Methanobrevibacter oralis Adriase
SLIIAYIGKKGCVMASDKRKIGYFGNKENLEILENELYNAVIRDDDEFLKRAEELGISIKITDDANKISIVENTIRGEVSTKGTFETKRKRIYGTTNGYQIVELLGSEVSSRQAGKSGIIIFGNKFAKQKAETLINRYWKSSQSLKYMGEIFGNILEEISKKTPSIGSQFDILVKSPNFDEMKAQKHLDVTIDHDIKVLTKFRQKLTEELIQQNLAIEMANKIIDKGEIGKVVSIEDNMIQVELNDKTQAMNGDWKQLAAPGEKVLMFSTSDDIKLGDKVIIDNEILCLKKNKSPLSCDIILCSL

SEQ ID NO: 189 - protein, 305 aa, Methanobrevibacter gottschalkii
  source 1..305: note = >WP_069573863.1 Methanobrevibacter Adriase
SLIIAYIGKKGCVMAADKRRIGYFGDKKNLDLLEEELYEGSIGNDEEFLARASELGISIKITDDANKLKTVGNCIRGEVSTKGTLETKRRRIYGTTNGYQIIELLGSDTKSRNAGENGIVIFGNNFAKQLAHNLISKKLKGSKSLRYMGEVFEDILKEVSSKTPTVGKNIDVLMQQPDFNKTEAQKHLNLTIDHDIKVLTKFRQELTEQIVQQSIEIEMANKIINKGSVGHVVNVDGNMLFIQLNDKTQAMDGNWKQLAAPGQNVLMFSESSNVEIGDEVVIENEELCLKKDKSSLKCDIILCSL

SEQ ID NO: 190 - protein, 305 aa, Methanobrevibacter thaueri
  source 1..305: note = >WP_116591264.1 Methanobrevibacter thaueri Adriase
SLIIAYVGKKGCVMASDKRKIGYFGDKENLKKLEDELYSGKITADEDFLKRAKELGISIKITDDASKLKIIGNTIRGEVSTKGTFETRRRRIYGTTSGYQIIELLGSDTQSRKAGKNGVIIFGNDYAKRMAETFIKREWKSSQSLRYIGEIFQGIIEDVASKTPTVGKNVDVMIQHPEFNEREAQKHLDITIDHDIKVLTKFRQELTEQIVQQQIEIDMVNKIMNEGTVGKVVNIDDNMLFVQLDKKVQAVDENWKQLAGPGQNVLMFTESDNVKIGDKVVIENEDLCLKKDKSSLKCDVILCSL

SEQ ID NO: 191 - protein, 305 aa, Methanobrevibacter sp.
  source 1..305: note = Methanobrevibacter sp. YE315; note = >WP_067043438.1 Methanobrevibacter sp. YE315 Adriase
SLIIAYIGKKGCVMASDKRKIGYFGDKQNLKTLEEELYSGAIDNDEDFLNRASELGISIKITDDANKLKVVGNTIRGEVSTKGTFETRRRRIYGTTNGYQIIELLGSETESRKAGEKGIVIFGNVYAKQLAEPLIKRKVKASQSLRYMGEVFESILAEVASKTPTVGDKFDVLMQQPVFSVSEAQKHLNITIDNDIKVLTKFRQELTEQLVQQSLAIDMASKIISKGDVGRVVSIDGNMLHVQLNDKTQAMDGNWKQLAAPGQNVLMFTESDNVKIGDKVVIENEDLCLKKDKSSLKCDIILCSL

SEQ ID NO: 192 - protein, 315 aa, Methanobacterium sp.
  source 1..315: note = >WP_048190775.1 Methanobacterium sp. SMA27 Adriase; note = Methanobacterium sp. SMA27
SLIITYIGSKGCVMAGDKRRIGFFGPEGPRENLEEKLYSGSINTKEELLKKADELGINIKISDDADKIHEIGDVVVGEVRSKTVYETKRKRIYATTGAYNIIELLGSDLKTVKSGGSSIIVFGNKYTKELAEKTIKRYWKSKLNLVTIRKIFESVMEEVAIETPTVSPEYDVMMKYPSIDTKEAKELLRVTILQDVKDLEEYRTQLKENLIQTSQNIQMATKIVIQGAIGNVAYVKGNEIGIILDKSIEALDTDWNTIAKPSELIEMTAQDGSKVSRGDLAVIENENLCINRTKEPLNCDFILCRADPDNKENIK

SEQ ID NO: 193 - protein, 308 aa, Methanobacterium sp.
  source 1..308: note = >WP_013644693.1 Methanobacterium lacus Adriase; note = Methanobacterium lacus
SLIVTYIGSKGCIMAGDKRRIGFYGSPEARETLEDELYAGKIKTEEDLIKKAEEQGINLKISDDTTKIREMDNVLVGEVISRATHETKRKRIYAISGAYSMVELTGSNIDTVKSGGRSIIVYGNKYTQELAEKTIKKYWKPKMKFVDVERIMELVMSEVSENTPSVSKSFDLMIKYNSMDQKLAKEVIRTTVLEDVKNLEAFRNQLREENIKATKNIQMATKIVIEGNIGTVTRVNGNDVEIILDQTVEALDFNWEPVAKPAERVILTSENPQNVVMGDLAVIENENLCIKRTKESLNCGFILCRSDD

SEQ ID NO: 194 - protein, 307 aa, Methanobacterium sp.
  source 1..307: note = >OPX57417.1 Methanobacterium sp. PtaB.Bin024 Adriase; note = Methanobacterium sp. PtaB.Bin024
SLIITYIGSKGCVMAGDKRSIGFLGDKNQREILEEELYSGKIKTKDQLTKRAHQLDINLKITDESEKVRKLDKVLVGEVKIRATHETRRKRIYATTNGYHQVELVGSEIKNVKSGKSSIVIFGNKLTKEITGKRLKKHWKSKISLNEVGEIFQKVMEDVAQVTPSVSPQYDLSIIHPQMDHKQAMELLRTTIISDVKELEKWREQLREDMLSKRQDIQMASRIITQGEIGRVKKAEGEKVEVILSEGVEALNMDWNVLAKSGDTIVMKAEQPSPLNLGDLVVIEEENLCIKKNKTPLSCDIILCKSE

SEQ ID NO: 195 - protein, 307 aa, unidentified
  source 1..307: note = >PKL73782.1 Methanobacteriales archaeon HGWMethanobacteriales2 Adriase; note = Methanobacteriales archaeon HGWMethanobacteriales2
SLIMTYIGSKGCVMAADKRSIGFLGDKNQRELLEEDLYSGKIDSDEKLLKRAKELDINLKISDQGEKIRNLGKVLVGEVKIRATHETKRKRIYATTNGYHQVELTGSEIQNVKSGQSSIVIFGNKITKEIAGKSLKKHFKSKISLNEVGKIFQKVMEDVAEATPSVSSEHDIFIIHPQMDHKQAMELLRTTIISDVKELEKWRNKLKEDMLAQRRDIQMASRIITQGEVGRVKKVEGDVVEVILSDGVEALNMDWNVLAKPGDTIFMKIEKPSPLNLGDLVVIEDENLCIKKNKTSLSCDIILCKAD

SEQ ID NO: 196 - protein, 307 aa, Methanobacterium sp.
  source 1..307: note = >WP_100906514.1 Methanobacterium Adriase
SLIMTYIGSKGCVMAADKRSIGFLGDKNQRELLEEDLYSGKIDSDEKLLKRARELDINLKISDQGEKIRNLGKVLVGEVKIRATHETKRKRIYTTTNGYHQVELTGSEIQNVKSGQSSIVIFGNKITKEIAGKSLKKHFKSKISLNEVGKIFQKVMEDVAEATPSVSSEHDIFIIHPQMDHKQAMELLRTTIISDVKELEKWRNKLKEDMLAQRRDIQMASRIITQGEVGRVKKVEGDVVEVILSDGVEALNMDWNVLAKPGDTIFMKIENPSPLNLGDLVVIEDENLCIKKNKTSLSCDIILCKTD

SEQ ID NO: 197 - protein, 308 aa, Methanobacterium sp.
  source 1..308: note = Methanobacterium sp. MB1; note = >WP_039376705.1 Methanobacterium sp. MB1 Adriase
SLIITYVGSKGCVMAGDKRSIGFLGDKEQREVLEEEMYSGKIKTTEELLQRAEQLDINLKITDDVEKVRKIGDVLVGEVKLRTTTETKRKRIYGTSGGYHEVELSGSQIKNAKSGQSSIVVFGNKFTKEIANKHLQKYWKSQTSIKEIAKIFQRIMEDVAQSTPSVSPQFDIYTVHPQLNHKQAMEILRTTIVKDVKELEKWREELKTQMLNQRRDIQMAARILTQGEVGKVLKVEGDEVEVILSPGVEALDMQWNVLSREGETIVMKMEEPSSLNMGDLVVIENENLCVKKDKTPLSCDVILCRTDK

SEQ ID NO: 198 - protein, 308 aa, Methanobacterium sp.; identical to SEQ ID NO: 197
  source 1..308: note = >CDG65302.1 Methanobacterium sp. MB1 Adriase; note = Methanobacterium sp. MB1

SEQ ID NO: 199 - protein, 308 aa, Methanobacterium formicicum
  source 1..308: note = >WP_048073463.1 Methanobacterium formicicum Adriase
SLIITYVGSKGCVMAGDKRSIGFLGDKEQREVLEEEMYRGKIKTTEELLQRAGQLDINLKITDSVEKVRNLGDVLVGEVKLRTTTETRRKRIYGTSSGYHQVELSGSEIKNVKSGQSSIVVFGNKLTKEITNKHLQKYWKSQTSLNEVSKIFQRIMEDVAQSTPSVSSQFDIYMVHPRLDHKQAMELLRTTIIEDVKELEKWREELKTQMLEQRRDIQMASRILTQGEVGRVRKVEGTEVEVILAPGVEALDMQWNVLSREGGTIVMKMDEPSPLSLGDLVVIEDENLCVKKSKTHLSCEIILCKSDK

SEQ ID NO: 200 - protein, 308 aa, Methanobacterium formicicum; identical to SEQ ID NO: 199
  source 1..308: note = >CEA14429.1 Methanobacterium formicicum Adriase

SEQ ID NO: 201 - protein, 308 aa, Methanobacterium formicicum
  source 1..308: note = >WP_048086016.1 Methanobacterium formicicum Adriase
SLIITYVGSKGCVMAGDKRSIGFLGDREQREVLEEEMYSGKIKTTEELLQRASQLDINLKITDSVEKVRNLGDVLVGEVKLRTTTETRRKRIYGTSSGYHQVELSGSEIKNVKSGQSSIVVFGNKLTKEIANKHLQKYWKSQTSLNEVSKIFQRIMEDVAQSTPSVSSQFDIYMVHPRLDHKQAMELLRTTIIKDVKELEKWREELKTQMLEQRRDIQMASRILTQGEVGRVRKVEGTEVEVILAPGVEALDMQWNVLSREGGTIVMKMDEPSPLSLGDLVVIEDENLCVKKSKTPLSCEIILCKSDK

SEQ ID NO: 202 - protein, 308 aa, Methanobacterium formicicum; identical to SEQ ID NO: 201
  source 1..308: note = >AIS32222.1 Methanobacterium formicicum Adriase

SEQ ID NO: 203 - protein, 308 aa, Methanobacterium formicicum
  source 1..308: note = >WP_060537561.1 Methanobacterium formicicum Adriase
SLIITYVGSKGCVMAGDKRSIGFLGDREQREVLEEEMYSGKIKTTEELLQRASQLDINLKITDSVEKVRNLGDVLVGEVKLRTTTETRRKRIYGTSSGYHQVELSGSEIKNVKSGQSSIVVFGNKLTKEIANKHLQKYWKSQTSLNEVSKIFQRIMEDVAQSTPSVSSQFDIYMVHPRLDHKQAMELLRTTIIEDVKELEKWREELKTQMLEQRRDIQMASRILTQGEVGRVRKVEGNEVEVILAPGVEALDMQWNVLSQEGETIVMKMNEPSPLSLGDLVVIEDENLCVKKSKTPLSCEIILCKSDK

SEQ ID NO: 204 - protein, 308 aa, Methanobacterium formicicum; identical to SEQ ID NO: 203
  source 1..308: note = >CEL24543.1 Methanobacterium formicicum Adriase

SEQ ID NO: 205 - protein, 308 aa, Methanobacterium sp.
  source 1..308: note = Methanobacterium sp. BAmetb5; note = >AXV39335.1 Methanobacterium sp. BAmetb5 Adriase
SLIITYVGSKGCVMAGDKRSIGFLGDREQREVLEEEMYSGKIKTTEELLQRASQLDINLKITDSVEKVRNLGDVLVGEVKLRTTTETRRKRIYGTSSGYHQVELSGSEIKNVKSGQSSIVVFGNKLTKEIANKHLQKYWKSQTSLNEVSKIFQRIMEDVAQSTPSVSSQFDIYMVHPRLDHKQAMELLRTTIIEDVKELEKWREELKTQMLEQRRDIQMASRILTQGEVGRVRKVEGNEVEVILAPGVEALDMQWNVLSREGGTIVMKMDEPSPLSLGDLVVIEDENLCVKKSKTPLSCEIILCKSDK

SEQ ID NO: 206 - protein, 307 aa, Methanobacterium sp.
  source 1..307: note = Methanobacterium sp. Maddingley MBC34; note = >EKQ53249.1 Methanobacterium sp. Maddingley MBC34 Adriase
SLIITYVGKKGCVMAGDKRSIGFLGDRKQRELLEEELYAGKIKTTEDLHKRAEQLDINLKITDNVEKVRNLGEVLVGEVKLRTTLETRRKRIYGTSSGYHQVELTGSEIRKVQSGQSSIVVFGNKITKEIANKHLKKYWKSKTSLDEVGKIFRRIMEDVAQATPSVSQQYDIFIIHPQLNHKQAMELLRTTIVGDVKELEKWREKLKQEMLEKTRDIQMASRILTQGEVGRVRKVEGDEVEVILSPGVEALNTRWDTLASEGETIIMKMETPTPLNLGDMVVIEDENLCIKKDKTSLSCDIILCKSS

SEQ ID NO: 207 - protein, 308 aa, Methanobacterium formicicum
  source 1..308: note = >WP_004031535.1 Methanobacterium formicicum Adriase
SLIITYVGSKGCVMAGDKRSIGFLGDKEQRELLEEEMYTGKIKTTEELHKRAKELEINLKITDNVEKVRNLGEVLVGEVKLRTTLETRRKRIYGTSSGYHQVELTGSEIKNVKSGQSSIVIFGNKITKEIANKHLKKHWKSKTSLNEVGKIFRNIMEDVAQLTPTVSSQYDIFTVHPQLDHKQAMELLRTTMIADVKQLQKWREDLKQEMLEKSRDIQMASRILTQGEVGRVRKTEGDEVEVILSPGVEALNTKWELLAGPGETITMKLSDHTSLNRGDLVIIEDENLCIKKDKTGLSCDIILCKSDQ

SEQ ID NO: 208 - protein, 308 aa, Methanobacterium sp.
  source 1..308: note = Methanobacterium sp. PtaU1.Bin097; note = >OPY22699.1 Methanobacterium sp. PtaU1.Bin097 Adriase
SLIICYTGSNGSVIIGDKRRIGFFGNEKKRELLEEEFYSGSIKTQEALLKRAEELGITLKITDDAEKVREIGDVITGEVKTTTPFETKRKRIYATTGSYSLVELSGSTIKNMEGGTTSIVVFGNKYTKEIANKAIQDNWKKKVSLKDVGEIFEKAMDEVSRQTPSVSPNYDLIIKHPSLNKKQARELLRTTILQDVKELEKWREELKEQMIKAAKGIEMSNKILDNGVVGKVSKVEGHQVEIILAEGVEALDLEWNLQAEAGEVVAMEVDEPDKISIGDMAVIKDENLCIMPSQCGLKCEVILCKTDK

SEQ ID NO: 209 - protein, 309 aa, Methanobacterium sp.
  source 1..309: note = >TMS41273.1 Methanobacterium sp. Adriase
SLIICYTGSNGSVIIGDKRRIGFLGNEKKRKLLEEELYSLSIKSQDELLKRAAELEITLKISDDAEKVRELGDVVVGEVKNTTPFETKRKRIYATTGSYNLVELTGSSIEKMDGGTTSIVVFGNKSTKKIANQVIEENWKKKLSLKDVAGIFKKAMEEVSRQTPSVSQDYDIIVKNPELNKNQAREILRTTIIQDVKDLEKWRSQLKEQMIKAAKGIEMSNKIMDQGVVGKISKITGDQVEITLATGVEALDLEWNLQAEAGEVVTMEVDSPDKVSLGDMAVIKDENLCIMPSQCGLKCEVILCKADDR

SEQ ID NO: 210 - protein, 306 aa, Methanobacterium sp.
  source 1..306: note = >WP_069583131.1 Methanobacterium Adriase
SLIMSYIGSNGCVMAGDKRRIGFFGDKEKREKLEEYLYSGAIQTDEELVKVAKEQEVTLKISDDARKIRSIADVVVGEVRFKTPFETKRKRIYGTTNGYIITELSGSDIKKVENGASSIVVFGNKVTKQMANEIIKKHWKSKASLENVESVFKKVMDEISGKTPSVSKDYDIIIKHSGMDKREAQKHLREILIKDVKELQKWREGLRQQLDTNAKSIQMASKIINEGEIGKVQSINGDKIGILLDKSVQALDMEWNVIAGPGGLIEMNTDDPSSVKIGDVAVIENENLCIRRTKCSLTCNVILCRS

SEQ ID NO: 211 - protein, 306 aa, Methanobacterium sp.
  source 1..306: note = >WP_048080675.1 Methanobacterium Adriase
SLIMSYIGSNGCVMAGDKRRIGFFGDKEKREKLEEYLYSGAIQTDEELVKVAKEQEVTLKISDDARKIRSIDDVVVGEVRFKTPFETKRKRIYGTTNGYIITELSGSDIKKIENGASSIVVFGNKVTKQMANEIIKKHWKSKASLENVESVFKRVMDEISTKTPSVSKEYDIITKHSGLDKKEAQKHLREILIKDVKELQKWREGLRQQLDTNAKSIQMASKIINEGEIGKVQSINGDKIGILLDKSVQALDMEWNVIAGPGGLIEMNTDDPSSVKIGDVAVIENENLCIRRTKCSLTCNVILCRS

SEQ ID NO: 212 - protein, 308 aa, Methanobacterium sp.
  source 1..308: note = >AXV36996.1 Methanobacterium sp. BRmetb2 Adriase; note = Methanobacterium sp. BRmetb2
SLIMTYIGSHGCVMIGDKRRIAFFGDKELIQKLEEELYSGKIKNDEELLERSKSLDVTIKVVDDAKKVRSLGDVVVGEVRFKTTFETRRKRIYATTNGYDIVELLGSKIDKIQKGESSIIVFGNKMTKKIANDTIRENWKPKTNLKDIGELFKVIMEEIALLTPSISKDYDFFVKSKNMNNNEAQKLLRETIVRDVKLLEKWRNKLKDDLLEKARSIEMAKKIIIEGQIGQVSGINDDNIEITLNNDIEALDIDWNVVAHTGEKVSMGINDPSQVKEGDLVVIEKENLCIKRTKSQLKCEVILCKADE

SEQ ID NO: 213 - protein, 306 aa, Methanothermobacter sp.
  source 1..306: note = Methanothermobacter sp. MT2; note = >BAW31310.1 Methanothermobacter sp. MT2 Adriase
SLIITYVSSKGCVIVGDKRKIAYFGDRENRKKLEKELYEGKIKNEKELRKRAEELGITLNIRDDAKKVRSIGDVVIGEVSLKTPFEAKRRRIYATTNAYQIVELVGSNITKLEKGESSIIIFGNKITKELTGKLLKKRWKKKTNLKEVSKLLEEVLEEVAKKTPTISQEHDKFIKTPRLDKRKAHKLLRETIVRDVKVLQKWRATLKMELIKKSEQIKMASKIIKEGEIGQVINIDEDKIEVKLSPKVEAYNIKWKKVAKPGENVLMIAEKPGKVKVGDLVVIEKENLQIKDKKIPLQCNVILCKI

SEQ ID NO: 214 - protein, 306 aa, Methanothermobacter sp.
  source 1..306: note = >WP_112094313.1 Methanothermobacter tenebrarum Adriase; note = Methanothermobacter tenebrarum
SLIITYTSSKGCVMIGDKRKIAYLGSPQKRRELERELYNGDIKTEEELRDRAEEFGIRLNIRDDARKVRSIGDVVVGEVSLKTPFEAKRRRIYATTGAYQIIELIGSNITKFEKGESSIVIFGNKITKKLASSLLKKYWKKKTSLKEVSKLLAKIVEEVAAKTPTISPEYDLYIKTPRLDKRKAHRILRETILRDVKTLQKWRAKLQKELLKKSEQIKMASKIIKEGEIGRVINIDNDKIEVKLAPNVQAFNIKWKKVAKPGENVLMLKEGPGEVKVGDIVVIEDENLQIKDKKIPLQCNVILCKT

SEQ ID NO: 215 - protein, 306 aa, unidentified
  source 1..306: note = >KUK01501.1 Methanobacteriaceae archaeon 41_258 Adriase; note = Methanobacteriaceae archaeon 41_258
SLIITYISSKGCVIIGDKRKIAYLGSAQNRRELERELYNGNIKTEEELRSRAEQLGITLNITDDAKKVRSIGDVVVGEVSLKTPFEAKRRRIYATTGAYQIIELIGSNITKFEKGESSIVIFGNKITKKLASKLLKKHWKKKTSLEGISKLLKKILEDVATKTPTISREYDMYIKTPRLDKRKAHKLLRETILRDVKTLQKWRAKLQKELIKKSEQIKMASKIIKEGEIGRVINIDNDKIEVKLAPNVEAFNIKWKRVAKPGENVLMLKEKPGEVNVGDLVIIEDENLQVKDKKIPLQCNVILCKT

SEQ ID NO: 216 - protein, 308 aa, Methanobacterium sp.
  source 1..308: note = Methanobacterium paludis; note = >WP_013825588.1 Methanobacterium paludis Adriase
SLIITYIGSKGCVMIADKRRIGFFGEEKTREKLEEELYSGAIKTDEELLKKASSLGITLKITDDAEKIREIGDIVVGEVRFKTPFETKRKRIYGTTGAYNVVELLGSEIKRIKTGESSIVVFGNKVTKEIANKYIQNHWKKKTNLKDIGEIFKAAMEEVAAKTPSVSQKYDLFIKHADIDKKEAKELLRTTIITDVKDLKKWREDLTDEMVKTAQTINVVSKIIDHGEVGKVTDVQGNEVEVTLKKGVEALDLNFNLLAKSGDTIKMEIKDSSKVKIGDKAVVMDENLCIKRTKAGLNCTVMLCKSDK

SEQ ID NO: 217 - protein, 308 aa, Methanobacterium congolense
  source 1..308: note = >WP_071906792.1 Methanobacterium congolense Adriase
SLIITYVGSKGCVMAGDKRRIGFFGDEANREKLEEKLYSGEIRTDEELLKRASELEITLKITDDAEKVREIDDVAVGEVRFKTPFKTKRKRIYGTTGAYNIVELLGSDIKTIKGGESSIVVFGNKMTKEIANKTIKDNWKNKTSLQDIAKLFSKVMEDVARQTPSVSQEHDVIIKHPSMEYKEAKELLRTTIIQDVKDLTEWRNSLKEELLKTTETIQMATRIISEGEVGKITSISGDELEVTLKKGVEALDMEWKPLKKAGESVTMQLAEAGEVNVGDKVVVEKENLCIKRSKAGLTCSVIICREEK

SEQ ID NO: 218 - protein, 307 aa, Methanobacterium sp.
  source 1..307: note = >RJS49378.1 Methanobacterium sp. Adriase
SLIITYIGAQGCVMAGDKRRIAFFGDKSNREILEEELYSGKIENESQLKKRASELEITLKITDDAVKLRSLGKVVVGEVSSKSSFETKRRRIYATTNGFQIIELTGSNITKMDKGESSIIVFGNKFTKKTASKILKKYWKSKTTLKEIETIFQKVLHEVSEKTPSVGSSCDLFIKTPALDKKESQKILRETVVRDVKLLQKWRQKLKKDLLEKAEAINMASRILTQGEVGRVVNVDEDRLEIVLGKDVQAFDVNWKQLATPGDKVMMFFNGEGDIIEGDLVVIENERLCLKRNQSPLRCDIILCKSD

SEQ ID NO: 219 - protein, 307 aa, unidentified
  source 1..307: note = >PKL67848.1 Methanobacteriales archaeon HGWMethanobacteriales1 Adriase; note = Methanobacteriales archaeon HGWMethanobacteriales1
SLIMSYIGSKGCVISADKRRIAYFGDKSNREQLEEELYAGAIINDKELQARALDLGITLKISDDVCKVRSVGEVVVGEVSVKSPFETKRRRIYGTTNGYQIVELSGSDIAKMEKGEISIVVFGNKKTKEVANEIIKKRWENKTSLKGIADIFKEVMVEVAKVSPSISSENDIFIKHPKLDKREAQKLLRETILRDVKLLKKWREKLQNDLLEKAESIKMAANILTEGEIGRVVSIDEDMLEIILGKNVRAFDTRWKPVAGSGEKILMYSDKPNDVLVGDVAVIENENLCLKRNNSALRCDVILCKTE

SEQ ID NO: 220 - protein, 308 aa, Methanothermobacter marburgensis
  source 1..308: note = >WP_013295962.1 Methanothermobacter marburgensis Adriase
SLIITYISTRGCVIAGDKRRIAYFGDKSKREKLEEELYSGKIKSDDELHRRASELGVSIKVTDDTAKVRSLGDVVVGEISQKTPFETKRRRIYATTGAYQIIDLTGSRITSMEKGETAIIVFGNKVAKELTNRFLKKRWKTKTTLKEVENLFRELMDYVSSRTPSVGSKYDIFMKSPSLDKRSAHKLLSDTIVRDVRLLQKWRAKLKQEMLDRREEMKLASRILTEGEVGRVISQDGNHVEVKLSDGVEAYDTRWKRVAGPGENVLMRLSDDVAVAAGEGVAVRNENLCVDGRDIKLECDVIICRTEE

SEQ ID NO: 221 - protein, 308 aa, Methanothermobacter wolfeii
  source 1..308: note = >WP_074359097.1 Methanothermobacter wolfeii Adriase
SLIVTYISTRGCVIIGDKRRIAYFGDRSAREKLEEELYSGRIKSDEELQRRASELGLNIKVTDDACKVRSIGDVVVGEISQKTPFETKRRRIYATTGAYQIIELTGSRITSMEKGDTAIIVFGNRIAKEITNGFLKKRWKTRTTLKEVSELLTELMEHVSSRTPSVGSKYDLFMKSPALDKKSAHRILSETIVRDVKVLQKWRAKLRQEMLERSEEIRLASRILTEGEVGRVTGQDGRHIEVKLSEGVEAYDTRWKHVAGAGEKVLMRIPGEGNVDVGDRVVVKDENLCVDGKDLSLECDVIICRKES

SEQ ID NO: 222 - protein, 308 aa, Methanothermobacter sp.
  source 1..308: note = >WP_048175474.1 Methanothermobacter sp. CaT2 Adriase; note = Methanothermobacter sp. CaT2
SLIITYISSKGCVIAGDKRRIAYFGDKSSREVLEEELYTGKIKSDEELQRRASELGVNIKVTDDTCKVRSLGDVVVGEISQKTPFETRRRRIYATTGAYQIIELTGSKITSMEKGDTAIIVFGNKIAKEITNRFLKKRWKTKTSLKDVADLFRELMDHVSSQTPSVGSEYDLFIKSPSLDKKSAHKLLSDTIVRDVRLLQKWRAKLKQEMLDRREEMKLASRILTEGEVGRVMRQDGSHVEVKLAGDVEAYDTRWKKVAGPGDMVLMRVADGGTISPGEKIAVRDENLCVEGRDIKVDCDVIICRREE

SEQ ID NO: 223 - protein, 308 aa, Methanothermobacter thermautotrophicus
  source 1..308: note = >WP_010876390.1 Methanothermobacter thermautotrophicus Adriase
SLIITYISSKGCVIAGDKRRIAYFGDKSSREVLEEELYTGKIKSDEELQRRASELGVNIKVTDDTCKVRSLGDVVVGEISQKTPFETRRRRIYATTGAYQIIELTGSRITSMEKGDTAIIVFGNKIAKEITNRFLKKRWKTKTSLKDVADLFRELMDHVSSQTPSVGSEYDLFIKSPSLDKKSAHRLLSDTIVRDVRLLQKWRAKLKQEMLDRREEIKLASRILTEGEVGRVMRQDGSHVEVKLAGDVEAYDTRWKKVAGPGDMVLMRISDGGTLSPGEKIAVRDENLCVEGRDIKVDCDVIICRREE

SEQ ID NO: 224 - protein, 308 aa, Methanothermobacter sp.
  source 1..308: note = >BAZ98793.1 Methanothermobacter sp. EMTCatA1 Adriase; note = Methanothermobacter sp. EMTCatA1
SLIITYISSKGCVIAGDKRRIAYFGDKSSREVLEEELYTGKIKSDEELQRRASELGVNIKVTDDTCKVRSLGDVVVGEISQKTPFETRRRRIYATTGAYQIIELTGSKITSMEKGDTAIIVFGNKIAKEITNRFLKKRWKTKTSLKDVADLFRELMDHVSSQTPSVGSEYDLFIKSPSLDKKSAHKLLSDTIVRDVRLLQKWRAKLKQEMLDRREEIKLASRILTEGEVGRVMRQDGRHVEVKLAGDVEAYDTRWKKVAGPGDMVLMRISDGGTLSPGEKIAVRDENLCVEGRDIKVDCDVIICRREE

SEQ ID NO: 225 - protein, 308 aa, Methanothermobacter defluvii
  source 1..308: note = >WP_115892198.1 Methanothermobacter defluvii Adriase
SLIITYISSKGCVIAGDKRRIAYFGDKSSREVLEEELYTGKIKSDEELQRRASELGVNIKVTDDTCKVRSLGDVVVGEISQKTPFETRRRRIYATTGAYQIIELTGSRITSMEKGDTAIIVFGNKIAKEITNRFLKKRWKTKTSLKDVADLFRELMDHVSSQTPSVGSEYDLFIKSPSLDKKSAHRLLSDTIVRDVRLLQKWRAKLKQEMLDRREEIKLASRILTEGEVGRVMRQDGRHVEVKLAGDVEAYDTRWKKVAGPGDMVLMRISDGGTLSPGEKIAVRDENLCVEGRDIKVDCDVIICRREE

SEQ ID NO: 226 - protein, 86 aa, Methanococcus voltae
  source 1..86: note = >WP_013180795.1 Methanococcus voltae OB
IKVQKLMLIEKKIATKGEIGYVNEGKLKLYDKYLAIDRICENPNLYYEIDITGDVKEGDIILIDENNLKVKNKDVIVSVNKIICNR

SEQ ID NO: 227 - protein, 86 aa, Methanococcus aeolicus
  source 1..86: note = >WP_011973890.1 Methanococcus aeolicus OB
IDIKKTMLIAEKIEINGEVGYIKNGKLVLDENHLAIDTICSNPNLYNEIEIKGEYEEGDIIYIDNGELKVKGKDSSVCVDKIICKA

SEQ ID NO: 228 - protein, 86 aa, Methanothermococcus thermolithotrophicus
  source 1..86: note = >WP_018153627.1 Methanothermococcus thermolithotrophicus OB
FEFGKIMTIVNKIANEGEIGTIKNGKLVLNDDHIAIDKVCPNPKLFTEIEVEGNFEDGDIVVIENGNMKIKGKNENITTKYIVCKK

SEQ ID NO: 229 - protein, 86 aa, unidentified
  source 1..86: note = >GBF37010.1 Methanococcales archaeon HHB OB
IDFRKVLAIVDKIENNGEVGIIKDGKLVLNEDHLAIDKICPNPRQFREIEIEGDVEDGDIVVIENGTLKVKGKDAILKTNYIICRK

SEQ ID NO: 230 - protein, 86 aa, unidentified; identical to SEQ ID NO: 229
  source 1..86: note = >WP_131007839.1 Methanococcales archaeon HHB OB

SEQ ID NO: 231 - protein, 86 aa, Methanothermococcus okinawensis
  source 1..86: note = >WP_013866916.1 Methanothermococcus okinawensis OB
IDFKKVMIIADKIENNGEVGIIKNGKLVLDDNHIAIDKVCPNPKLFNEIEIEGDVEDGDVVLIEDGSLKIKGKDIPLAINHIICKK

SEQ ID NO: 232 - protein, 86 aa, Methanotorris igneus
  source 1..86: note = >WP_013799607.1 Methanotorris igneus OB
IDFGKVMTIVNKIVKNGEIGVIKNGKLVLYDKYIAIDKICPNPKMFNEIEVKGDVEDGDVIVIENGEMKVKGKNTPVFVDYTICEK

SEQ ID NO: 233 - protein, 86 aa, Methanotorris formicicus
  source 1..86: note = >WP_007043881.1 Methanotorris formicicus OB
IDFSKVMTIVNKIVKNGEVGVVKNGKLVLYDKYIAIDKICPNPNIFNDIEIRGDVEEGDVVVIENGEMKIKGKNTPVFVDHIICEK

SEQ ID NO: 234 - protein, 86 aa, Methanocaldococcus infernus
  source 1..86: note = >WP_013100627.1 Methanocaldococcus infernus OB
VDFGKVMDIVNRIVKNGEIGEVKNGKLHLFDDYIAVDSIKPNPKTYKVIDIEGAEDGDIIVIENGEMKVKGKDKKVNTNYIIIKSW

SEQ ID NO: 235 - protein, 86 aa, Methanocaldococcus villosus
  source 1..86: note = >WP_004589885.1 Methanocaldococcus villosus OB
IDFGKVMSIVNKIVKNGEVGVIKEGKLHLYDQYIAIDKISPNFKTFRIIDVKGDVEDGDIVVIENGDMKIKNKGIKVMTDYIICYK

SEQ ID NO: 236 - protein, 86 aa, Methanocaldococcus vulcanius
  source 1..86: note = >WP_015733029.1 Methanocaldococcus vulcanius OB
IDFGKVMSIVNKIVKNGEIGVIKDGKLHLYDEYIAVDKIDPNPNTFKIIEVKGNFKDGDVIVIENGDMKVKGTDERVETDYIIISK

SEQ ID NO: 237 - protein, 85 aa, Methanocaldococcus fervens
  source 1..85: note = >WP_015791349.1 Methanocaldococcus fervens OB
IEFGKVMTIINKIVKNGEIGVIKDGKLHLYDNYIAIDKIDPPNVFKVVEVEGNFKDGDVVVIENGEMKIKGTNEKIMTKYIICHK

SEQ ID NO: 238 - protein, 86 aa, Methanocaldococcus sp.
  source 1..86: note = >WP_012979926.1 Methanocaldococcus sp. FS40622 OB; note = Methanocaldococcus sp. FS40622
IDFGKVMSIVNKIVKNGEIGVIKDGKLHLYDNYIAIDKIDPNPKVFKVVEVEGNFKDGDIVVIENGDMKIKGTNEKLTTNYIIIHK

SEQ ID NO: 239 - protein, 86 aa, Methanocaldococcus jannaschii
  source 1..86: note = >WP_010870052.1 Methanocaldococcus jannaschii OB
IDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 240 - protein, 86 aa, Methanocaldococcus sp.
  source 1..86: note = Methanocaldococcus bathoardescens; note = >WP_048201244.1 Methanocaldococcus bathoardescens OB
INFGKIMTIVNKIVKNGEIGVIKDGKLHLYDNYVAIDKIDPNPNVFKVVEVEGNFKDGDIIVIENGNMKIKGTDERVTTKYIICHK

SEQ ID NO: 241 - protein, 86 aa, Methanococcus vannielii
  source 1..86: note = >WP_012066013.1 Methanococcus vannielii OB
AEMQKLMTIVNKIAKSGEIGIIKNGSLVLNESFLAIDKICLNPEIYSEIDINGNFKEGDLIEIQNGELTVKGTNNYVSVQKIICNR

SEQ ID NO: 242 - protein, 86 aa, Methanococcus maripaludis
  source 1..86: note = >WP_011869407.1 Methanococcus maripaludis OB
VEMQKVMMIADKIVKIGDVGKIENGNLVLYDDFLAIDKICPEPTIYCEIEITGEFIEGDVITIDNESLKVKRTGNPVVVEKIICKK

SEQ ID NO: 243 - protein, 86 aa, Methanococcus maripaludis
  source 1..86: note = >WP_012193694.1 Methanococcus maripaludis OB
IEMQKVMMIADKIVKIGDVGKIENGSLVLYDEFLAIDRICPEPTLYCEIGITGEFIEGDVITIDSESLKVKRTGNPAVVEKIICRK

SEQ ID NO: 244 - protein, 86 aa, Methanococcus maripaludis
  source 1..86: note = >WP_011977398.1 Methanococcus maripaludis OB
VEMQKVMMIADKIVKIGDVGEIENGNLVLYDEFLAIDKICPEPTIYCEIGITGEFAEGDIITIDNESLKVKRTGSPVVVEKIICRK

SEQ ID NO: 245 - protein, 86 aa, Methanococcus maripaludis
  source 1..86: note = >WP_104837987.1 Methanococcus maripaludis OB
AEMQILTMIAEKIVKIGDVGIIKNGTLVLYDEFLAINKICPEPEIYSEIEIKGEFIEGDVITIDNESLKVKRTGNPVVVQKIICKK

SEQ ID NO: 246 - protein, 86 aa, Methanococcus maripaludis
  source 1..86: note = >WP_013998520.1 Methanococcus maripaludis OB
AEMQILTMIAEKIVKIGDVGVIKNGTLVLYDEFLAINKICPEPEIYSEIEITGEFIEGDIITIDNESLKVKRTGTPVVVQKIICKK

SEQ ID NO: 247 - protein, 86 aa, Methanococcus maripaludis
  source 1..86: note = >WP_011169965.1 Methanococcus maripaludis OB
AEMQILTMIAEKIVKIGDVGVIKNGTLVLYDEFLAINKICPEPEIYSEIEITGEFIEGDIITIDNESLKVKRTGSPVAVQKIICKK

SEQ ID NO: 248 - protein, 86 aa, Methanococcus maripaludis; identical to SEQ ID NO: 247
  source 1..86: note = >WP_119720453.1 Methanococcus maripaludis OB

SEQ ID NO: 249 - protein, 86 aa, Methanococcus maripaludis; identical to SEQ ID NO: 247
  source 1..86: note = >BAP60138.1 Methanococcus maripaludis KA1 OB; note = Methanococcus maripaludis KA1

SEQ ID NO: 250 - protein, 108 aa, Methanopyrus sp.
  source 1..108: note = >WP_088335494.1 Methanopyrus sp. KOL6 OB; note = Methanopyrus sp. KOL6
LKHIREGYDILKEGVVGEVVEVGTEEEGEGVEDVPPERRIVVRLVEDVDARHMGDVVAGPGEEIVMAVEGDPEKVEPGDTVVVKDGIMKIDGKDLPVVTGYSICRTRR

SEQ ID NO: 251 - protein, 108 aa, Methanopyrus kandleri
  source 1..108: note = >AAM01502.1 Methanopyrus kandleri AV19 OB; note = Methanopyrus kandleri AV19
LKHIREGYDILKEGVVGEVVEVGTEEEGKGVEDVPPERRIVVRLAEDVDARHMGDVVAGPGEEIVMAVEGNPEEVEPGDRVVVKDGVMKIDGKDLPVITGYSICRTRR

SEQ ID NO: 252 - protein, 99 aa, Methanothermus fervidus
  source 1..99: note = >WP_013413908.1 Methanothermus fervidus OB
IKAAKEITLASKIITEGDVGEIVDIKDGKIMVKLNQDVCAYDLNWEKVANPGEIVTMTADNPSEIKVGDKVVIENEELYVERCKEPLQCEVILTYCNRG

SEQ ID NO: 253 - protein, 108 aa, Methanobrevibacter curvatus
  source 1..108: note = >WP_067092528.1 Methanobrevibacter curvatus OB
FDKSEEIKIASKIINEGYIGVVSKIKNNFIYVKLNYNVQAYNTRWKLLVKPNEEALMFLGEDSIKTHNSNNTQENIVKLGDKILIENEIIVIERNKTPLNCDIILTSV

SEQ ID NO: 254 - protein, 97 aa, Methanobrevibacter olleyae
  source 1..97: note = >WP_067146985.1 Methanobrevibacter olleyae OB
LKQNETIKLASTIIDEGPIGIVDSIDENLIQVKLNPDVRAFDINWKLLAKPGENVIMFVEGEDAVSLKDQVVIENEALCIKGNKANLKCDIILCHLE

SEQ ID NO: 255 - protein, 97 aa, Methanobrevibacter ruminantium
  source 1..97: note = >WP_012956362.1 Methanobrevibacter ruminantium OB
MEQNENIKLASTIIEEGSVGEIVIIDDTLIEVKLNPDVRAFDVNWKVLAGPGENVLMVVDDSEGLDKGDEIVIEDEVLCVKKNKANLKCDIILCHLE

SEQ ID NO: 256 - protein, 108 aa, Methanobrevibacter sp.
  source 1..108: note = >WP_016358763.1 Methanobrevibacter sp. AbM4 OB; note = Methanobrevibacter sp. AbM4
EEQRESIEVANKIINKGTIGYISKIEGKMLNVKLTDKVNAYDTNLKKVAGSNEEVIMFLDKDTEGNEIKEADVSSYAKVNDKVVIKNENLCIDKNNIPLNCGIILCNE

SEQ ID NO: 257 - protein, 108 aa, Methanobrevibacter sp.; identical to SEQ ID NO: 256
  source 1..108: note = Methanobrevibacter boviskoreani; note = >WP_040681637.1 Methanobrevibacter boviskoreani OB

SEQ ID NO: 258 - protein, 108 aa, Methanobrevibacter wolinii
  source 1..108: note = >WP_042707893.1 Methanobrevibacter wolinii OB
IKQQKSVELAGKIVTKGDVGFVESNEGKILKVKLAKDVQAFDMDWKVLAKPGEEIIMFLDKDNNGNEIKDTEVSNYVQKFDKVVIKDENLCLEKNNIKLNCNIILCNT

SEQ ID NO: 259 - protein, 108 aa, Methanobrevibacter sp.
  source 1..108: note = Methanobrevibacter sp. 87.7; note = >WP_088539534.1 Methanobrevibacter sp. 87.7 OB
LSQQKSIEFASKIITKGDVGFVNSYEGKILKIKLADNVQAFDMDWKVLAKPGEEIIMFIDKDNNGNEIKDIEVSNYVQKFDKVVVKDENLCLDKNNIKLNCDIILCNT

SEQ ID NO: 260 - protein, 96 aa, Methanobrevibacter filiformis
  source 1..96: note = >WP_066972212.1 Methanobrevibacter filiformis OB
LEQSEAIQMATKIINQGFVGKIANIENNMLEVQLNKNVQGFDHNWKLLVKPGEKAFMFAPENIDVKIGDKIVIQNETLCVEQNNVQLSCNIILCHL

SEQ ID NO: 261 - protein, 95 aa, Methanobrevibacter smithii
  source 1..95: note = >WP_019266968.1 Methanobrevibacter smithii OB
IQKNKEIALADKIIDKGDIGEVVSVDGKMLHVKLNSKTQAFDGNWKQIAKPNETVFMFSDNGDAELGDKVVIQNEKLCLKKDKSNLKCDIILCGL

SEQ ID NO: 262 - protein, 95 aa, Methanobrevibacter smithii
  source 1..95: note = >WP_019262821.1 Methanobrevibacter smithii OB
IQKNKEIALADKIINKGDVGEVVSVDGKMLHVKLNSKTQAFDGNWKQIAKPNETVFMFSDHNHVELGDKVVIQDEKLCLKKDKSNLKCNIILCSL

SEQ ID NO: 263 - protein, 95 aa, Methanobrevibacter sp.
  source 1..95: note = >WP_004035387.1 Methanobrevibacter OB
IQKNKEIALADKIINKGDVGEVVSVDGKMLHVKLNSKTQAFDGNWKQIAKPNETVFMFSDHNHVELGDKVVIQDEKLCLKKDKSNLKCDIILCSL

SEQ ID NO: 264 - protein, 95 aa, Methanobrevibacter smithii; identical to SEQ ID NO: 263
  source 1..95: note = >WP_004033346.1 Methanobrevibacter smithii OB

SEQ ID NO: 265 - protein, 95 aa, Methanobrevibacter sp.
  source 1..95: note = Methanobrevibacter sp. NOE; note = >RBQ22740.1 Methanobrevibacter sp. NOE OB
LEQNETIQLASKIIDEGDIGLVSSADGSILQVTLNDDVQAYDTNWKQLAKPGELVVMISDEKEVEVGDEIVIENETLCIKRNKANLKCDIILCDL

SEQ ID NO: 266 - protein, 95 aa, Methanobrevibacter sp.
  source 1..95: note = >WP_042704220.1 Methanobrevibacter arboriphilus OB; note = Methanobrevibacter arboriphilus
LEQNETVQLAAKIINEGDIGLVSNTDGPILQVTLNKDVKAYDTNWKQLAKPGEVVVMISNQGDVEIGDEVVIENEILCIKRNKSSLKCDIILCDI

SEQ ID NO: 267 - protein, 95 aa, Methanobrevibacter sp.
  source 1..95: note = Methanobrevibacter arboriphilus; note = >WP_080459963.1 Methanobrevibacter arboriphilus OB
LEQNETIQLATKIINEGDIGVVSTTDGPILQVTLNKDVQAYDTNWKQLAKPGEIVVMISNPGDVEIGDEVVIENEILCIKRNKSHLKCDIILCDT

SEQ ID NO: 268 - protein, 95 aa, Methanobrevibacter woesei
  source 1..95: note = >WP_116669733.1 Methanobrevibacter woesei OB
IEKSREIELASKIINEGDIGEVSSIEGNMLQVTLNDKTQAFDNNWRQLAKPKGKVIMFCETDDVKIGDKVVIENENLCLEKNKSNLSCNIILCNL

SEQ ID NO: 269 - protein, 95 aa, Methanobrevibacter millerae
  source 1..95: note = WP_058739601.1 Methanobrevibacter millerae OB
IQKTREIELANKIIDDGPIGKVESIDGKMVEVKLNDKTQAFNFNWKPLAGPNGNVIMFADTDDIKIGDKVIIKDEVLCLKKNSSPLSCNIILCSL

SEQ ID NO: 270 - protein, 95 aa, Methanobrevibacter oralis
  source 1..95: note = >WP_042694031.1 Methanobrevibacter oralis OB
IQQNLAIEMANKIIDKGEIGKVVSIEDNMIQVELNDKTQAMNGDWKQLAAPGEKVLMFSTSDDIKLGDKVIIDNEILCLKKNKSPLSCDIILCSL

SEQ ID NO: 271 - protein, 95 aa, Methanobrevibacter sp.
  source 1..95: note = >WP_069573863.1 Methanobrevibacter OB
VQQSIEIEMANKIINKGSVGHVVNVDGNMLFIQLNDKTQAMDGNWKQLAAPGQNVLMFSESSNVEIGDEVVIENEELCLKKDKSSLKCDIILCSL

SEQ ID NO: 272 - protein, 95 aa, Methanobrevibacter thaueri
  source 1..95: note = >WP_116591264.1 Methanobrevibacter thaueri OB
VQQQIEIDMVNKIMNEGTVGKVVNIDDNMLFVQLDKKVQAVDENWKQLAGPGQNVLMFTESDNVKIGDKVVIENEDLCLKKDKSSLKCDVILCSL

SEQ ID NO: 273 - protein, 95 aa, Methanobrevibacter sp.
  source 1..95: note = Methanobrevibacter sp. YE315; note = >WP_067043438.1 Methanobrevibacter sp. YE315 OB
VQQSLAIDMASKIISKGDVGRVVSIDGNMLHVQLNDKTQAMDGNWKQLAAPGQNVLMFTESDNVKIGDKVVIENEDLCLKKDKSSLKCDIILCSL

SEQ ID NO: 274 - protein, 105 aa, Methanobacterium sp.
  source 1..105: note = Methanobacterium sp. SMA27; note = >WP_048190775.1 Methanobacterium sp. SMA27 OB
IQTSQNIQMATKIVIQGAIGNVAYVKGNEIGIILDKSIEALDTDWNTIAKPSELIEMTAQDGSKVSRGDLAVIENENLCINRTKEPLNCDFILCRADPDNKENIK

SEQ ID NO: 275 - protein, 98 aa, Methanobacterium sp.
  source 1..98: note = Methanobacterium lacus; note = >WP_013644693.1 Methanobacterium lacus OB
IKATKNIQMATKIVIEGNIGTVTRVNGNDVEIILDQTVEALDFNWEPVAKPAERVILTSENPQNVVMGDLAVIENENLCIKRTKESLNCGFILCRSDD

SEQ ID NO: 276 - protein, 97 aa, Methanobacterium sp.
  source 1..97: note = Methanobacterium sp. PtaB.Bin024; note = >OPX57417.1 Methanobacterium sp. PtaB.Bin024 OB
LSKRQDIQMASRIITQGEIGRVKKAEGEKVEVILSEGVEALNMDWNVLAKSGDTIVMKAEQPSPLNLGDLVVIEEENLCIKKNKTPLSCDIILCKSE

SEQ ID NO: 277 - protein, 97 aa, unidentified
  source 1..97: note = >PKL73782.1 Methanobacteriales archaeon HGWMethanobacteriales2 OB; note = Methanobacteriales archaeon HGWMethanobacteriales2
LAQRRDIQMASRIITQGEVGRVKKVEGDVVEVILSDGVEALNMDWNVLAKPGDTIFMKIEKPSPLNLGDLVVIEDENLCIKKNKTSLSCDIILCKAD

SEQ ID NO: 278 - protein, 97 aa, Methanobacterium sp.
  source 1..97: note = >WP_100906514.1 Methanobacterium OB
LAQRRDIQMASRIITQGEVGRVKKVEGDVVEVILSDGVEALNMDWNVLAKPGDTIFMKIENPSPLNLGDLVVIEDENLCIKKNKTSLSCDIILCKTD

SEQ ID NO: 279 - protein, 98 aa, Methanobacterium sp.
  source 1..98: note = >WP_039376705.1 Methanobacterium sp. MB1 OB; note = Methanobacterium sp. MB1
LNQRRDIQMAARILTQGEVGKVLKVEGDEVEVILSPGVEALDMQWNVLSREGETIVMKMEEPSSLNMGDLVVIENENLCVKKDKTPLSCDVILCRTDK

SEQ ID NO: 280 - protein, 98 aa, Methanobacterium sp.; identical to SEQ ID NO: 279
  source 1..98: note = Methanobacterium sp. MB1; note = >CDG65302.1 Methanobacterium sp. MB1 OB

SEQ ID NO: 281 - protein, 98 aa, Methanobacterium formicicum
  source 1..98: note = >WP_048073463.1 Methanobacterium formicicum OB
LEQRRDIQMASRILTQGEVGRVRKVEGTEVEVILAPGVEALDMQWNVLSREGGTIVMKMDEPSPLSLGDLVVIEDENLCVKKSKTHLSCEIILCKSDK

SEQ ID NO: 282 - protein, 98 aa, Methanobacterium formicicum; identical to SEQ ID NO: 281
  source 1..98: note = >CEA14429.1 Methanobacterium formicicum OB

SEQ ID NO: 283 - protein, 98 aa, Methanobacterium formicicum
  source 1..98: note = >WP_048086016.1 Methanobacterium formicicum OB
LEQRRDIQMASRILTQGEVGRVRKVEGTEVEVILAPGVEALDMQWNVLSREGGTIVMKMDEPSPLSLGDLVVIEDENLCVKKSKTPLSCEIILCKSDK

SEQ ID NO: 284 - protein, 98 aa, Methanobacterium formicicum; identical to SEQ ID NO: 283
  source 1..98: note = >AIS32222.1 Methanobacterium formicicum OB

SEQ ID NO: 285 - protein, 98 aa, Methanobacterium formicicum
  source 1..98: note = >WP_060537561.1 Methanobacterium formicicum OB
LEQRRDIQMASRILTQGEVGRVRKVEGNEVEVILAPGVEALDMQWNVLSQEGETIVMKMNEPSPLSLGDLVVIEDENLCVKKSKTPLSCEIILCKSDK

SEQ ID NO: 286 - protein, 98 aa, Methanobacterium formicicum; identical to SEQ ID NO: 285
  source 1..98: note = >CEL24543.1 Methanobacterium formicicum OB

SEQ ID NO: 287 - protein, 98 aa, Methanobacterium sp.
  source 1..98: note = Methanobacterium sp. BAmetb5; note = >AXV39335.1 Methanobacterium sp. BAmetb5 OB
LEQRRDIQMASRILTQGEVGRVRKVEGNEVEVILAPGVEALDMQWNVLSREGGTIVMKMDEPSPLSLGDLVVIEDENLCVKKSKTPLSCEIILCKSDK

SEQ ID NO: 288 - protein, 97 aa, Methanobacterium sp.
  source 1..97: note = Methanobacterium sp. Maddingley MBC34; note = >EKQ53249.1 Methanobacterium sp. Maddingley MBC34 OB
LEKTRDIQMASRILTQGEVGRVRKVEGDEVEVILSPGVEALNTRWDTLASEGETIIMKMETPTPLNLGDMVVIEDENLCIKKDKTSLSCDIILCKSS

SEQ ID NO: 289 - protein, 98 aa, Methanobacterium formicicum
  source 1..98: note = >WP_004031535.1 Methanobacterium formicicum OB
LEKSRDIQMASRILTQGEVGRVRKTEGDEVEVILSPGVEALNTKWELLAGPGETITMKLSDHTSLNRGDLVIIEDENLCIKKDKTGLSCDIILCKSDQ

SEQ ID NO: 290 - protein, 98 aa, Methanobacterium sp.
  source 1..98: note = Methanobacterium sp. PtaU1.Bin097; note = >OPY22699.1 Methanobacterium sp. PtaU1.Bin097 OB
IKAAKGIEMSNKILDNGVVGKVSKVEGHQVEIILAEGVEALDLEWNLQAEAGEVVAMEVDEPDKISIGDMAVIKDENLCIMPSQCGLKCEVILCKTDK

SEQ ID NO: 291 - protein, 99 aa, Methanobacterium sp.
  source 1..99: note = >TMS41273.1 Methanobacterium sp. OB
IKAAKGIEMSNKIMDQGVVGKISKITGDQVEITLATGVEALDLEWNLQAEAGEVVTMEVDSPDKVSLGDMAVIKDENLCIMPSQCGLKCEVILCKADDR

SEQ ID NO: 292 - protein, 96 aa, Methanobacterium sp.
  source 1..96: note = >WP_069583131.1 Methanobacterium OB
DTNAKSIQMASKIINEGEIGKVQSINGDKIGILLDKSVQALDMEWNVIAGPGGLIEMNTDDPSSVKIGDVAVIENENLCIRRTKCSLTCNVILCRS

SEQ ID NO: 293 - protein, 96 aa, Methanobacterium sp.; identical to SEQ ID NO: 292
  source 1..96: note = >WP_048080675.1 Methanobacterium OB

SEQ ID NO: 294 - protein, 98 aa, Methanobacterium sp.
  source 1..98: note = >AXV36996.1 Methanobacterium sp. BRmetb2 OB; note = Methanobacterium sp. BRmetb2
LEKARSIEMAKKIIIEGQIGQVSGINDDNIEITLNNDIEALDIDWNVVAHTGEKVSMGINDPSQVKEGDLVVIEKENLCIKRTKSQLKCEVILCKADE

SEQ ID NO: 295 - protein, 96 aa, Methanothermobacter sp.
  source 1..96: note = BAW31310.1 Methanothermobacter sp. MT2 OB; note = Methanothermobacter sp. MT2
IKKSEQIKMASKIIKEGEIGQVINIDEDKIEVKLSPKVEAYNIKWKKVAKPGENVLMIAEKPGKVKVGDLVVIEKENLQIKDKKIPLQCNVILCKI

SEQ ID NO: 296 - protein, 96 aa, Methanothermobacter sp.
  source 1..96: note = >WP_112094313.1 Methanothermobacter tenebrarum OB; note = Methanothermobacter tenebrarum
LKKSEQIKMASKIIKEGEIGRVINIDNDKIEVKLAPNVQAFNIKWKKVAKPGENVLMLKEGPGEVKVGDIVVIEDENLQIKDKKIPLQCNVILCKT

SEQ ID NO: 297 - protein, 96 aa, unidentified
  source 1..96: note = >KUK01501.1 Methanobacteriaceae archaeon 41_258 OB; note = Methanobacteriaceae archaeon 41_258
IKKSEQIKMASKIIKEGEIGRVINIDNDKIEVKLAPNVEAFNIKWKRVAKPGENVLMLKEKPGEVNVGDLVIIEDENLQVKDKKIPLQCNVILCKT

SEQ ID NO: 298 - protein, 98 aa, Methanobacterium sp.
  source 1..98: note = Methanobacterium paludis; note = >WP_013825588.1 Methanobacterium paludis OB
VKTAQTINVVSKIIDHGEVGKVTDVQGNEVEVTLKKGVEALDLNFNLLAKSGDTIKMEIKDSSKVKIGDKAVVMDENLCIKRTKAGLNCTVMLCKSDK

SEQ ID NO: 299 - protein, 98 aa, Methanobacterium congolense
  source 1..98: note = >WP_071906792.1 Methanobacterium congolense OB
LKTTETIQMATRIISEGEVGKITSISGDELEVTLKKGVEALDMEWKPLKKAGESVTMQLAEAGEVNVGDKVVVEKENLCIKRSKAGLTCSVIICREEK

SEQ ID NO: 300 - protein, 97 aa, Methanobacterium sp.
  source 1..97: note = >RJS49378.1 Methanobacterium sp. OB
LEKAEAINMASRILTQGEVGRVVNVDEDRLEIVLGKDVQAFDVNWKQLATPGDKVMMFFNGEGDIIEGDLVVIENERLCLKRNQSPLRCDIILCKSD

SEQ ID NO: 301 - protein, 97 aa, unidentified
  source 1..97: note = Methanobacteriales archaeon HGWMethanobacteriales1; note = >PKL67848.1 Methanobacteriales archaeon HGWMethanobacteriales1 OB
LEKAESIKMAANILTEGEIGRVVSIDEDMLEIILGKNVRAFDTRWKPVAGSGEKILMYSDKPNDVLVGDVAVIENENLCLKRNNSALRCDVILCKTE

SEQ ID NO: 302 - protein, 98 aa, Methanothermobacter marburgensis
  source 1..98: note = >WP_013295962.1 Methanothermobacter marburgensis OB
LDRREEMKLASRILTEGEVGRVISQDGNHVEVKLSDGVEAYDTRWKRVAGPGENVLMRLSDDVAVAAGEGVAVRNENLCVDGRDIKLECDVIICRTEE

SEQ ID NO: 303 - protein, 98 aa, Methanothermobacter wolfeii
  source 1..98: note = >WP_074359097.1 Methanothermobacter wolfeii OB
LERSEEIRLASRILTEGEVGRVTGQDGRHIEVKLSEGVEAYDTRWKHVAGAGEKVLMRIPGEGNVDVGDRVVVKDENLCVDGKDLSLECDVIICRKES

SEQ ID NO: 304 - protein, 98 aa, Methanothermobacter sp.
  source 1..98: note = >WP_048175474.1 Methanothermobacter sp. CaT2 OB; note = Methanothermobacter sp. CaT2
LDRREEMKLASRILTEGEVGRVMRQDGSHVEVKLAGDVEAYDTRWKKVAGPGDMVLMRVADGGTISPGEKIAVRDENLCVEGRDIKVDCDVIICRREE

SEQ ID NO: 305 - protein, 98 aa, Methanothermobacter thermautotrophicus
  source 1..98: note = >WP_010876390.1 Methanothermobacter thermautotrophicus OB
LDRREEIKLASRILTEGEVGRVMRQDGSHVEVKLAGDVEAYDTRWKKVAGPGDMVLMRISDGGTLSPGEKIAVRDENLCVEGRDIKVDCDVIICRREE

SEQ ID NO: 306 - protein, 98 aa, Methanothermobacter sp.
  source 1..98: note = >BAZ98793.1 Methanothermobacter sp. EMTCatA1 OB; note = Methanothermobacter sp. EMTCatA1
LDRREEIKLASRILTEGEVGRVMRQDGRHVEVKLAGDVEAYDTRWKKVAGPGDMVLMRISDGGTLSPGEKIAVRDENLCVEGRDIKVDCDVIICRREE

SEQ ID NO: 307 - protein, 98 aa, Methanothermobacter defluvii; identical to SEQ ID NO: 306
  source 1..98: note = >WP_115892198.1 Methanothermobacter defluvii OB

SEQ ID NO: 308 - protein, 5 aa, synthetic construct
  source 1..5: note = Adriase recongition motif
RDPAA

SEQ ID NO: 309 - protein, 5 aa, synthetic construct
  source 1..5: note = Adriase recognition motif
KDPAA

SEQ ID NO: 310 - protein, 5 aa, Methanoculleus thermophilus
  source 1..5: note = Adriase motif in some Methanomicrobiaceae
RDPGA

SEQ ID NO: 311 - protein, 5 aa, Methanocaldococcus jannaschii
  source 1..5: note = Adriase motif in most Methanogens
KDPGA

SEQ ID NO: 312 - DNA, 930 bp, Methanothermobacter thermautotrophicus
  source 1..930: mol_type = genomic DNA; note = Methanothermobacter thermautotrophicus Adriase gene
atgagtctcatcataacctacataagtagcaagggatgcgtcatagctggagataaacggagaatagcctattttggtgataaatcaagtagagaggtacttgaggaggagctgtacacaggcaagataaaaagtgatgaggaactccagaggagggcgtcggagcttggcgtaaatataaaggtcacagatgacacatgcaaggtcagaagccttggcgacgttgttgtgggtgaaataagccagaaaacaccctttgagacaagaagaagaaggatatacgcaacaacaggggcctatcagataattgaactcacaggttcaaggataaccagcatggaaaagggtgatactgcaataatagtcttcggaaataagatagccaaggagattacaaacaggttccttaaaaagaggtggaagacaaagacaagccttaaggatgttgctgacctcttcagggaactcatggaccatgtatcctcacagacaccatctgttggtagtgaatatgatctgttcataaagagcccctcactggataagaagagcgcccacaggcttctgagtgatacaatcgtgagggacgtccgcctcctacagaaatggagggccaaactcaaacaggagatgctagaccgacgggaggagataaaactggcctcaaggatcctcacagagggtgaggtcggcagggttatgaggcaggatgggagccacgtggaggttaaacttgccggtgacgttgaggcctatgacactcgctggaagaaggttgcaggtccaggggacatggtactcatgaggatatcagatggcggcaccctttcacccggcgagaaaatagctgtaagggatgagaacctctgcgtggaggggagagatataaaggtcgactgtgacgtcataatctgtcgcagggaggaataa

SEQ ID NO: 313 - DNA, 921 bp, Methanobrevibacter smithii
  source 1..921: mol_type = genomic DNA; note = Methanobrevibacter smithii Adriase gene
atgagtttgattattgcttatattggaaaaaaaggatgtgtaatggctagtgataaaagaagaattggttattttgggaataaagagcaattaaatgcattggaatctgaattatacagtggtaaaataaaaaccgatgaggaatttaaaaagaaagctgatgaatacggcatctcaattaagttaactgaagatgctactaaaataaccactgtgggaaattgcgtaaggggagaagtcacaaccaaaaaagtttttgaaacatacaggaaaagaatttacggaactacaatgggttaccagatagtggaattgtcaggttctgaaactgtttcaagggaagccggtgaaaaggcaattattgtatttggtaataaatttgccaaacaggaagcagaaaagctaataaacaaaaaatggaaaccttcattaagtttaaaatatatgggtgatatctttgaagagatacttaccgaaatttctagaaaaacaccaacaattggaaatacttttgatgttttaatcaaacagcctaaattcaacaaatcagaagctcaaagacatcttaatgtaagtattgataaggatattaaggttttatctaaatttagacaaaagcttcaggaagatttaatccaaaaaaataaggaaatagctttggctgataaaattattaataaaggagatgttggtgaagttgtttcagtagatgggaaaatgcttcatgtcaaattaaatagtaaaacacaggcttttgatggaaattggaaacagatagctaagccaaatgaaactgttttcatgttcagtgaccataaccatgtggaacttggagataaggtagttatccaggatgaaaagttatgtcttaaaaaggacaaatctaatcttaaatgtgatataattttatgcagtttataa

SEQ ID NO: 314 - protein, 264 aa, Escherichia coli
  source 1..264: note = Escherichia coli BL21(DE3); note = >ACT42067.1 METHIONINE AMINOPEPTIDASE [ESCHERICHIA COLI BL21(DE3)]
MAISIKTPEDIEKMRVAGRLAAEVLEMIEPYVKPGVSTGELDRICNDYIVNEQHAVSACLGYHGYPKSVCISINEVVCHGIPDDAKLLKDGDIVNIDVTVIKDGFHGDTSKMFIVGKPTIMGERLCRITQESLYLALRMVKPGINLREIGAAIQKFVEAEGFSVVREYCGHGIGRGFHEEPQVLHYDSRETNVVLKPGMTFTIEPMVNAGKKEIRTMKDGWTVKTKDRSLSAQYEHTIVVTDNGCEILTLRKDDTIPAIISHDE

SEQ ID NO: 315 - protein, 30 aa, Methanofollis ethanolicus
  source 1..30: note = Adriase recognition motif
EDLGAIKAKIAELKGKDPGAFGEAPIVVEV

SEQ ID NO: 316 - protein, 30 aa, Methanoculleus sp.
  source 1..30: note = Methanoculleus horonobensis; note = Adriase recognition motif
EDMGAIKAKMDELKAKDPGAFGAEPMIVEV

SEQ ID NO: 317 - protein, 30 aa, Methanoculleus sp.
  source 1..30: note = Methanoculleus sediminis / Methanoculleus marisnigri JR1 / Methanoculleus sp. MH98A / Methanoculleus sp. SDB Adriase recognition motif
EDMGAIKAKIDELKARDPGAFGAEPMIVEV

SEQ ID NO: 318 - protein, 30 aa, Methanoculleus thermophilus
  source 1..30: note = Adriase recognition motif
EDMGTIKAKINELKARDPGAFGAEPMVVEV

SEQ ID NO: 319 - protein, 30 aa, Methanoculleus bourgensis
  source 1..30: note = Methanoculleus bourgensis / Methanoculleus bourgensis BA1 / Methanoculleus bourgensis MS2 Adriase recognition motif
EDMGTIKAKIDELKAKDPGAFPAEPMIVEV

SEQ ID NO: 320 - protein, 30 aa, Methanolinea tarda
  source 1..30: note = Adriase recognition motif
EDLGAIKAKIAELKGRDPGAFAADPMVVEV

SEQ ID NO: 321 - protein, 30 aa, unidentified
  source 1..30: note = Methanosaeta harundinacea; note = Adriase recognition motif
EDTGTIVSKIKECISKDPGALDVEPMTVEI

SEQ ID NO: 322 - protein, 30 aa, Methanothrix soehngenii
  source 1..30: note = Adriase recognition motif
EDIGAIAGKVKELASKDPGALDVEPMAIEI

SEQ ID NO: 323 - protein, 30 aa, Methanothrix thermoacetophila
  source 1..30: note = Adriase recognition motif; note = Methanothrix thermoacetophila PT
EDMGAITAKIKELVAKDPGALDVEPMIVEI

SEQ ID NO: 324 - protein, 30 aa, unidentified
  source 1..30: note = Adriase recognition motif; note = Candidatus Methanoperedens sp.; note = Candidatus Methanoperedens sp. BLZ2
EDENAIIAKIKELAAKDPGAFDGEPMIIQV

SEQ ID NO: 325 - protein, 30 aa, Methanosarcina thermophila
  source 1..30: note = Methanosarcina thermophila / Methanosarcina thermophila CHTI-55 Adriase recognition motif
EDMGVITAKVRELASKDPGAFDAEPMIVEI

SEQ ID NO: 326 - protein, 30 aa, Methanosarcina sp.
  source 1..30: note = Methanosarcina flavescens; note = Adriase recognition motif
EDMGAITAKVRELASKDPGAFDAEPMIVEI

SEQ ID NO: 327 - protein, 30 aa, Methanosarcina sp.
  source 1..30: note = Methanosarcina mazei Go1 / Methanosarcina sp. Kolksee / Methanosarcina vacuolata / Methanosarcina vacuolata Z-761 Adriase recognition motif
EDMGAITSKVRELASKDPGAFDAEPLVVEI

SEQ ID NO: 328 - protein, 30 aa, Methanosarcina sp.
  source 1..30: note = Methanosarcina mazei / Methanosarcina mazei C16 / Methanosarcina siciliae C2 / Methanosarcina sp. MSH10X1 Adriase recognition motif
EDMGAITSKVRELASKDPGAFDADPLVVEI

SEQ ID NO: 329 - protein, 30 aa, Methanosarcina barkeri
  source 1..30: note = Methanosarcina barkeri CM1 / Methanosarcina barkeri str. Fusaro Adriase recognition motif
EDMGAITSKVKELASKDPGAFDAEPMIVEI

SEQ ID NO: 330 - protein, 30 aa, Methanosarcina sp.
  source 1..30: note = Adriase recognition motif; note = Methanosarcina sp. MTP4
EDMGVITAKVKELVSKDPGAFDAEPLVVEI

SEQ ID NO: 331 - protein, 30 aa, Methanosarcina sp.
  source 1..30: note = Methanosarcina sp. 1.H.T.1A.1 / Methanosarcina sp. 2.H.T.1A.3 Adriase recognition motif
EDMGAITAKVRELVSKDPGAFDAEPMVVEI

SEQ ID NO: 332 - protein, 30 aa, Methanoculleus sp.
  source 1..30: note = Adriase recognition motif; note = Methanoculleus sp. SDB
EDMGAISAKISELVGKDPGAFDADPMVVEV

SEQ ID NO: 333 - protein, 30 aa, Methanosarcina siciliae
  source 1..30: note = Adriase recognition motif
EDMGAITAKVRELVSKDPGAFDAEPLVVEI

SEQ ID NO: 334 - protein, 30 aa, Methanosarcina sp.
  source 1..30: note = Methanosarcina sp. Ant1; note = Adriase recognition motif
EDMGAITSKIRELVSKDPGAFDADPLILQI

SEQ ID NO: 335 - protein, 30 aa, Methanosarcina acetivorans
  source 1..30: note = Adriase recognition motif
EDMGAITAKVRELVAKDPGAFDAEPMVVEI

SEQ ID NO: 336 - protein, 30 aa, Methanosarcina horonobensis
  source 1..30: note = Adriase recognition motif
EDMGAITAKVRELVAKDPGAFDADPLVVEI

SEQ ID NO: 337 - protein, 30 aa, Methanosarcina sp.
  source 1..30: note = Methanosarcina lacustris / Methanosarcina sp. WWM596 Adriase recognition motif
EDMGAITAKVRELVSKDPGAFDAEPMVVVI

SEQ ID NO: 338 - protein, 30 aa, Methanopyrus sp.
  source 1..30: note = Methanopyrus kandleri / Methanopyrus sp. KOL6 Adriase recognition motif
EDVDEIVKAIEECVEKDPGAYEEGPMTISL

SEQ ID NO: 339 - protein, 30 aa, Methanococcus voltae
  source 1..30: note = Adriase recognition motif
EDKGKIQQTVKDCISKDPGAFEEDAMVIDL

SEQ ID NO: 340 - protein, 30 aa, Methanococcus aeolicus
  source 1..30: note = Adriase recognition motif
EDTGKISSAIKDCISKDPGAFEEDPMILDL

SEQ ID NO: 341 - protein, 30 aa, Methanococcus maripaludis
  source 1..30: note = Methanococcus maripaludis / Methanococcus maripaludis OS7 / Methanococcus maripaludis X1 / Methanococcus maripaludis KA1 Adriase recognition motif
EDTGKISDAIKNCISKDPGAFEEDPMVIEL

SEQ ID NO: 342 - protein, 30 aa, Methanothermococcus okinawensis
  source 1..30: note = Adriase recognition motif
EDTGKISSAVKECISKDPGAIDEDPFIIEL

SEQ ID NO: 343 - protein, 30 aa, Methanocaldococcus infernus
  source 1..30: note = Adriase recognition motif
EDIGKITQAIKECLSKDPGAIDEDPYIIQL

SEQ ID NO: 344 - protein, 30 aa, Methanocaldococcus sp.
  source 1..30: note = Methanocaldococcus sp. FS406-22; note = Adriase recognition motif
EDVGKITQAIKECLSKDPGAIDEDPFIIEL

SEQ ID NO: 345 - protein, 30 aa, Methanocaldococcus vulcanius
  source 1..30: note = Adriase recognition motif
EDVGKITQAIKECLSKDPGAIDEDPFIIQL

SEQ ID NO: 346 - protein, 30 aa, Methanocaldococcus jannaschii
  source 1..30: note = Adriase recognition motif
EDIGKITQAIKECLSKDPGAIDEDPFIIEL

SEQ ID NO: 347 - protein, 30 aa, Methanocaldococcus villosus
  source 1..30: note = Adriase recognition motif
EDINKITQAIKECISKDPGAIDEEPMILEL

SEQ ID NO: 348 - protein, 30 aa, Methanocaldococcus fervens
  source 1..30: note = Adriase recognition motif
EDVNKITQAIKECISKDPGAIDEEPIVLEL

SEQ ID NO: 349 - protein, 30 aa, Methanococcus vannielii
  source 1..30: note = Adriase recognition motif
EDKGKITEAIKNCISKDPGALEEDAMVVEL

SEQ ID NO: 350 - protein, 30 aa, Methanotorris igneus
  source 1..30: note = Adriase recognition motif
EDVSKITAAIKECISKDPGAIDEEPMVLDL

SEQ ID NO: 351 - protein, 30 aa, Methanobrevibacter thaueri
  source 1..30: note = Methanobrevibacter thaueri / Methanobrevibacter sp. YE315 Adriase recognition motif
EDIGAIQSKINECVEKDPGAFEEEAMVISV

SEQ ID NO: 352 - protein, 30 aa, Methanobrevibacter smithii
  source 1..30: note = Methanobrevibacter smithii / Methanobrevibacter smithii ATCC 35061 / Methanobrevibacter smithii CAG:186 / Methanobrevibacter smithii DSM 2374 Adriase recognition motif
EDVGAIQAKINECVEKDPGAFEEEAMVISV

SEQ ID NO: 353 - protein, 30 aa, Methanobrevibacter sp.
  source 1..30: note = Methanobrevibacter millerae / Methanobrevibacter oralis Adriase recognition motif
EDIGTIQSKINECVEKDPGAFEEEAMVISV

SEQ ID NO: 354 - protein, 30 aa, Methanobrevibacter woesei
  source 1..30: note = Adriase recognition motif
EDVGAINAKISECVEKDPGAFEEEAMVISV

SEQ ID NO: 355 - protein, 30 aa, Methanothermus fervidus
  source 1..30: note = Adriase recognition motif
EDADQIKEKVKECIEKDPGAFEEEAMVVKV

SEQ ID NO: 356 - protein, 30 aa, Methanothermobacter wolfeii
  source 1..30: note = Methanothermobacter wolfeii / Methanothermobacter marburgensis Adriase recognition motif
EDADAIKAKVKECIEKDPGAFEEEAMIIKV

SEQ ID NO: 357 - protein, 30 aa, Methanobacterium sp.
  source 1..30: note = Adriase recognition motif; note = Methanobacterium sp. SMA-27
EDAAAIQAKVKECIEKDPGAIEEEAMVIVI

SEQ ID NO: 358 - protein, 30 aa, Methanobacterium sp.
  source 1..30: note = Adriase recognition motif; note = Methanobacterium lacus
EDAAAIQAKVKECIEKDPGAIEEEAMVIEI

SEQ ID NO: 359 - protein, 30 aa, Methanobrevibacter curvatus
  source 1..30: note = Adriase recognition motif
EDAGAIESKVKECISKDPGAFEEEALVISV

SEQ ID NO: 360 - protein, 30 aa, Methanobacterium sp.
  source 1..30: note = Methanobacterium paludis; note = Adriase recognition motif
EDPAAISSKIKECIEKDPGAFEEEAMVIEV

SEQ ID NO: 361 - protein, 30 aa, Methanobrevibacter olleyae
  source 1..30: note = Adriase recognition motif
EDGGAITSKVKECIEKDPGAFEEDAMVIEV

SEQ ID NO: 362 - protein, 30 aa, Methanobrevibacter sp.
  source 1..30: note = Methanobrevibacter wolinii / Methanobrevibacter sp. 87.7 Adriase recognition motif
EDGGAITSKVKECIEKDPGAFEEEALVVEV

SEQ ID NO: 363 - protein, 30 aa, Methanobrevibacter ruminantium
  source 1..30: note = Adriase recognition motif
EDGGAITAKVKECIEKDPGAFEEDAMVIEV

SEQ ID NO: 364 - protein, 30 aa, unidentified
  source 1..30: note = Adriase recognition motif; note = Methanobacteriaceae archaeon 41_258
EDPGQIQAKVKECIEKDPGAFEEEAMVIEV

SEQ ID NO: 365 - protein, 30 aa, Methanobrevibacter sp.
  source 1..30: note = Methanobrevibacter arboriphilus / Methanobrevibacter sp. NOE Adriase recognition motif
EDAGAIEAKVKECIEKDPGAFEEEAYVISL

SEQ ID NO: 366 - protein, 30 aa, synthetic construct
  source 1..30: note = Adriase consensus recognition motif
EDMGAITAKVKECISKDPGAFEEEPMVIEV

SEQ ID NO: 367 - protein, 31 aa, synthetic construct
  SITE 1: note = N-term Fluorescein-5-isothiocyanate
  source 1..31: note = (15)KDPGA(10)
  MOD_RES 1: note = Aminohexanoic acid
XEDIGKITQAIKECLSKDPGAIDEDPFIIEL

SEQ ID NO: 368 - protein, 26 aa, synthetic construct
  SITE 1: note = N-term Fluorescein-5-isothiocyanate
  source 1..26: note = (15)KDPGA(5)
  MOD_RES 1: note = Aminohexanoic acid
XEDIGKITQAIKECLSKDPGAIDEDP

SEQ ID NO: 369 - protein, 21 aa, synthetic construct
  SITE 1: note = N-term Fluorescein-5-isothiocyanate
  source 1..21: note = (15)KDPGA(0)
  MOD_RES 1: note = Aminohexanoic acid
XEDIGKITQAIKECLSKDPGA

SEQ ID NO: 370 - protein, 26 aa, synthetic construct
  SITE 26: note = C-term Fluorescein-5-isothiocyanate
  source 1..26: note = (10)KDPGA(10)
ITQAIKECLSKDPGAIDEDPFIIELK

SEQ ID NO: 371 - protein, 21 aa, synthetic construct
  SITE 21: note = C-term Fluorescein-5-isothiocyanate
  source 1..21: note = C-terminal Lys-FITC modification
KECLSKDPGAIDEDPFIIELK

SEQ ID NO: 372 - protein, 16 aa, synthetic construct
  SITE 16: note = C-term Fluorescein-5-isothiocyanate
  source 1..16: note = (0)KDPGA(10)
KDPGAIDEDPFIIELK

SEQ ID NO: 373 - protein, 20 aa, synthetic construct
  source 1..20: note = PGA(17)
PGAIDEDPFIIELEGGKGGG

SEQ ID NO: 374 - protein, 13 aa, synthetic construct
  source 1..13: note = PGA(10)
PGAIDEDPFIIEL

SEQ ID NO: 375 - protein, 10 aa, synthetic construct
  source 1..10: note = PGA(8)
PGAIDEDPFI

SEQ ID NO: 376 - DNA, 582 bp, Methanosarcina mazei
  source 1..582: mol_type = genomic DNA; note = MM_2909 gene; note = Methanosarcina mazei Go1
atgacccttgttattgcttttattggaaagaacggtgcagtcatggcaggagatatgcgcgaaattacttttgaaggggaaaagcccgatagagagaagctcgaaaaagagctttacagcggatcaatagttacggatgaggaaatgcaaaagaaagcagaggagtttggggtcaaaataacggttgcggattgcaaggagaaagtctcggaaagaaacggtgtccttgtaggggaagtttcttcggctgaaggcggagtcgttaaaaaaagaaggctgtatgcctcggcaggaaatttcgcaatagccgaactgatcaatactgagatgactcttacttcgcaggggaagggaagcaatttcatagcattcggaaacgagtttaccaaacaggtagcaaacaagtgttttaaagataactggactaaaaaaagcaatctgcaggatgcggtgaagatcctcatcctgtgcatggaaactgtggccagaaaaacagcttctgtgagcaaacagttcatgattgtgcagactgcatcgaatgctgatgttttgaaagttgtggaaaaggacagaaactgctga

SEQ ID NO: 377 - DNA, 723 bp, Methanosarcina mazei
  source 1..723: mol_type = genomic DNA; note = Methanosarcina mazei Go1; note = MM_1543 gene
atggcagataaaagagaaccagccccaggatggcctatcctgaaaggagaatacgaagtaggcgatgtaaagaactctgtactggtaattacctgcggctcacaccttccggggaagccgattcttgatgcaggtgcagcctgtaccgggtcatgtaagacagaaaaccttggtattgaaaaggtcgtcgcccatatcatctcaaacccgaacatcagatacctgcttgtaaccggctctgaagttaaggggcacattaccggacagtccatgatgagcctccacgcaaacggtgtaaaagaaaacaggattgcaggagcattgggtgcaattccgtatgtggaaaaccttaatgcagccgcagttgcccgtttccaggaacaggttcaggtcgtcaatctgctcgatactgaagatatgggggctatcacctccaaggtgagagaactggcctcaaaagaccctggtgcctttgatgcggacccactggttgtagaaatcagcgaagagggtgaagaagaagaggaaggtggcgttgtaaggccggtttccggagagatcgctgttctccgtagcaggttgaaagcaattgaagcccgcatgatggatataggaaacttgaacaagttccactcaggagttcacgcaggaaaagtcgaaggcgccatgattgggttgacaataaccatatccctgcttgggttattactgctagggaggtaa

SEQ ID NO: 378 - DNA, 882 bp, Methanocaldococcus jannaschii
  source 1..882: mol_type = genomic DNA; note = MJ_0548 gene
gtgagtttaattatttgctactatggtaaaaatggggctgtaattggaggagatagaaggcaaatattttttagaggtagtgaggagaatagaaagattttagaagaaaaactatacagtggggagataaaatctgaagaggaactttataaattggcagaaaagcttaacattaaaattataattgaagatgatagggaaaaagttagaaagatatctgattcagtagtatgtggagaagttaggagcttaggaattgatgcaaagagaaggagggtttatgcaacaaaagggaaatgtgccattgttgatatattaaacgacacagttacaaatcaaacaataaaagaaggttttggaattgtagttcttggaaacagattcttaaaaaagaaagctgaggaggaattaaaaagaacagctaaattattcccaatgatgcctatacaacagatagaagatgcaataaaagaaatttttgagaaattaaagtggcatcctacagtgagtaaagagtatgacatttacagtgtgaataaatatgaaaagaactttgaggaagttattaaaaaggatattgagagcctatttaaatatagggaacagttgaggaaacaactcatagattttggaaaggttatgagtatagtcaataaaattgtaaaaaatggagaaattggagttattaaagatggaaaacttcacttatatgatgattatatagctatcgataagatagacccaaatccaaaggtatttaaagttgtggatgtggaaggcaactttaaagatggtgatatagtagttattgaaaatggagatatgaaaataaaagggactaatgagaaagtaacaaccaaatatatcataattcataaataa

SEQ ID NO: 379 - DNA, 738 bp, Methanocaldococcus jannaschii
  source 1..738: mol_type = genomic DNA; note = MJ_0851 gene
atggtgatgtctatggcaaataaaagagaaccagcaccaggatggccaattgtctctggtgaatatgttgttggaaatccggaaagttgtgttggagttgtaactttaggttctcacggtttggagcaagcatgtatcgatgcaggggctgctatagcaggaccttgccacacagaaaacttgggtattgaaaaggttgtagcaaactatatatcaaacccaaacattaggtttatgattctctgtggttcagaagtccaagggcatataactggacagtgttttaaagcattatgggaaaatggcattggagacgatggaggtattattggagctaaaggggccataccattcttagagaacgtgaataaagaagcagttgaaagatttagaaggcagatagttgaagttgttgatttaattgactgtgaagatattggtaaaataacacaagcaataaaagagtgtttaagtaaagacccaggagctattgacgaagacccatttataattgagttagaaggaggaaaaggaggaggagaagaggaagagggtgttataaaaccaataacaccagaaatggcaataattgagagtagaatgagattaattggaaatgaaatgtgctataatggattgttagcaaagtggcaggcaggatattataatggtaagattcaaggaattgccactggtttgttcttaatgctactaattatgggaatcttaatgttctaa

SEQ ID NO: 380 - protein, 200 aa, synthetic construct
  source 1..200: note = M. mazei Adriase with C-terminal His-tag
TLVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNCGSHHHHHH

SEQ ID NO: 381 - protein, 202 aa, synthetic construct
  source 1..202: note = M. mazei Adriase with C-terminal Strep-tag
TLVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNCGSWSHPQFEK

SEQ ID NO: 382 - protein, 204 aa, synthetic construct
  source 1..204: note = M. mazei Adriase with C-terminal Myc-tag
TLVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNCGSEQKLISEEDL

SEQ ID NO: 383 - protein, 203 aa, synthetic construct
  source 1..203: note = M. mazei Adriase with C-terminal HA-tag
TLVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNCGSYPYDVPDYA

SEQ ID NO: 384 - protein, 200 aa, synthetic construct
  source 1..200: note = M. mazei Adriase T1A mutant with C-terminal His-tag
ALVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNCGSHHHHHH

SEQ ID NO: 385 - protein, 300 aa, synthetic construct
  source 1..300: note = M. jannaschii Adriase with C-terminal His-tag
SLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 386 - protein, 300 aa, synthetic construct
  source 1..300: note = M. jannaschii Adriase S1A mutant with C-terminal His-tag
ALIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 387 - protein, 209 aa, synthetic construct
  source 1..209: note = M. jannaschii Adriase without OB, with C-terminal His-tag
SLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQGSHHHHHH

SEQ ID NO: 388 - protein, 270 aa, synthetic construct
  source 1..270: note = M. jannaschii Adriase 28-57, C-terminal His6
SLIICYYGKNGAVIGGDRRQIFFRGSGKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 389 - DNA, 969 bp, Methanopyrus kandleri
  source 1..969: mol_type = genomic DNA; note = Methanopyrus kandleri Adriase gene
ttgaccctggtactggcctacgccgggacggacggtgctctcgtggccggggaccgtcgcaccctagtcgctcgtatggacgaggaaaagatgcggaaagtggaagaaaaactgtactccggtgagataaggaccgaagaagaattggaatcgttcctgaaagatctcgacgtcgaggatggttacttcgagttccacgacgataggaagaaagtctggaaagtgaacgacgaggtagtggccggagaagtcggcgtgagatcggccaaaggcgttcggaggcgccgcgtttacgcgactcccggcgcccacgctatcgttgagcttgaaggggaaaaggtcctctccaagaatttcggcggccccgccctgatcgtggaaggccccaaggtcgtcaaggaactggtgatcgagttcgtgaactccgagctcggagggaaacccgacctggaatccctccggaacgccctggatgacctcttcgagtacgtgtcctccggtacgatcctcgtgagctccgagtacgatgcgtacgaggtgaagggcaaggcggaccccctggctcgagcccgcctccagaaggccatcgacgaggacatcgaacggctccgcgagcacagacggcgactcgccgaggagatgctcaagcatatccgggaaggttacgatatcttgaaggaaggagtcgtcggcgaggtcgtcgaggtcggcaccgaggaagagggtaaaggggtggaggacgttccccccgagcggcggatcgtcgtcaggctcgcggaggacgtcgacgccaggcacatgggggacgtggtcgccggtcccggtgaggagatcgtcatggccgtcgaaggtaatcctgaggaggtggagccgggagatagggtggtggtgaaagacggggtaatgaagatagatggaaaggacctgcccgtgatcacggggtactcaatctgccgaacgagacgttga

SEQ ID NO: 390 - protein, 229 aa, synthetic construct
  source 1..229: note = M. mazei MtrA delta 219-240 with N-terminal Strep-tag
MWSHPQFEKGSMADKREPAPGWPILKGEYEVGDVKNSVLVITCGSHLPGKPILDAGAACTGSCKTENLGIEKVVAHIISNPNIRYLLVTGSEVKGHITGQSMMSLHANGVKENRIAGALGAIPYVENLNAAAVARFQEQVQVVNLLDTEDMGAITSKVRELASKDPGAFDADPLVVEISEEGEEEEEGGVVRPVSGEIAVLRSRLKAIEARMMDIGNLNKFHSGVHAGK

SEQ ID NO: 391 - protein, 233 aa, synthetic construct
  source 1..233: note = M. jannaschii MtrA delta 225-245 with N-terminal His6-tag
MHHHHHHGSMVMSMANKREPAPGWPIVSGEYVVGNPESCVGVVTLGSHGLEQACIDAGAAIAGPCHTENLGIEKVVANYISNPNIRFMILCGSEVQGHITGQCFKALWENGIGDDGGIIGAKGAIPFLENVNKEAVERFRRQIVEVVDLIDCEDIGKITQAIKECLSKDPGAIDEDPFIIELEGGKGGGEEEEGVIKPITPEMAIIESRMRLIGNEMCYNGLLAKWQAGYYNG

SEQ ID NO: 392 - protein, 105 aa, synthetic construct
  source 1..105: note = Ub-KDPGA(10)
MHHHHHHGSMKIKIVPAVGGGSPLELEVAPNATVGAVRTKVCAMKKLPPDTTRLTYKGRALKDTETLESLGVADGDKFVLITRTVGGCGEKDPGAIDEDPFIIEL

SEQ ID NO: 393 - DNA, 264 bp, synthetic construct
  source 1..264: mol_type = other DNA; note = Caldiarchaeum subterraneum ubiquitin gene (Altered codon usage)
atgaaaattaaaattgtacccgccgtgggtggagggagccctctcgaacttgaagtagctccaaacgcgaccgtgggtgcggtgcgtaccaaggtctgtgcgatgaaaaagttgccgccggatacaacgcgcttaacttataaagggcgcgctctgaaagataccgagactctggagtcgctgggcgtcgcagatggtgacaaatttgttctgatcacccgtacggttggcggctgcggcgaaccgatccgccgggccgcataa

SEQ ID NO: 394 - DNA, 35 bp, synthetic construct
  source 1..35: mol_type = other DNA; note = forward M. mazei Adriase Primer
ggatcgcatatgacccttgttattgcttttattgg

SEQ ID NO: 395 - DNA, 42 bp, synthetic construct
  source 1..42: mol_type = other DNA; note = forward M. mazei Adriase T1A Primer
ggatcgcatatggcccttgttattgcttttattggaaagaac

SEQ ID NO: 396 - DNA, 58 bp, synthetic construct
  source 1..58: mol_type = other DNA; note = reverse M. mazei Adriase-His Primer
ggtaacctcgagttagtggtggtggtggtggtgagaaccgcagtttctgtccttttcc

SEQ ID NO: 397 - DNA, 64 bp, synthetic construct
  source 1..64: mol_type = other DNA; note = reverse M. mazei Adriase-Strep Primer
ggtaacctcgagttacttttcgaactgaggatgagaccaagaaccgcagtttctgtccttttcc

SEQ ID NO: 398 - DNA, 70 bp, synthetic construct
  source 1..70: mol_type = other DNA; note = reverse M. mazei Adriase-Myc Primer
ggtaacctcgagttacaagtcctcctctgaaatcaatttttgctcagaaccgcagtttctgtccttttcc

SEQ ID NO: 399 - DNA, 67 bp, synthetic construct
  source 1..67: mol_type = other DNA; note = reverse M. mazei Adriase-HA Primer
ggtaacctcgagttaagcataatcagggacatcatacgggtaagaaccgcagtttctgtccttttcc

SEQ ID NO: 400 - DNA, 44 bp, synthetic construct
  source 1..44: mol_type = other DNA; note = forward M. jannaschii Adriase Primer
cgtagtcatatgagtttaattatttgctactatggtaaaaatgg

SEQ ID NO: 401 - DNA, 46 bp, synthetic construct
  source 1..46: mol_type = other DNA; note = forward M. jannaschii Adriase S1A Primer
cgtagtcatatggcgttaattatttgctactatggtaaaaatgggg

SEQ ID NO: 402 - DNA, 594 bp, Methanoculleus marisnigri
  source 1..594: mol_type = genomic DNA; note = Methanoculleus marisnigri Adriase gene
atgacgcttgtgatcgcctttatcggaaaacagggcgccgtgatggcgggggatatgcgggagatcgcattcggaggggacgattcctccatcgaagacctggagcgcgaactctacggcggctcgatcgcctccgacagcgacctcgcaaaacgggcagacgagatcggggtgacgatccagctcagggacgacaaaaccaaggtctcgcagcgggacggggtgctcgtcggcgaggtgaccgagacggagggggcgaagacctcgagaaaaaggctctacgctacgaaaggaagttacgtgattgccgagatcgtcgattcccggctgcgggtgatgcaaaaggggcaggcgagcaacttcgtggtgctgggaaacgataccacgaaagaggttgccaaccagtgcatccaggggatgtgggaagggggaacgatccaggatgccctgcgtctcatcatgctcaccatgcagatagcggcaagcgtaacggcgtcggtgagcagaacgttcgtactggtgcataccgatatcgcggcggatgtcccggccgctatcgctaacgattctcgaaaagaacaaaacccggattga

SEQ ID NO: 403 - DNA, 83 bp, synthetic construct
  source 1..83: mol_type = other DNA; note = reverse M. jannaschii Adriase-His Primer
ggtaacctcgagttagtggtggtggtggtggtgagaacctttatgaattatgatatatttggttgttactttctcattagtcc

SEQ ID NO: 404 - DNA, 72 bp, synthetic construct
  source 1..72: mol_type = other DNA; note = reverse M. jannaschii Adriase- OB-His Primer
ggtaacctcgagttagtggtggtggtggtggtgagaaccctgttccctatatttaaataggctctcaatatc

SEQ ID NO: 405 - DNA, 60 bp, synthetic construct
  source 1..60: mol_type = other DNA; note = forward M. jannaschii Adriase delta 28-57
gcaaatattttttagaggtagtggcaaaattataattgaagatgatagggaaaaagttag

SEQ ID NO: 406 - DNA, 60 bp, synthetic construct
  source 1..60: mol_type = other DNA; note = reverse M. jannaschii Adriase delta28-57
ctaactttttccctatcatcttcaattataattttgccactacctctaaaaaatatttgc

SEQ ID NO: 407 - DNA, 65 bp, synthetic construct
  source 1..65: mol_type = other DNA; note = forward M. mazei Strep-MtrA Primer
ggatcgcatatgtggtctcatcctcagttcgaaaagggttctatggcagataaaagagaaccagc

SEQ ID NO: 408 - DNA, 35 bp, synthetic construct
  source 1..35: mol_type = other DNA; note = reverse M. mazei MtrA delta219-240 Primer
ggtaacctcgagttattttcctgcgtgaactcctg

SEQ ID NO: 409 - DNA, 64 bp, synthetic construct
  source 1..64: mol_type = other DNA; note = forward M. janaschii His-MtrA Primer
ggatcgcatatgcaccaccaccaccaccacggttctatggtgatgtctatggcaaataaaagag

SEQ ID NO: 410 - DNA, 38 bp, synthetic construct
  source 1..38: mol_type = other DNA; note = reverse M. jannaschii MtrA delta 225-240 Primer
ggtaacctcgagttaaccattataatatcctgcctgcc

SEQ ID NO: 411 - DNA, 63 bp, synthetic construct
  source 1..63: mol_type = other DNA; note = forward His-Ub-KDPGA(10) Primer
ggatcgcatatgcaccaccaccaccaccacggttctatgaaaattaaaattgtacccgccgtg

SEQ ID NO: 412 - DNA, 78 bp, synthetic construct
  source 1..78: mol_type = other DNA; note = everse Ub-KDPGA(10) Primer
ggtaacctcgagttacagttcgatgatgaacgggtcttcgtcgatagcacccgggtctttttcgccgcagccgccaac

SEQ ID NO: 413 - protein, 38 aa, synthetic construct
  SITE 1: note = N-term Fluorescein-5-isothiocyanate
  source 1..38: note = M. jannaschii (F15)KDPGA(17)
  MOD_RES 1: note = Aminohexanoic acid
XEDIGKITQAIKECLSKDPGAIDEDPFIIELEGGKGGG

SEQ ID NO: 414 - protein, 14 aa, synthetic construct
  SITE 14: note = C-term Fluorescein-5-isothiocyanate
  source 1..14: note = M. jannaschii PGA(10F)
PGAIDEDPFIIELK

SEQ ID NO: 415 - protein, 32 aa, synthetic construct
  source 1..32: note = M. jannaschii (10)KDPGA(17)
ITQAIKECLSKDPGAIDEDPFIIELEGGKGGG

SEQ ID NO: 416 - protein, 37 aa, synthetic construct
  source 1..37: note = M. jannaschii (15)KDPGA(17)
EDIGKITQAIKECLSKDPGAIDEDPFIIELEGGKGGG

SEQ ID NO: 417 - protein, 27 aa, synthetic construct
  source 1..27: note = M. jannaschii (5)KDPGA(17)
KECLSKDPGAIDEDPFIIELEGGKGGG

SEQ ID NO: 418 - protein, 31 aa, synthetic construct
  SITE 26: note = C-term Fluorescein-5-isothiocyanate
  source 1..31: note = M. jannaschii (15)KDPGA(10F)
EDIGKITQAIKECLSKDPGAIDEDPFIIELK

SEQ ID NO: 419 - protein, 30 aa, synthetic construct; identical to SEQ ID NO: 346
  source 1..30: note = M. jannaschii (15)KDPGA(10)

SEQ ID NO: 420 - protein, 202 aa, Methanocaldococcus jannaschii
  source 1..202: note = M. jannaschii His-MtrA delta 194-245
MHHHHHHGSMVMSMANKREPAPGWPIVSGEYVVGNPESCVGVVTLGSHGLEQACIDAGAAIAGPCHTENLGIEKVVANYISNPNIRFMILCGSEVQGHITGQCFKALWENGIGDDGGIIGAKGAIPFLENVNKEAVERFRRQIVEVVDLIDCEDIGKITQAIKECLSKDPGAIDEDPFIIELEGGKGGGEEEEGVIKPITPE

SEQ ID NO: 421 - protein, 20 aa, synthetic construct
  source 1..20: note = M. jannaschii (5)KDPGA(10)
KECLSKDPGAIDEDPFIIEL

SEQ ID NO: 422 - protein, 87 aa, unidentified
  source 1..87: note = Candidatus Caldiarchaeum subterraneum; note = Caldiarchaeum subterraneum Ubiquitin
MKIKIVPAVGGGSPLELEVAPNATVGAVRTKVCAMKKLPPDTTRLTYKGRALKDTETLESLGVADGDKFVLITRTVGGCGEPIRRAA

SEQ ID NO: 423 - protein, 245 aa, Methanocaldococcus jannaschii
  source 1..245: note = M. jannaschii MtrA
MVMSMANKREPAPGWPIVSGEYVVGNPESCVGVVTLGSHGLEQACIDAGAAIAGPCHTENLGIEKVVANYISNPNIRFMILCGSEVQGHITGQCFKALWENGIGDDGGIIGAKGAIPFLENVNKEAVERFRRQIVEVVDLIDCEDIGKITQAIKECLSKDPGAIDEDPFIIELEGGKGGGEEEEGVIKPITPEMAIIESRMRLIGNEMCYNGLLAKWQAGYYNGKIQGIATGLFLMLLIMGILMF

SEQ ID NO: 424 - protein, 64 aa, Methanosarcina mazei
  source 1..64: note = M. mazei MtrA 155-218; note = Methanosarcina mazei Go1
PGAFDADPLVVEISEEGEEEEEGGVVRPVSGEIAVLRSRLKAIEARMMDIGNLNKFHSGVHAGK

SEQ ID NO: 425 - protein, 11 aa, synthetic construct
  source 1..11: note = KDTLVIAFIGK
KDTLVIAFIGK

SEQ ID NO: 426 - protein, 10 aa, synthetic construct
  source 1..10: note = DTLVIAFIGK
DTLVIAFIGK

SEQ ID NO: 427 - protein, 182 aa, synthetic construct
  source 1..182: note = MtrAN-UbC Fusion
MHHHHHHGSMVMSMANKREPAPGWPIVSGEYVVGNPESCVGVVTLGSHGLEQACIDAGAAIAGPCHTENLGIEKVVANYISNPNIRFMILCGSEVQGHITGQCFKALWENGIGDDGGIIGAKGAIPFLENVNKEAVERFRRQIVEVVDLIDCEDIGKITQAIKECLSKDPGAIDEDPFIIEL

SEQ ID NO: 428 - protein, 125 aa, synthetic construct
  source 1..125: note = UbN-MtrAC Fusion
MHHHHHHGSMKIKIVPAVGGGSPLELEVAPNATVGAVRTKVCAMKKLPPDTTRLTYKGRALKDTETLESLGVADGDKFVLITRTVGGCGEKDPGAIDEDPFIIELEGGKGGGEEEEGVIKPITPE

SEQ ID NO: 429 - protein, 318 aa, synthetic construct
  SITE 1: note = N-term Fluorescein-5-isothiocyanate
  source 1..318: note = M. jannaschii (F15)KD-Adriase
  MOD_RES 1: note = Aminohexanoic acid
XEDIGKITQAIKECLSKDSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 430 - DNA, 876 bp, Methanococcus vannielii
  source 1..876: mol_type = genomic DNA; note = Methanococcus vannielii Adriase gene
atgagcgtaatcatcggttactatggaaaaaacggcgtagtgattgcaggagataaaagaaccattttatttaatggcgaagaaaaaaaacgtgaaatgcttgaagaacttctttattctggaaaaataaaaaacgatgatgatttatcagaaaaatcagccgaattttctgtaaaagtccatattcttgatgaccagaaaaaaattaaacagcttgaaaatacccttattggtgaagtaagaactattggaaaagattcaaagcgaaaaaaaatgtatctttcaaaggacatttgtgtaatactcgatattgaaaacgacaatattgtaaaaaaatcagtaaaaaccggaaccggaatagttgtttttgggaataaatacgtgaaaaaaattgttgaattagaacttaaaaaatacttatccgaaatttcaaagatggatgttttaaaaatcagaacaatttttgaaaaaatattagttggaattgaaaacccgacattgagtaaaggttatgattttttatactgtcaaaatgctgaaaaagatattgaaagcataattaaaaaagatttaaatgatttacgtgaatatcgggaagaactatcgataaaaatggcggaaatgcagaaattaatgactattgtaaacaaaatcgcaaaatcaggcgaaattggaattataaaaaatggtagcttagttttaaatgaaagtttccttgcaattgataaaatttgtttaaaccccgaaatctactctgaaatagatataaacggtaattttaaagaaggagatcttattgaaatccaaaacggggaattaactgttaaaggaacgaataattatgtttctgttcaaaaaataatatgtaatcgttaa

SEQ ID NO: 431 - protein, 365 aa, Methanosarcina mazei
  source 1..365: note = Methanosarcina mazei Go1; note = M. mazei MtrA_N-Adriase conjugate
MWSHPQFEKGSMADKREPAPGWPILKGEYEVGDVKNSVLVITCGSHLPGKPILDAGAACTGSCKTENLGIEKVVAHIISNPNIRYLLVTGSEVKGHITGQSMMSLHANGVKENRIAGALGAIPYVENLNAAAVARFQEQVQVVNLLDTEDMGAITSKVRELASKDTLVIAFIGKNGAVMAGDMREITFEGEKPDREKLEKELYSGSIVTDEEMQKKAEEFGVKITVADCKEKVSERNGVLVGEVSSAEGGVVKKRRLYASAGNFAIAELINTEMTLTSQGKGSNFIAFGNEFTKQVANKCFKDNWTKKSNLQDAVKILILCMETVARKTASVSKQFMIVQTASNADVLKVVEKDRNCGSHHHHHH

SEQ ID NO: 432 - protein, 64 aa, Methanosarcina mazei; identical to SEQ ID NO: 424
  source 1..64: note = Methanosarcina mazei Go1; note = M. mazei MtrA_C fragment

SEQ ID NO: 433 - protein, 392 aa, synthetic construct
  source 1..392: note = M. jannaschii Ub_N-Adriase intermediate
MHHHHHHGSMKIKIVPAVGGGSPLELEVAPNATVGAVRTKVCAMKKLPPDTTRLTYKGRALKDTETLESLGVADGDKFVLITRTVGGCGEKDSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 434 - protein, 469 aa, Methanocaldococcus jannaschii
  source 1..469: note = M. jannaschii MtrA_N-Adriase intermediate
MHHHHHHGSMVMSMANKREPAPGWPIVSGEYVVGNPESCVGVVTLGSHGLEQACIDAGAAIAGPCHTENLGIEKVVANYISNPNIRFMILCGSEVQGHITGQCFKALWENGIGDDGGIIGAKGAIPFLENVNKEAVERFRRQIVEVVDLIDCEDIGKITQAIKECLSKDSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 435 - protein, 224 aa, Methanocaldococcus jannaschii
  source 1..224: note = >WP_010870749.1
MDVMKGTTTVGLICDDAVILATDKRASLGNLVADKEAKKLYKIDDYIAMTIAGSVGDAQAIVRLLIAEAKLYKMRTGRNIPPLACATLLSNILHSSRMFPFLTQIIIGGYDLLEGAKLFSLDPLGGMNEEKTFTATGSGSPIAYGVLEAGYDRDMSVEEGIKLALNALKSAMERDTFSGNGISLAVITKDGVKIFEDEEIEKILDSMKAKPKKKTTKRSRRKSK

SEQ ID NO: 436 - protein, 241 aa, unidentified
  source 1..241: note = Candidatus Methanoperedens sp. BLZ2; note = Candidatus Methanoperedens sp. BLZ2 MtrA; note = Candidatus Methanoperedens sp.
MANKVKPAAGWPVVKGEYDAGNPENCVAVTTCGSHLKGAPQIAAGACITGPHKTENLGIEKIVANIISNPNIRFLLVTGAEVKGHISGEAIIMLHKNGIKDNRIVGSTGAIPYIENLPDDAVKRFQAQVQIVEMMGTEDENAIIAKIKELAAKDPGAFDGEPMIIQVGEAKEAEELGGVKPMSAELASIQARIKGVQQQTIDIGNMNKLMSGIYAGKIEGMMIGLVVGLTILGILIFGGGV

SEQ ID NO: 437 - protein, 239 aa, Methanobacterium formicicum
  source 1..239: note = Methanobacterium formicicum MtrA
MAEKKSPAEGWPVINGDYVVGDPESPVAATTLASHIEQIAVDAGAAISGPCKTENLGIEKMLANLISNPNIRFLILTGSEVQGHITGQSIEALHENGVDPEKRKIVGATGAIPFIENIPDEGIERFQQQMEIVSIIDVEDAATIQSKVKECIDKDPGAFEEEAMVIVVEEDGEEEEGEEVPPVAPETALIEARMRNIQTQVKSIGGLNRMAAGMYSGKVQGIMIGLVFILTIGTILLLK

SEQ ID NO: 438 - protein, 238 aa, Methanobrevibacter smithii
  source 1..238: note = Methanobrevibacter smithii MtrA
MADKKEPAAGWPVISGDYIVGDPESPVAVTTLASHIEAELSGAAIAGPCKTENLGVEKVVANIISNPNIRFLILAGAEVQGHITGQSFKALHENGADPDKKKIIGATGAIPFVENVPLEGVERFQQQLEIIDLIDTEDVGAIQAKINECVEKDPGAFEEEAMVISVEGDDGEEDDGEEIKVVSAETALIEARMRNINTKIDMVGSIQRNLAGNYAGKVQGIMIGLAFSLVIGALFLLF

SEQ ID NO: 439 - protein, 239 aa, Methanococcus maripaludis
  source 1..239: note = Methanococcus maripaludis MtrA
MADKKAPAAGWPVANGEYVVGNPESCVAVVTLGSHGLDQAAIDAGAAISGPCHTENLGIEKVVANYISNPNIRFMVITGSEVQGHITGQCIKALYENGIGDDGGIIGAKGAIPFMENVGTEPVGRLQSQIVECIDLIDVEDTGKISDAIKNCISKDPGAFEEEPMVIELEGGAAAAGEESTSIKPTSPEMALLEARMRIVSEKMNEAAMIAKFNSGYYNGKIQGIAIGLFLSIVIFSLL

SEQ ID NO: 440 - protein, 240 aa, Methanoculleus marisnigri
  source 1..240: note = Methanoculleus marisnigri MtrA
MVEKKSPASGWPIVQGDFHTGDAQSCVGVVTMGSHLDEQGICDAGAAIAGSCKTENLGLEKIIANVISNPNIRFILCCGTEVKGHLSGQSFIALHEGGVSGGKIVGAQGAIPFIENLSDDAISRFQEQIEIVNIMESEDMGAIKAKIDELKARDPGAFGAEPMIVEVKEAGGAAEEATGEVQPLSGELALVHARMKVIERMVTDIGYRNKFAAGVYSGKIEGLMIGLIVSFVILGFILLG

SEQ ID NO: 441 - protein, 242 aa, Methanofollis ethanolicus
  source 1..242: note = Methanofollis ethanolicus MtrA
MADKTSPASGWPLVKGDFHSGDANSCVAVVTMGSHLDEQAICDAGAALCGSCKTENLGLEKVIANIVANPNIRFVLLCGTEVKGHLSGQTLKALHESGIEGGKVVGSQGAIPFIENLDDAAIKRFQAQTEFVDIMESEDLGAIKAKIAELKGKDPGAFGEAPIVVEVKEEGGGAEEEGGEIVEMNGDLALIHARFKTIEKMVTDIGYRNRFAAGVYSGKVEGLMIGLIVSFALLGFLWMGAL

SEQ ID NO: 442 - protein, 241 aa, Methanolinea tarda
  source 1..241: note = Methanolinea tarda MtrA
MAEKKSPASGWPIVQGDFHAGDPQSPVAVMTCGSHLDEKAICDGGAAIAGSCKTENLGLEKVIANIISNPNIRFLLLCGTEVKGHLAGQTMAALHKNGVKDGRVVGAEGAIPFIENLKEDAIKRFQEQVELVNIMEAEDLGAIKAKIAELKGRDPGAFAADPMVVEVKEAAGGGAEEAGGVVQPMSGELALIHARMKVIEKMVTDIGYRDKFAAGVYSGKIEGIMIGLIVSFAILGFMLMG

SEQ ID NO: 443 - protein, 252 aa, Methanopyrus kandleri
  source 1..252: note = Methanopyrus kandleri MtrA
MLEVPEKAEPAEGWPVVEGDYVVGDPEAPVHVVTLGSHIEEDILKAAGEDKVAIAGPCKTENIGIEKVIANVIANPNIRFGVLCGAEVTGHLTGQCFKAMYENGVDPDSGEIIGAEGAIPYLENIPEEAVERYRDQIVELVDLIDVEDVDEIVKAIEECVEKDPGAYEEGPMTISLEEEEEEELAEVAGMPVSAETVTVEYRINDVRVGVKSIGAMQRYMAGYLSGRTMGLLIGIISGMIFLFLPMVVLGGV

SEQ ID NO: 444 - protein, 242 aa, unidentified
  source 1..242: note = Methanosaeta harundinacea MtrA; note = Methanosaeta harundinacea
MANKKPTSDPWPWLTGEYVVGNPESPVAVVTCGSHLNNAPFIDAGSALAGPCKTENIGIEKMVANIISNPNIRFLIITGMEVKGHITGQAIEAFAANGIDQEGRIVGATGAIPFIQNLTPEAIERFKQQITTINIIDTEDTGTIVSKIKECVSKDPGALDVEPMAVEISEGEAEEEAHGLRPMAGEIANIRARMRQIDAAVMNMGIQNKYSAGIYAGKIEGIAAGLTIVLVLFGLILSIGGM

SEQ ID NO: 445 - protein, 240 aa, Methanosarcina mazei
  source 1..240: note = Methanosarcina mazei MtrA
MADKREPAPGWPILKGEYEVGDVKNCVLVITCGSHLPGKPILDAGAACTGSCKTENLGIEKVVAHIISNPNIRYLLVTGSEVKGHITGQSMMSLHANGVKENRIAGALGAIPYVENLNAAAVARFQEQVQVVNLLDTEDMGAITSKVRELASKDPGAFDADPLVVEISEEGEEEEEGGVVRPVSGEIAVLRSRLKAIEARMMDIGNLNKFHSGVHAGKVEGAMIGLTITISLLGLLLLGR

SEQ ID NO: 446 - protein, 238 aa, Methanothermobacter thermautotrophicus
  source 1..238: note = Methanothermobacter thermautotrophicus MtrA
MVEKKSPAEGWPVVNGDYIVGDPESPVAATTLASHIEDVPVEAGAAIAGPCKTENLGIEKMIANLISNPNIRFLILCGSEVQGHITGQSIEALHQNGVDPDKRNIIGATGAIPYIENIPDEGIERFQKQLEIVNLIDVEDADAIKAKVKECIEKDPGAFEEEAMVIKVEEGGEEEEGEEVKPVAPETALIEARMRNIQTQVKMIGSTNRMFAGMYSGKVQGIMIGLAFTLTLGILLLV

SEQ ID NO: 447 - protein, 237 aa, Methanothermus fervidus
  source 1..237: note = Methanothermus fervidus MtrA
MAEKKEPAEGWPVVSGDYIVGDPESPVAAVTLASHIEDIPIDAGAAIAGPCKTENLGIEKVIANIISNPNIRFLVLCGSEVQGHITGQSIKALHKNGVDDKKKIIGAKGAIPYIENLPEEALERFQKQVEIVDLIDVEDADQIKEKVKECIEKDPGAFEEEAMVVKVEEEEEEGEAEKINPISPETALIEARIRDMQTQIMMVGGIQRMWAGMYAGKIQGTGIGLALTLLIGFILLI

SEQ ID NO: 448 - protein, 243 aa, Methanothrix thermoacetophila
  source 1..243: note = Methanothrix thermoacetophila PT; note = Methanothrix thermoacetophila PT MtrA
MVLKKKPAEPWPVITGEYEVGNPESPVAVVTCGSHLNNAELVAAGAALAGPCKTENIGIEKMVANVISNPNIRYLIITGMEVKGHITGQAIEAFTASGIDKEGRIVGAKGAIPFIQNLTPEAIERWRQQITTINMIDTEDMGAITAKIKELVAKDPGALDVEPMIVEIKEGAGEEGEAGLRPMAAEVVEVRSRIRAMDMAVTNNGLLNKFQAGVYVGKIEGIAVGMTLTLAIFGIILKMVGGS

SEQ ID NO: 449 - protein, 243 aa, Methanotorris igneus
  source 1..243: note = Methanotorris igneus MtrA
MANKKAPAAGWPIVSGEYVVGNPESCVGVVTLGSHGLEQACIDAGAAIAGPCHTENLGIEKVVANYISNPNIRFMVLCGSEVQGHITGQCFKALWENGIGDDGGIIGAKGAIPFLENVGKDAVERFQRQIVEVVDLIDCEDVSKITAAIKECISKDPGAIDEEPMVLDLEGGGAGGEEEGATVKPTSPEIALIEARMRLISEKINDAAILAKYNAGYYNGKVQGIAIGLFLSLLLFSLIHPLL

SEQ ID NO: 450 - protein, 313 aa, Methanocaldococcus jannaschii
  source 1..313: note = N-terminally modified M. jannaschii Adriase
MGSSHHHHHHSSGLVPRGSHMSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 451 - DNA, 35 bp, Methanocaldococcus jannaschii
  source 1..35: mol_type = other DNA; note = forward N-terminally modified M. jannaschii Adriase Primer
ggcggccatatgagtttaattatttgctactatgg

SEQ ID NO: 452 - DNA, 35 bp, Methanocaldococcus jannaschii
  source 1..35: mol_type = other DNA; note = reverse N-terminally modified M. jannaschii Adriase Primer
cgcctcgagttatttatgaattatgatatatttgg

SEQ ID NO: 453 - protein, 21 aa, synthetic construct
  SITE 21: note = C-term Fluorescein-5-isothiocyanate
  source 1..21: note = M. mazei (5)KDPGA(10F)
RELASKDPGAFDADPLVVEIK

SEQ ID NO: 454 - protein, 13 aa, synthetic construct
  source 1..13: note = M. mazei PGA(10)
PGAFDADPLVVEI

SEQ ID NO: 455 - protein, 13 aa, synthetic construct
  source 1..13: note = M. mazei PGA(10F)
PGAFDADPLVVEI

SEQ ID NO: 456 - protein, 26 aa, synthetic construct
  SITE 1: note = N-term Fluorescein-5-isothiocyanate
  source 1..26: note = M. mazei (F15)KDPGA(10)
  MOD_RES 1: note = Aminohexanoic acid
XITSKVRELASKDPGAFDADPLVVEI

SEQ ID NO: 457 - protein, 28 aa, synthetic construct
  source 1..28: note = M. mazei PGA(25)
PGAFDADPLVVEISEEGEEEEEGGVVRP

SEQ ID NO: 458 - protein, 13 aa, synthetic construct
  SITE 1: note = N-term Fluorescein-5-isothiocyanate
  source 1..13: note = M. mazei (F10)KD
  MOD_RES 1: note = Aminohexanoic acid
XITSKVRELASKD

SEQ ID NO: 459 - protein, 41 aa, synthetic construct
  SITE 1: note = N-term Fluorescein-5-isothiocyanate
  source 1..41: note = M. mazei (F10)KDPGA(25)
  MOD_RES 1: note = Aminohexanoic acid
XITSKVRELASKDPGAFDADPLVVEISEEGEEEEEGGVVRP

SEQ ID NO: 460 - protein, 30 aa, Methanobrevibacter filiformis
  source 1..30: note = Adriase recognition motif
EDAAAIESKVKECIEKDPGAFEEEALVISV

SEQ ID NO: 461 - protein, 30 aa, Methanobacterium sp.
  source 1..30: note = Adriase recognition motif; note = Methanobacterium sp. PtaU1.Bin097
EDAAAIQSKIKECIEKDPGAFEEEAMVVKV

SEQ ID NO: 462 - protein, 30 aa, Methanobacterium sp.
  source 1..30: note = Methanobacterium sp. PtaU1.Bin097; note = Secondary Adriase recognition motif
EDRSEITAQIQGCIEKDPGAVEEETVVIKL

SEQ ID NO: 463 - protein, 30 aa, unidentified
  source 1..30: note = Methanobacteriales archaeon HGW-Methanobacteriales-1; note = Adriase recognition motif
EDAAAIQSKVKECIEKDPGAFEEEAMVIKV

SEQ ID NO: 464 - protein, 30 aa, Methanobacterium sp.
  source 1..30: note = Methanobacterium sp. BRmetb2; note = Adriase recognition motif
EDAAAIQSKVKECVEKDPGAFEEEAMVIEI

SEQ ID NO: 465 - protein, 30 aa, Methanobacterium sp.
  source 1..30: note = Methanobacterium sp. Maddingley MBC34 / Methanobacterium sp. PtaB.Bin024 / Methanobacteriales archaeon HGW-Methanobacteriales-2 / Methanobacterium subterraneum Adriase recognition motif
EDAATIQSKVKECIDKDPGAFEEEAMVIEV

SEQ ID NO: 466 - protein, 30 aa, Methanobacterium sp.
  source 1..30: note = Methanobacterium sp. Maddingley MBC34; note = Secondary Adriase recognition motif
EDAAAIQSKVKDCIEKDPGAVESETMIIKL

SEQ ID NO: 467 - protein, 30 aa, Methanobacterium sp.
  source 1..30: note = Methanobacterium sp. PtaB.Bin024; note = Secondary Adriase recognition motif
EDAAAIQSQVKECIEKDPGAAEEETMVIEI

SEQ ID NO: 468 - protein, 30 aa, Methanobacterium subterraneum
  source 1..30: note = Secondary Adriase recognition motif
EDATTIQSKVKECIEKDPGAVESETMVIEL

SEQ ID NO: 469 - protein, 30 aa, Methanobacterium sp.
  source 1..30: note = Methanobacterium formicicum DSM 3637 / Methanobacterium sp. BAmetb5 / Methanobacterium sp. MB1 Adriase recognition motif
EDAATIQSKVKECIDKDPGAFEEEAMVIKV

SEQ ID NO: 470 - protein, 30 aa, Methanobacterium formicicum
  source 1..30: note = Methanobacterium formicicum /Methanobacterium formicicum BRM9 / Methanobacterium formicicum DSM1535 / Methanobacterium formicicum MB9 Adriase recognition motif
EDAATIQSKVKECIDKDPGAFEEEAMVIVV

SEQ ID NO: 471 - protein, 30 aa, Methanothermobacter sp.
  source 1..30: note = Methanothermobacter defluvii / Methanothermobacter sp. CaT2 / Methanothermobacter sp. EMTCatA1 / Methanothermobacter thermautotrophicus Adriase recognition motif
EDADAIKAKVKECIEKDPGAFEEEAMVIKV

SEQ ID NO: 472 - protein, 30 aa, Methanobacterium sp.
  source 1..30: note = Adriase recognition motif
EDAAAIQSKVKECVEKDPGAFEEEAMVIKV

SEQ ID NO: 473 - protein, 30 aa, Methanobrevibacter sp.
  source 1..30: note = Adriase recognition motif; note = Methanobrevibacter arboriphilus; note = Methanobrevibacter arboriphilus DSM 1125
EDAGAIEAKVKECIEKDPGAFEEDAYVISL

SEQ ID NO: 474 - protein, 30 aa, Methanobacterium bryantii
  source 1..30: note = Adriase recognition motif
EDAGQIQAKVKECIEKDPGAFEEEAMVIKV

SEQ ID NO: 475 - protein, 30 aa, Methanobacterium bryantii
  source 1..30: note = Secondary Adriase recognition motif
EDAGQIQGKVKECIEKDPGAYEEEPIAIRL

SEQ ID NO: 476 - protein, 30 aa, Methanobrevibacter wolinii
  source 1..30: note = Secondary Adriase recognition motif
EDGGAITSKVKECIEKDPGAFEEKSMVISI

SEQ ID NO: 477 - protein, 30 aa, Methanobrevibacter sp.
  source 1..30: note = Methanobrevibacter sp. YE315 / Methanobrevibacter thaueri Secondary Adriase recognition motif
EDIGAIQSKINECVEKDPGALEEDPIVMEV

SEQ ID NO: 478 - protein, 30 aa, unidentified
  source 1..30: note = Adriase recognition motif; note = Methanococcales archaeon HHB
EDIGKITQAIKECISKDPGATEEDAEEIDL

SEQ ID NO: 479 - protein, 30 aa, Methanobrevibacter gottschalkii
  source 1..30: note = Adriase recognition motif
EDIGNIQSKINECIEKDPGALEEDPIVMEV

SEQ ID NO: 480 - protein, 30 aa, Methanococcus maripaludis
  source 1..30: note = Methanococcus maripaludis C5; note = Adriase recognition motif
EDKGKIADAIKNCISKDPGAFEEDPMVIEL

SEQ ID NO: 481 - protein, 30 aa, unidentified
  source 1..30: note = Adriase recognition motif; note = Methanoregulaceae archaeon PtaU1.Bin059
EDLGAIKAKINELKGRDPGAFAADPMVVEV

SEQ ID NO: 482 - protein, 30 aa, Methanoculleus chikugoensis
  source 1..30: note = Adriase recognition motif
EDMGAIKAKIDELKAKDPGAFGAEPMIVEV

SEQ ID NO: 483 - protein, 30 aa, Methanoculleus sp.
  source 1..30: note = Methanoculleus sediminis; note = Secondary Adriase recognition motif
EDMGAIKAKIDELKARDPGAFGAEPMVVEV

SEQ ID NO: 484 - protein, 30 aa, Methanoculleus sp.
  source 1..30: note = Methanoculleus sp. MH98A; note = Secondary Adriase recognition motif
EDMGVIKAKIDELKARDPGAFGAEPMVVEV

SEQ ID NO: 485 - protein, 30 aa, unidentified; identical to SEQ ID NO: 465
  source 1..30: note = Methanobacteriales archaeon HGW-Methanobacteriales-2; note = Adriase recognition motif

SEQ ID NO: 486 - protein, 30 aa, Methanoculleus sp.; identical to SEQ ID NO: 332
  source 1..30: note = Secondary Adriase recognition motif; note = Methanoculleus sp. SDB

SEQ ID NO: 487 - protein, 30 aa, Methanoculleus sp.
  source 1..30: note = Secondary Adriase recognition motif; note = Methanoculleus horonobensis
EDMGAIKAKMDELKARDPGAFGAEPMIVEV

SEQ ID NO: 488 - protein, 30 aa, Methanoculleus sp.
  source 1..30: note = Methanoculleus taiwanensis; note = Adriase recognition motif
EDMGAISAKIKELTARDPGAFGADPMIIEV

SEQ ID NO: 489 - protein, 30 aa, Methanoculleus sp.
  source 1..30: note = Methanoculleus taiwanensis; note = Secondary Adriase recognition motif
EDMGAISAKIKELTARDPGAFGAEPMIIEV

SEQ ID NO: 490 - protein, 30 aa, Methanosarcina sp.
  source 1..30: note = Methanosarcina sp. 1.H.A.2.2 / Methanosarcina sp. 2.H.A.1B.4 Adriase recognition motif
EDMGAITAKVRELASKDPGAFDAEPMVVVI

SEQ ID NO: 491 - protein, 30 aa, unidentified
  source 1..30: note = Methanosaeta sp. PtaU1.Bin112; note = Adriase recognition motif; note = Methanosaeta sp.
EDMGAITGKIKELVSKDPGALDVDPMTIEI

SEQ ID NO: 492 - protein, 30 aa, Methanobacterium sp.
  source 1..30: note = Adriase recognition motif
EDPATISAKVKECIEKDPGAFEEEAMVVEV

SEQ ID NO: 493 - protein, 30 aa, Methanosarcina barkeri
  source 1..30: note = Methanosarcina barkeri 3; note = Adriase recognition motif
EDMGAITSKVRELASKDPGAFEAEPLVVEI

SEQ ID NO: 494 - protein, 30 aa, Methanoculleus bourgensis
  source 1..30: note = Secondary Adriase recognition motif; note = Methanoculleus bourgensis MAB1
EDMGTIKAKIDELKAKDPGAFPEEPMVVEV

SEQ ID NO: 495 - protein, 30 aa, Methanoculleus sp.; identical to SEQ ID NO: 494
  source 1..30: note = Adriase recognition motif; note = Methanoculleus sp. MAB1

SEQ ID NO: 496 - protein, 30 aa, Methanobacterium congolense
  source 1..30: note = Adriase recognition motif
EDPASIQSKVKECIEKDPGAFEEEAMVIEI

SEQ ID NO: 497 - protein, 30 aa, Methanothermobacter sp.
  source 1..30: note = Methanothermobacter tenebrarum; note = Adriase recognition motif
EDPEQIKAKVKECIEKDPGAFEEEAMVIEV

SEQ ID NO: 498 - protein, 30 aa, Methanothermobacter sp.
  source 1..30: note = Methanothermobacter sp. MT-2; note = Adriase recognition motif
EDPGPIQSKVKECIEKDPGAFEEEAMVIEV

SEQ ID NO: 499 - protein, 30 aa, Methanococcus maripaludis
  source 1..30: note = Methanococcus maripaludis C7; note = Adriase recognition motif
EDTAKIAAAIKNCTSQDPDAIDEEPMVVDL

SEQ ID NO: 500 - protein, 30 aa, Methanococcus maripaludis
  source 1..30: note = Secondary Adriase recognition motif; note = Methanococcus maripaludis C7
EDTGKISAAVKSCISKDPGAFDAEPMIIEL

SEQ ID NO: 501 - protein, 30 aa, Methanococcus maripaludis
  source 1..30: note = Methanococcus maripaludis C6; note = Adriase recognition motif
EDTAKIAAAIKNCTSQDPDALDEEPMVVDL

SEQ ID NO: 502 - protein, 30 aa, Methanococcus maripaludis
  source 1..30: note = Methanococcus maripaludis C6; note = Secondary Adriase recognition motif
EDTGKIAAAVKNCISKDPGAFDGEPMIIEL

SEQ ID NO: 503 - protein, 30 aa, Methanococcus maripaludis
  source 1..30: note = Adriase recognition motif; note = Methanococcus maripaludis S2
EDTGKISDAIKNCISKDPGAFEEEPMVIEL

SEQ ID NO: 504 - protein, 30 aa, Methanococcus aeolicus
  source 1..30: note = Secondary Adriase recognition motif; note = Methanococcus aeolicus Nankai-3
EDTGKISSVIKDCISKDPGAFEEDPMILDL

SEQ ID NO: 505 - protein, 30 aa, Methanocaldococcus sp.
  source 1..30: note = Methanocaldococcus bathoardescens; note = Adriase recognition motif
EDTGKITQAIKECISKDPGAIDEDPMVIDL

SEQ ID NO: 506 - protein, 30 aa, Methanotorris formicicus
  source 1..30: note = Methanotorris formicicus Mc-S-70; note = Adriase recognition motif
EDTGKITSAIKECISKDPGAIDEESMVLEL

SEQ ID NO: 507 - protein, 30 aa, Methanotorris formicicus
  source 1..30: note = Methanotorris formicicus Mc-S-70; note = Secondary Adriase recognition motif
EDTGKITSAIKECISKDPGAIDEEPMVLEL

SEQ ID NO: 508 - protein, 30 aa, unidentified
  source 1..30: note = Adriase recognition motif; note = Methanosaeta harundinacea; note = Methanosaeta harundinacea 6Ac
EDTGTIVSKIKECVSKDPGALDVEPMAVEI

SEQ ID NO: 509 - protein, 30 aa, unidentified
  source 1..30: note = Methanosaeta sp.; note = Adriase recognition motif; note = Methanosaeta sp. PtaB.Bin087
EDTGTIVSRIKECISKDPGALDAEPMTIVI

SEQ ID NO: 510 - protein, 30 aa, Methanobrevibacter sp.
  source 1..30: note = Methanobrevibacter boviskoreani / Methanobrevibacter sp. AbM4 Adriase recognition motif
EDVGAITAKVKECIEKDPGAIEEEAMVIDV

SEQ ID NO: 511 - protein, 312 aa, Methanocaldococcus jannaschii
  source 1..312: note = N-terminally modified M. jannaschii Adriase delta 1
GSSHHHHHHSSGLVPRGSHMSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 512 - protein, 304 aa, Methanocaldococcus jannaschii
  source 1..304: note = N-terminally modified M. jannaschii Adriase delta 8
HSSGLVPRGSHMSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 513 - protein, 302 aa, Methanocaldococcus jannaschii
  source 1..302: note = N-terminally modified M. jannaschii Adriase delta10
SGLVPRGSHMSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 514 - protein, 301 aa, Methanocaldococcus jannaschii
  source 1..301: note = N-terminally modified M. jannaschii Adriase delta 11
GLVPRGSHMSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 515 - protein, 292 aa, Methanocaldococcus jannaschii; identical to SEQ ID NO: 159
  source 1..292: note = N-terminally modified M. jannaschii Adriase delta21

SEQ ID NO: 516 - DNA, 5422 bp, synthetic construct
  source 1..5422: mol_type = other DNA; note = vectors
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 517 - protein, 154 aa, Methanosarcina mazei
  source 1..154: note = M. mazei MtrA 1-154; note = Methanosarcina mazei Go1
MADKREPAPGWPILKGEYEVGDVKNSVLVITCGSHLPGKPILDAGAACTGSCKTENLGIEKVVAHIISNPNIRYLLVTGSEVKGHITGQSMMSLHANGVKENRIAGALGAIPYVENLNAAAVARFQEQVQVVNLLDTEDMGAITSKVRELASKD

SEQ ID NO: 518 - protein, 181 aa, Methanosarcina mazei
  source 1..181: note = His6- M. mazei MtrA delta 174-245 with N-terminal His-tag; note = Methanosarcina mazei Go1
MHHHHHHGSMVMSMANKREPAPGWPIVSGEYVVGNPESCVGVVTLGSHGLEQACIDAGAAIAGPCHTENLGIEKVVANYISNPNIRFMILCGSEVQGHITGQCFKALWENGIGDGGIIGAKGAIPFLENVNKEAVERFRRQIVEVVDLIDCEDIGKITQAIKECLSKDPGAIDEDPFIIEL

SEQ ID NO: 519 - protein, 102 aa, Methanosarcina mazei
  source 1..102: note = Methanosarcina mazei Go1; note = Adriase NTN with C-terminal His-tag
MEQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHKGSHHHHHH

SEQ ID NO: 520 - protein, 112 aa, synthetic construct
  source 1..112: note = Ub-(5)KDPGA(15) with N-terminal His-tag
MHHHHHHGSMKIKIVPAVGGGSPLELEVAPNATVGAVRTKVCAMKKLPPDTTRLTYKGRALKDTETLESLGVADGDKFVLITRTVGGRELASKDPGAFDADPLVVEISEEGE

SEQ ID NO: 521 - protein, 107 aa, synthetic construct
  source 1..107: note = PGA(15)-Ub with C-terminal His-tag
PGAFDADPLVVEISEEGEMKIKIVPAVGGGSPLELEVAPNATVGAVRTKVCAMKKLPPDTTRLTYKGRALKDTETLESLGVADGDKFVLITRTVGGCGEGSHHHHHH

SEQ ID NO: 522 - protein, 122 aa, synthetic construct
  source 1..122: note = Ub-(5)KDPGA(15) with N-terminal His- and C-terminal Strep-tag
MHHHHHHGSMKIKIVPAVGGGSPLELEVAPNATVGAVRTKVCAMKKLPPDTTRLTYKGRALKDTETLESLGVADGDKFVLITRTVGGRELASKDPGAFDADPLVVEISEEGEGSWSHPQFEK

SEQ ID NO: 523 - protein, 94 aa, synthetic construct
  source 1..94: note = Ub-(5)KD with N-terminal His-tag
MHHHHHHGSMKIKIVPAVGGGSPLELEVAPNATVGAVRTKVCAMKKLPPDTTRLTYKGRALKDTETLESLGVADGDKFVLITRTVGGRELASKD

SEQ ID NO: 524 - protein, 107 aa, synthetic construct
  source 1..107: note = Ub-(5)KDPGA(10) with N-terminal His-tag
MHHHHHHGSMKIKIVPAVGGGSPLELEVAPNATVGAVRTKVCAMKKLPPDTTRLTYKGRALKDTETLESLGVADGDKFVLITRTVGGRELASKDPGAFDADPLVVEI

SEQ ID NO: 525 - protein, 102 aa, synthetic construct
  source 1..102: note = PGA(10)-Ub with C-terminal His-tag
PGAFDADPLVVEIMKIKIVPAVGGGSPLELEVAPNATVGAVRTKVCAMKKLPPDTTRLTYKGRALKDTETLESLGVADGDKFVLITRTVGGCGEGSHHHHHH

SEQ ID NO: 526 - protein, 107 aa, synthetic construct
  source 1..107: note = Ub-(5)KDPGA(10) with N-terminal His-tag [M. jannaschii MtrA derived]
MHHHHHHGSMKIKIVPAVGGGSPLELEVAPNATVGAVRTKVCAMKKLPPDTTRLTYKGRALKDTETLESLGVADGDKFVLITRTVGGKECLSKDPGAIDEDPFIIEL

SEQ ID NO: 527 - protein, 107 aa, synthetic construct
  source 1..107: note = PGA(15) with C-terminal His-tag [M. jannaschii MtrA derived]
PGAIDEDPFIIELEGGKGMKIKIVPAVGGGSPLELEVAPNATVGAVRTKVCAMKKLPPDTTRLTYKGRALKDTETLESLGVADGDKFVLITRTVGGCGEGSHHHHHH

SEQ ID NO: 528 - protein, 18 aa, Methanosarcina mazei
  source 1..18: note = M. mazei PGA(15); note = Methanosarcina mazei Go1
PGAFDADPLVVEISEEGE

SEQ ID NO: 529 - protein, 8 aa, Methanosarcina mazei
  source 1..8: note = M. mazei PGA(5); note = Methanosarcina mazei Go1
PGAFDADP

SEQ ID NO: 530 - protein, 112 aa, synthetic construct
  source 1..112: note = M. mazei MtrA derived PGA(20) motif
PGAFDADPLVVEISEEGEEEEEGMKIKIVPAVGGGSPLELEVAPNATVGAVRTKVCAMKKLPPDTTRLTYKGRALKDTETLESLGVADGDKFVLITRTVGGCGEGSHHHHHH

SEQ ID NO: 531 - protein, 15 aa, Methanosarcina mazei
  source 1..15: note = Methanosarcina mazei Go1; note = M. mazei (0)KDPGA(10)
KDPGAFDADPLVVEI

SEQ ID NO: 532 - protein, 20 aa, Methanosarcina mazei
  source 1..20: note = Methanosarcina mazei Go1; note = M. mazei (5)KDPGA(10)
RELASKDPGAFDADPLVVEI

SEQ ID NO: 533 - protein, 25 aa, Methanosarcina mazei
  source 1..25: note = Methanosarcina mazei Go1; note = M. mazei (10)KDPGA(10)
ITSKVRELASKDPGAFDADPLVVEI

SEQ ID NO: 534 - protein, 25 aa, Methanosarcina mazei
  source 1..25: note = M. mazei (5)KDPGA(15); note = Methanosarcina mazei Go1
RELASKDPGAFDADPLVVEISEEGE

SEQ ID NO: 535 - protein, 156 aa, Staphylococcus aureus
  source 1..156: note = SrtA5* delta 1-59 with C-terminal His-tag
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 536 - protein, 94 aa, synthetic construct
  source 1..94: note = Ub-GGSLPETGGG
MKIKIVPAVGGGSPLELEVAPNATVGAVRTKVCAMKKLPPDTTRLTYKGRALKDTETLESLGVADGDKFVLITRTVGGGGSLPETGGGHHHHHH

SEQ ID NO: 537 - protein, 96 aa, synthetic construct
  source 1..96: note = GGG-Ub
MHHHHHHGSENLYFQGGGMKIKIVPAVGGGSPLELEVAPNATVGAVRTKVCAMKKLPPDTTRLTYKGRALKDTETLESLGVADGDKFVLITRTVGG

SEQ ID NO: 538 - protein, 120 aa, synthetic construct
  source 1..120: note = M. mazei Ub-(5)KDPGA(10) with N-terminal Strep and C-terminal His-tag
MWSHPQFEKGSMKIKIVPAVGGGSPLELEVAPNATVGAVRTKVCAMKKLPPDTTRLTYKGRALKDTETLESLGVADGDKFVLITRTVGGCGERELASKDPGAFDADPLVVEIGSHHHHHH

SEQ ID NO: 539 - protein, 96 aa, synthetic construct
  source 1..96: note = M. mazei PGA(15)-Ub
PGAFDADPLVVEISEEGEMKIKIVPAVGGGSPLELEVAPNATVGAVRTKVCAMKKLPPDTTRLTYKGRALKDTETLESLGVADGDKFVLITRTVGG

SEQ ID NO: 540 - DNA, 47 bp, synthetic construct
  source 1..47: mol_type = other DNA; note = M. jannaschii MtrA delta 174-240 with N-terminal His6-tag rvs Primer
ggtaacctcgagttataactcaattataaatgggtcttcgtcaatag

SEQ ID NO: 541 - DNA, 37 bp, synthetic construct
  source 1..37: mol_type = other DNA; note = M. jannaschii Adriase without NTN, with C-terminal His-tag fwd primer
cgtagtcatatggaacagttgaggaaacaactcatag

SEQ ID NO: 542 - DNA, 83 bp, synthetic construct; identical to SEQ ID NO: 403
  source 1..83: mol_type = other DNA; note = M. jannaschii Adriase without NTN, with C-terminal His-tag rvs primer

SEQ ID NO: 543 - DNA, 63 bp, synthetic construct; identical to SEQ ID NO: 411
  source 1..63: mol_type = other DNA; note = His-Ub fwd Primer

SEQ ID NO: 544 - DNA, 57 bp, synthetic construct
  source 1..57: mol_type = other DNA; note = Ub-His rvs primer
ggtaacctcgagttagtggtggtggtggtggtgagaaccttcgccgcagccgccaac

SEQ ID NO: 545 - DNA, 105 bp, synthetic construct
  source 1..105: mol_type = other DNA; note = M. mazei Ub-(5)KDPGA(15) with N-terminal His-tag rvs Primer
ggtaacctcgagttattcaccttcttcagagatttcaacaaccagcgggtcagcgtcgaaagcacccgggtctttagaagccagttcacggccgccaaccgtacg

SEQ ID NO: 546 - DNA, 90 bp, synthetic construct
  source 1..90: mol_type = other DNA; note = M. mazei Ub-(5)KDPGA(10) with N-terminal His-tag rvs Primer
ggtaacctcgagttagatttcaacaaccagcgggtcagcgtcgaaagcacccgggtctttagaagccagttcacggccgccaaccgtacg

SEQ ID NO: 547 - DNA, 51 bp, synthetic construct
  source 1..51: mol_type = other DNA; note = M. mazei Ub-(5)KD with N-terminal His-tag rvs Primer
ggtaacctcgagttagtctttagaagccagttcacggccgccaaccgtacg

SEQ ID NO: 548 - DNA, 76 bp, synthetic construct
  source 1..76: mol_type = other DNA; note = M. mazei PGA(10)-Ub with C-terminal His-tag fwd Primer
ggatcgcatatgccgggtgctttcgacgctgacccgctggttgttgaaatcatgaaaattaaaattgtacccgccg

SEQ ID NO: 549 - DNA, 91 bp, synthetic construct
  source 1..91: mol_type = other DNA; note = M. mazei PGA(15)-Ub with C-terminal His-tag fwd Primer
ggatcgcatatgccgggtgctttcgacgctgacccgctggttgttgaaatctctgaagaaggtgaaatgaaaattaaaattgtacccgccg

SEQ ID NO: 550 - DNA, 106 bp, synthetic construct
  source 1..106: mol_type = other DNA; note = M. mazei PGA(20)-Ub with C-terminal His-tag fwd Primer
ggatcgcatatgccgggtgctttcgacgctgacccgctggttgttgaaatctctgaagaaggtgaagaagaagaagaaggtatgaaaattaaaattgtacccgccg

SEQ ID NO: 551 - protein, 40 aa, unidentified
  source 1..40: note = (15)KDPGA(20); note = Candidatus Methanoperedens sp. BLZ2; note = Candidatus Methanoperedens sp.
EDENAIIAKIKELAAKDPGAFDGEPMIIQVGEAKEAEELG

SEQ ID NO: 552 - protein, 40 aa, unidentified
  source 1..40: note = (15)KDPGA(20); note = Methanobacteriaceae archaeon 41_258
EDPGQIQAKVKECIEKDPGAFEEEAMVIEVEEEEEEEVGE

SEQ ID NO: 553 - protein, 40 aa, unidentified
  source 1..40: note = (15)KDPGA(20); note = Methanobacteriales archaeon HGW-Methanobacteriales-1
EDAAAIQSKVKECIEKDPGAFEEEAMVIKVEEKGGEEEEG

SEQ ID NO: 554 - protein, 40 aa, unidentified
  source 1..40: note = Methanobacteriales archaeon HGW-Methanobacteriales-2; note = Adriase recognition motif
EDAATIQSKVKECIDKDPGAFEEEAMVIEVEEDGEEEEGE

SEQ ID NO: 555 - protein, 40 aa, Methanobacterium arcticum
  source 1..40: note = (15)KDPGA(20)
EDAGQIQAKVKECIEKDPGAFEEEAMVIKVEEGGAEEEEG

SEQ ID NO: 556 - protein, 40 aa, Methanobacterium congolense
  source 1..40: note = (15)KDPGA(20)
EDPASIQSKVKECIEKDPGAFEEEAMVIEISEEDGEEEEG

SEQ ID NO: 557 - protein, 40 aa, Methanobacterium formicicum
  source 1..40: note = >Methanobacterium formicicum
EDAATIQSKVKECIDKDPGAFEEEAMVIVVEEDGEEEEGE

SEQ ID NO: 558 - protein, 40 aa, Methanobacterium sp.
  source 1..40: note = Methanobacterium lacus; note = >Methanobacterium lacus
EDAAAIQAKVKECIEKDPGAIEEEAMVIEIEEEGEEEEGE

SEQ ID NO: 559 - protein, 40 aa, Methanobacterium sp.
  source 1..40: note = >Methanobacterium paludis
EDPAAISSKIKECIEKDPGAFEEEAMVIEVEEEGEEEEGE

SEQ ID NO: 560 - protein, 40 aa, Methanobacterium sp.
  source 1..40: note = >Methanobacterium sp.
EDPATISAKVKECIEKDPGAFEEEAMVVEVEEGGEEEEEG

SEQ ID NO: 561 - protein, 40 aa, Methanobacterium sp.
  source 1..40: note = >Methanobacterium sp. BAmetb10; note = Methanobacterium sp. BAmetb10
EDAAAIQSKVKECVEKDPGAFEEEAMVIKVEEGGDEEEGE

SEQ ID NO: 562 - protein, 40 aa, Methanobacterium sp.
  source 1..40: note = >Methanobacterium sp. BAmetb5; note = Methanobacterium sp. BAmetb5
EDAATIQSKVKECIDKDPGAFEEEAMVIKVEEGDGEEEEG

SEQ ID NO: 563 - protein, 40 aa, Methanobacterium sp.
  source 1..40: note = Methanobacterium sp. BRmetb2; note = >Methanobacterium sp. BRmetb2
EDAAAIQSKVKECVEKDPGAFEEEAMVIEIEEEGEEDEGE

SEQ ID NO: 564 - protein, 40 aa, Methanobacterium sp.
  source 1..40: note = Methanobacterium sp. MB1; note = >Methanobacterium sp. MB1
EDAATIQSKVKECIDKDPGAFEEEAMVIKVEEGEGDEEEG

SEQ ID NO: 565 - protein, 40 aa, Methanobacterium sp.
  source 1..40: note = Methanobacterium sp. PtaB.Bin024; note = >Methanobacterium sp. PtaB.Bin024
EDAATIQSKVKECIDKDPGAFEEEAMVIEVEEGDGEEEEG

SEQ ID NO: 566 - protein, 40 aa, Methanobacterium sp.
  source 1..40: note = >Methanobacterium sp. PtaU1.Bin097; note = Methanobacterium sp. PtaU1.Bin097
EDAAAIQSKIKECIEKDPGAFEEEAMVVKVEEGGEEEEEG

SEQ ID NO: 567 - protein, 40 aa, Methanobacterium sp.
  source 1..40: note = >Methanobacterium sp. SMA-27; note = Methanobacterium sp. SMA-27
EDAAAIQAKVKECIEKDPGAIEEEAMVIVIEEEGEEEEGE

SEQ ID NO: 568 - protein, 40 aa, Methanobrevibacter sp.
  source 1..40: note = Methanobrevibacter arboriphilus; note = >Methanobrevibacter arboriphilus
EDAGAIEAKVKECIEKDPGAFEEEAYVISLDDSGDEEDEG

SEQ ID NO: 569 - protein, 40 aa, Methanobrevibacter sp.
  source 1..40: note = Methanobrevibacter arboriphilus; note = >Methanobrevibacter arboriphilus DSM 1125; note = Methanobrevibacter arboriphilus DSM 1125
EDAGAIEAKVKECIEKDPGAFEEDAYVISLDDSGDEEDEG

SEQ ID NO: 570 - protein, 40 aa, Methanobrevibacter sp.
  source 1..40: note = >Methanobrevibacter boviskoreani /Methanobrevibacter sp. AbM4; note = Methanobrevibacter boviskoreani
EDVGAITAKVKECIEKDPGAIEEEAMVIDVSEGGEAEEDE

SEQ ID NO: 571 - protein, 40 aa, Methanobrevibacter curvatus
  source 1..40: note = >Methanobrevibacter curvatus
EDAGAIESKVKECISKDPGAFEEEALVISVDDAGGDDDDG

SEQ ID NO: 572 - protein, 40 aa, Methanobrevibacter filiformis
  source 1..40: note = >Methanobrevibacter filiformis
EDAAAIESKVKECIEKDPGAFEEEALVISVDDDGGDDDDD

SEQ ID NO: 573 - protein, 40 aa, Methanobrevibacter gottschalkii
  source 1..40: note = >Methanobrevibacter gottschalkii
EDIGNIQSKINECIEKDPGALEEDPIVMEVDEENQKLVIV

SEQ ID NO: 574 - protein, 40 aa, Methanobrevibacter millerae
  source 1..40: note = >Methanobrevibacter millerae
EDIGTIQSKINECVEKDPGAFEEEAMVISVDDDDGDEDEG

SEQ ID NO: 575 - protein, 40 aa, Methanobrevibacter olleyae
  source 1..40: note = >Methanobrevibacter olleyae
EDGGAITSKVKECIEKDPGAFEEDAMVIEVSEGDDDEDDG

SEQ ID NO: 576 - protein, 40 aa, Methanobrevibacter oralis
  source 1..40: note = >Methanobrevibacter oralis
EDIGTIQSKINECVEKDPGAFEEEAMVISVEGDDEEADSG

SEQ ID NO: 577 - protein, 40 aa, Methanobrevibacter ruminantium
  source 1..40: note = >Methanobrevibacter ruminantium
EDGGAITAKVKECIEKDPGAFEEDAMVIEVKEGDDDEDEG

SEQ ID NO: 578 - protein, 40 aa, Methanobrevibacter smithii
  source 1..40: note = >Methanobrevibacter smithii
EDVGAIQAKINECVEKDPGAFEEEAMVISVEGDDGEEDDG

SEQ ID NO: 579 - protein, 40 aa, Methanobrevibacter sp.
  source 1..40: note = Methanobrevibacter sp. 87.7; note = >Methanobrevibacter sp. 87.7
EDGGAITSKVKECIEKDPGAFEEEALVVEVSDDDDEEDSG

SEQ ID NO: 580 - protein, 40 aa, Methanobrevibacter sp.
  source 1..40: note = >Methanobrevibacter sp. NOE; note = Methanobrevibacter sp. NOE
EDAGAIEAKVKECIEKDPGAFEEEAYVISLDENGEEEDDG

SEQ ID NO: 581 - protein, 40 aa, Methanobrevibacter sp.
  source 1..40: note = >Methanobrevibacter sp. YE315; note = Methanobrevibacter sp. YE315
EDIGAIQSKINECVEKDPGAFEEEAMVISVDDDGGDEEEG

SEQ ID NO: 582 - protein, 40 aa, Methanobrevibacter thaueri
  source 1..40: note = >Methanobrevibacter thaueri
EDIGAIQSKINECVEKDPGAFEEEAMVISVDDDGGEEEEG

SEQ ID NO: 583 - protein, 40 aa, Methanobrevibacter woesei
  source 1..40: note = >Methanobrevibacter woesei
EDVGAINAKISECVEKDPGAFEEEAMVISVEDDDGDADDG

SEQ ID NO: 584 - protein, 40 aa, Methanobrevibacter wolinii
  source 1..40: note = >Methanobrevibacter wolinii
EDGGAITSKVKECIEKDPGAFEEEALVVEVSDDDEEEDSG

SEQ ID NO: 585 - protein, 40 aa, Methanocaldococcus sp.
  source 1..40: note = Methanocaldococcus bathoardescens; note = >Methanocaldococcus bathoardescens
EDTGKITQAIKECISKDPGAIDEDPMVIDLEGGGAKEGGE

SEQ ID NO: 586 - protein, 40 aa, Methanocaldococcus fervens
  source 1..40: note = >Methanocaldococcus fervens AG86
EDVNKITQAIKECISKDPGAIDEEPIVLELESGGEGTEEE

SEQ ID NO: 587 - protein, 40 aa, Methanocaldococcus infernus
  source 1..40: note = Methanocaldococcus infernus ME; note = >Methanocaldococcus infernus ME
EDIGKITQAIKECLSKDPGAIDEDPYIIQLEGGGGEEEGK

SEQ ID NO: 588 - protein, 40 aa, Methanocaldococcus jannaschii
  source 1..40: note = >Methanocaldococcus jannaschii DSM 2661; note = Methanocaldococcus jannaschii DSM 2661
EDIGKITQAIKECLSKDPGAIDEDPFIIELEGGKGGGEEE

SEQ ID NO: 589 - protein, 40 aa, Methanocaldococcus sp.
  source 1..40: note = >Methanocaldococcus sp. FS406-22; note = Methanocaldococcus sp. FS406-22
EDVGKITQAIKECLSKDPGAIDEDPFIIELEGGKGGGEEE

SEQ ID NO: 590 - protein, 40 aa, Methanocaldococcus villosus
  source 1..40: note = Methanocaldococcus villosus KIN24-T80; note = >Methanocaldococcus villosus KIN24-T80
EDINKITQAIKECISKDPGAIDEEPMILELEGGAKGEEGE

SEQ ID NO: 591 - protein, 40 aa, Methanocaldococcus vulcanius
  source 1..40: note = Methanocaldococcus vulcanius M7; note = >Methanocaldococcus vulcanius M7
EDVGKITQAIKECLSKDPGAIDEDPFIIQLEGGKGGEEEE

SEQ ID NO: 592 - protein, 40 aa, unidentified
  source 1..40: note = >Methanococcales archaeon HHB / Methanofervidicoccus abyssi HHB
EDIGKITQAIKECISKDPGATEEDAEEIDLDKKESGLPTV

SEQ ID NO: 593 - protein, 40 aa, Methanococcus aeolicus
  source 1..40: note = Methanococcus aeolicus Nankai-3; note = >Methanococcus aeolicus Nankai-3
EDTGKISSAIKDCISKDPGAFEEDPMILDLEGGGGEAGAD

SEQ ID NO: 594 - protein, 40 aa, Methanococcus maripaludis
  source 1..40: note = >Methanococcus maripaludis
EDTGKISDAIKNCISKDPGAFEEDPMVIELEGGAAAAGEE

SEQ ID NO: 595 - protein, 40 aa, Methanococcus maripaludis
  source 1..40: note = Methanococcus maripaludis C5; note = >Methanococcus maripaludis C5
EDKGKIADAIKNCISKDPGAFEEDPMVIELEGGAAAAGDE

SEQ ID NO: 596 - protein, 40 aa, Methanococcus maripaludis
  source 1..40: note = >Methanococcus maripaludis C6; note = Methanococcus maripaludis C6
EDTAKIAAAIKNCTSQDPDALDEEPMVVDLEGGEAVAGTE

SEQ ID NO: 597 - protein, 40 aa, Methanococcus maripaludis
  source 1..40: note = Methanococcus maripaludis C7; note = >Methanococcus maripaludis C7
EDTAKIAAAIKNCTSQDPDAIDEEPMVVDLEGGEAVANTE

SEQ ID NO: 598 - protein, 40 aa, Methanococcus maripaludis
  source 1..40: note = >Methanococcus maripaludis S2; note = Methanococcus maripaludis S2
EDTGKISDAIKNCISKDPGAFEEEPMVIELEGGAAAAGEE

SEQ ID NO: 599 - protein, 40 aa, Methanococcus vannielii
  source 1..40: note = Methanococcus vannielii SB; note = >Methanococcus vannielii SB
EDKGKITEAIKNCISKDPGALEEDAMVVELEGGKKVAGED

SEQ ID NO: 600 - protein, 40 aa, Methanococcus voltae
  source 1..40: note = Methanococcus voltae A3; note = >Methanococcus voltae A3
EDKGKIQQTVKDCISKDPGAFEEDAMVIDLEGKAGGAGEE

SEQ ID NO: 601 - protein, 40 aa, Methanoculleus bourgensis
  source 1..40: note = >Methanoculleus bourgensis
EDMGTIKAKIDELKAKDPGAFPAEPMIVEVKEAGGAGAEE

SEQ ID NO: 602 - protein, 38 aa, Methanoculleus chikugoensis
  source 1..38: note = >Methanoculleus chikugoensis
MGAIKAKIDELKAKDPGAFGAEPMIVEVKEAAGAGAEE

SEQ ID NO: 603 - protein, 40 aa, Methanoculleus sp.
  source 1..40: note = Methanoculleus horonobensis; note = >Methanoculleus horonobensis
EDMGAIKAKMDELKAKDPGAFGAEPMIVEVKEAGGAAEEA

SEQ ID NO: 604 - protein, 40 aa, Methanoculleus marisnigri
  source 1..40: note = Methanoculleus marisnigri JR1; note = >Methanoculleus marisnigri JR1
EDMGAIKAKIDELKARDPGAFGAEPMIVEVKEAGGAAEEA

SEQ ID NO: 605 - protein, 40 aa, Methanoculleus sp.
  source 1..40: note = Methanoculleus sediminis; note = >Methanoculleus sediminis / Methanoculleus sp. MH98A
EDMGAIKAKIDELKARDPGAFGAEPMIVEVKEAGGAGAEE

SEQ ID NO: 606 - protein, 40 aa, Methanoculleus sp.
  source 1..40: note = Methanoculleus sp. MAB1; note = >Methanoculleus sp. MAB1
EDMGTIKAKIDELKAKDPGAFPEEPMVVEVKEAGGGAAEV

SEQ ID NO: 607 - protein, 40 aa, Methanoculleus sp.
  source 1..40: note = Methanoculleus sp. SDB; note = >Methanoculleus sp. SDB
EDMGAISAKISELVGKDPGAFDADPMVVEVKEEGAGGGAA

SEQ ID NO: 608 - protein, 40 aa, Methanoculleus sp.
  source 1..40: note = Methanoculleus taiwanensis; note = >Methanoculleus taiwanensis
EDMGAISAKIKELTARDPGAFGADPMIIEVKEAGGAEEEV

SEQ ID NO: 609 - protein, 40 aa, Methanoculleus thermophilus
  source 1..40: note = >Methanoculleus thermophilus
EDMGTIKAKINELKARDPGAFGAEPMVVEVKEAGGEGAEE

SEQ ID NO: 610 - protein, 40 aa, Methanofollis ethanolicus
  source 1..40: note = >Methanofollis ethanolicus
EDLGAIKAKIAELKGKDPGAFGEAPIVVEVKEEGGGAEEE

SEQ ID NO: 611 - protein, 40 aa, Methanolinea sp.
  source 1..40: note = Methanolinea sp. SDB; note = >Methanolinea sp. SDB
EDMGAIKAKIDELKGKDPGAFAADPMVVEVKEAEGGIEVA

SEQ ID NO: 612 - protein, 40 aa, Methanolinea tarda
  source 1..40: note = >Methanolinea tarda
EDLGAIKAKIAELKGRDPGAFAADPMVVEVKEAAGGGAEE

SEQ ID NO: 613 - protein, 40 aa, Methanopyrus kandleri
  source 1..40: note = Methanopyrus kandleri AV19; note = >Methanopyrus kandleri AV19 / Methanopyrus sp. KOL6
EDVDEIVKAIEECVEKDPGAYEEGPMTISLEEEEEEELAE

SEQ ID NO: 614 - protein, 40 aa, unidentified
  source 1..40: note = >Methanoregulaceae archaeon PtaU1.Bin059; note = Methanoregulaceae archaeon PtaU1.Bin059
EDLGAIKAKINELKGRDPGAFAADPMVVEVKEAAGGVEET

SEQ ID NO: 615 - protein, 40 aa, unidentified
  source 1..40: note = >Methanosaeta harundinacea; note = Methanosaeta harundinacea
EDTGTIVSKIKECISKDPGALDVEPMTVEISEGGAEEEAH

SEQ ID NO: 616 - protein, 40 aa, unidentified
  source 1..40: note = Methanosaeta sp.; note = >Methanosaeta sp. PtaB.Bin087; note = Methanosaeta sp. PtaB.Bin087
EDTGTIVSRIKECISKDPGALDAEPMTIVISEGAAEEEAH

SEQ ID NO: 617 - protein, 40 aa, unidentified
  source 1..40: note = Methanosaeta sp. PtaU1.Bin112; note = >Methanosaeta sp. PtaU1.Bin112; note = Methanosaeta sp.
EDMGAITGKIKELVSKDPGALDVDPMTIEIKEGGEEESAG

SEQ ID NO: 618 - protein, 40 aa, Methanosarcina acetivorans
  source 1..40: note = >Methanosarcina acetivorans C2A; note = Methanosarcina acetivorans C2A
EDMGAITAKVRELVAKDPGAFDAEPMVVEISEEGEEEEEG

SEQ ID NO: 619 - protein, 40 aa, Methanosarcina barkeri
  source 1..40: note = >Methanosarcina barkeri CM1 / Methanosarcina barkeri str. Fusaro; note = Methanosarcina barkeri CM1
EDMGAITSKVKELASKDPGAFDAEPMIVEISEEGEEEEEG

SEQ ID NO: 620 - protein, 40 aa, Methanosarcina barkeri
  source 1..40: note = >Methanosarcina barkeri 3; note = Methanosarcina barkeri 3
EDMGAITSKVRELASKDPGAFEAEPLVVEISEEGGEEEEG

SEQ ID NO: 621 - protein, 40 aa, Methanosarcina sp.
  source 1..40: note = >Methanosarcina flavescens
EDMGAITAKVRELASKDPGAFDAEPMIVEISEEGEEEEEG

SEQ ID NO: 622 - protein, 40 aa, Methanosarcina horonobensis
  source 1..40: note = >Methanosarcina horonobensis
EDMGAITAKVRELVAKDPGAFDADPLVVEISEEGEEEEEG

SEQ ID NO: 623 - protein, 40 aa, Methanosarcina lacustris
  REGION 1..40: note = >Methanosarcina lacustris Z-7289
  source 1..40: note = Methanosarcina lacustris Z-7289; note = >Methanosarcina lacustris Z-7289
EDMGAITAKVRELVSKDPGAFDAEPMVVVISDEGEEEEDA

SEQ ID NO: 624 - protein, 40 aa, Methanosarcina siciliae
  source 1..40: note = Methanosarcina siciliae C2; note = >Methanosarcina mazei / Methanosarcina siciliae C2
EDMGAITSKVRELASKDPGAFDADPLVVEISEEGEEEEEG

SEQ ID NO: 625 - protein, 40 aa, Methanosarcina siciliae
  source 1..40: note = >Methanosarcina siciliae
EDMGAITAKVRELVSKDPGAFDAEPLVVEISEEGEEEEEG

SEQ ID NO: 626 - protein, 40 aa, Methanosarcina sp.
  source 1..40: note = >Methanosarcina sp. 1.H.A.2.2 / Methanosarcina sp. 2.H.A.1B.4; note = Methanosarcina sp. 1.H.A.2.2
EDMGAITAKVRELASKDPGAFDAEPMVVVISDEGEEEEEA

SEQ ID NO: 627 - protein, 40 aa, Methanosarcina sp.
  source 1..40: note = >Methanosarcina sp. 1.H.T.1A.1 /Methanosarcina sp. 2.H.T.1A.3; note = Methanosarcina sp. 1.H.T.1A.1
EDMGAITAKVRELVSKDPGAFDAEPMVVEISEEGEEEEEG

SEQ ID NO: 628 - protein, 40 aa, Methanosarcina sp.
  source 1..40: note = Methanosarcina sp. Ant1; note = >Methanosarcina sp. Ant1
EDMGAITSKIRELVSKDPGAFDADPLILQIAAGEEEEEVS

SEQ ID NO: 629 - protein, 40 aa, Methanosarcina sp.
  source 1..40: note = >Methanosarcina spelaei /Methanosarcina vacuolata /Methanosarcina sp. Kolksee; note = Methanosarcina spelaei
EDMGAITSKVRELASKDPGAFDAEPLVVEISEEGGEEEEG

SEQ ID NO: 630 - protein, 40 aa, Methanosarcina sp.
  source 1..40: note = Methanosarcina sp. MSH10X1; note = >Methanosarcina sp. MSH10X1
EDMGAITSKVRELASKDPGAFDADPLVVEISEEGGEEEEG

SEQ ID NO: 631 - protein, 40 aa, Methanosarcina sp.
  source 1..40: note = Methanosarcina sp. MTP4; note = >Methanosarcina sp. MTP4
EDMGVITAKVKELVSKDPGAFDAEPLVVEISEEGEGEEDV

SEQ ID NO: 632 - protein, 40 aa, Methanosarcina sp.
  source 1..40: note = Methanosarcina sp. WWM596; note = >Methanosarcina sp. WWM596
EDMGAITAKVRELVSKDPGAFDAEPMVVVISEEGEEEEEG

SEQ ID NO: 633 - protein, 40 aa, Methanosarcina thermophila
  source 1..40: note = >Methanosarcina thermophila
EDMGVITAKVRELASKDPGAFDAEPMIVEISEEGEGEEEE

SEQ ID NO: 634 - protein, 40 aa, Methanothermobacter defluvii
  source 1..40: note = >Methanothermobacter defluvii / Methanothermobacter sp. CaT2 / Methanothermobacter sp. EMTCatA1 / Methanothermobacter thermautotrophicus
EDADAIKAKVKECIEKDPGAFEEEAMVIKVEEGGEEEEGE

SEQ ID NO: 635 - protein, 40 aa, Methanothermobacter marburgensis
  source 1..40: note = >Methanothermobacter marburgensis /Methanothermobacter wolfeii
EDADAIKAKVKECIEKDPGAFEEEAMIIKVEEGGEEEEGE

SEQ ID NO: 636 - protein, 40 aa, Methanothermobacter sp.
  source 1..40: note = >Methanothermobacter sp. MT-2; note = Methanothermobacter sp. MT-2
EDPGPIQSKVKECIEKDPGAFEEEAMVIEVEEEGEEEVGE

SEQ ID NO: 637 - protein, 40 aa, Methanothermobacter sp.
  source 1..40: note = Methanothermobacter tenebrarum; note = >Methanothermobacter tenebrarum
EDPEQIKAKVKECIEKDPGAFEEEAMVIEVEEEAEEEVGE

SEQ ID NO: 638 - protein, 30 aa, Methanothermus fervidus
  source 1..30: note = >Methanothermus fervidus DSM 2088; note = Methanothermus fervidus DSM 2088
EDADQIKEKVKECIEKDPGAFEEEEVILRL

SEQ ID NO: 639 - protein, 40 aa, Methanothrix soehngenii
  source 1..40: note = >Methanothrix soehngenii
EDIGAIAGKVKELASKDPGALDVEPMAIEIKEGGEEEEAG

SEQ ID NO: 640 - protein, 40 aa, Methanothrix thermoacetophila
  source 1..40: note = Methanothrix thermoacetophila PT; note = >Methanothrix thermoacetophila PT
EDMGAITAKIKELVAKDPGALDVEPMIVEIKEGAGEEGEA

SEQ ID NO: 641 - protein, 40 aa, Methanotorris formicicus
  source 1..40: note = Methanotorris formicicus Mc-S-70; note = >Methanotorris formicicus Mc-S-70
EDTGKITSAIKECISKDPGAIDEESMVLELEGGGTAGEEG

SEQ ID NO: 642 - protein, 40 aa, Methanotorris igneus
  source 1..40: note = >Methanotorris igneus Kol 5; note = Methanotorris igneus Kol 5
EDVSKITAAIKECISKDPGAIDEEPMVLDLEGGGAGGEEE

SEQ ID NO: 643 - protein, 40 aa, Methanobacterium formicicum
  source 1..40: note = >Methanobacterium formicicum DSM 3637; note = Methanobacterium formicicum DSM 3637
EDAATIQSKVKECIDKDPGAFEEEAMVIKVEEGDGDEEEG

SEQ ID NO: 644 - protein, 40 aa, Methanothermococcus okinawensis
  source 1..40: note = >Methanothermococcus okinawensis IH1; note = Methanothermococcus okinawensis IH1
EDTGKISSAVKECISKDPGAIDEDPFIIELEGGAGGEEEE

SEQ ID NO: 645 - protein, 35 aa, Methanobacterium sp.
  source 1..35: note = Methanobacterium sp. PtaU1.Bin097 secondary (15)KDPGA(20); note = Methanobacterium sp. PtaU1.Bin097
EDRSEITAQIQGCIEKDPGAVEEETVVIKLPEKKG

SEQ ID NO: 646 - protein, 35 aa, Methanobacterium sp.
  source 1..35: note = Methanobacterium sp. Maddingley MBC34 secondary (15)KDPGA(20); note = Methanobacterium sp. Maddingley MBC34
EDAAAIQSKVKDCIEKDPGAVESETMIIKLNEDNN

SEQ ID NO: 647 - protein, 35 aa, Methanobacterium sp.
  source 1..35: note = Methanobacterium sp. PtaB.Bin024; note = Methanobacterium sp. PtaB.Bin024 secondary (15)KDPGA(20)
EDAAAIQSQVKECIEKDPGAAEEETMVIEIEEEGE

SEQ ID NO: 648 - protein, 35 aa, Methanobacterium subterraneum
  source 1..35: note = Methanobacterium subterraneum secondary (15)KDPGA(20)
EDATTIQSKVKECIEKDPGAVESETMVIELEEEGS

SEQ ID NO: 649 - protein, 35 aa, Methanobacterium bryantii
  source 1..35: note = Methanobacterium bryantii secondary (15)KDPGA(20)
EDAGQIQGKVKECIEKDPGAYEEEPIAIRLDELEK

SEQ ID NO: 650 - protein, 35 aa, Methanobrevibacter wolinii
  source 1..35: note = Methanobrevibacter wolinii secondary (15)KDPGA(20)
EDGGAITSKVKECIEKDPGAFEEKSMVISIDEDSE

SEQ ID NO: 651 - protein, 35 aa, Methanobrevibacter thaueri
  source 1..35: note = Methanobrevibacter thaueri secondary (15)KDPGA(20)
EDIGAIQSKINECVEKDPGALEEDPIVMEVDEENQ

SEQ ID NO: 652 - protein, 35 aa, Methanobrevibacter sp.
  source 1..35: note = Methanobrevibacter sp. YE315 secondary (15)KDPGA(20); note = Methanobrevibacter sp. YE315
EDIGAIQSKINECVEKDPGALEEDPIVMEVDEENE

SEQ ID NO: 653 - protein, 35 aa, Methanoculleus sp.
  source 1..35: note = Methanoculleus sp. MH98A; note = Methanoculleus sp. MH98A secondary (15)KDPGA(20)
EDMGVIKAKIDELKARDPGAFGAEPMVVEVKEAGG

SEQ ID NO: 654 - protein, 35 aa, Methanoculleus sp.
  source 1..35: note = Methanoculleus sp. SDB secondary (15)KDPGA(20); note = Methanoculleus sp. SDB
EDMGAISAKISELVGKDPGAFDADPMVVEVKEEGA

SEQ ID NO: 655 - protein, 35 aa, Methanoculleus sp.
  source 1..35: note = Methanoculleus horonobensis; note = Methanoculleus horonobensis secondary (15)KDPGA(20)
EDMGAIKAKMDELKARDPGAFGAEPMIVEVKEAGG

SEQ ID NO: 656 - protein, 35 aa, Methanoculleus sp.
  source 1..35: note = Methanoculleus taiwanensis secondary (15)KDPGA(20); note = Methanoculleus taiwanensis
EDMGAISAKIKELTARDPGAFGAEPMIIEVKEAGG

SEQ ID NO: 657 - protein, 35 aa, Methanoculleus sp.
  source 1..35: note = Methanoculleus sp. MAB1; note = Methanoculleus sp. MAB1 secondary (15)KDPGA(20)
EDMGTIKAKIDELKAKDPGAFPEEPMVVEVKEAGG

SEQ ID NO: 658 - protein, 35 aa, Methanococcus maripaludis
  source 1..35: note = Methanococcus maripaludis C7 secondary (15)KDPGA(20); note = Methanococcus maripaludis C7
EDTGKISAAVKSCISKDPGAFDAEPMIIELDGGGA

SEQ ID NO: 659 - protein, 35 aa, Methanococcus maripaludis
  source 1..35: note = Methanococcus maripaludis C6 secondary (15)KDPGA(20); note = Methanococcus maripaludis C6
EDTGKIAAAVKNCISKDPGAFDGEPMIIELDGGGA

SEQ ID NO: 660 - protein, 35 aa, Methanococcus aeolicus
  source 1..35: note = Methanococcus aeolicus Nankai-3; note = Methanococcus aeolicus Nankai-3 secondary (15)KDPGA(20)
EDTGKISSVIKDCISKDPGAFEEDPMILDLEGGGG

SEQ ID NO: 661 - protein, 35 aa, Methanotorris formicicus
  source 1..35: note = Methanotorris formicicus Mc-S-70; note = Methanotorris formicicus Mc-S-70 secondary (15)KDPGA(20)
EDTGKITSAIKECISKDPGAIDEEPMVLELEGGGA

SEQ ID NO: 662 - protein, 159 aa, Staphylococcus aureus
  source 1..159: note = His6-SrtA 1-59
MRGSHHHHHHGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 663 - protein, 155 aa, Lama glama
  source 1..155: note = PGA(10)-sdAb
MPGAFDADPLVVEIMAEVQLVESGGGLVQAGDSLRLSCTASGRTLGDYGVAWFRQAPGKEREFVSVISRSTIITDYADSVRGRFSISADSAKNTVYLQMNSLKPEDTAVYYCAVIANPVYATSRNSDDYGHWGQGTQVTVSSAAALEGSHHHHHH

SEQ ID NO: 664 - protein, 203 aa, Rhinopithecus roxellana
  source 1..203: note = PGA(10)-CyP
MPGAFDADPLVVEIMGFADAAAEEILVVLAMVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLEGSHHHHHH

SEQ ID NO: 665 - protein, 246 aa, Escherichia coli
  source 1..246: note = PGA(10)-GST
MPGAFDADPLVVEIMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSSGSSHHHHHH

SEQ ID NO: 666 - protein, 81 aa, unidentified
  source 1..81: note = Caldiarchaeum subterraneum; note = GGG-Ub
GGGMKIKIVPAVGGGSPLELEVAPNATVGAVRTKVCAMKKLPPDTTRLTYKGRALKDTETLESLGVADGDKFVLITRTVGG

SEQ ID NO: 667 - protein, 296 aa, Methanocaldococcus jannaschii
  source 1..296: note = N-terminally modified M. jannaschii Adriase after Thrombin-cleavage
GSHMSLIICYYGKNGAVIGGDRRQIFFRGSEENRKILEEKLYSGEIKSEEELYKLAEKLNIKIIIEDDREKVRKISDSVVCGEVRSLGIDAKRRRVYATKGKCAIVDILNDTVTNQTIKEGFGIVVLGNRFLKKKAEEELKRTAKLFPMMPIQQIEDAIKEIFEKLKWHPTVSKEYDIYSVNKYEKNFEEVIKKDIESLFKYREQLRKQLIDFGKVMSIVNKIVKNGEIGVIKDGKLHLYDDYIAIDKIDPNPKVFKVVDVEGNFKDGDIVVIENGDMKIKGTNEKVTTKYIIIHK

SEQ ID NO: 668 - protein, 9 aa, synthetic construct
TLVIAFIGK

SEQ ID NO: 669 - protein, 16 aa, synthetic construct
GGSLPETGGGHHHHHH

SEQ ID NO: 670 - protein, 10 aa, synthetic construct
GGSLPETGGG

SEQ ID NO: 671 - protein, 28 aa, synthetic construct
DPGAFDADPLVVEISEEGEEEEEGGVVR

SEQ ID NO: 672 - protein, 22 aa, synthetic construct
FQEQVQVVNLLDTEDMGAITSK

SEQ ID NO: 673 - protein, 38 aa, synthetic construct
DPGAFDADPLVVEISEEGEEEEEGGVVRPVSGEIAVLR

SEQ ID NO: 674 - protein, 9 aa, synthetic construct
MMDIGNLNK

SEQ ID NO: 675 - protein, 15 aa, synthetic construct
ELASKDTLVIAFIGK

SEQ ID NO: 676 - protein, 10 aa, synthetic construct
NGAVMAGDMR

SEQ ID NO: 677 - protein, 5 aa, synthetic construct
  VARIANT 1: note = X can be K or R
XDPGA

SEQ ID NO: 678 - protein, 6 aa, synthetic construct
HHHHHH

SEQ ID NO: 679 - protein, 7 aa, synthetic construct
  VARIANT 1: note = X can be any amino acid
  VARIANT 7: note = X can be any amino acid
XKDGPAX

SEQ ID NO: 680 - protein, 5 aa, synthetic construct
  VARIANT 3: note = X can be any amino acid
LPXTG

SEQ ID NO: 681 - protein, 7 aa, synthetic construct
  VARIANT 1: note = X can be any amino acid
  VARIANT 7: note = X can be any amino acid
XKDPGAX

SEQ ID NO: 682 - protein, 5 aa, synthetic construct
LPETG